(19) United States
(12) Patent Application Publication
(10) Pub. No.: US 2025/0064988 A1
(43) Pub. Date: Feb. 27, 2025

(54) MRI-AGENT LOADED PLATELET COMPOSITIONS AND METHODS OF PREPARING AND USING THE SAME

(71) Applicant: Cellphire, Inc., Rockville, MD (US)

(72) Inventors: Daniel Allen SHEIK, West Lafayette, IN (US); Keith Andrew MOSKOWITZ, Westfield, IN (US); Benjamin J. KUHN, Norfolk, VA (US); Emanuel Joseph VACCHIANO, Burr Ridge, IL (US)

(73) Assignee: Cellphire, Inc., Rockville, MD (US)

(21) Appl. No.: 18/943,861

(22) Filed: Nov. 11, 2024

Related U.S. Application Data
(63) Continuation-in-part of application No. PCT/US2023/066904, filed on May 11, 2023.
(60) Provisional application No. 63/599,945, filed on Nov. 16, 2023.
(60) Provisional application No. 63/364,621, filed on May 12, 2022.
(60) Provisional application No. 63/365,704, filed on Jun. 1, 2022.

(51) Int. Cl. A61K 49/08; A61K 47/69; A61K 49/10

(52) U.S. Cl. CPC A61K 49/085; A61K 47/6901; A61K 49/108

(57) ABSTRACT

Provided herein are imaging agent-loaded cryopreserved platelets and platelet derivatives, such as MRI agent-loaded platelets and platelet derivatives, and methods of preparing and using the same. In some embodiments, methods of loading MRI agents into platelets include contacting platelets with an MRI agent and a cell penetrating peptide. In some embodiments, methods of preparing MRI agent-loaded cryopreserved platelets or platelet derivatives in a dried powder, wherein the MRI agent-loaded cryopreserved platelets or the MRI agent-loaded platelet derivatives comprise an MRI agent complex covalently bonded to the surface of the cryopreserved platelets or the platelet derivatives.

### CROSS-REFERENCE TO RELATED APPLICATIONS

This application claims priority to U.S. Provisional Application Ser. No. 63/599,945, filed on Nov. 16, 2023, and International Application No. PCT/US2023/066904, filed on May 11, 2023. International Application No. PCT/US2023/066904 claims priority to U.S. Provisional Application Ser. No. 63/364,621, filed on May 12, 2022, and U.S. Provisional Application Ser. No. 63/365,704, filed on Jun. 1, 2022. Each of the aforementioned applications is incorporated herein by reference in its entirety.

### SEQUENCE LISTING

This application hereby incorporates by reference the material of the electronic Sequencing Listing filed concurrently herewith. The materials in the electronic Sequence Listing is submitted as an .xml file entitled “CLPH.182.US.02NSCIP_Sequence_Listing_2024_11_08.xml” created on 11/08/2024, which has a file size of 108 KB, and is herein incorporated by reference in its entirety.

### TECHNICAL FIELD

Provided herein are compositions and methods for use of platelets, platelet derivatives, or thrombosomes (e.g., freeze-dried platelet derivatives) as biological carriers of cargo, such as MRI agents, also referred to herein as MRI agent-loaded platelets, platelet derivatives, or thrombosomes. Also provided herein are methods of preparing platelets, platelet derivatives, or thrombosomes loaded with the MRI agent of interest.

MRI agent-loaded platelets described herein can be stored under typical ambient conditions, refrigerated, cryopreserved, for example with dimethyl sulfoxide (DMSO), and/or lyophilized after stabilization (e.g., to form thrombosomes)

### BACKGROUND

Alzheimer's disease (AD) is the most prevalent form of age-related dementia in the world, and its main pathological features consist of amyloid-β (amyloid beta; Aβ) plaque deposits and neurofibrillary tangles formed by hyperphosphorylated tau protein. Recently, disease-modifying therapies (DMTs) that can change the underlying pathophysiology of AD, with anti-Aβ monoclonal antibodies (mabs) (e.g., donanemab, aducanumab, bapineuzumab, gantenerumab, solanezumab, and lecanemab) have been developed successively and conducted in clinical trials based on the theory that a systemic failure of cell-mediated Aβ clearance contributes to AD occurrence and progression (See e.g., Shi M, et al. Impact of Anti-amyloid-β Monoclonal Antibodies on the Pathology and Clinical Profile of Alzheimer's Disease: A Focus on Aducanumab and Lecanemab. Front Aging Neurosci. 2022 Apr. 12; 14:870517).

These anti-Aβ monoclonal antibodies (mabs) as well as small molecules that target Aβ as well as other therapeutics are emerging disease-modifying therapies for Alzheimer disease that require brain magnetic resonance imaging (MRI) for eligibility assessment as well as for monitoring for amyloid-related imaging abnormalities (ARIA). Amyloid-related imaging abnormalities result from treatment-related loss of vascular integrity and may occur in 2 forms. Amyloid-related imaging abnormalities with edema or effusion (ARIA-E) are transient, treatment-induced edema or sulcal effusion, identified on T-FLAIR. Amyloid-related imaging abnormalities with hemorrhage (ARIA-H) are treatment-induced microhemorrhages or superficial siderosis identified on T2* gradient recalled-echo MRI. As monoclonal antibodies become more widely available, treatment screening and monitoring brain MRI examinations may greatly increase neuroradiology practice volumes (Cogswell P M, et al. Amyloid-Related Imaging Abnormalities with Emerging Alzheimer Disease Therapeutics: Detection and Reporting Recommendations for Clinical Practice. AJNR Am J Neuroradiol. 2022 September; 43(9):E19-E35 and Roytman, M., et al. “Amyloid-related imaging abnormalities: An update.” American Journal of Roentgenology 220.4 (2023): 562-574).

Although a few anti-Aβ monoclonal antibodies (mabs) are now FDA approved, one with full approval, and others that are nearing approval or in late stage trials, there have been severe and even fatal brain bleeding incidents in subjects receiving these therapies. Thus, there is a need for effective treatment for these brain bleeding incidents and for better diagnostics that can help identify patients who are more susceptible to severe brain bleeding.

Current methods of detecting ARIA-H have numerous limitations: There are both patient-related and acquisition-related interpretation pitfalls or difficulties with ARIA-H and, in particular, microhemorrhage detection. Blurring due to patient motion may impair detection of small microhemorrhages. Areas of prominent air-tissue susceptibility effects may induce punctate artifacts that look similar to microhemorrhages, especially near the frontal sinuses, mastoid air cells, and skull base. Susceptibility-related signal loss from physiologic mineralization in the basal ganglia may be misinterpreted as microhemorrhages and should not be incorporated into the overall microhemorrhage count. Bulk susceptibility effects that produce signal loss may preclude evaluation of the inferior temporal and anterior frontal lobes. Partial volume effects may cause a small microhemorrhage to be poorly seen or to have a variable appearance on serial examinations. Thick-section acquisitions may also make it difficult to distinguish a microhemorrhage from a vessel flow void. Reader biases may also affect interpretation, as readers have been found to undercall possible microhemorrhages in a patient without other microhemorrhages and to overcall in patients with many. Thus, there is a need for improved methods for detecting ARIA-H.

### SUMMARY OF THE INVENTION

To overcome the above mentioned and additional problems in the art, the present disclosure provides in one aspect, a method for or administering rehydrated platelet derivatives, in illustrative embodiments rehydrated freeze-dried platelet derivatives to a mammalian subject, said method comprising: administering an effective dose of a rehydrated composition comprising the rehydrated freeze-dried platelet derivatives to the mammalian subject, wherein the mammalian subject can have one or more of the following properties:|
a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In some embodiments, the mammalian subject has one or more sites of bleeding in their brain, and in some embodiments, the ARIA detected comprises ARIA-H. In some embodiments, the mammalian subject is afflicted with Alzheimer's Disease.

In another aspect, provided herein, is a method for treating brain bleeding in a mammalian subject, said method comprising: administering an effective dose of a composition comprising rehydrated platelet derivatives, in illustrative embodiments rehydrated freeze-dried platelet derivatives to the mammalian subject, wherein the mammalian subject has one or more of the following properties:
a. is being, was, or will be administered a therapeutic agent capable of binding beta amyloid and/or oligomers thereof, and/or plaques thereof; comprises the therapeutic agent capable of binding amyloid beta and/or oligomers thereof, and/or plaques thereof;
b. is afflicted with Alzheimer's Disease;
c. has been diagnosed with Alzheimer's Disease;
d. has been subjected to MRI and amyloid-related imaging abnormalities (ARIA) were detected;
e. and has amyloid beta deposits on their brain.

In some embodiments, the mammalian subject has been subjected to MRI and amyloid-related imaging abnormalities (ARIA) were detected. and in n some embodiments, the ARIA detected comprises ARIA-H.

In another aspect, provided herein is a method for delivering an MRI agent to the brain of a mammalian subject, comprising administering to the mammalian subject, an effective dose of a rehydrated freeze-dried platelet derivative composition comprising MRI agent-loaded platelet derivatives typically having a compromised plasma membrane, wherein typically at least 50% of the MRI agent-loaded platelet derivatives are CD 41-positive platelet derivative, and wherein the mammalian subject has one or more of the following properties:
a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In another aspect, provided herein is a method for delivering an MRI agent to the brain of a mammalian subject, comprising administering to the mammalian subject, an effective dose of a) a platelet composition comprising MRI agent-loaded cryopreserved platelets, and/or b) a platelet derivative composition comprising MRI agent-loaded platelet derivatives, wherein the mammalian subject administered the effective dose of a) and/or b), has one or more of the following properties:
a. a. is being, was, or will be administered a therapeutic agent capable of binding beta amyloid and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In illustrative embodiments, the platelet derivatives or MRI agent-loaded platelet derivatives for any aspect herein have one, two, three, four, five or more properties for platelet derivatives and/or MRI agent-loaded platelet derivatives provided herein. In illustrative embodiments, the platelet derivatives are freeze-dried platelet derivatives.

In some embodiments, the mammalian subject has one or more sites of bleeding in their brain, and in some embodiments, the subject has ARIA-A and/or ARIA-H, in illustrative embodiments, ARIA-H. In illustrative embodiments, the mammalian subject is afflicted with Alzheimer's Disease.

Further details regarding aspects and embodiments of the present disclosure are provided throughout this patent application. Sections and section headers are for ease of reading and are not intended to limit combinations of disclosure, such as methods, compositions, or other functional elements therein across sections.

### DESCRIPTION OF DRAWINGS

FIG. 1 shows pooled apheresis platelets incubated with FITC labeled TAT peptide in loading buffer.

FIGS. 2A-C shows platelets analyzed by flow cytometry for FITC-TAT loading in either HMTA or loading buffer at two concentrations (25 μM or 50 μM) by mean fluorescence intensity (FIG. 2A). FIGS. 2B and 2C show pooled apheresis platelets incubated FITC-labeled TAT peptide in either HMTA or loading buffer at either 50 μM FITC-labeled TAT (FIG. 2B) or 25 μM FITC-labeled TAT (FIG. 2C).

FIG. 3 is a flow cytometry histogram of samples incubated with 100 μM FITC-TAT in the presence of platelet anti-aggregation compounds PGE1, GR144053, and eptifibatide. PGE1 appears to be associated with improved platelet loading little to no effect is observed with GR144053 or eptifibatide on the distribution of FITC-CPP.

FIG. 4 shows the effect of different buffers on FITC-TAT loading into platelets as measured by fluorescence intensity.

FIGS. 5A-C shows brightfield, FITC, and overlaid microscopy images of non-loaded platelets (FIG. 5A), 100 μM fluorescein (FIG. 5B), and 100 μM FITC-labeled TAT (FIG. 5C).

FIG. 6 is a flow cytometry histogram of SAMPLS incubated with either loading buffer (left peak) or a solution of FITC-labeled magnetic nanoparticles (right peak).

FIGS. 7A-D shows Texas Red (FIG. 7A), FITC (FIG. 7B), brightfield (FIG. 7C), and overlaid (FIG. 7D) images of samples incubated with FITC-labeled magnetic nanoparticles.

FIG. 8 is a flow cytometry histogram of samples incubated with either loading buffer (left peak) or a 50 μM FITC-CPP-Gd-DOTA solution (right peak) for 30 minutes.

FIGS. 9A-9B show a schematic (FIG. 9A) and magnetic resonance imaging (FIG. 9B). As shown in FIG. 9A, samples 1A, 1B, and 2B are negative controls, sample 2A includes Gd-DOTA-FITC-CPP with platelets (400K/μL), samples indicated with 100 mM or 100 μM GdCl₃ are positive controls.

FIG. 10 is a graph showing post-cryopreservation occlusion time of platelets loaded with Gd-DOTA-FITC-CPP with plasma only (negative control), pooled, unloaded platelets (positive control), and Gd-DOTA-FITC-CPP loaded platelets.

FIG. 11 shows exemplary flow cytometry data of thrombosomes unstained (dark data points) or stained (light data points) with an anti-CD-41 antibody.

FIG. 12 shows an exemplary histogram of thrombosomes incubated with annexin V with (light data points) and without (dark data points) calcium.

FIG. 13 shows an exemplary histogram of thrombosomes incubated with an anti-CD62 antibody (light data points) or with an isotype control (dark data points).

FIG. 14 shows a plot of thrombin peak height for thrombosomes in the presence of PRP Reagent containing tissue factor and phospholipids (solid line and long dashes) and control cephalin (dots).

FIG. 15A shows the aggregation of 250,000/μL TFF thrombosomes in buffer vs. agonist, demonstrating the aggregation response of thrombosomes in the presence of agonists, but in the absence of fresh platelets.

FIG. 15B shows the aggregation of 250,000 platelets/μL of platelet rich plasma (PRP), demonstrating the aggregation response of platelet-rich plasma (PRP) in the presence of agonists, but in the absence of fresh platelets.

FIG. 15C shows an ADP comparison, demonstrating the comparison of aggregation of thrombosomes and PRP in the presence of 20 μM ADP.

FIG. 15D shows a collagen aggregation comparison, demonstrating a comparison of aggregation of thrombosomes and PRP in the presence of 10 μg/ml collagen.

FIG. 15E shows an epinephrine aggregation comparison, demonstrating the comparison of aggregation of thrombosomes and PRP in the presence of 300 μM epinephrine.

FIG. 15F shows the comparison of aggregation of thrombosomes and PRP in the presence of 1 mg/ml ristocetin.

FIG. 15G shows a Thrombin Receptor Activator Peptide 6 (TRAP-6) aggregation comparison, demonstrating the comparison of aggregation of thrombosomes and PRP in the presence of 10 μM TRAP-6.

FIG. 15H shows an arachidonic acid (AA) aggregation comparison data and graph, showing the comparison of aggregation of thrombosomes and PRP in the presence of 5 mg/ml arachidonic acid.

FIG. 16A is a representative graph of experiments where apheresis platelets were used to confirm TRAP activity, showing that TRAP-6 peptide is capable of promoting platelet activation by observing expression of CD62P on the apheresis platelets.

FIG. 16B shows that TRAP does not increase AV binding to TFF Thrombosomes, demonstrating that TRAP-6 peptide is not able to increase the expression of CD62P on FDPDs.

FIG. 17 shows the measurement of thrombospondin (TSP-1) by flow cytometry in terms of mean fluorescent intensity (MFI) in resting fresh platelets, activated fresh platelets, and different lots of thrombosomes.

FIG. 18 shows the measurement of von Willebrand factor (vWF) by flow cytometry in terms of mean fluorescent intensity (MFI) in resting fresh platelets, activated fresh platelets, and different lots of thrombosomes.

FIG. 19A shows the measurement of vWF by flow cytometry in terms of mean fluorescent intensity (MFI) in fixed lyophilized platelets, and thrombosomes.

FIG. 19B shows the measurement of TSP-1 by flow cytometry in terms of mean fluorescent intensity (MFI) in fixed lyophilized platelets, and thrombosomes.

FIG. 19C shows the measurement of fibrinogen by flow cytometry in terms of mean fluorescent intensity (MFI) in fixed lyophilized platelets, and thrombosomes.

FIG. 20 shows the forward scatter (FSC) measured by flow cytometry of apheresis platelets, and thrombosomes.

FIG. 21 is a comparison of the pre-lyo, CPP, and FDPD platelet count for the six sample group using Beckman Coulter Ac⋅T Diff2 Hematology Analyzer.

FIG. 22 shows the flow cytometry measurements of mean fluorescence intensity (MFI) in the FITC channel for all six samples of FDPDs and CPP. Average values are presented with the unloaded product value subtracted out as background. Error bars are the standard deviation of measurements. NovoCyte Quanteon flow cytometer was used to take measurements.

FIG. 23 is a graph of the Thrombin generation results for all six samples of FDPDs and CPP using the CLARIOstar Plus microplate reader. Thrombin generation potency (TGPU) are equivalent to NIH Units of Thrombin per 1 million particles.

FIG. 24 shows the flow cytometry measurements of forward scatter for all six samples of FDPDs and CPP. Average values are presented. Error bars are the standard deviation of measurements. NovoCyte Quanteon flow cytometer was used to take measurements.

FIGS. 25A and 25B shows the Total Thrombus System (T-TAS®) results for the six samples of cryopreserved platelet (FIG. 25A) and six samples of FDPDs (FIG. 25B).

### Definitions

As used herein, the term “platelets” has its ordinary meaning in the art.

As used herein, “cryopreserved platelets” are frozen platelets that when thawed are in a liquid state regardless of whether any liquid is added to the frozen platelets after thawing. Accordingly, cryopreserved platelets are not fresh platelets and they are not freeze-dried platelet derivatives. During processing cryopreserved platelets are not dried. The term “cryopreserved platelets” does not imply any minimum length of time such platelets are present in a frozen state. However, cryopreserved platelets are typically stable for at least 1, 2, 3, 4, 5, 6, 9, or 12 months, and in illustrative embodiments are stable for at least 18, 24, 36, or 48 hours. Cryopreserved platelets are typically suspended in a cryoprotectant in a frozen state, until thawing before use.

As used herein, “hemostatic properties” include the following properties: (a) the ability to generate thrombin in a thrombin generation assay, for example in the presence of tissue factor and phospholipids; (b) the ability to occlude a collagen-coated microchannel in vitro, for example under conditions in which fresh platelets can occlude a collagen-coated microchannel in vitro; (c) the capability of thrombin-induced trapping in the presence of thrombin. As demonstrated in Examples herein, platelet derivatives, such as a freeze-dried platelet derivatives (e.g., thrombosomes) herein, in illustrative embodiments are hemostats, and thus have one, two, or all of the aforementioned hemostatic properties.

As used herein, the term “MRI agent-loaded platelets” is inclusive of MRI agent-loaded cryopresereved platelets, MRI agent-loaded platelet derivatives, or MRI agent-loaded thrombosomes, unless the context clearly dictates a particular form.

As used herein, “platelet derivatives” are particles that have some characteristics of fresh platelets but are surrounded by a compromised plasma membrane (i.e., lack an integrated membrane around them), and as such include pores that are larger than pores found in living platelets. Thus, in illustrative embodiments, platelet derivatives herein exhibit an increased permeability to IgG antibodies. In illustrative embodiments, platelet derivatives, or aggregates thereof found in platelet compositions, are at least 0.5 μm or between 0.5 μm and 25 μm in diameter as determined by dynamic light scattering. Thus, such subsets of platelet-derivative particles are distinguishable from platelet-derivative microparticles, which have a diameter of less than 0.5 μm.

In illustrative embodiments, platelet derivatives herein have a reduced ability to, or are unable to transduce signals from the external environment into a response inside the particle that are typically transduced in living platelets. However, platelet derivatives herein (e.g., thrombosomes) can retain some metabolic activity, for example, as evidenced by lactate dehydrogenase (LDH) activity and/or esterase activity.

As used herein, “particle size” refers to the diameter of a particle, unless indicated otherwise. In some embodiments of any of the aspects and embodiments herein that include a platelet derivative composition in a powdered form, the size of the particles is determined after rehydrating the platelet derivative composition with an appropriate solution. In some embodiments, the amount of solution for rehydrating a platelet derivative composition is equal to the amount of buffer or preparation agent present at the step of freeze-drying. The particle size distribution and microparticle content of a composition can be measured by any appropriate method, for example, by flow cytometry using sizing standards, or in illustrative embodiments by dynamic light scattering (DLS). As used herein, a content (e.g., ratio or percent) of microparticles in illustrative embodiments refers to the microparticle content based on the scattering intensity of all particles from about 1 nm to about 60,000 nm in radius in the composition. In some cases, the viscosity of a sample used for DLS can be at about 1.060 cP (or adjusted to be so), as this is the approximate viscosity of plasma. It will be understood that the measured size of particles can vary depending on the technology used to perform the measurement. Particle sizes provided herein are typically as determined by DLS unless the context indicates otherwise. For example if a percent surface marker content of a composition that includes particles, is recited as a percent of particles or platelet derivatives within a certain size range, flow cytometry is typically used to measure both biomarker content and particle size. In some embodiments, the platelet derivative composition as per any aspects, or embodiments comprises a population of platelet derivatives greater than 0.5 μm, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, illustrative or target platelet derivatives have a diameter in the range of 0.5-2.5 μm using flow cytometry, or a diameter of 0.5-25 μm using DLS, and microparticles have a diameter less than 0.5 μm by either method.

As used herein, “thrombosomes” (sometimes also herein called “Tsomes” or “Ts”, particularly in the Examples and FIGs.) are platelet derivatives that have been contacted with an incubating agent (e.g., any of the incubating agents described herein) and lyopreserved (e.g., freeze-dried). Thus, thrombosomes are illustrative or target freeze-dried platelet derivatives (FDPDs). Illustrative or target freeze-dried platelet derivative compositions herein (e.g. thrombosomes) typically have at least 1 hemostatic property, and thus can function as hemostatic agents. Therefore, such illustrative or target FDPDs and compositions herein comprising the same, can also be referred to as hemostat(s), hemostatic product(s), freeze-dried platelet derived hemostat(s) (FDPDH or FPDH), freeze-dried platelet hemostat(s) (FDPH or FPH), or dry platelet derivative hemostat(s) (PDH). In some cases, illustrative or target FDPDs such as thrombosomes can be prepared from pooled platelets. Illustrative or target FDPDs such as thrombosomes can have a shelf life of 2-3 years in dry form at ambient temperature and can be rehydrated with sterile water within minutes (e.g. 1, 2, 3, 4, 5, 10 15, 20, 25, or 30 minutes) for immediate infusion. One example of thrombosomes are THROMBOSOMES® freeze-dried platelet derivatives (Cellphire Inc., Rockville, MD), which are in clinical trials for the treatment of acute hemorrhage in thrombocytopenic patients and are a product of Cellphire, Inc. In non-limiting illustrative embodiments, FDPD compositions, or illustrative or target freeze-dried platelet-derivative (i.e. “FDPD”) compositions herein, such as those prepared according to Example 2 herein, are compositions that include a population of platelet derivatives having a reduced propensity to aggregate such that no more than 10% of the platelet derivatives in the population aggregate under aggregation conditions comprising an agonist but no platelets, and in illustrative embodiments, no divalent cations. Furthermore, such illustrative or target platelet derivatives typically have the ability to generate thrombin in an in vitro thrombin generation assay and/or have the ability to occlude a collagen-coated microchannel in vitro.

In illustrative embodiments, illustrative or target platelet derivatives are CD41 positive and/or CD42 positive. Platelets derivatives (e.g., thrombosomes) herein, in some embodiments, are dry platelet derivatives, or dry platelet derived particles. A skilled artisan will understand that most properties of such dry platelet derivatives are analyzed after the platelet derivatives are rehydrated. Dry platelet derivatives are typically present in a dried substance that includes other components (e.g., saccharides such as, for example, trehalose and/or polysucrose) present along with the platelet derivatives when they were dried. In illustrative platelet derivative compositions herein, less than 5% of the particles are microparticles having a diameter of less than 0.5 μm. In illustrative platelet derivative compositions herein, at least 90% of the particles therein are at least 0.5 μm in diameter. Furthermore, in illustrative embodiments, between 75% and 95% of the platelet derivatives or particles therein are CD41 positive, between 75% and 95% of the platelet derivatives or particles therein are CD42 positive, and less than 5% of the CD 41-positive platelet derivatives or particles therein are microparticles having a diameter of less than 0.5 μm. It will be understood that in such percent calculations, particles are only intended to cover those that can be detected for example by the instrument (e.g., flow cytometer) used to detect CD41 or CD42 or any surface marker.

In some examples of such illustrative embodiments, the platelet derivatives have a potency of at least 1.5 thrombin generation potency units (TGPU) per 10⁶ platelet derivatives. In non-limiting illustrative embodiments, FDPD compositions, or illustrative FDPD compositions herein, such as those prepared according to Example 2 herein, are compositions that include illustrative or target platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and typically such compositions have the ability to generate thrombin in an in vitro thrombin generation assay and/or have the ability to occlude a collagen-coated microchannel in vitro. In illustrative embodiments, the platelet derivatives in such compositions have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the illustrative or target platelet derivatives are between 0.5 and 2.5 μm in diameter by flow cytometry or between 0.5 and 25.0 μm in diameter by dynamic light scattering.

As used herein and in the appended claims, the term “fresh platelet” can include day of use platelets.

As used herein and in the appended claims the term “stored platelet” can include platelets stored for approximately 24 hours or longer before use.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”. When multiple low and multiple high values for ranges are given that overlap, a skilled artisan will recognize that a selected range will include a low value that is less than the high value.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference in their entirety to disclose and describe the methods and/or materials in connection with which the publications are cited. The present disclosure is controlling to the extent it conflicts with any incorporated publication.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein.

### DETAILED DESCRIPTION

To overcome the above mentioned and additional problems in the art, the present disclosure provides in one aspect, a method for or administering platelet derivatives, in illustrative embodiments rehydrated platelet derivatives, in illustrative embodiments rehydrated platelet derivatives having any of the properties herein, in illustrative embodiments freeze-dried platelet derivatives, to a mammalian subject, said method comprising: administering an effective dose of a rehydrated composition comprising the rehydrated freeze-dried platelet derivatives to the mammalian subject, wherein the mammalian subject can have one or more of the following properties:|
a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In some embodiments, the mammalian subject has one or more sites of bleeding in their brain, and in some embodiments, the subject has ARIA-A and/or ARIA-H, in illustrative embodiments, ARIA-H. In illustrative embodiments, the mammalian subject is afflicted with Alzheimer's Disease.

In another aspect, provided herein is a method for delivering an MRI agent to the brain of a mammalian subject, comprising administering to the mammalian subject, an effective dose of a platelet derivative composition, a freeze-dried platelet derivative composition, or in illustrative embodiments a rehydrated freeze-dried platelet derivative composition, comprising MRI agent-loaded platelet derivatives typically having a compromised plasma membrane, wherein typically at least 50% of the MRI agent-loaded platelet derivatives are CD 41-positive platelet derivative, and wherein the mammalian subject has one or more of the following properties:
a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In illustrative embodiments, the MRI agent-loaded platelet derivatives have one, two, three, four, five or more properties for platelet derivatives and/or MRI agent-loaded platelet derivatives provided herein. In illustrative embodiments, the platelet derivatives are freeze-dried platelet derivatives. Furthermore, in illustrative embodiments, the subject has Alzheimer's Disease.

In some aspects and embodiments, the present disclosure provides solutions to address problems related to accurate targeting of a site(s) of interest in a subject, especially where such site(s) of interest are or include inflamed, diseased or compromised blood vessels. Imaging agent-loaded, and in illustrative embodiments magnetic resonance imaging (MRI) agent-loaded platelet derivatives and cryopreserved platelets, provided herein allow targeted delivery of the imaging agent (e.g. MRI agent) to sites of interest. Accordingly, imaging agent-loaded, in illustrative embodiments MRI agent-loaded platelets or platelet derivatives, can be used to image blood vessels and inflamed or diseased tissue where blood vessels have become compromised (e.g., “leaky”) or otherwise damaged. Targeted delivery of imaging agent-loaded, in illustrative embodiments, MRI agent-loaded platelets, such as cryopreserved platelets, or platelet derivatives, such as freeze-dried platelet derivatives (FDPDs), which sometimes can be called freeze-dried platelets herein, can be used to enhance diagnosis of a condition in a subject, especially a condition related to inflamed, diseased, or compromised blood vessels, thus facilitating detection and diagnosis, including in some cases, early or earlier detection and/or diagnosis, thus aiding in higher chances of successful treatment. Owing to the properties of platelet derivatives, or cryopreserved platelets that are used as a means to deliver MRI agent/imaging agent to a subject, imaging agent-loaded, in illustrative embodiments MRI agent-loaded platelet derivatives or cryopreserved platelets can be used to treat a subject having a condition/indication/disease as disclosed, wherein the imaging agent (e.g. MRI agent) are useful to detect, analyze in vivo, confirm delivery of, and/or localize the platelet derivatives or cryopreserved platelets.

Accordingly, in one aspect, provided herein is a composition comprising imaging agent-loaded, in illustrative embodiments MRI agent-loaded platelets, such as cryopreserved platelets, and platelet derivatives, such freeze-dried platelet derivatives, wherein the imaging agent-loaded, in illustrative embodiments MRI agent-loaded platelets or platelet derivatives are coupled to a cell penetrating peptide (CPP). Further, the present disclosure provides a composition comprising imaging agent-loaded, and in illustrative embodiments MRI-agent loaded platelets, such as cryopreserved platelets, and platelet derivatives, such as freeze-dried platelet derivatives, wherein the imaging agent, in illustrative embodiments MRI agent are part of a complex that is covalently bonded to the surface of the platelets, such as the cryopreserved platelets, or the platelet derivatives, such as the freeze-dried platelet derivatives, wherein the imaging agent complex or MRI agent complex comprises the imaging agent and/or MRI agent, and a chelator, and a linker in certain embodiments when the MRI agent complex is not covalently attached to a platelet derivative or cryopreserved platelet. In some embodiments, the loading of an imaging agent, in illustrative embodiments MRI agent in the platelets can mitigate systemic side effects associated with the imaging agent (e.g. MRI agent) and can shield the imaging agent (e.g. MRI agent) from natural clearance mechanisms during migration to the site of interest, such as a site of injury. In some embodiments, the accumulation of imaging agent-loaded platelets or platelet derivatives (e.g. MRI agent-loaded platelets or platelet derivatives) at the site of injury can enhance the resolution of images (e.g. magnetic resonance images) and allow for earlier detection and/or improved disease diagnoses. Provided herein are methods of delivering imaging agent-loaded, which in non-limiting illustrative embodiments are MRI agent-loaded platelets, such as cryopreserved platelets, and/or platelet derivatives, such as FDPDs, to image, and/or aid in imaging a site(s) of interest in a subject.

Provided herein is a method of detecting, diagnosing, enhancing detection and/or diagnosis of a disease, wherein the method includes administering an effective amount or a therapeutically effective amount of a composition comprising imaging agent-loaded, in illustrative embodiments, MRI agent-loaded platelets, such as cryopreserved platelets, or platelet derivatives, such as FDPDs of any of the aspects or embodiments disclosed herein, or a composition prepared by a process of any of the aspects or embodiments disclosed herein, and imaging and/or detecting the imaging agent (e.g. MRI agent), thereby detecting or diagnosing, or enhancing detection or and/or diagnosis of, the disease.

Provided herein is a method for detecting a site(s) of bleeding in a subject, comprising: (a) administering an effective amount or a therapeutically effective amount of a composition comprising imaging agent-loaded (e.g. MRI agent-loaded) platelets, such as cryopreserved platelets, or platelet derivatives such as FDPDs, according to any of the aspects or embodiments herein, or prepared by any process disclosed herein, to the subject; and (b) detecting/locating the site of the imaging agent-loaded (e.g. MRI agent-loaded) platelet or platelet derivatives, thereby detecting the site of bleeding in the subject.

Provided herein is a process for preparing imaging agent-loaded (e.g. MRI agent-loaded) platelets, comprising: (a) providing platelets; and (b) treating the platelets with an imaging agent (e.g. an MRI agent), to form the imaging agent-loaded (e.g. the MRI agent-loaded) platelets.

Provided herein is a process for preparing MRI agent/imaging agent-loaded platelet derivatives, comprising: a) providing platelets; (b) treating the platelets with an MRI agent/imaging agent, to form MRI agent/imaging agent-loaded platelets; and (c) lyophilizing the MRI agent/imaging agent-loaded platelets, to form MRI agent/imaging agent-loaded platelet derivatives.

Provided herein is a process for preparing MRI agent/imaging agent-loaded cryopreserved platelets, comprising: a) providing platelets; (b) treating the platelets with an MRI agent/imaging agent, to form MRI agent/imaging agent-loaded platelets; and (c) cryopreserving the MRI agent/imaging agent-loaded platelets, to form MRI agent/imaging agent-loaded cryopreserved platelets.

In some embodiments, a process as provided herein can further comprise a step of rehydrating the MRI agent-loaded platelet derivatives. In some embodiments, rehydrating the MRI agent-loaded platelet derivatives includes adding to dried platelet derivatives, an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension.

In some embodiments, the rehydrated platelets have coagulation factor levels showing all individual factors (e.g., Factors VII, VIII and IX) associated with blood clotting at 40 international units (IU) or greater.

In some embodiments, the dried platelets, such as freeze-dried platelets, have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets, have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the MRI agent-loaded platelets and the dried platelets, such as freeze-dried platelets, having a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.0 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets and/or the dried platelets, such as freeze-dried platelets, have a particle size in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

Methods of Administering Platelet Derivatives to a Subject Afflicted with Alzheimer's Disease

The present invention provides in some aspects, methods for treating brain bleeding in subjects that have abnormal amounts and/or types of beta amyloid in their brain. For example, such subjects can have amyloid beta oligomers, deposits, and plaque on their brain, can suffer from cognitive impairment, and in illustrative embodiments have Alzheimer's Disease (AD). Such AD can be mild, moderate or advanced AD. Such methods utilize the fact that certain platelet derivatives having properties disclosed herein, in illustrative embodiments, freeze-dried platelet derivatives are highly effective at migrating to sites of bleeding in the brains of mammalian subjects.

Accordingly, the present disclosure provides in one aspect, a method for or administering rehydrated platelet derivatives, in illustrative embodiments rehydrated platelet derivatives having any of the properties herein, in illustrative embodiments freeze-dried platelet derivatives, to a mammalian subject, said method comprising: administering an effective dose of a rehydrated composition comprising the rehydrated freeze-dried platelet derivatives to the mammalian subject, wherein the mammalian subject can have one or more of the following properties:|
a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In some embodiments, the mammalian subject has one or more sites of bleeding in their brain, and in some embodiments, the subject has ARIA-A and/or ARIA-H, in illustrative embodiments, ARIA-H. In illustrative embodiments, the mammalian subject is afflicted with Alzheimer's Disease.

In certain aspects, the subject is being treated with, or has been treated with anti-Aβ therapeutics, such as small-molecules, or monoclonal antibodies (mabs), such as, but not limited to, donanemab, aducanumab, bapineuzumab, gantenerumab, solanezumab, and lecanemab.

Anti-Aβ monoclonal antibodies (mabs) as well as small molecules that target Aβ as well as other therapeutics are emerging disease-modifying therapies for Alzheimer disease that require brain magnetic resonance imaging (MRI) for eligibility assessment as well as for monitoring for amyloid-related imaging abnormalities (ARIA). Amyloid-related imaging abnormalities result from treatment-related loss of vascular integrity and may occur in 2 forms. Amyloid-related imaging abnormalities with edema or effusion (ARIA-E) are transient, treatment-induced edema or sulcal effusion, identified on T-FLAIR Amyloid-related imaging abnormalities with hemorrhage (ARIA-H) are treatment-induced microhemorrhages or superficial siderosis identified on T2* gradient recalled-echo MRI.

Accordingly, provided herein in one aspect, is a method for administering rehydrated platelet derivatives, in illustrative embodiments rehydrated freeze-dried platelet derivatives to a mammalian subject, said method comprising: administering an effective dose of a rehydrated composition comprising the rehydrated freeze-dried platelet derivatives to the mammalian subject, wherein the mammalian subject is being, or will be administered a therapeutic agent capable of binding amyloid beta, oligomers, and/or plaques, and combinations thereof. In some embodiments, the mammalian subject comprises the therapeutic agent capable of binding amyloid beta and/or oligomers thereof, and/or plaques thereof. In some embodiments, the rehydrated freeze-dried platelet derivatives are administered to a mammalian subject that is afflicted with Alzheimer's disease, and/or has been diagnosed with Alzheimer's disease. In some embodiments, the mammalian subject has been subjected to MRI and amyloid-related imaging abnormalities (ARIA) were detected. In some embodiments, the mammalian subject has amyloid beta deposits in the brain. In some embodiments, the mammalian subject has one or more sites of bleeding in their brain, and in some embodiments, has been subjected to MRI and ARIA were detected, and in some embodiments, the ARIA detected can be ARIA-H.

In another aspect, provided herein is a method for treating brain bleeding in a mammalian subject, the method comprising administering an effective dose of a composition comprising rehydrated platelet derivatives, and in illustrative embodiments, rehydrated freeze dried platelet derivatives to the mammalian subject, wherein in such embodiments, the mammalian subject is being, was, or will be administered a therapeutic agent capable of binding amyloid beta, oligomers, peptides, and/or plaques, and combinations thereof. In some embodiments, the mammalian subject comprises the therapeutic agent capable of binding amyloid beta and/or oligomers thereof, and/or plaques thereof. In illustrative embodiments, the mammalian subject being treated is afflicted with Alzheimer's disease or has been diagnosed with Alzheimer's disease. In some embodiments, the mammalian subject has been subjected to MRI and amyloid-related imaging abnormalities (ARIA) were detected, and in illustrative embodiments, the ARIA detected comprises ARIA-H. In some embodiments, the mammalian subject has amyloid beta deposits in the brain, and in some embodiments, is afflicted with Alzheimer's disease.

### Methods for Administering MRI-Agent Loaded Platelets/Platelet Derivatives/Cryopreserved Platelets to Detect Sites of Bleeding in the Brain

Anti-Aβ monoclonal antibodies (mabs) as well as small molecules that target Aβ as well as other therapeutics are emerging disease-modifying therapies for Alzheimer disease that require brain magnetic resonance imaging (MRI) for eligibility assessment as well as for monitoring for amyloid-related imaging abnormalities (ARIA). Amyloid-related imaging abnormalities result from treatment-related loss of vascular integrity and may occur in 2 forms. Amyloid-related imaging abnormalities with edema or effusion (ARIA-E) are transient, treatment-induced edema or sulcal effusion, identified on T-FLAIR. Amyloid-related imaging abnormalities with hemorrhage (ARIA-H) are treatment-induced microhemorrhages or superficial siderosis identified on T2* gradient recalled-echo MRI. However, there are a number of problems that remain to be solved with existing MRI methods for detecting ARIA, for example as summarized in the Background section herein.

The present invention provides in some aspects, methods for detecting sites of brain bleeds in subjects that have abnormal amounts and/or types of beta amyloid in their brain. In illustrative embodiments, methods herein provide a dual function of both detecting and reducing or eliminating bleeding from sites of brain bleeds. Such subjects in certain embodiments, can have amyloid beta oligomers, deposits, and plaque on their brain, can suffer from cognitive impairment, and in illustrative embodiments have Alzheimer's Disease (AD). Such AD can be mild, moderate or advanced AD. Such methods utilize the fact that certain platelet derivatives having properties disclosed herein, in illustrative embodiments, freeze-dried platelet derivatives are highly effective at migrating to sites of bleeding in the brains of mammalian subjects.

In another aspect, provided herein is a method for delivering an MRI agent to the brain of a mammalian subject, comprising administering to the mammalian subject, an effective dose of a rehydrated freeze-dried platelet derivative composition comprising MRI agent-loaded platelet derivatives typically having a compromised plasma membrane, wherein typically at least 50% of the MRI agent-loaded platelet derivatives are CD 41-positive platelet derivative, and wherein the mammalian subject has one or more of the following properties:
a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In illustrative embodiments, the MRI agent-loaded platelet derivatives have one, two, three, four, five or more properties for platelet derivatives and/or MRI agent-loaded platelet derivatives provided herein. In illustrative embodiments, the platelet derivatives are freeze-dried platelet derivatives.

In another aspect, provided herein is a method for delivering an MRI agent to the brain of a mammalian subject, comprising administering to the mammalian subject, an effective dose of a) a platelet composition comprising MRI agent-loaded cryopreserved platelets, and/or b) a platelet derivative composition comprising MRI agent-loaded platelet derivatives, wherein the mammalian subject administered the effective dose of a) and/or b), has one or more of the following properties:
a. a. is being, was, or will be administered a therapeutic agent capable of binding amyloid beta and/or oligomers thereof, and/or plaques thereof;
b. comprises the therapeutic agent capable of binding amyloid beta and/or oligomers thereof, and/or plaques thereof;
c. is afflicted with Alzheimer's Disease;
d. has been diagnosed with Alzheimer's Disease;
e. has been subjected to MRI and amyloid-related imaging abnormalities (ARIA) were detected; and
f. has amyloid beta deposits on their brain.

In some embodiments, the mammalian subject has one or more sites of bleeding in their brain, and in some embodiments, the subject has ARIA-A and/or ARIA-H, in illustrative embodiments, ARIA-H. In illustrative embodiments, the mammalian subject is afflicted with Alzheimer's Disease.

In certain aspects, the subject is being treated with, or has been treated with anti-Aβ therapeutics, such as small-molecules, or monoclonal antibodies (mabs), such as, but not limited to, donanemab, aducanumab, bapineuzumab, gantenerumab, solanezumab, and lecanemab.

In some embodiments of any of the aspects or embodiments herein that include MRI agent-loaded platelets/platelet derivatives such as FDPDs/cryopreserved platelets or methods to obtain any of the above products/composition, provided herein is a method of delivering MRI agent-loaded platelets/FDPDs/cryopreserved platelets comprising administering an effective amount or a therapeutically effective amount of a composition comprising MRI agent-loaded platelets, MRI agent-loaded cryopreserved platelets or MRI agent-loaded platelet derivatives of any of the aspects or embodiments disclosed herein, or the composition prepared by the process of any of the aspects or embodiments disclosed herein. Delivering of MRI agent-loaded compositions as disclosed herein, in some embodiments, can allow targeted delivery of the MRI agents to sites of interest. MRI agents can be used to image blood vessels and inflamed or diseased tissue where blood vessels have become compromised (e.g., “leaky”), in some illustrative embodiments to detect sites of bleeding. Certain conditions where such delivery methods can be used in the detection or diagnosis, and may aid in the treatment include conditions where damage to a tissue or vessels, any disruption to a tissue or vessels, or any vascular damage occurs. In some embodiments, a delivery method as disclosed herein can provide enhanced diagnosis of a disease or a condition, such as cancer (any type of cancers as disclosed herein), stroke, brain injury, embolism, or hemorrhage. In some embodiments, provided herein is a method for targeted delivery of an MRI agent to a site or sites of interest in a subject, comprising administering to a subject an effective amount or a therapeutically effective amount of the MRI agent-loaded compositions or products disclosed in any of the aspects or embodiments herein.

In some embodiments, provided herein is a method of detecting, diagnosing, or enhancing diagnosis of a disease comprising administering an effective amount or a therapeutically effective amount of a composition comprising MRI agent-loaded platelets, MRI agent-loaded cryopreserved platelets or MRI agent-loaded platelet derivatives of any of the aspects or embodiments disclosed herein, or the composition prepared by the process of any of the aspects or embodiments disclosed herein, and imaging and/or detecting the MRI agent for enhancing diagnosis of a disease. The subject to which such imaging agent-loaded (e.g. MRI agent-loaded) platelet or platelet derivatives are administered can be a subject suspected of having compromised vessels and/or tissues. The disease can be any of the diseases that include compromised vessels or tissues. Providing MRI agent-loaded products/compositions as disclosed herein to a subject can prove advantageous as it can efficiently help in imaging a particular site that enhances the diagnosis of a disease.

In some embodiments, provided herein is a method for detecting site of bleeding in a subject, comprising: (a) administering an effective amount or a therapeutically effective amount of a composition comprising MRI agent loaded platelets, MRI agent-loaded cryopreserved platelets or MRI agent-loaded platelet derivatives as disclosed in any of the aspects or embodiments herein, or the composition prepared by the process as disclosed in any of the aspects or embodiments herein, to the subject; and (b) detecting/locating/imaging the site of the MRI agent-loaded compositions/products for detecting image blood vessels and inflamed or diseased tissue, or the site of bleeding in the subject. In some embodiments, a method for detecting/locating/imaging an MRI agent well known in the art can be used herein for detecting the site of bleeding in a subject after the administration of MRI agent-loaded compositions/products as disclosed herein. In some embodiments, detecting the site of bleeding can be performed after at least 30 seconds, 1 minute, 2 minutes, 5 minutes, 7 minutes, 10 minutes, 15 minutes, 20 minutes, or 30 minutes of administering an effective amount or a therapeutically effective amount of MRI agent-loaded compositions/products. In some embodiments, detecting the site of bleeding can be performed after 1 minute to 24 hours, 1 minute to 20 hours, 1 minute to 15 hours, 1 minute to 12 hours, 1 minute to 10 hours, 1 minute to 8 hours, 1 minute to 6 hours, 1 minute to 3 hours, 1 minute to 1 hour, 2 minutes to 20 hours, 5 minutes to 15 hours, 10 minutes to 10 hours, 5 minutes to 5 hours, 5 minutes to 1 hour, or 5 minutes to 30 minutes of administering an effective amount or a therapeutically effective amount of MRI agent-loaded compositions/products.

In some embodiments, provided herein is a method for imaging compromised blood vessels or inflamed tissues in a subject, comprising: (a) administering to a subject an effective amount or a therapeutically effective amount of the MRI agent-loaded compositions or products disclosed in any of the aspects or embodiments herein; and (b) imaging the MRI agent-loaded platelets for imaging the compromised blood vessels or inflamed tissues in the subject.

In some embodiments, provided herein is a method for treating a subject having a condition/indication/disease, comprising: (a) administering to a subject a therapeutically effective amount of MRI agent-loaded compositions or products disclosed in any of the aspects or embodiments herein. MRI agent-loaded products or compositions can include MRI agent-loaded platelets, MRI agent-loaded platelet derivatives such as FDPDs, or MRI agent-loaded cryopreserved platelets. In some embodiments, disease/condition/indication can include types of cancer as disclosed herein. In some embodiments, disease/condition/indication can be selected from the group consisting of Acute lymphoblastic leukemia (ALL), Acute myeloid leukemia (AML), Breast cancer, Gastric cancer, Hodgkin lymphoma, Neuroblastoma, Non-Hodgkin lymphoma, Ovarian cancer, Cervical cancer, Small cell lung cancer, Non-small cell lung cancer (NSCLC), Soft tissue and bone sarcomas, Thyroid cancer, Transitional cell bladder cancer, Wilms tumor Neuroendocrine tumors, Pancreatic cancer, Multiple myeloma, Renal cancer, Glioblastoma Prostate cancer, Sarcoma, Colon cancer, Melanoma, Colitis, Chronic inflammatory demyelinating polyneuropathy, Guillain-Barre syndrome, Immune Thrombocytopenia, Kawasaki disease, Lupus, Multiple Sclerosis, Myasthenia gravis, Myositis, Cirrhosis with refractory ascites, Hepatorenal syndrome, Nephrotic syndrome, Organ transplantation, Paracentesis, Hypovolemia, Aneurysms, Artherosclerosis, Cancer, Cardiovascular diseases, Genetic disorders, Infectious diseases, Metabolic diseases, Neoangiogenesis, Opthalmic conditions, Hypercholesterolemia, Pulmonary hypertension, and combinations thereof. In some embodiments, disease/condition/indication can be selected from the group consisting of Von Willebrand disease, Immune thrombocytopenia, Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof. In some embodiments, MRI agent-loaded products/compositions as disclosed herein can be used for enhancing diagnosis of any of the disease/condition/indication as disclosed herein. In some embodiments, MRI agent-loaded products/compositions as disclosed herein can be used for delivering MRI agent to a subject having any of the disease/condition/indication as disclosed herein. In some embodiments, MRI agent-loaded products/compositions as disclosed herein can be used for imaging compromised blood vessels or inflamed tissue in a subject having any of the disease/condition/indication as disclosed herein.

In some embodiments, an effective amount or a therapeutically effective amount of MRI agent-loaded products/compositions disclosed herein can be administered or delivered for a number of applications including for treating to a subject afflicted with any one or combination of indications/diseases as disclosed herein, for detecting site of bleeding in a subject, for imaging compromised blood vessels or inflamed tissue in a subject, for targeted delivery of MRI agent to a subject, or for enhancing diagnosis of a condition/indication in a subject, and the dose/amount of MRI agent-loaded platelets/FDPDs/cryopreserved platelets can be in the range of 1.0×10⁷ to 1.0×10¹¹ particles/kg of the subject, or 1.0×10⁷ to 1.0×10¹⁴ particles/kg of the subject. For example, in some embodiments, a dose of a composition comprising MRI agent-loaded platelets, platelet derivatives (e.g., FDPDs), or cryopreserved platelets can include between about or exactly 1.0×10⁷ to 1.0×10¹¹ particles/kg of a subject, 1.0×10⁷ to 1.0×10¹⁰ particles/kg of a subject, 1.6×10⁷ to 1.0×10¹⁰ particles (e.g. 1.6×10⁷ to 5.1×10⁹ particles/kg of a subject, 1.6×10⁷ to 3.0×10⁹ particles/kg of a subject, 1.6×10⁷ to 1.0×10⁹ particles/kg of a subject, 1.6×10⁷ to 5.0×10⁸ particles/kg of a subject, 1.6×10⁷ to 1.0×10⁸ particles/kg of a subject, 1.6×10⁷ to 5.0×10⁷ particles/kg of a subject, 5.0×10⁷ to 1.0×10⁸ particles/kg of a subject, 1.0×10⁸ to 5.0×10⁸ particles/kg of a subject, 5.0×10⁸ to 1.0×10⁹ particles/kg of a subject, 1.0×10⁹ to 5.0×10⁹ particles/kg of a subject, or 5.0×10⁹ to 1.0×10¹⁰ particles/kg of a subject). In some embodiments of any aspect, or embodiment herein a therapeutically effective dose or effective dose or amount of the MRI agent-loaded platelet derivatives in a platelet derivative composition or amount of the MRI agent-loaded cryopreserved platelets in a cryopreserved platelet composition is in the range of 1.0×10⁷ to 1.0×10¹⁴ particles/kg of the subject, 1.6×10⁷ to 1.0×10¹⁴ particles (e.g. FDPDs, FPH, or cryopreserved platelets/kg of subject, 1.6×10⁷ to 8×10¹³ particles, 1.6×10⁷ to 5.1×10¹³ particles, 1.6×10⁷ to 3.0×10¹³ particles/kg of a subject, 1.6×10⁷ to 1.0×10¹³ particles/kg of a subject, 1.6×10⁷ to 8.0×10¹² particles/kg of a subject, 1.6×10⁷ to 5.0×10¹² particles/kg of a subject, 1.6×10⁷ to 3.0×10¹² particles/kg of a subject, 1.6×10⁷ to 1.0×10¹¹ particles/kg of a subject, 1.6×10⁷ to 8.0×10¹¹ particles/kg of a subject, 1.6×10⁷ to 5.0×10¹¹ particles/kg of a subject, 1.6×10⁷ to 3.0×10¹¹ particles/kg of a subject, 1.6×10⁷ to 1.0×10¹¹ particles/kg of a subject, 1.6×10⁷ to 8.0×10¹⁰ particles/kg of a subject, 1.6×10⁷ to 5.0×10¹⁰ particles/kg of a subject, 1.6×10⁷ to 3.0×10¹⁰ particles/kg of a subject, 1.6×10⁷ to 8.0×10¹⁰ particles/kg of a subject, 1.6×10⁷ to 5.0×10¹⁰ particles/kg of a subject, 1.6×10⁷ to 3.0×10¹⁰ particles/kg of a subject, 1.6×10⁷ to 8.0×10¹⁹ particles (e.g. FDPDs)/kg of a subject, 5.0×10⁷ to 1.0×10¹⁴ particles (e.g. FDPDs)/kg of a subject, 8.0×10⁷ to 1.0×10¹⁴ particles (e.g. FDPDs)/kg of a subject, 1.0×10⁸ to 1.0×10¹⁴ particles (e.g. FDPDs)/kg of a subject, 3.0×10⁸ to 1.0×10¹⁴ articles (e.g. FDPDs)/kg of a subject, 5.0×10⁸ to 1.0×10¹⁴ particles (e.g. FDPDs)/kg of a subject), 8.0×10⁸ to 1.0×10¹⁴ particles (e.g. FDPDs)/kg of a subject), 1.0×10⁹ to 1.0×10¹⁴ particles (e.g. FDPDs)/kg of a subject), 3.0×10⁹ to 1.0×10¹⁴ particles (e.g. FDPDs)/kg of a subject), 5.0×10⁹ to 1.0×10¹⁴ particles (e.g. FDPDs)/kg of a subject), 8.0×10⁹ to 1.0×10¹⁴ particles (e.g. FDPDs)/kg of a subject), 1.0×10¹⁰ to 1.0×10¹⁴ particles (e.g. FDPDs)/kg of a subject), 3.0×10¹⁰ to 1.0×10¹⁴ particles (e.g. FDPDs)/kg of a subject), 5.0×10¹⁰ to 1.0×10¹⁴ particles (e.g. FDPDs)/kg of a subject), 8.0×10¹⁰ to 1.0×10¹⁴ particles (e.g. FDPDs)/kg of a subject), 1.0×10¹¹ to 1.0×10¹⁴ particles (e.g. FDPDs)/kg of a subject), 5.0×10¹¹ to 1.0×10¹⁴ particles (e.g. FDPDs)/kg of a subject), 8.0×10¹¹ to 1.0×10¹⁴ particles (e.g. FDPDs)/kg of a subject), 1.0×10¹² to 1.0×10¹⁴ particles (e.g. FDPDs)/kg of a subject), 3.0×10¹² to 1.0×10¹⁴ particles (e.g. FDPDs)/kg of a subject), 5.0×10¹² to 1.0×10¹⁴ particles (e.g. FDPDs)/kg of a subject), or 8.0×10¹² to 1.0×10¹⁴ particles (e.g. FDPDs)/kg of a subject).

A person of skill in the art can contemplate the effective dose or a therapeutically effective dose of MRI agent-loaded platelets/FDPDs/cryopreserved platelets that can be required to deliver into a subject can vary based on the utility of MRI agent-loaded products/compositions as per the requirements. For example, the dose can differ for treating a subject having an indication/disease as compared to administering MRI agent-loaded products/compositions for enhancing diagnosis in a patient. Further, the dose can vary for detecting a site of bleeding in a subject. The need may differ based on the condition of the subject. The effective dosage can be categorized into a) low dosage; b) medium dosage; and c) high dosage. In some embodiments, a medicament or a method of treating a subject can have the effective dose as low, medium, or high dosage of MRI agent-loaded platelets/FDPDs/cryopreserved platelets that can broadly range from 1.0×10⁷ to 1.0×10¹⁴/kg of In some embodiments, the dose can be in the range of 250 and 5000 TGPU per kg of the subject. For example, administering can be performed until the bleeding potential of the subject is reduced as compared to the bleeding potential before the administering, or until any one of the applications as disclosed herein is achieved, for example, until the detection/diagnosis/imaging or targeted delivery to the site of interest is confirmed. In some embodiments, the administering can be performed until the bleeding stops.

A person of skill in the art can contemplate treating a subject or using platelet derivatives as described herein as a medicament in several doses in a span of time for treating the subject, or for decreasing or ceasing bleeding in brain of the subject. In some embodiments, administering of platelet derivatives, as disclosed herein, is performed for at least, or at a maximum of 2, 3, 4, 5, 6, 7, 8, 9, or 10 doses in a 72-hour period of treatment. In some embodiments, administering of platelet derivatives as disclosed herein, can be performed as a continuous infusion procedure. For example, a specific dose of platelet derivatives can be decided as per the requirement of a subject and the specific dose can be provided to the subject as a continuous infusion procedure with or without an interval. The dose can be any of the doses as disclosed herein. For example, from 1.0×10′ on the low end of the range to 1.0×10¹⁰/kg, 1.0×10¹¹/kg or 1.0×10¹²/kg of the subject on the high end of the range. A particular dose can be any dose as disclosed herein, and the dose can vary during the time interval for which the platelet derivatives are administered to a subject or a recipient in need thereof. In some embodiments, administering can be performed as a continuous infusion procedure until the bleeding potential of the subject is reduced as compared to the bleeding potential before the administering. In some embodiments, administering can be performed as a continuous infusion until the bleeding in the subject is stopped. In some embodiments, administering of platelet derivatives as described herein can be performed at regular intervals. For example, a single, double, triple or more doses of platelet derivatives as described herein and as per the requirement of a subject, for example to reduce or stop bleeding of the subject, can be administered to the recipient subject every 10 minutes, 20 minutes, 30 minutes, 1 hour, 2 hours, 3 hours, 4 hours, 5 hours, 6 hours, 7 hours, 8 hours, 9 hours, 10 hours, 11 hours, or 12 hours depending on whether bleeding is reduced to a satisfactory level, for example such that it is no longer considered life-threatening, or no longer considered severe or serious, or continued until the bleeding is mild, or stops. In some embodiments, the doses can be administered at a regular interval for 36 hours, 48 hours, or 72 hours from the start of the first dose. In some embodiments, administering of platelet derivatives as described herein can be performed as a mixed procedure in which the continuous infusion can be interrupted with a specific dose of platelet derivatives followed by a specific interval as per the requirement. In some embodiments, administering of platelet derivatives as described herein can be performed in a maximum of 2, 3, 4, 5, 6, 7, 8, 9, or 10 doses in a 24-hour period. In some embodiments, administering of platelet derivatives as described herein is performed in a maximum of 2, 3, 4, 5, 6, 7, 8, 9, or 10 doses in a 72-hour period of treatment. In some embodiments, administering of platelet derivatives can be performed at a frequency of at least one dose every 15 minutes or more frequently. For example, administering can be performed at a frequency of at least one dose every 15 minutes or more frequently starting from the first dose until the bleeding potential of the subject is reduced as compared to the bleeding potential before the administering. In some embodiments, the administering can be performed until the bleeding stops. In some embodiments, the administering can be performed for at least 1, 10, 15, 30, 45, or 60 minutes. In some embodiments, the administering can be performed at a frequency of at least one dose in every 20 minutes, 30 minutes, 45 minutes, 60 minutes, 2 hours, 3 hours, 4 hours, 5 hours, 6 hours, 7 hours, 8 hours, 9 hours, 10 hours, 12 hours, 15 hours, 18 hours, 24 hours, 30 hours, or 36 hours or more frequently. Further, in some embodiments, the subject, patient, or recipient administered FDPDs herein, or involved in the treatment or a medication process satisfies certain criteria involving one or more of: minimum age; minimum weight; total circulating platelets (TCP); confirmed diagnosis of hematologic malignancy, myeloproliferative disorder, myelodysplastic syndrome or aplasia; undergoing chemotherapy, immunotherapy, radiation therapy or hematopoietic stem cell transplantation; refractory to platelet transfusion, for example having two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets. In some embodiments, the administering is performed during surgery.

### Covalent Linking of MRI Agent Complex on Platelets, Platelet Derivatives, or Cryopreserved Platelets

In one aspect, provided herein is a composition comprising MRI agent-loaded platelets, MRI agent-loaded cryopreserved platelets or MRI agent-loaded platelet derivatives in a dried powder, wherein the MRI agent-loaded platelets, MRI agent-loaded cryopreserved platelets or the MRI agent-loaded platelet derivatives comprise an MRI agent complex covalently bonded to the surface of the platelets, cryopreserved platelets or the platelet derivatives, wherein the MRI agent complex comprises an MRI agent, and a chelator. Not to be limited by theory, it is believed that the MRI agent complex is covalently bonded to proteins on the surface of the platelets. The MRI agent complex comprising an MRI agent, a chelator, and a linker, typically, interact with a protein molecule on the surface of platelets, cryopreserved platelets, or platelet derivatives via the linker moiety, and typically, during the interaction the linker moiety, such as NHS gets released and the chelator moiety, such as DOTA gets covalently bonded to the protein via a stable amide bond. Not to be limited by theory, the chelator is understood to chelate the MRI agent such that the toxicity of the MRI agent is reduced. In some embodiments, an MRI agent complex is covalently bonded to the external surface of platelets, cryopreserved platelets, or platelet derivatives. In some embodiments, an MRI agent complex is covalently bonded to the plasma membrane of platelets, cryopreserved platelets, or platelet derivatives. In some embodiments, an MRI agent complex is covalently bonded to any structure, membrane, solid component, or other portion of platelets, cryopreserved platelets, or platelet derivatives.

### MRI Agent Complex

In some embodiments, an MRI agent complex can comprise an MRI agent, and a moiety to mask the toxic effect of the MRI agent. In some embodiments, an MRI agent complex can comprise an MRI agent and a moiety that can effectuate bonding of the MRI agent to platelets, cryopreserved platelets, or platelet derivatives. In illustrative embodiments, an MRI agent complex comprises an MRI agent, a chelator, and a linker. In some embodiments, an MRI agent complex can comprise an MRI agent and a linker. In illustrative embodiments, an MRI agent complex can comprise an MRI agent and a chelator.

In some embodiments, a chelator can be any chelator known in the art to chelate an MRI agent. In some embodiments, a chelator can be selected from the group consisting of dodecane tetra acetic acid (DOTA), diethylenetriaminepentaacetic acid (DTPA), 4-Carboxy-5,8,11-tris(carboxymethyl)-1-phenyl-2-oxa-5,8,11-triazatridecan-13-oic acid (BOPTA). Ethylenediaminetetraacetic acid (EDTA), and 1,4,7,10-tetraazacyclododecane-1,4,7-tetracetic acid (DO3A). In some embodiments, a means for chelating ions, such as cations, for example divalent cations, is any of the chelators listed in the preceding sentence. In some embodiments, a chelator is associated with an MRI agent for example through a non-covalent ionic interaction, and/or through a bond other than a covalent bond, such as an ionic bond. Chelators can be associated with an MRI agent, and in illustrative embodiments, the chelator is covalently attached to a platelet, cryopreserved platelet, or platelet derivative, or in the case where the MRI agent is loaded onto and/or into platelets using a CPP rather than by an MRI agent-complex. A person of skill in the art can appreciate that any type of chelator that reduces the toxicity of an MRI agent can be used in an MRI agent-complex as well as for CPP loading of MRI agent onto and/or into platelets. Any appropriate chelator as disclosed in the publication—Wangler, B., et al. “Chelating agents and their use in radiopharmaceutical sciences.” Mini Reviews in Medicinal Chemistry 11.11 (2011): 968-983 (Wangler et al 2011), incorporated in its entirety herein, by reference, can be used for the purposes of the present invention.

In some embodiments, a linker can be any linker known in the art that can effectuate covalent bonding for example of a chelator, with a protein, for example a protein on the surface of a platelet, cryopreserved platelet or platelet derivative. In some embodiments, for example before an MRI agent complex is contacted with a platelet derivative or cryopreserved platelet, an MRI agent complex herein comprises a linker covalently attached to a chelator, which is associated with an MRI agent, for example through an ionic interaction. In further illustrative embodiments herein, an MRI agent complex is covalently attached to a platelet, a cryopreserved platelet, or a platelet derivative via a chelator, which is associated with an MRI agent, for example through an ionic interaction, such as an ionic bond. In some embodiments, a linker can be selected from the group consisting of a compound having sulfhydryl reactive groups, such as maleimides and haloacetyl derivatives, amine reactive groups, such as isothiocyanates, succinimidyl esters, and sulfonyl halides, and carbodiimide reactive groups, such as carboxyl and amino groups. In illustrative embodiments, a linker is a compound having an amine reactive group, such as, succinimidyl ester, such as, N-Hydroxysuccinimide (NHS) ester.

In illustrative embodiments, an MRI agent complex comprises MRI agent such as, gadolinium, a chelator such as, DOTA, and a linker such as, NHS. Not to be limited by theory, in such an MRI agent, gadolinium is chelated by DOTA and DOTA is conjugated with, for example covalently bonded to NHS. When the MRI agent complex comes into contact with proteins on the surface of platelets, the NHS linker selectively reacts with primary aliphatic amine groups on proteins. Upon completion of the reaction with the primary aliphatic amine groups on proteins, NHS linker is released in the reaction, and the chelator is covalently bonded to proteins on the surface of platelets via a stable amide linkage.

In some embodiments, provided herein is a method for preparing a composition comprising MRI agent-loaded cryopreserved platelets or MRI agent-loaded platelet derivatives in a powder, comprising: (a) providing platelets; (b) contacting the platelets with an MRI agent complex comprising an MRI agent, a chelator, and a linker, to form MRI agent-loaded platelets; and (c) cryopreserving or lyophilizing the MRI agent-loaded platelets to form the composition comprising the MRI agent-loaded cryopreserved platelets or the MRI agent-loaded platelet derivatives. In some embodiments, the MRI agent is associated with the chelator, and the chelator is covalently linked to the surface of the platelets. In some embodiments, the MRI agent is associated with a surface of the cryopreserved platelets or the platelet derivatives. In some embodiments, the MRI agent is associated with the external surface of the cryopreserved platelets or the platelet derivatives. In some embodiments, the MRI agent is associated with the surface of the cryopreserved platelets or the surface of the platelet derivatives via the chelator. In some embodiments, the chelator is covalently attached to the surface of the cryopreserved platelets or the surface of the platelet derivatives. In some embodiments, the linker is covalently bonded to the chelator in the MRI agent complex. In some embodiments, the MRI agent is associated with the chelator through an ionic interaction. In some embodiments, contacting the platelets with MRI agent complex such that the MRI agent complex is covalently bound to the platelets, to form MRI agent-loaded platelets is done in the presence of a buffer comprising a salt, a base, a loading agent, and optionally at least one organic solvent. In some embodiments, a method can comprise of steps that make use of lyophilized or cryopreserved platelets as a starting material for loading.

### MRI Agents

MRI agents are contrast agents are used to improve the visibility of internal body structures in magnetic resonance imaging (MRI). MRI agents as per the present invention can be any MRI agent that are known in the art. In some embodiments, an MRI agent is selected from the group consisting of a superparamagnetic contrast agent, a diamagnetic agent, or combinations thereof. In some embodiments, an MRI agent can be a superparamagnetic contrast agent selected from the group consisting of Gd(III), Fe(III), Mn(II and III), Cr(III), Cu(II), Dy(III), Tb(III and IV), Ho(III), Er(III), Pr(III) and Eu(II and III). In some embodiments, an MRI agent can be selected from the group consisting of metal ions with atomic numbers 21-29, 39-47, or 57-83. In illustrative embodiments, an MRI agent is a gadolinium-based compound. In illustrative embodiments, an MRI agent can include gadolinium. In other illustrative embodiments, an MRI agent is Gd(III) that can be coupled with a chelator such as, DOTA.

### MRI-Agent Loaded Platelets

It will be understood that disclosed herein (e.g. aspects and embodiments) related to MRI agent loaded platelets or platelet derivatives, can be applied to imaging agent loaded platelets or platelet derivatives more generally. MRI agents are widely used to increase the contrast difference between normal and abnormal tissues. For the purposes of their application, MRI agents may be categorized according to magnetic properties, chemical composition, the presence or absence of metal atoms, route of administration, effect on the magnetic resonance image, and biodistribution. Certain aspects provided herein are or include MRI agent-loaded platelets. In some embodiments, the MRI agent-loaded platelets can be MRI agent-loaded cryopreserved platelets. In some embodiments, the MRI agent-loaded platelets can be MRI agent-loaded platelet derivatives. Certain aspects provided herein are or include MRI agent-loaded freeze-dried platelet derivatives (FDPDs). In some embodiments, TFF processing methods as disclosed herein can be used to process the platelets prior to loading or after loading with imaging agent(s) such as MRI agent(s). In some embodiments, the lyophilization or cryopreservation methods as disclosed herein can be used to process the platelets prior to loading or after loading with imaging agent(s) such as MRI agent(s). In some embodiments, imaging agent-loaded (MRI agent-loaded) FDPDs can retain one, two, three or all of the properties of FDPDs as disclosed herein.

In some embodiments, imaging agent-loaded (e.g MRI agent-loaded) platelet derivatives, imaging agent-loaded (e.g. MRI agent-loaded) FDPDs, or imaging-agent loaded (e.g. MRI agent-loaded) cryopreserved platelets are capable of retaining the properties of FDPDs that were present before loading with imaging agent (e.g. MRI agent) as disclosed elsewhere herein. MRI agent-loaded platelet derivatives or MRI agent-loaded FDPDs in some embodiments, have a higher propensity to co-aggregate in the presence of fresh platelets and an agonist, while having a reduced propensity to aggregate in the absence of fresh platelets and an agonist, compared to the propensity of fresh platelets to aggregate under these conditions.

In some embodiments, MRI agent-loaded platelet derivatives, MRI agent-loaded FDPDs, or MRI agent-loaded cryopreserved platelets at a concentration of about 4.8×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of a reagent containing tissue factor (e.g., at 0.25 μM, 0.5 μM, 1 μM, 2 μM, 5 μM or 10 μM) and optionally phospholipids.

In some embodiments, MRI agent-loaded platelet derivatives, MRI agent-loaded FDPDs, or MRI agent-loaded cryopreserved platelets can have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles. For example, in some cases, platelets or platelet derivatives (e.g., FDPDs) can have a potency of between 1.2 and 2.5 TPGU per 10⁶ particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TPGU per 10⁶ particles). In some embodiments, MRI agent-loaded FDPDs can have a potency of at least 1.5, 2, 2.5, 3, or 4 TGPU per 10⁶ particles. In some embodiments, MRI agent-loaded FDPDs can have a potency in the range of 1.5 to 10, 2 to 10, 2 to 9, 2 to 7, 2 to 6, 1 to 6, or 3 to 6 TGPU per 10⁶ particles. In some embodiments, MRI agent-loaded cryopreserved platelets can have a potency that is higher than MRI agent-loaded FDPDs. In some embodiments, MRI agent-loaded cryopreserved platelets can have a potency of at least 5, 6, 7, 8, 9, or 10 TGPU per 10⁶ particles. In some embodiments, MRI agent-loaded cryopreserved platelets can have a potency in the range of 5 to 25, 5 to 22, 5 to 20, 6 to 25, 7 to 25, or 7 to 16 TGPU per 10⁶ particles.

In some embodiments, MRI agent-loaded platelet derivatives, MRI agent-loaded FDPDs, or MRI agent-loaded cryopreserved platelets when at a concentration of at least 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 80) of less than 14 minutes (e.g., less than 13.5, 13, 12.5, 12, 11.5, or 11 minutes), for example, in platelet-reduced citrated whole blood.

In some embodiments, MRI agent-loaded platelet derivatives, MRI agent-loaded FDPDs, or MRI agent-loaded cryopreserved platelets as described herein can have a percent thrombin-induced trapping of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of thrombin. In some cases, platelets or platelet derivatives (e.g., FDPDs) as described herein can have a percent thrombin-induced trapping of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of thrombin.

In some embodiments, MRI agent-loaded platelet derivatives, MRI agent-loaded FDPDs, or MRI agent-loaded cryopreserved platelets as described herein can have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets.

In some embodiments, MRI agent-loaded platelet derivatives, MRI agent-loaded FDPDs, or MRI agent-loaded cryopreserved platelets as described herein can have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets.

In some embodiments, MRI agent-loaded platelet derivatives, MRI agent-loaded FDPDs, or MRI agent-loaded cryopreserved platelets as described herein are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist.

In some embodiments, MRI agent-loaded platelet derivatives, or MRI agent-loaded FDPDs as described herein are surrounded by a compromised plasma membrane. In these further illustrative aspects and embodiments, the platelet derivatives lack an integrated membrane around them. Instead, the membrane surrounding such platelet derivatives (e.g. FDPDs) comprises pores that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets.

In some embodiments, MRI agent-loaded platelet derivatives, MRI agent-loaded FDPDs, or MRI agent-loaded cryopreserved platelets as described herein can have a particle size (e.g., diameter, max dimension) of at least about 0.5 μm (e.g., at least about at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, a composition comprising MRI agent-loaded platelet derivatives, MRI agent-loaded FDPDs, or MRI agent-loaded cryopreserved platelets as described herein can have a microparticle content that contributes to less than about 5.0% (e.g., less than about 4.5%, 4.0%, 3.5%, 3.0%, 2.5%, 2.0%, 1.5%, 1.0%, or 0.5%) of the total scattering intensity of all particles from about 1 nm to about 60,000 nm in radius in the composition.

In some embodiments, MRI agent-loaded platelets, MRI agent-loaded platelet derivatives, or MRI agent-loaded thrombosomes may shield the MRI agent from exposure in circulation, thereby reducing or eliminating systemic toxicity (e.g. cardiotoxicity) associated with the MRI agent. In some embodiments, MRI agent-loaded platelets, MRI agent-loaded platelet derivatives, or MRI agent-loaded thrombosomes may also protect the MRI agent from metabolic degradation or inactivation. In some embodiments, MRI agent delivery with MRI agent-loaded platelets, MRI agent-loaded platelet derivatives, or MRI agent-loaded thrombosomes may therefore be advantageous in treatment of diseases such as cancer, since MRI agent-loaded platelets, MRI agent-loaded platelet derivatives, or MRI agent-loaded thrombosomes facilitate targeting of cancer cells while mitigating systemic side effects. In some embodiments, MRI agent-loaded platelets, MRI agent-loaded platelet derivatives, or MRI agent-loaded thrombosomes may be used in any therapeutic setting in which expedited healing process is required or advantageous.

Flow cytometry can be used to obtain a relative quantification of loading efficiency by measuring the mean fluorescence intensity of the MRI agent in the MRI agent-loaded platelets. Platelets can be evaluated for functionality by adenosine diphosphate (ADP), collagen, arachidonic acid, thrombin receptor activating peptide (TRAP), and/or any other platelet agonist known in the art for stimulation post-loading.

In some embodiments, the MRI agent-loaded platelets are lyophilized. In some embodiments, the MRI agent-loaded platelets are cryopreserved.

In some embodiments, MRI agent-loaded platelets such as MRI agent-loaded platelet derivatives retain the loaded MRI agent upon rehydration and release the MRI agent or the MRI agent complex upon stimulation by endogenous platelet activators.

In some embodiments, MRI agent-loaded cryopreserved platelets as disclosed herein retain the loaded MRI agent upon thawing and release the MRI agent or the MRI agent complex upon stimulation by endogenous platelet activators.

In some embodiments, the dried platelets (such as MRI agent-loaded freeze-dried platelets) retain the loaded MRI agent upon rehydration and release the MRI agent upon stimulation by endogenous platelet activators. In some embodiments, at least about 10%, such as at least about 20%, such as at least about 30% of the MRI agent is retained. In some embodiments, from about 10% to about 30%, or from about 20% to about 30% of the MRI agent is retained. In some embodiments, more than 30% of the MRI agent is retained.

In some embodiments herein that include a composition, a method, or a use of a composition therein, MRI agent-loaded cryopreserved platelets upon thawing form thawed MRI agent-loaded platelets, such that the thawed MRI agent-loaded platelets retain at least 5%, 10%, 15%, 20%, 25%, 30%, 40%, 50% or more of the loaded MRI agent upon thawing. In some embodiments, the thawed MRI agent-loaded platelets retain 5%-90%, 5%-80%, 5%-70%, 5-60%, 10-80%, 10-75%, 10-60%, 15-90%, 20-90%, or 25-90% of the loaded MRI agent upon thawing. In some embodiments that include a method for preparing MRI agent-loaded cryopreserved platelets, the MRI agent-loaded cryopreserved platelets retain at least 10%, 20%, 30%, 40%, 50%, 60%, 70%, 80%, or more of the MRI agent loaded on platelets before the step of cryopreserving. In some embodiments, the MRI agent-loaded cryopreserved platelets retain 5%-90%, 5%-80%, 5%-70%, 5-60%, 10-80%, 10-75%, 10-60%, 15-90%, 20%-90%, or 25-90% of the MRI agent loaded on platelets before the step of cryopreserving.

In some embodiments, MRI agent-loaded platelet derivatives upon rehydrating form rehydrated MRI agent-loaded platelet derivatives, such that the rehydrated MRI agent-loaded platelet derivatives retain at least 5%, 10%, 15%, 20%, 25%, 30%, 40%, 50% or more of the loaded MRI agent upon rehydrating. In some embodiments, the rehydrated MRI agent-loaded platelet derivatives retain 5%-90%, 5%-80%, 5%-70%, 5-60%, 10-80%, 10-75%, 10-60%, 15-90%, 20-90%, or 25-90% of the loaded MRI agent upon rehydrating. In some embodiments that include a method for preparing MRI agent-loaded platelet derivatives, the MRI agent-loaded platelet derivatives retain at least 10%, 20%, 30%, 40%, 50%, 60%, 70%, 80%, or more of the MRI agent loaded on platelets before the step of lyophilizing. In some embodiments, the MRI agent-loaded platelet derivatives retain 5%-90%, 5%-80%, 5-70%, 5-60%, 10-80%, 10-75%, 10-60%, 15-90%, 20-90%, or 25-90% of the MRI agent loaded on platelets before the step of lyophilizing.

In some embodiments, MRI agent-loaded cryopreserved platelets can have higher retention of MRI agent as compared to MRI agent-loaded platelet derivatives such as FDPDs.

Any suitable MRI agent can be loaded in a platelet. For example, any agent suitable for magnetic resonance imaging can be loaded into a platelet.

In some embodiments, the MRI agent can include Gadolinium. In some embodiments, Gadolinium (Gd(III)) can be coupled with a chelator. In some embodiments, the chelator can be DOTA (tetrxetan). In some embodiments, the MRI agent can be a complex of Gd and DOTA (e.g., Gd-DOTA).

In some embodiments, the MRI agent can be a nanoparticle. Any suitable nanoparticle (e.g., magnetic nanoparticle) can be loaded into the platelet. In some embodiments, the nanoparticle is a FeO₃ nanoparticle.

In some embodiments, the nanoparticle can be about 15 nm to about 100 nm in diameter. In some embodiments, the nanoparticle can be about 20 nm to about 90 nm in diameter. In some embodiments, the nanoparticle can be about 30 nm to about 80 nm in diameter. In some embodiments, the nanoparticle can be about 40 nm to about 70 nm in diameter. In some embodiments, the nanoparticle can be about 50 nm to about 60 nm in diameter. In some embodiments, the nanoparticle can be about 20 nm to about 30 nm in diameter.

In some embodiments, the nanoparticles can be at a concentration of about 1×10⁻²⁰ to about 1×10⁻¹⁴ particles/mL. In some embodiments, the nanoparticles can be at a concentration of about 1×10⁻¹⁹ to about 1×10⁻¹⁵ particles/mL. In some embodiments, the nanoparticles can be at a concentration of about 1×10⁻¹⁸ to about 1×10⁻¹⁶ particles/mL.

In some embodiments, the MRI agent loaded platelets can be coupled (e.g., conjugated) with a cell penetrating peptide. Cell penetrating peptides are peptides that can facilitate cellular uptake of various cargo (e.g., nucleic acid, protein, metabolites, lipids, nanoparticles, metals, etc.). Generally, cell penetrating peptides can cross a cellular membrane by direct penetration in the membrane, endocytosis mediated entry, or translocation through the formation of a transitory structure, although additional mechanisms are known.

The HIV Tat protein is an example of a cell penetrating peptide. The Tat protein includes between 86 and 101 amino acids depending on the subtype. Tat is a regulatory protein that enhances the viral transcription efficiency. Tat also contains a protein transduction domain which functions as a cell-penetrating domain allowing Tat to cross cellular membranes.

In some embodiments, an MRI agent can be coupled with any functional fragment of an HIV Tat protein. In some embodiments, the full-length of HIV-TAT protein is as set forth in SEQ ID NO: 1. In some embodiments, an MRI agent can be coupled with a portion/functional fragment of an HIV Tat protein. In some embodiments, an MRI agent can be coupled with a portion of the Tat protein: L-Tat₄₉₋₅₇ as described in Mishra, R., et. al., Cell-Penetrating Peptides and Peptide Nucleic Acid-Coupled MRI Contrast Agents: Evaluation of Cellular Delivery and Target Binding, Bioconjugate Chem. 20, 1860-1868 (2009).

In some embodiments, the MRI agent can be coupled with a lipophilic moiety. Some non-limiting examples include a lipid coated nanoparticle or a liposome.

In some embodiments, the MRI agent can be coupled with a cyclodextrin cage.

In some embodiments, the one or more other components that are loaded in the platelets include Prostaglandin E1.

In some embodiments, the one or more other components that are loaded in the platelets do not include Prostaglandin E1.

In some embodiments, the one or more other components that are loaded in the platelets include a glycoprotein IIb/IIIa inhibitor (GP IIb/IIIa). Non-limiting examples of GP IIb/IIIa inhibitors include GR144053, eptifibatide, ethylenediaminetetraacetic acid (EDTA), abciximab, tirofiban.

In some embodiments, the one or more other components that are loaded in the platelets include GR144053.

In some embodiments, the one or more other components that are loaded in the platelets do not include GR144053.

In some embodiments, the one or more other components that are loaded in the platelets include eptifibatide.

In some embodiments, the one or more other components that are loaded in the platelets do not include eptifibatide.

### Loading of MRI Agents Using a Cell Penetrating Peptide

Platelets, platelet derivatives, FDPDs, or cryopreserved platelets as disclosed herein that are loaded with an MRI agent are termed as MRI agent-loaded platelets, MRI agent-loaded platelet derivatives, MRI agent-loaded FDPDs, or MRI agent-loaded cryopreserved platelets, respectively. In one aspect, provided herein is a composition comprising MRI agent-loaded platelets, MRI agent-loaded platelet derivatives, MRI agent-loaded FDPDs, or MRI agent-loaded cryopreserved platelets comprising MRI agent coupled to a cell penetrating peptide (CPP). In some embodiments, MRI agent coupled to a CPP can further comprise a chelator. In some embodiments, a chelator can be any chelator that is described elsewhere in the specification. In illustrative embodiments, the chelator is DOTA, the MRI agent is gadolinium, and the CPP is a TAT peptide that is effective at penetrating a cell.

As described here and in the Examples below, one non-limiting exemplary method of preparing FDPDs loaded with an MRI agent is as follows: Prepare the MRI agent (e.g., FITC-CPP-Ga-DOTA or FITC-labeled nanoparticles) in aqueous buffer at room temperature. Incubate the FITC-CPP-Ga-DOTA or FITC-labeled nanoparticles with platelets up to 3 hours at 37° C. on a rocker with low frequency agitation. Transfected platelets may be lyophilized to create FDPDs with an MRI agent. Fluorescently labeled FITC-CPP-Ga-DOTA or FITC-labeled nanoparticles can be detected via flow cytometry and visualized using fluorescence microscopy.

### Cell Penetrating Peptide (CPP)

Cell penetrating peptides (CPPs) are peptides that can facilitate cellular uptake of various cargo (e.g., nucleic acid, protein, metabolites, lipids, nanoparticles, metals, etc.). They are included in some of the aspects and embodiments herein typically to facilitate uptake of imaging agents, which in illustrative embodiments are MRI agents. Cargo can be coupled (e.g., conjugated) to a cell penetrating peptide either covalently or non-covalently. A cell penetrating peptide conjugated to cargo can transport the cargo across a cellular membrane, generally via endocytosis, however other mechanisms are known in the art. CPPs are typically between 5 and 30, and in some illustrative embodiments between 10 and 30 amino acids in length, or are oligomers thereof that can include for example, 2 tandem copies or between 2 and 10, 9, 8, 7, 6, 5, 4, or 3 tandem repeats of the CPP, that can optionally be separated by a 1-3 amino acid peptide linker, that are capable of crossing the cytoplasmic membrane efficiently. In some embodiments, a CPP included in any aspect or embodiment herein can be any peptide having 5 to 150, 5 and 100, 5 to 75, 50 to 50, or 5 to 30 amino acids that are capable of crossing the cytoplasmic membrane. In some embodiments, a CPP is any of the peptides that are disclosed in Kersemans et al 2008 (Kersemans, Veerle et al. “Cell penetrating peptides for in vivo molecular imaging applications.” Current pharmaceutical design vol. 14,24 (2008): 2415-47) incorporated in its entirety herein by reference. In some embodiments, CPP can be a protein-derived CPP, a synthetic CPP, or a mixed/chimeric CPP. In some embodiments, a protein-derived CPP is derived from a naturally occurring protein such as, but not limiting to, TAT protein, and penetratin. In some embodiments, a synthetic CPP such as, but not limiting to polyarginines can be a CPP that is developed by known techniques, such as, phage display method. In some embodiments, a mixed or a chimeric CPP can be a CPP which is a combination of naturally occurring (protein derived) CPP, and synthetic CPP, such as transportan CPP, or can be a combination of the N-terminal fragment of the neuropeptide gelanin and the membrane-interacting wasp venom peptide, mastoparan. In some embodiments, a CPP can be considered as any peptide that is capable of penetrating a cell membrane without the involvement of energy-dependent processes. A person of skill in the art will appreciate that a CPP in illustrative embodiments is a peptide that is capable of, has the property of, and/or is adapted to penetrate a cell membrane without the involvement of energy-dependent processes. In some embodiments, a CPP can have at least 3, 4, 5, 6, 7, 8, 9, 10, 11, 12, 13, 14, 15, 16, 17, 18, 19, 20, 21, 22, 23, 24, 25, 26, 27, 28, 29, or 30 amino acids and in illustrative embodiments less than or equal to 75 amino acids. In some embodiments, a CPP can have 3 to 75, 4 to 65, 5 to 50, 5 to 40, 5 to 30, 10 to 75, 10 to 65, 10 to 50, 10 to 40, or 10 to 30 amino acids. In some embodiments, a CPP can be a positively charged peptide (cationic peptide). In some embodiments, a cationic CPP can comprise multiple lysine and/or arginine residues. In some embodiments, a cationic CPP can comprise at least 4, 5, 6, 7, 8, 9, or 10 lysine and/or arginine residues. In some embodiments, a cationic CPP comprises between 4 and 15, 14, 13, 12, 11, or 10 lysine and/or arginine residues. In some embodiments, a cationic CPP can comprise a nuclear localization sequence (NLS). In some embodiments, a CPP can be an amphipathic CPP that can comprise alternating regions of hydrophilic and hydrophobic amino acids, and the amphipathic CPP can have a resulting charge that can be positive, negative, or neutral.

In some embodiments, an amphipathic CPP can be proline-rich amphipathic peptide. In some embodiments, a CPP can be a hydrophobic CPP having hydrophobic properties, one or more hydrophobic domains, and comprising hydrophobic residues. Such proteins, such as, as a stretch of between 2, 3, 4, 5, or 6 but not limiting to, alanine, leucine, isoleucine, phenylalanine, tryptophan, methionine, and tyrosine. In some embodiments, a CPP can be any of the CPPs or a functional fragment of any of the CPPs that has been disclosed in the publication—Böhmová, E et al. “Cell-penetrating peptides: a useful tool for the delivery of various cargoes into cells.” Physiological research vol. 67, Suppl 2 (2018): S267-S279 (B6hmova et al 2018), incorporated in its entirety by reference herein, and the publication Bôhmova et al 2018 incorporated in its entirety herein by reference. In some embodiments, a CPP can be any one of the CPPs or a functional fragment of any of the CPPs that has been disclosed in the publication—Ramaker, E et al. “Cell penetrating peptides: a comparative transport analysis for 474 sequence motifs” Drug Delivery, 25:1, 928-937 (2018) (Ramaker et al 2018), incorporated in its entirety by reference herein.

In some embodiments, a protein-derived CPP is selected from the group consisting of Pep-1, penetratin, TAT peptide (e. g. amino acid residues 49-57 of SEQ ID NO:1) (amino acid numbering is with respect to the full-length HIV-TAT peptide as set forth in SEQ ID NO: 1), TAT peptide (e.g. amino acid resides 48-60 of SEQ ID NO:1) (amino acid numbering is with respect to the full-length HIV-TAT peptide as set forth in SEQ ID NO: 1), calcitonin-derived CPP, nuclear localization sequences, new polybasic CPPs, N-terminal repetitive domain of maize gamma-zein, peptides from gp41 fusion sequence, preS2-TLM, signal-sequence hydrophobic region (SSHR), pVEC, Vpr, VP22, Human integrin b3 signal sequence, gp41 fusion sequence, Caiman crocodylus Ig(v) light chain, hCT derived peptide, Kaposi FGF signal sequences, CPP from pestivirus envelope glycoprotein, CPP derived from the prion protein, Yeast PRP6 (129-144), Phi21 N (12-29), Delta N (1-22), FHV coat (35-49), BMV Gag (7-25), HTLV-II Rex (4-16), HIV-1 Rev (9-20), RSG-1.2, Lambda-N(48-62), SV40 NLS, Bipartite, Nucleoplasmin (155-170), NLS, Herpesvirus, 8 k8 protein (124-135), Buforin-II (20-36), Magainin, PDX-1-PTD, crotamine, pIs1, SynB1, Fushi-tarazu (254-313), and Engrailed (454-513). In certain illustrative embodiments, a protein-derived CPP is penetratin. In certain illustrative embodiments, a CPP can be any functional fragment of penetratin peptide. In certain illustrative embodiments, a protein-derived CPP is TAT peptide (e.g. comprising, consisting essentially of, or consisting of amino acid residues 49-57 of SEQ ID NO:1). In certain illustrative embodiments, a protein-derived CPP is TAT peptide (e.g. comprising, consisting essentially of, or consisting of amino acid residues 48-60 of SEQ ID NO:1). In certain illustrative embodiments, a CPP can be any functional fragment of TAT peptide. In certain illustrative embodiments, a protein-derived CPP can be any peptide disclosed in the publication Kersemans et al 2008.

In some embodiments, a synthetic and/or mixed and/or chimeric CPP is selected from the group consisting of transportan, polyarginine CPPs, poly-d-arginine, KLAL peptide/model amphipathic peptide (MAP), KALA model amphipathic peptide, modeled Tat peptide, Loligomer, b-sheet-forming peptide, retro-inverso forms of established CPPs, W/R penetratin, MPG, Pep-1, Signal-sequence-based peptides (I), Signal-sequence-based peptides (II), Carbamate 9, PTD-4, P1D-5, RSV-A9, CTP-512, and U2AF. In certain illustrative embodiments, a synthetic and/or mixed CPP can be any peptide described in the publication Kersemans et al 2008.

In some embodiments, a CPP can comprise a functional fragment of any of the CPPs disclosed herein including those in any of the cited references incorporated herein. A functional fragment can be any fragment of a CPP that retains the ability to penetrate a cell membrane and deliver a cargo molecule inside the cell, which in illustrative embodiments can include an imaging agent such as an MRI agent. In some embodiments, a CPP can be, comprise, consistent essentially of, or consist of any one or more of the peptides as set forth in amino acid residues 49-57 of SEQ ID NO:1, amino acid resides 48-60 of SEQ ID NO:1, or the peptide whose sequence is provided in SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or a functional fragment of any of the foregoing peptides. In some embodiments, a CPP can comprise, consist essentially of, or consist of any peptide that is at least 75%, 80%, 85%, 90%, 92%, 95%, 97.5%, 99%, or 99.5% identical to any of the foregoing sequences or a functional fragment thereof. In certain illustrative embodiments, a CPP can be any peptide that is at least 75%, 80%, 85%, 90%, 92%, 95%, 97.5%, 99%, or 99.5% identical to any peptide or its functional fragments as set forth in SEQ ID NO: 1 to SEQ ID NO: 87. In some illustrative embodiments, a CPP can be any one of the peptides as set forth in SEQ ID NO: 2 to 7, or functional fragments thereof. In some illustrative embodiments, a CPP can be any one of the peptides as set forth in SEQ ID NO: 8 to 11, or functional fragments thereof. A CPP in certain illustrative embodiments herein, is a functional fragment of HIV-TAT protein (SEQ ID NO: 1). In some embodiments, a CPP as per the present disclosure can be derived from the peptide as set forth in SEQ ID NO: 1. In some embodiments, a CPP as per the present disclosure can be a functional fragment of the peptide as set forth in SEQ ID NO: 1. In some embodiments, a CPP as per the present disclosure can be a functional fragment having at least 3, 4, 5, 6, 7, 8, 9, 10 amino acids of the peptide as set forth in SEQ ID NO: 1. In some embodiments, the functional fragment can be derived from SEQ ID NO: 1 and identified using mutational techniques. In some embodiments, a CPP can be any peptide that is at least 25%, 30%, 35%, 40%, 45%, 50%, 55%, 60%, 65%, 70%, 75%, 80%, 85%, 90%, 92%, 95%, 97.5%, 99%, or 99.5% identical to a consecutive stretch of at least 10 amino acids or the entire peptide as set forth in SEQ ID NO: 1.

In some embodiments, a CPP can form a complex/linked with an MRI agent/imaging agent that needs to be delivered to a cell. In some embodiments, a CPP linked with an imaging agent, such as an MRI agent, can be a CPP-MRI/Imaging agent complex and can have a size that is effective for the complex to achieve endocytic uptake. In some embodiments, a CPP-MRI/Imaging agent complex can have a size of at most 500 nm, 450 nm, 400 nm, 350 nm, 300 nm, 250 nm, or 200 nm.

In some embodiments a CPP can be any means provided herein, typically any peptide means provided herein, for penetrating a cell membrane for delivering a cargo molecule inside the cell, for example a platelet or platelet derivative. The cargo can comprise an imaging agent, in illustrative embodiments an MRI agent. Such means is typically any one or more peptides consisting of SEQ ID NOs: 2 to 87 with oligomeric numbers as provided in the Exemplary CPPs Table.

In some embodiments, provided herein is a method for preparing a composition comprising MRI agent-loaded cryopreserved platelets or MRI agent-loaded platelet derivatives in a powder, comprising: (a) providing platelets; (b) contacting the platelets with an MRI agent coupled to a cell penetrating peptide, and a buffer comprising a salt, a base, a loading agent, and optionally at least one organic solvent, to form MRI agent-loaded platelets; and (c) cryopreserving the MRI agent-loaded platelets to form the composition comprising the MRI agent-loaded cryopreserved platelets or lyophilizing the MRI agent-loaded platelets to form the composition comprising the MRI agent-loaded platelet derivatives. In some embodiments, a method can comprise of steps that make use of lyophilized or cryopreserved platelets as a starting material for loading.

The “Exemplary CPPs Table” below discloses a non-limiting set of CPPs that can be included in any of the aspects or embodiments of the present disclosure. The CPPs include certain fragments of SEQ ID NO: 1 as indicated herein, and SEQ ID Nos:22-87 with oligomeric numbers indicated in the Table if indicated after the sequence.

### Exemplary CPPs Table

| Sequence Nos | Name | Sequence | Reference |
|---|---|---|---|
| SEQ ID | HIV-TAT | MEPVDPRLEPWKHPGSQPKTACTTCYC | UniProt KB Accession |
| NO: 1 | Full | KKCCFHCQVCFTTKALGISYGRKKRRQR | No. P04610 |
| | Length | RRPPQ | |
| | Protein | GSQTHQVSLSKQPTSQPRGDPTGPKE | |
| SEQ ID | TAT | KLRKKRRQRRR | |
| NO: 2 | Functional | | |
| | Fragment/ | | |
| | Derivative | | |
| SEQ ID | TAT | GRKKRRQRRRPQ | |
| NO: 3 | Functional | | |
| | Fragment | | |
| SEQ ID | Tat47-60 | YGRKKRRQRRRPPQ | |
| NO: 4 | Functional | | |
| | Fragment | | |
| SEQ ID | Tat47-57 | YGRKKRRQRRR | |
| NO: 5 | Functional | | |
| | Fragment | | |
| SEQ ID | Tat49-57 | RKKRRQRRR | Kersemans et al 2008 |
| NO: 6 | Functional | | |
| | Fragment | | |
| SEQ ID | Tat48-60 | GRKKRRQRRRPPQ | Kersemans et al 2008 |
| NO: 7 | Functional | | |
| | Fragment | | |
| SEQ ID | Pep-1 | KETWWETWWTEWSQPKKKRKV | Kersemans et al 2008 |
| NO: 8 | | | |
| SEQ ID | Pep-7 | SDLWEMMMVSLACQY | |
| NO: 9 | | | |
| SEQ ID | Penetratin | RQIKIWFQNRRMKWKK | Kersemans et al 2008 |
| NO: 10 | | | |
| SEQ ID | Short | RRMKWKK | |
| NO: 11 | Penetratin | | |
| SEQ ID | Calcitonin | LGTYTQDFNKFHTFPQTAIGVGAP | Kersemans et al 2008 |
| NO: 12 | derived | | |
| | CPP | | |
| SEQ ID | nuclear | VQRKRQKLMP | Kersemans et al 2008 |
| NO: 13 | localization | | |
| | sequences | | |
| SEQ ID | nuclear | SKKKKTKV | Kersemans et al 2008 |
| NO: 14 | localization | | |
| | sequences | | |
| SEQ ID | nuclear | GRKRKKRT | Kersemans et al 2008 |
| NO: 15 | localization | | |
| | sequences | | |
| SEQ ID | new | RRRERRAEK | Kersemans et al 2008 |
| NO: 16 | polybasic | | |
| | CPPs | | |
| SEQ ID | new | KCPSRRPKR | Kersemans et al 2008 |
| NO: 17 | polybasic | | |
| | CPPs | | |
| SEQ ID | N-terminal | (VRLPPP)n, n = 1 to 3 | Kersemans et al 2008 |
| NO: 18 | repetitive | | |
| | domain of | | |
| | maize | | |
| | gamma-zein | | |
| SEQ ID | N-terminal | (VHLPPP)n, n = 1 to 3 | Kersemans et al 2008 |
| NO: 19 | repetitive | | |
| | domain of | | |
| | maize | | |
| | gamma-zein | | |
| SEQ ID | N-terminal | (VKLPPP)n, n = 1 to 3 | Kersemans et al 2008 |
| NO: 20 | repetitive | | |
| | domain of | | |
| | maize | | |
| | gamma-zein | | |
| SEQ ID | peptides | AVGAIGALFLGFLGAAG | Kersemans et al 2008 |
| NO: 21 | from gp41 | | |
| | fusion | | |
| | sequence | | |
| SEQ ID | preS2- | PLSSIFSRIGDP | Kersemans et al 2008 |
| NO: 22 | TLM | | |
| SEQ ID | signal- | AAVALLPAVLLALLAP | Kersemans et al 2008 |
| NO: 23 | sequence | | |
| | hydrophobic | | |
| | region | | |
| | (SSHR) | | |
| SEQ ID | signal- | VTVLALGALAGVGVG | Kersemans et al 2008 |
| NO: 24 | sequence | | |
| | hydrophobic | | |
| | region | | |
| | (SSHR) | | |
| SEQ ID | pVEC | IAARIKLRSRQHIKLRHL | Kersemans et al 2008 |
| NO: 25 | | | |
| SEQ ID | Vpr | DTWPGVEALIRILQQLLFIH FRIGCQH | Kersemans et al 2008 |
| NO: 26 | | | |
| SEQ ID | VP22 | DAATATRGRSAASRPTERPRAPARSASR | Kersemans et al 2008 |
| NO: 27 | | PRRPVD | |
| SEQ ID | gp41 | GALFLGWLGAAGSTMGA | Kersemans et al 2008 |
| NO: 28 | fusion | | |
| | sequence | | |
| SEQ ID | Caiman | MGLGLHLLVLAAALQGA | Kersemans et al 2008 |
| NO: 29 | crocodylus | | |
| | Ig(v) | | |
| | light chain | | |
| SEQ ID | Kaposi | AAVLLPVLLAAP | Kersemans et al 2008 |
| NO: 30 | FGF | | |
| | signal | | |
| | sequences | | |
| SEQ ID | CPP from | RQGAARVTSWLGRQLRIAGKRLEGRSK | Kersemans et al 2008 |
| NO: 31 | pestivirus | | |
| | envelope | | |
| | glycoprotein | | |
| SEQ ID | CPP | MANLGYWLLALFVTMWTDVGLC | Kersemans et al 2008 |
| NO: 32 | derived | KKRPKP | |
| | from the | | |
| | prion | | |
| | protein | | |
| SEQ ID | Yeast | TRRNKRNRIQEQLNRK | Kersemans et al 2008 |
| NO: 33 | PRP6 | | |
| | (129-144) | | |
| SEQ ID | Phi21 N | TAKTRYKARRAELIAERR | Kersemans et al 2008 |
| NO: 34 | (12-29) | | |
| SEQ ID | Delta N | MDAQTRRRERRAEKQAQWKAAN | Kersemans et al 2008 |
| NO: 35 | (1-22) | | |
| SEQ ID | FHV coat | RRRRNRTRRNRRRVR | Kersemans et al 2008 |
| NO: 36 | (35-49) | | |
| SEQ ID | BMV Gag | KMTRAQRRAAARRNRWTAR | Kersemans et al 2008 |
| NO: 37 | (7-25) | | |
| SEQ ID | HTLV-II | TRRQRTRRARRNR | Kersemans et al 2008 |
| NO: 38 | Rex (4-16) | | |
| SEQ ID | HIV-1 | RQARRNRRRRWR | Kersemans et al 2008 |
| NO: 39 | Rev (9-20) | | |
| SEQ ID | RSG-1.2 | DRRRRGSRPSGAERRRRRAAAA | Kersemans et al 2008 |
| NO: 40 | | | |
| SEQ ID | Lambda-N | QTRRRERRAEKQAQW | Kersemans et al 2008 |
| NO: 41 | (48-62) | | |
| SEQ ID | SV40 | PKKKRKV | Kersemans et al 2008 |
| NO: 42 | NLS | | |
| SEQ ID | Bipartite, | KRPAAIKKAGQAKKKK | Kersemans et al 2008 |
| NO: 43 | Nucleo- | | |
| | plassmin | | |
| | (155-170), | | |
| | NLS | | |
| SEQ ID | Herpesvirus, | TRRSKRRSHRKF | Kersemans et al 2008 |
| NO: 44 | 8 k8 | | |
| | protein | | |
| | (124-135) | | |
| SEQ ID | Buforin-II | RAGLQFPVGRVHRLLRK | Kersemans et al 2008 |
| NO: 45 | (20-36) | | |
| SEQ ID | Magainin | TRSSRAGLQFPVGRVHRLLRK | Kersemans et al 2008 |
| NO: 46 | | | |
| SEQ ID | PDX-1- | RHIKIWFQNRRMKWKK | Kersemans et al 2008 |
| NO: 47 | PTD | | |
| SEQ ID | crotamine | YKQCHKKGGHCFPKEKICLPPSSDFGKM | Kersemans et al 2008 |
| NO: 48 | | DCRWRWKCCKKGSG | |
| SEQ ID | pIsl | RVIRVWFQNKRCKDKK | Kersemans et al 2008 |
| NO: 49 | | | |
| SEQ ID | SynB1 | RGGRLSYSRRRFSTSTGR | Kersemans et al 2008 |
| NO: 50 | | | |
| SEQ ID | Fushi- | SKRTRQTYTRYQTLELEKEFHFNRYITRR | Kersemans et al 2008 |
| NO: 51 | tarazu | RRIDIANALSLSERQIKIWFQNRRMKSKK | |
| | (254-313) | DR | |
| SEQ ID | Engrailed | EKRPRTAFSSEQLARLKREFNENRYLTE | Kersemans et al 2008 |
| NO: 52 | (454-513) | RRRQQLSSELGLNEAQIKIWFQNKRAKI | |
| | | KKST | |
| SEQ ID | transportan | GWTLNSAGYLLGKINLKALAALAKKIL | Kersemans et al 2008 |
| NO: 53 | | | |
| SEQ ID | polyarginine | RRRRRRRR | Kersemans et al 2008 |
| NO: 54 | CPPs | | |
| SEQ ID | poly-d- | RRRRRRRRR | Kersemans et al 2008 |
| NO: 55 | arginine | | |
| SEQ ID | KLAL | KLALKLALKALKAALKLA | Kersemans et al 2008 |
| NO: 56 | peptide/ | | |
| | model | | |
| | amphipathic | | |
| | peptide | | |
| | (MAP) | | |
| SEQ ID | KALA | WEAKLAKALAKALAKHLAKALAKALK | Kersemans et al 2008 |
| NO: 57 | model | ACEA | |
| | amphipathic | | |
| | peptide | | |
| SEQ ID | modeled | YARAAARQARA | Kersemans et al 2008 |
| NO: 58 | Tat | | |
| | peptide | | |
| SEQ ID | Loligomer | (TPPKKKRKVEDPKKKKK)8 | Kersemans et al 2008 |
| NO: 59 | | | |
| SEQ ID | b-sheet- | DPKGDPPKGVTVTVTVTVTG KGDPKPD | Kersemans et al 2008 |
| NO: 60 | forming | | |
| | peptide | | |
| SEQ ID | retro- | KKWKMRRNQFWVRVQR | Kersemans et al 2008 |
| NO: 61 | inverso | | |
| | forms of | | |
| | established | | |
| | CPPs | | |
| SEQ ID | W/R | RRWRRWWWRRWWRRWRR | Kersemans et al 2008 |
| NO: 62 | penetratin | | |
| SEQ ID | MPG | GALFLGFLGAAGSTMGAWSQPKSKRKV | Kersemans et al 2008 |
| NO: 63 | | C | |
| SEQ ID | Signal- | GALFLGWLGAAGSTMGAWSQPKKKRK | Kersemans et al 2008 |
| NO: 64 | sequence- | V | |
| | based | | |
| | peptides | | |
| | (I) | | |
| SEQ ID | PTD-5 | RRQRRTSKLMKR | Kersemans et al 2008 |
| NO: 65 | | | |
| SEQ ID | RSV-A9 | RRIPNRRPRR | Kersemans et al 2008 |
| NO: 66 | | | |
| SEQ ID | CTP-512 | YGRRARRRRRR | Kersemans et al 2008 |
| NO: 67 | | | |
| SEQ ID | U2AF | SQMTRQARRLYV | Kersemans et al 2008 |
| NO: 68 | | | |
| SEQ ID | Type of | WLRRIKAWLRRIKALNRQLGVAA | Ramaker et al 2018 |
| NO: 69 | CPP | | |
| SEQ ID | Type of | VKRKKKPALWKTLLKKVLKA | Ramaker et al 2018 |
| NO: 70 | CPP | | |
| SEQ ID | Type of | KTVLLRKLLKLLVRKI | Ramaker et al 2018 |
| NO: 71 | CPP | | |
| SEQ ID | Type of | KKKKKKKKKKKKKKKKKKK | Ramaker et al 2018 |
| NO: 72 | CPP | | |
| SEQ ID | Type of | KLALKLALKALKAALK | Ramaker et al 2018 |
| NO: 73 | CPP | | |
| SEQ ID | Type of | RQARRNRRRALWKTLLKKVLKA | Ramaker et al 2018 |
| NO: 74 | CPP | | |
| SEQ ID | Type of | LLKKRKVVRLIKFLLK | Ramaker et al 2018 |
| NO: 75 | CPP | | |
| SEQ ID | Type of | LIRLWSHLIHIWFQNRRLKWKKK | Ramaker et al 2018 |
| NO: 76 | CPP | | |
| SEQ ID | Type of | LNSAGYLLGKINLKALAALAKKIL | Ramaker et al 2018 |
| NO: 77 | CPP | | |
| SEQ ID | Type of | CWKKKKKKKK | Ramaker et al 2018 |
| NO: 78 | CPP | | |
| SEQ ID | Type of | YTAIAWVKAFIRKLRK | Ramaker et al 2018 |
| NO: 79 | CPP | | |
| SEQ ID | Type of | PKKKRKVALWKTLLKKVLKA | Ramaker et al 2018 |
| NO: 80 | CPP | | |
| SEQ ID | Type of | GLWRALWRALRSLWKLKRKV | Ramaker et al 2018 |
| NO: 81 | CPP | | |
| SEQ ID | Type of | GLWRALWRGLRSLWKKKRKV | Ramaker et al 2018 |
| NO: 82 | CPP | | |
| SEQ ID | Type of | GLWRALWRGLRSLWKLKRKV | Ramaker et al 2018 |
| NO: 83 | CPP | | |
| SEQ ID | Type of | KALAKALAKLWKALAKAA | Ramaker et al 2018 |
| NO: 84 | CPP | | |
| SEQ ID | Type of | KLAAALLKKWKKLAAALL | Ramaker et al 2018 |
| NO: 85 | CPP | | |
| SEQ ID | Type of | GLFKALLKLLKSLWKLLLKA | Ramaker et al 2018 |
| NO: 86 | CPP | | |
| SEQ ID | Type of | KLALKLALKAWKAALKLA | Ramaker et al 2018 |
| NO: 87 | CPP | | |

### Process for Preparing MRI Agent or Imaging Agent Loaded Platelets

In some embodiments, provided herein is a process for preparing MRI agent-loaded platelets, comprising: (a) providing platelets; and (b) treating the platelets with an MRI agent, to form MRI agent-loaded platelets.

In some embodiments, provided herein is a process for preparing MRI agent-loaded platelet derivatives, comprising: a) providing platelets; (b) treating the platelets with an MRI agent, to form MRI agent-loaded platelets; and (c) lyophilizing the MRI agent-loaded platelets, to form MRI agent-loaded platelet derivatives. In some embodiments, MRI agent-loaded platelet derivatives can be MRI agent-loaded FDPDs. In some embodiments, a process as disclosed herein can further comprise a step of rehydrating the MRI agent-loaded platelet derivatives.

In some embodiments, provided herein is a process for preparing MRI agent-loaded cryopreserved platelets, comprising: a) providing platelets; (b) treating the platelets with an MRI agent, to form MRI agent-loaded platelets; and (c) cryopreserving the MRI agent-loaded platelets, to form MRI agent-loaded cryopreserved platelets. In some embodiments, a process as disclosed herein can further comprise a step of thawing the MRI agent-loaded cryopreserved platelets.

In some embodiments, an MRI agent is present in a complex, referred herein as MRI agent complex as defined elsewhere in the present disclosure. In embodiments as disclosed herein, wherein an MRI agent is present in the form of MRI agent complex, the MRI agent complex is covalently bonded to platelets. In some embodiments, an MRI agent is coupled to a cell penetrating peptide. In embodiments as disclosed herein, wherein an MRI agent is coupled to a CPP, the CPP functions as a means to penetrate a cell membrane and facilitate uptake of MRI agent by the cells/platelets.

In some embodiments, platelets are isolated prior to treating (e.g., contacting) the platelets with an MRI agent.

Accordingly, in some embodiments, the methods for preparing an MRI agent-loaded platelets includes: step (a) isolating platelets, for example in a liquid medium; and step (b) treating the platelets with an MRI agent coupled to a cell penetrating peptide and with a loading buffer comprising a salt, a base, a loading agent, and optionally ethanol, to form the MRI agent-loaded platelets.

Accordingly, in some embodiments, the methods for preparing an MRI agent-loaded platelets includes: step (a) isolating platelets, for example in a liquid medium; and step (b) contacting the platelets with an MRI agent coupled to a cell penetrating peptide and with a loading buffer comprising a salt, a base, a loading agent, and optionally ethanol, to form the MRI agent-loaded platelets.

Accordingly, in some embodiments, the methods for preparing MRI agent-loaded platelets includes: step (a) isolating platelets, for example in a liquid medium; step (b) treating the platelets with an MRI agent coupled with a cell penetrating peptide to form a first composition; and step (c) treating the first composition with a buffer comprising a salt, a base, a loading agent, and optionally at least one organic solvent to form the MRI agent-loaded platelets.

Accordingly, in some embodiments, the methods for preparing MRI agent-loaded platelets includes: step (a) isolating platelets, for example in a liquid medium; step (b) contacting the platelets with an MRI agent coupled with a cell penetrating peptide to form a first composition; and step (c) contacting the first composition with a buffer comprising a salt, a base, a loading agent, and optionally at least one organic solvent to form the MRI agent-loaded platelets.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof.

Accordingly, in some embodiments, the methods for preparing MRI agent-loaded platelets includes: step (a) isolating platelets, for example in a liquid medium; step (b) treating the platelets with a buffer comprising a salt, a base, a loading agent, and optionally at least one organic solvent, to form a first composition; and step (c) treating the first composition with an MRI agent coupled to a cell penetrating peptide, to form the MRI agent-loaded platelets.

Accordingly, in some embodiments, the methods for preparing MRI agent-loaded platelets includes: step (a) isolating platelets, for example in a liquid medium; step (b) contacting the platelets with a buffer comprising a salt, a base, a loading agent, and optionally at least one organic solvent, to form a first composition; and step (c) contacting the first composition with an MRI agent coupled to a cell penetrating peptide, to form the MRI agent-loaded platelets.

In some embodiments, isolating platelets includes isolating platelets from blood.

In some embodiments, platelets are donor-derived platelets. In some embodiments, platelets are obtained by a process that includes an apheresis step. In some embodiments, platelets are fresh platelets. In some embodiments, platelets are stored platelets.

In some embodiments, platelets are derived in vitro. In some embodiments, platelets are derived or prepared in a culture prior to treating the platelets with an MRI agent. In some embodiments, preparing the platelets includes deriving or growing the platelets from a culture of megakaryocytes. In some embodiments, preparing the platelets includes deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, the methods for preparing MRI agent-loaded platelets includes: step (a) providing platelets; and step (b) treating the platelets with an MRI agent reagent, and with a loading buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; and step (b) contacting the platelets with an MRI agent reagent, and with a loading buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; step (b) treating the platelets with an MRI agent to form a first composition; and step (c) treating the first composition with a buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; step (b) contacting the platelets with an MRI agent to form a first composition; and step (c) contacting the first composition with a buffer comprising a salt, a base, a loading agent, and optionally at least one organic solvent, to form the MRI agent-loaded platelets.

Accordingly, in some embodiments, the methods for preparing MRI agent-loaded platelets includes: step (a) providing platelets; step (b) treating the platelets with a buffer comprising a salt, a base, a loading agent, and optionally at least one organic solvent, to form a first composition; and step (c) treating the first composition with an MRI agent, to form the MRI agent-loaded platelets.

Accordingly, in some embodiments, the methods for preparing MRI agent-loaded platelets includes: step (a) providing platelets; step (b) contacting the platelets with a buffer comprising a salt, a base, a loading agent, and optionally at least one organic solvent, to form a first composition; and step (c) contacting the first composition with an MRI agent, to form the MRI agent-loaded platelets.

In some embodiments, no solvent is used. Tus, in some embodiments, the method for preparing MRI agent-loaded platelets comprises:
a) isolating platelets, for example in a liquid medium;
and
b) treating the platelets with an MRI agent and with a loading buffer comprising a salt, a base, and a loading agent, to form the MRI agent-loaded platelets,
wherein the method does not comprise treating the platelets with an organic solvent such as ethanol.

In some embodiments, no solvent is used. Tus, in some embodiments, the method for preparing MRI agent-loaded platelets comprises:
a) isolating platelets, for example in a liquid medium;
   and
b) contacting the platelets with an MRI agent and with a loading buffer comprising a salt, a base, and a loading agent, to form the MRI agent-loaded platelets, wherein the method does not comprise contacting the platelets with an organic solvent such as ethanol.

Thus, in some embodiments, the method for preparing MRI agent-loaded platelets comprises:
a) isolating platelets, for example in a liquid medium;
b) treating the platelets with an MRI agent to form a first composition; and
c) treating the first composition with a buffer comprising a salt, a base, and a loading agent, to form the MRI agent-loaded platelets,
   wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) isolating platelets, for example in a liquid medium;
b) contacting the platelets with an MRI agent to form a first composition; and
c) contacting the first composition with a buffer comprising a salt, a base, and a loading agent, to form the MRI agent-loaded platelets, wherein the method does not comprise contacting the platelets with an organic solvent such as ethanol and the method does not comprise treating the first composition with an organic solvent such as ethanol.

Thus, in some embodiments, the method for preparing MRI agent-loaded platelets comprises:
a) isolating platelets, for example in a liquid medium;
b) treating the platelets with a buffer comprising a salt, a base, and a loading agent, to form a first composition; and
c) treating the first composition with an MRI agent, to form the MRI agent-loaded platelets.

Wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) isolating platelets, for example in a liquid medium;
b) contacting the platelets with a buffer comprising a salt, a base, and a loading agent, to form a first composition; and
c) contacting the first composition with an MRI agent, to form the MRI agent-loaded platelets.
   Wherein the method does not comprise contacting the platelets with an organic solvent such as ethanol and the method does not comprise contacting the first composition with an organic solvent such as ethanol.

In some embodiments, the method for preparing MRI agent-loaded platelets comprises:
a) providing platelets;
and
b) treating the platelets with an MRI agent-loaded and with a loading buffer comprising a salt, a base,
and a loading agent, to form the MRI agent-loaded platelets,
wherein the method does not comprise treating the platelets with an organic solvent such as ethanol.

In some embodiments, the method for preparing MRI agent-loaded platelets comprises:
a) providing platelets;
   and
b) contacting the platelets with an MRI agent-loaded and with a loading buffer comprising a salt, a base, and a loading agent, to form the MRI agent-loaded platelets,
wherein the method does not comprise contacting the platelets with an organic solvent such as ethanol.

Thus, in some embodiments, the method for preparing MRI agent-loaded platelets comprises:
a) providing platelets;
b) treating the platelets with an MRI agent to form a first composition; and
c) treating the first composition with a buffer comprising a salt, a base, and a loading agent, to form the MRI agent-loaded platelets,
wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) providing platelets;
b) contacting the platelets with an MRI agent to form a first composition; and
c) contacting the first composition with a buffer comprising a salt, a base, and a loading agent, to form the MRI agent-loaded platelets,
wherein the method does not comprise contacting the platelets with an organic solvent such as ethanol and the method does not comprise contacting the first composition with an organic solvent such as ethanol.

Thus, in some embodiments, the method for preparing MRI agent-loaded platelets comprises:
a) providing platelets;
b) treating the platelets with a buffer comprising a salt, a base, and a loading agent, to form a first composition; and
c) treating the first composition with an MRI agent, to form the MRI agent-loaded platelets.

Wherein the method does not comprise treating the platelets with an organic solvent such as ethanol and the method does not comprise treating the first composition with an organic solvent such as ethanol.

Thus, in some embodiments, the method for preparing MRI agent-loaded platelets comprises:
a) providing platelets;
b) contacting the platelets with a buffer comprising a salt, a base, and a loading agent, to form a first composition; and
c) contacting the first composition with an MRI agent, to form the MRI agent-loaded platelets,
wherein the method does not comprise contacting the platelets with an organic solvent such as ethanol and the method does not comprise contacting the first composition with an organic solvent such as ethanol.

In some embodiments, the loading agent is a saccharide. In some embodiments, the saccharide is a monosaccharide. In some embodiments, the saccharide is a disaccharide. In some embodiments, the saccharide is a non-reducing disaccharide. In some embodiments, the saccharide is sucrose, maltose, trehalose, glucose (e.g., dextrose), mannose, or xylose. In some embodiments, the loading agent is a starch.

As used herein, the term “MRI agent” is any agent that is suitable for magnetic resonance imaging described herein or known in the art.

In some embodiments, an MRI agent loaded into platelets is modified. For example, an MRI agent can be modified to increase its stability during the platelet loading process, while the MRI agent is loaded into the platelet, and/or after the MRI agent's release from a platelet. In some embodiments, the modified MRI agent's stability is increased with little or no adverse effect on its activity. For example, the modified MRI agent can have at least 70%, 75%, 80%, 85%, 90%, 95%, 96%, 97%, 98%, 99% or more of the activity of the corresponding unmodified MRI agent. In some embodiments, the modified MRI agent has 100% (or more) of the activity of the corresponding unmodified MRI agent. Various modifications that stabilize MRI agents are known in the art. In some embodiments, the MRI agent is stabilized by one or more of a stabilizing oligonucleotide (see, e.g., U.S. Application Publication No. 2018/0311176), a backbone/side chain modification (e.g., a 2-sugar modification such as a 2′-fluor, methoxy, or amine substitution, or a 2′-thio (—SH), 2′-azido (—N3), or 2′-hydroxymethyl (—CH2OH) modification), an unnatural nucleic acid substitution (e.g., an S-glycerol, cyclohexenyl, and/or threose nucleic acid substitution, an L-nucleic acid substitution, a locked nucleic acid (LNA) modification (e.g., the ribose moiety of an LNA nucleotide is modified with an extra bridge connecting the 2′ oxygen and 4′ carbon), conjugation with PEG, a nucleic acid bond modification or replacement (e.g., a phosphorothioate bond, a methylphosphonate bond, or a phosphorodiamidate bond), a reagent or reagents (e.g., intercalating agents such as coralyne, neomycin, and ellipticine; also see US Publication Application Nos. 2018/0312903 and 2017/0198335, each of which are incorporated herein by reference in their entireties, for further examples of stabilizing reagents).

In some embodiments, an MRI agent loaded into platelets is modified to include an imaging agent. For example, an MRI agent can be modified with an imaging agent in order to image the MRI agent loaded platelet in vivo. In some embodiments, an MRI agent can be modified with two or more imaging agents (e.g., any two or more of the imaging agents described herein). In some embodiments, an MRI agent loaded into platelets is modified with a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ⁵²Fe, ⁵⁵Co, ⁹⁴Tc or ⁶⁸Ga; or gamma-emitters such as ¹⁷¹Tc, ¹¹¹In, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to ¹²³I, ¹³¹I or ⁷⁷Br. For example, a positron-emitting radioactive non-metal can include, but is not limited to ¹¹C, ¹³N, ¹⁵O, ¹⁷F, ¹⁸F, ⁷⁵Br, ⁷⁶Br or ¹²⁴I. For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y, ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, an MRI agent loaded into platelets can be associated with gold or other equivalent metal particles (such as nanoparticles). For example, a metal particle system can include, but is not limited to gold nanoparticles (e.g., Nanogold™).

In some embodiments, an MRI agent loaded into platelets that is modified with an imaging agent is imaged using an imaging unit. The imaging unit can be configured to image the MRI agent loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen, Z., et. Al., Advance of Molecular Imaging Technology and Targeted Imaging Agent in Imaging and Therapy, Biomed Res Int., 819324, doi: 10.1155/2014/819324 (2014) have described various imaging techniques and which is incorporated by reference herein in its entirety.

In some embodiments, such as embodiments wherein the platelets are treated (e.g., contacted) with the MRI agent and the loading buffer sequentially as disclosed herein, the MRI agent may be loaded in a liquid medium that may be modified to change the proportion of media components or to exchange components for similar products, or to add components necessary for a given application.

In some embodiments, the loading buffer and/or the liquid medium include one or more of a) water or a saline solution, b) one or more additional salts, or c) a base. In some embodiments, the loading buffer, and/or the liquid medium, may include one or more of a) DMSO, b) one or more salts, or d) a base.

In some embodiments, the loading agent is loaded into the platelets in the presence of an aqueous medium. In some embodiments, the loading agent is loaded in the presence of a medium comprising DMSO. As an example, one embodiment of the methods herein includes treating (e.g., contacting) platelets with an MRI agent coupled with a cell penetrating peptide and with an aqueous loading buffer comprising a salt, a base, a loading agent, and optionally at least one organic solvent, to form the MRI agent-loaded platelets. As an example, one embodiment of the methods herein includes treating (e.g., contacting) platelets with an MRI agent coupled with a cell penetrating peptide and with a loading buffer comprising DMSO and comprising a salt, a base, a loading agent, and optionally ethanol, to form the MRI agent-loaded platelets.

In some embodiments, the loading buffer and/or the liquid medium, include one or more salts selected from phosphate salts, sodium salts, potassium salts, calcium salts, magnesium salts, and any other salt that can be found in blood or blood products, or that is known to be useful in drying platelets, or any combination of two or more of these.

Preferably, these salts are present in the composition at an amount that is about the same as is found in whole blood.

In some embodiments, the MRI agent-loaded platelets are prepared by incubating the platelets with the MRI agent in the liquid medium for different durations at or at different temperatures from about 15-45° C., or about 22° C. In some embodiments, the MRI agent-loaded platelets are prepared by incubating the platelets with the MRI agent in the liquid medium at a temperature from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the MRI agent-loaded platelets are prepared by incubating the platelets with the MRI agent in the liquid medium at a temperature from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the MRI agent-loaded platelets are prepared by incubating the platelets with the MRI agent in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs.

In some embodiments, the platelets are at a concentration from about 1,000 platelets/μl to about 10,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 200,000,000 platelets/μl.

In some embodiments, the MRI agent-loaded platelets are prepared by incubating the platelets with the MRI agent in the liquid medium for different durations. The step of incubating the platelets to load one or more MRI agent(s) includes incubating the platelets for a time suitable for loading, as long as the time, taken in conjunction with the temperature, is sufficient for the MRI agent to come into contact with the platelets and, preferably, be incorporated, at least to some extent, into the platelets. For example, in some embodiments, the MRI agent-loaded platelets are prepared by incubating the platelets with the MRI agent in the liquid medium for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the MRI agent-loaded platelets are prepared by incubating the platelets with the MRI agent in the liquid medium for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the MRI agent-loaded platelets are prepared by incubating the platelets with the MRI agent in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs.

In some embodiments, the MRI agent-loaded platelets are prepared by incubating the platelets with the MRI agent in the liquid medium at different temperatures. The step of incubating the platelets to load one or more MRI agent(s), includes incubating the platelets with the MRI agent in the liquid medium at a temperature that, when selected in conjunction with the amount of time allotted for loading, is suitable for loading. In general, the platelets with the MRI agent in the liquid medium are incubated at a suitable temperature (e.g., a temperature above freezing) for at least a sufficient time for the MRI agent to come into contact with the platelets. In some embodiments, incubation is conducted at 22° C. In certain embodiments, incubation is performed at 4° C. to 45° C., such as 15° C. to 42° C. For example, in some embodiments, incubation is performed from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for 110 to 130 (e.g., 120) minutes and for as long as 24-48 hours.

In some embodiments of the methods of preparing MRI agent-loaded platelets disclosed herein, the methods further include acidifying the platelets, or pooled platelets, to a pH of about 6.0 to about 7.4, prior to a treating (e.g., contacting) step disclosed herein. In some embodiments, the methods include acidifying the platelets to a pH of about 6.5 to about 6.9. In some embodiments, the methods include acidifying the platelets to a pH of about 6.6 to about 6.8. In some embodiments, the acidifying includes adding to the pooled platelets a solution comprising Acid Citrate Dextrose.

In some embodiments, the platelets are isolated prior to a treating (e.g., contacting) step. In some embodiments, the methods further include isolating platelets by using centrifugation. In some embodiments, the centrifugation occurs at a relative centrifugal force (RCF) of about 800 g to about 2000 g. In some embodiments, the centrifugation occurs at relative centrifugal force (RCF) of about 1300 g to about 1800 g. In some embodiments, the centrifugation occurs at relative centrifugal force (RCF) of about 1500 g. In some embodiments, the centrifugation occurs for about 1 minute to about 60 minutes. In some embodiments, the centrifugation occurs for about 10 minutes to about 30 minutes. In some embodiments, the centrifugation occurs for about 20 minutes.

In some embodiments, the platelets are at a concentration from about 1,000 platelets/μl to about 10,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 2,000,000 platelets/μl.

In some embodiments, the buffer is a loading buffer comprising the components as listed in Buffer A herein. In some embodiments, the loading buffer includes one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the loading buffer includes from about 0.5 mM to about 100 mM of the one or more salts. In some embodiments, the loading buffer includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the loading buffer includes about 5 mM, about 75 mM, or about 80 mM of the one or more salts.

In some embodiments, the loading buffer includes one or more buffers, e.g., N-2-hydroxyethylpiperazine-N′-2-ethanesulfonic acid (HEPES), and/or sodium-bicarbonate (NaHCO₃). In some embodiments, the loading buffer includes from about 5 to about 100 mM of the one or more buffers. In some embodiments, the loading buffer includes from about 5 to about 50 mM (e.g., from about 5 mM to about 40 mM, from about 8 mM to about 30 mM, about 10 mM to about 25 mM) about of the one or more buffers. In some embodiments, the loading buffer includes about 10 mM, about 20 mM, about 25 mM, or about 30 mM of the one or more buffers.

In some embodiments, the loading buffer includes one or more saccharides, such as monosaccharides and disaccharides, including sucrose, maltose, trehalose, glucose, mannose, dextrose, and xylose. In some embodiments, the loading buffer includes from about 10 mM to about 1,000 mM of the one or more saccharides. In some embodiments, the loading buffer includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM.

In some embodiments, the loading buffer includes adding an organic solvent, such as ethanol, to the loading solution. In such a loading buffer, the solvent can range from about 0.1% (v/v) to about 5.0% (v/v), such as from about 0.3% (v/v) to about 3.0% (v/v), or from about 0.5% (v/v) to about 2% (v/v).

In some embodiments, the MRI agent includes one MRI agent. In some embodiments, the MRI agent includes one or more MRI agents.

In some embodiments, the methods further include incubating the MRI agent in the presence of the loading buffer prior to the treatment (e.g., contacting) step. In some embodiments, the methods further include incubating the loading buffer and a solution comprising the MRI agent and water at about 37° C. using different incubation periods. In some embodiments, the solution includes a concentration of about 0.1 nM to about 150 μM of the MRI agent. In some embodiments, the solution includes a concentration of about 1 nM to about 100 μM of the MRI agent. In some embodiments, the solution includes a concentration of about 10 nM to 50 μM of the MRI agent. In some embodiments, the solution includes a concentration of about 500 nM to about 50 μM of the MRI agent. In some embodiments, the solution includes a concentration of about 1 μM to about 30 μM of the MRI agent. In some embodiments, the solution includes a concentration of about 10 μM to about 30 μM of the MRI agent. In some embodiments, the incubation of the MRI agent in the presence of the loading buffer is performed from about 1 minute to about 2 hours. In some embodiments, the incubation is performed at an incubation period of from about 5 minutes to about 1 hour. In some embodiments, the incubation is performed at an incubation period of from about 10 minutes to about 30 minutes. In some embodiments, the incubation is performed at an incubation period of about 20 minutes.

In some embodiments, the methods further include incubating the MRI agent in the presence the loading buffer prior to the treatment (e.g., contacting) step.

In some embodiments, the methods further include mixing the platelets and the coupled cell penetrating peptide (CPP) and MRI agent (e.g., MRI agent-CPP) in the presence of the loading buffer at about room temperature (e.g., at about 20° C. to about 25° C.).

As used herein, “coupled,” describes attaching (e.g., complexing, conjugating) a cell penetrating peptide to an MRI agent. The coupling of the cell penetrating peptide and the MRI agent can be a covalent or a non-covalent coupling.

In some embodiments, the incubation is performed at an incubation period of from about 5 minutes to about 12 hours. In some embodiments, the incubation is performed at an incubation period of from about 10 minutes to about 6 hours. In some embodiments, the incubation is performed at an incubation period of from about 15 minutes to about 3 hours. In some embodiments, the incubation is performed at an incubation period of about 2 hours. In some embodiments, the final product includes platelets and the MRI-agent CPP at a volume ratio of 10:1, with a range in volume ratio of about 1 to about 50.

In some embodiments, the concentration of MRI agent in the MRI agent-loaded platelets is from about 0.1 nM to about 10 μM. In some embodiments, the concentration of MRI agent in the MRI agent-loaded platelets is from about 1 nM to about 1 μM. In some embodiments, the concentration of MRI agent in the MRI agent-loaded platelets is from about 10 nM to 10 JIM. In some embodiments, the concentration of MRI agent in the MRI-loaded platelets is about 100 nM.

In some embodiments, the methods further include drying the MRI agent-loaded platelets. In some embodiments, the drying step includes freeze-drying the MRI agent-loaded platelets. In some embodiments, the methods further include rehydrating the MRI agent-loaded platelets obtained from the drying step.

In some embodiments, MRI agent-loaded platelets are prepared by using any of the variety of methods provided herein.

In some embodiments, rehydrated MRI agent-loaded platelets are prepared by any one method comprising rehydrating the MRI agent-loaded platelets provided herein.

The MRI agent-loaded platelets may be used, for example, in therapeutic applications as disclosed herein. Additionally or alternatively, the MRI agent-loaded platelets may be employed in functional assays. In some embodiments, the MRI agent-loaded platelets are cold stored, cryopreserved, or lyophilized (to produce thrombosomes) prior to use in therapy or in functional assays. In some embodiments, process for loading MRI agent on to platelets as disclosed herein to form MRI agent-loaded platelets/platelet derivatives/cryopreserved platelets/FDPDs is also applicable for loading imaging agent on to platelets that does not involve prior loading with an MRI agent. In some embodiments, provided herein is a method for preparing a composition comprising MRI agent-loaded cryopreserved platelets or MRI agent-loaded platelet derivatives in a powder, comprising: (a) providing platelets; (b) contacting the platelets with an MRI agent complex such that the MRI agent complex is covalently bound to the platelets, to form MRI agent-loaded platelets; and (c) cryopreserving or lyophilizing the MRI agent-loaded platelets to form the composition comprising the MRI agent-loaded cryopreserved platelets or the MRI agent-loaded platelet derivatives. In some embodiments, contacting the platelets with MRI agent complex such that the MRI agent complex is covalently bound to the platelets, to form MRI agent-loaded platelets is done in the presence of a buffer comprising a salt, a base, a loading agent, and optionally at least one organic solvent. In some embodiments, a method can comprise steps that make use of lyophilized or cryopreserved platelets as a starting material for loading.

In some embodiments, provided herein is a process/method for forming MRI agent-loaded platelets/platelet derivatives/cryopreserved platelets/FDPDs comprising: (a) providing platelets; (b) contacting the platelets with an MRI agent coupled to a cell penetrating peptide, and a buffer comprising a salt, a base, a loading agent, and optionally at least one organic solvent, to form MRI agent-loaded platelets; and (c) cryopreserving the MRI agent-loaded platelets to form the composition comprising the MRI agent-loaded cryopreserved platelets or lyophilizing the MRI agent-loaded platelets to form the composition comprising the MRI agent-loaded platelet derivatives.

A person of skill in the art can use any known method for isolating platelets to be used in step (a) or the step of providing the platelets of the process as disclosed herein. In some embodiments, platelets can be provided by isolating the platelets using TFF method as disclosed herein. In some embodiments, a process using the platelets isolated/purified by TFF method and that further undergoes lyophilization after loading with MRI agents are also called as MRI agent-loaded TFF-FDPDs or MRI agent-loaded FDPDs. In some embodiments, platelets can be provided by isolating the platelets using a centrifugation based method as disclosed herein. In some embodiments, a process using the platelets isolated/purified by centrifugation based method and that further undergoes lyophilization after loading with MRI agents are also called as MRI agent-loaded FDPDs or MRI agent-loaded platelet derivatives. In some embodiments, a process using the platelets isolated/purified by centrifugation based method or by TFF method and that further undergoes cryopreservation after loading with MRI agents are also called as MRI agent-loaded cryopreserved platelets.

In some embodiments that include a process/method for forming MRI agent-loaded platelets/platelet derivatives/cryopreserved platelets/FDPDs, platelets that are to be used for loading with MRI agents are isolated/purified using centrifugation based methods. In some embodiments, a centrifugation based method can include the steps of: (a) pooling apheresis platelet units to form pooled platelet rich plasma; (b) acidifying the pooled platelet rich plasma to form pooled acidified pooled platelet rich plasma; (c) centrifuging the acidified pooled platelet rich plasma and discarding the supernatant comprising platelet pooled plasma; and (d) resuspending the remaining solution obtained after centrifuging, in a buffer comprising at least one saccharide, at least one salt, and optionally at least one organic solvent. In some embodiments, resuspending can also be done in a buffer that further comprises at least one cryoprotectant, or platelets can be incubated with the buffer comprising at least one saccharide, isolated out of that solution using for example, TFF or centrifugation, and resuspended in a buffer comprising a cryoprotectant. Exemplary cryoprotectants include but are not limiting to, polysugars, Ficoll, DMSO, bovine serum albumin, human serum albumin, dextran, polyvinyl 64ehydrate64e (PVP), starch, hydroxyethyl starch (HES). In some illustrative embodiments, polysugar can comprise polysucrose, Ficoll 70, and Ficoll 400.

In some embodiments, provided herein is a process/method for forming MRI agent-loaded platelets/platelet derivatives/cryopreserved platelets/FDPDs comprising: (a) providing cryopreserved platelets or rehydrated platelet derivatives; and (b) contacting the cryopreserved platelets or the rehydrated platelet derivatives with an MRI agent coupled to a cell penetrating peptide, to form the composition comprising MRI agent-loaded cryopreserved platelets or MRI agent-loaded platelet derivatives.

### Imaging Agent-Loaded Platelets

Imaging agents such as a radioactive metal ion, an MRI agent (such as, but not limiting to, a paramagnetic metal ion, a superparamagnetic metal ion, and a diamagnetic metal ion), a gamma-emitting radioactive halogen, a positron-emitting radioactive non-metal, a hyperpolarized NMR-active nucleus, a reporter suitable for in vivo optical imaging, or a beta-emitter suitable for intravascular detection can be of importance in the field of diagnostics and therapy. Platelets, platelet derivatives such as FDPDs, or cryopreserved platelets can be loaded with any of the imaging agents known in the art to form imaging agent-loaded platelets, platelet derivatives, or cryopreserved platelets. For example, a radioactive metal ion can include, but is not limited to, positron emitters such as ⁵⁴Cu, ⁴⁸V, ⁵²Fe, ⁵⁵Co, ⁹⁴Tc or ⁶⁸Ga; or gamma-emitters such as ¹⁷¹Tc, ¹¹¹In, ¹¹³In, or ⁶⁷Ga. In some embodiments, imaging agent can include radiometal nuclides that serve as diagnostic markers in molecular imaging or can be used in therapeutic settings. In some embodiments, an imaging agent can be an Indium isotope, such as, In³⁺. In some embodiments, an imaging agent can be an Yttrium isotope, such as, Y³⁺. In some embodiments, an imaging agent can be a Lutetium isotope, such as, Lu³⁺. In some embodiments, an imaging agent can be a copper isotope, such as, Cu²⁺. Any appropriate imaging agent, such as, radiometal nuclides as disclosed in the publication—Wangler, B., et al. “Chelating agents and their use in radiopharmaceutical sciences.” Mini Reviews in Medicinal Chemistry 11.11 (2011): 968-983 (Wangler et al 2011) can be used for the purposes of the present invention. The publication Wangler et al 2011 is also incorporated herein in its entirety.

In some embodiments, an imaging agent can be present in a complex along with a chelator for loading on to platelets. A chelator acts to reduce the toxicity of an imaging agent that is been loaded on to the platelets. A person of skill in the art can use any chelator known in the art for forming a complex with an imaging agent that further can be loaded onto platelets. Any appropriate chelator as disclosed in Wangler et al 2011 can be used for the purposes of the present invention. In some embodiments, any of chelators as disclosed elsewhere in this invention can be used for forming a complex with an imaging agent.

In some embodiments, imaging agent-loaded platelets/FDPDs/cryopreserved platelets can be prepared using loading with a CPP as disclosed for MRI agent-loaded platelets/FDPDs/cryopreserved platelets as disclosed elsewhere in this disclosure. A person of skill in the art can appreciate that any type of CPP that can efficiently cross the plasma membrane can be used for loading platelets with an imaging agent. In some embodiments, a CPP is any type of CPP that is disclosed for MRI agent-loaded platelets/FDPDs/cryopreserved platelets elsewhere in this invention.

In some embodiments, imaging agent-loaded platelets/FDPDs/cryopreserved platelets can be prepared using an imaging agent-complex that comprises an imaging agent, a chelator for reducing toxicity of the imaging agent, and a linker for covalently binding to a protein molecule on the surface of platelets. Any appropriate linker known in the art can be used for preparing imaging agent-loaded platelets/FDPDs/cryopreserved platelets. In some embodiments, a linker can be selected from the group consisting of a compound having sulfhydryl reactive groups, such as maleimides and haloacetyl derivatives, amine reactive groups, such as isothiocyanates, succinimidyl esters, and sulfonyl halides, and carbodiimide reactive groups, such as carboxyl and amino groups. In illustrative embodiments, a linker is a compound having an amine reactive group, such as, succinimidyl ester, such as, N-Hydroxysuccinimide (NHS) ester.

### Process for Preparing a Platelet Derivative Composition

In some embodiments, the platelets or pooled platelets may be acidified to a pH of about 5.5 to about 8.0 prior to TFF or being diluted with the preparation agent. In some embodiments, the method comprises acidifying the platelets to a pH of about 6.5 to about 6.9. In some embodiments, the method comprises acidifying the platelets to a pH of about 6.6 to about 6.8. In some embodiments, the method comprises acidifying the platelets to a pH of about 6.6 to 7.5. In some embodiments, the acidifying comprises adding to the pooled platelets a solution comprising Acid Citrate Dextrose (ACD).

In some embodiments, the platelets are isolated prior to the step comprising tangential flow filtration (TFF) or being diluted with the preparation agent. In some embodiments, the method further comprises isolating platelets by using centrifugation. In some embodiments, the centrifugation occurs at a relative centrifugal force (RCF) of about 1000×g to about 2000×g. In some embodiments, the centrifugation occurs at relative centrifugal force (RCF) of about 1300×g to about 1800×g. In some embodiments, the centrifugation occurs at relative centrifugal force (RCF) of about 1500×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, platelets are isolated, for example in a liquid medium, prior to treating a subject.

In some embodiments, platelets are donor-derived platelets. In some embodiments, platelets are obtained by a process that comprises an apheresis step. In some embodiments, platelets are pooled platelets.

In some embodiments, platelets are pooled from a plurality of donors. Such platelets pooled from a plurality of donors may be also referred herein to as pooled platelets. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35. Pooled platelets can be used to make any of the compositions described herein. The platelets can be pooled wherein the platelets are donated by human subjects. In some other embodiments, the donor can be a non-human animal. In some embodiments, the donor can be a canine subject. In some embodiments, the donor can be an equine subject. In some embodiments, the donor can be a feline subject.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PSCs), including embryonic stem cells (ESCs) and/or induced pluripotent stem cells (iPSCs).

Accordingly, in some embodiments, platelets or platelet derivatives (e.g., thrombosomes) are prepared prior to treating a subject as described herein. In some embodiments, the platelets or platelet derivatives (e.g., thrombosomes) are lyophilized. In some embodiments, the platelets or platelet derivatives (e.g., thrombosomes) are cryopreserved. For example, in some embodiments, the platelets or platelet derivatives can be cryopreserved in plasma and DMSO (e.g., 3-9% DMSO (e.g., 6% DMSO)). In some embodiments, the platelets or platelet derivatives are cryopreserved as described in U.S. Patent Application Publication No. 2020/0046771 A1, published on Feb. 13, 2020, incorporated herein by reference in its entirety.

In some embodiments, platelets (e.g., apheresis platelet, platelets isolated from whole blood, pooled platelets, or a combination thereof) form a suspension in a preparation agent comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, the method further comprises drying the platelets or platelet derivatives (e.g., thrombosomes). In some embodiments, the drying step comprises lyophilizing the platelets or platelet derivatives (e.g., thrombosomes). In some embodiments, the drying step comprises freeze-drying the platelets or platelet derivatives (e.g., thrombosomes). In some embodiments, the method further comprises rehydrating the platelets or platelet derivatives (e.g., thrombosomes) obtained from the drying step.

In some embodiments, the platelets or platelet derivatives (e.g., thrombosomes) are cold stored, cryopreserved, or lyophilized (e.g., to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and 8,097,403. An exemplary spray-drying method includes: combining nitrogen, as a drying gas, with a loading buffer according to the present disclosure, then introducing the mixture into GEA Mobile Minor spray dryer from GEA Processing Engineering, Inc. (Columbia MD, USA), which has a Two-Fluid Nozzle configuration, spray drying the mixture at an inlet temperature in the range of 150C to 190FC, an outlet temperature in the range of 65C to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | Step | Temp. Set | Type | Duratio | | Pressure Set |
|---|---|---|---|---|---|---|
| Freezing Step | F1 | −50° C. | Ramp | Var | | N/A |
| | F2 | −50° C. | Hold | 3 | Hrs | N/A |
| Vacuum Pulldown | F3 | −50° | Hold | Var | | N/A |
| Primary Dry | P1 | −40° | Hold | 1.5 | Hrs | 0 mT |
| | P2 | −35° | Ramp | 2 | Hrs | 0 mT |
| | P3 | −25° | Ramp | 2 | Hrs | 0 mT |
| | P4 | −17° C. | Ramp | 2 | Hrs | 0 mT |
| | P5 | 0° C. | Ramp | 1.5 | Hrs | 0 mT |
| | P6 | 27° C. | Ramp | 1.5 | Hrs | 0 mT |
| | P7 | 27° C. | Hold | 16 | Hrs | 0 mT |
| Secondary Dry | S1 | 27° C. | Hold | >8 | Hrs | 0 mT |

In some embodiments, the step of drying the MRI agent-loaded platelets that are obtained as disclosed herein, such as the step of freeze-drying the MRI agent-loaded platelets that are obtained as disclosed herein, includes incubating the platelets with a lyophilizing agent. In some embodiments, the lyophilizing agent is polysucrose. In some embodiments, the lyophilizing agent is a non-reducing disaccharide. Accordingly, in some embodiments, the methods for preparing MRI agent-loaded platelets further include incubating the MRI agent-loaded platelets with a lyophilizing agent. In some embodiments, the lyophilizing agent is a saccharide. In some embodiments, the saccharide is a disaccharide, such as a non-reducing disaccharide.

In some embodiments, the platelets and/or platelet derivatives are incubated with a lyophilizing agent for a sufficient amount of time and at a suitable temperature to incubate the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl 69ehydrate69e (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., polysucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%. In some embodiments, polysucrose is used in the range of 2% to 8%%, or 2.25-7.75%, or 2.5-7.5%, or 2.5-6.5%. In an exemplary embodiment, the composition comprises 3% polysucrose. In another exemplary embodiment, the composition comprises 6% polysucrose. In some embodiments of the composition, wherein the composition comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 Da to 400,000 Da. In some embodiments, polysucrose has a molecular weight in the range of 80,000 Da to 350,000 Da, or 100,000 Da to 300.00 Da. In some exemplary embodiments, polysucrose has a molecular weight in the range of 120,000 Da to 200,000 Da. In some exemplary embodiments, polysucrose has a molecular weight of 150,000 Da, or 160,000 Da, or 170,000 Da, or 180,000 Da, 190,000 Da, or 200,000 Da.

In some embodiments, the process for preparing a composition includes adding an organic solvent, such as ethanol, to the loading solution. In such a loading solution, the solvent can range from 0.1% to 5.0% (v/v).

Within the process provided herein for making the compositions provided herein, addition of the lyophilizing agent can be the last step prior to drying. However, in some embodiments, the lyophilizing agent is added at the same time or before the MRI agent, the cryoprotectant, or other components of the loading composition. In some embodiments, the lyophilizing agent is added to the loading solution, thoroughly mixed to form a drying solution, dispensed into a drying vessel (e.g., a glass or plastic serum vial, a lyophilization bag), and subjected to conditions that allow for drying of the solution to form a dried composition.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In some embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

The step of incubating the platelets to load them with a cryoprotectant or as a lyophilizing agent includes incubating the platelets for a time suitable for loading, as long as the time, taken in conjunction with the temperature, is sufficient for the cryoprotectant or lyophilizing agent to come into contact with the platelets and, preferably, be incorporated, at least to some extent, into the platelets. In embodiments, incubation is carried out for about 1 minute to about 180 minutes or longer.

The step of incubating the platelets to load them with a cryoprotectant or lyophilizing agent includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant or lyophilizing agent to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6 cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mLto about 9.0 cm²/mL, from about 3.1 cm²/mL to about 8.9 cm²/mL, from about 3.2 cm²/mLto about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, the lyophilizing agent as disclosed herein may be a high molecular weight polymer. By “high molecular weight” it is meant a polymer having an average molecular weight of about or above 70 kDa and up to 1,000,000 kDa Non-limiting examples are polymers of sucrose and epichlorohydrin (polysucrose). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrrolidone (PVP), starch, and hydroxyethyl starch (HES).

In some embodiments, the loading buffer includes an organic solvent, such as an alcohol (e.g., ethanol). In such a loading buffer, the amount of solvent can range from 0.1% to 5.0% (v/v).

In some embodiments, the MRI agent-loaded platelets prepared as disclosed herein have a storage stability that is at least about equal to that of the platelets prior to the loading of the MRI agent.

The loading buffer may be any buffer that is non-toxic to the platelets and provides adequate buffering capacity to the solution at the temperatures at which the solution will be exposed during the process provided herein. Thus, the buffer may include any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may include one or more of the following buffers: propane-1,2,3-tricarboxylic (tricarballylic); benzenepentacarboxylic; maleic; 2,2-dimethylsuccinic; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-aminoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-tricarboxylic (beta-methytricarballylic); 2-(N-morpholino)propane-sulfonic acid (MOPS); phosphoric; and N-tris(hydroxymethyl)methyl-2-aminoethane sulfonic acid (TES).

### Different Steps in Processing of a Starting Material, and TFF

In some embodiments, the method can include an initial dilution step, for example, a starting material (e.g., an unprocessed blood product (e.g., donor apheresis material (e.g., pooled donor apheresis material)) can be diluted with a preparation agent (e.g., any of the preparation agents described herein) to form a diluted starting material. In some cases, the initial dilution step can include dilution with a preparation agent with a mass of preparation agent equal to at least about 10% of the mass of the starting material (e.g., at least about 15%, 25%, 50%, 75%, 100%, 150%, or 200% of the mass of the starting material. In some embodiments, an initial dilution step can be carried out using the TFF apparatus.

In some embodiments, the method can include concentrating (e.g., concentrating platelets) (e.g., concentrating a starting material or a diluted starting material) to form a concentrated platelet composition. For example, concentrated can include concentrating to a about 1000×10³ to about 4000×10³ platelets/μL (e.g., about 1000×10³ to about 2000×10³, about 2000×10³ to about 3000×10³, or about 4000×10³ platelets/μL). In some embodiments, a concentration step can be carried out using the TFF apparatus.

The concentration of platelets or platelet derivatives (e.g., thrombosomes) can be determined by any appropriate method. For example, a counter can be used to quantitate concentration of blood cells in suspension using impedance (e.g., a Beckman Coulter AcT 10 or an AcT diff 2).

In some embodiments, TFF can include diafiltering (sometimes called “washing”) of a starting material, a diluted starting material, a concentrated platelet composition, or a combination thereof. In some embodiments, diafiltering can include washing with at least 2 (e.g., at least 3, 4, 5, 6, 7, 8, 9, 10, or more) diavolumes. In some embodiments, TFF can include buffer exchange. In some embodiments, a buffer can be used in TFF. A buffer can be any appropriate buffer. In some embodiments, the buffer can be a preparation agent (e.g., any of the preparation agents described herein). In some embodiments, the buffer can be the same preparation agent as was used for dilution. In some embodiments, the buffer can be a different preparation than was used for dilution. In some embodiments, a buffer can Include a lyophilizing agent, including a buffering agent, a base, a loading agent, optionally a salt, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof. A buffering agent can be any appropriate buffering agent. In some embodiments, a buffering agent can be HEPES ((4-(2-hydroxyethyl)-1-piperazineethanesulfonic acid). A base can be any appropriate base. In some embodiments, a base can be sodium bicarbonate. In some embodiments, a saccharide can be a monosaccharide. In some embodiments, a loading agent can be a saccharide. In some embodiments, a saccharide can include sucrose, maltose, trehalose, glucose (e.g., dextrose), mannose, or xylose. In some embodiments, a monosaccharide can be trehalose. In some embodiments, the loading agent can include polysucrose. A salt can be any appropriate salt. In some embodiments, a salt can be selected from the group consisting of sodium chloride (NaCl), potassium chloride (KCl), or a combination thereof.

In some embodiments, a membrane with a pore size of about 0.1 μm to about 1 μm (e.g., about 0.1 μm to about 1 μm, about 0.1 μm to about 0.5 μm, about 0.2 to about 0.45 μm, about 0.45 to about 1 μm, about 0.1 μm, about 0.2 μm, about 0.45 μm, about 0.65 μm, or about 1 μm) can be used in TFF. A membrane can be made from any appropriate material. In some cases, a membrane can be a hydrophilic membrane. In some embodiments, a membrane can be a hydrophobic membrane. In some embodiments, a membrane with a nominal molecular weight cutoff (NMWCO) of at least about 100 kDa (e.g., at least about 200, 300 kDa, 500 kDa, or 1000 kDa) can be used in TFF. The TFF can be performed with any appropriate pore size within the range of 0.1 μm to 1.0 μm with the aim of reducing the microparticles content in the composition and increasing the content of platelet derivatives in the composition. A skilled artisan can appreciate the required optimization of the pore size in order to retain the platelet derivatives and allow the microparticles to pass through the membrane. The pore size in illustrative embodiments, is such that the microparticles pass through the membrane allowing the TFF-treated composition to have less than 5% microparticles. The pore size in illustrative embodiments is such that a maximum of platelet derivatives gets retained in the process allowing the TFF-treated composition to have a concentration of the platelet derivatives in the range of 100×103 to 20,000×103. The pore size during the TFF process can be exploited to obtain a higher concentration of platelet derivatives in the platelet derivative composition such that a person administering the platelet derivatives to a subject in need has to rehydrate/reconstitute fewer vials, therefore, being efficient with respect to time and effort during the process of preparing such platelet derivatives for a downstream procedure, for example a method of treating provided herein. TFF can be performed at any appropriate temperature. In some embodiments, TFF can be performed at a temperature of about 20° C. to about 37° C. (e.g., about 20° C. to about 25° C., about 20° C. to about 30° C., about 25° C. to about 30° C., about 30° C. to about 35° C., about 30° C. to about 37° C., about 25° C. to about 35° C., or about 25° C. to about 37° C.). In some embodiments, TFF can be carried out at a flow rate (e.g., a circulating flow rate) of about 100 ml/min to about 800 ml/min (e.g., about 100 to about 200 ml/min, about 100 to about 400 ml/min, about 100 to about 600 ml/min, about 200 to about 400 ml/min, about 200 to about 600 ml/min, about 200 to about 800 ml/min, about 400 to about 600 ml/min, about 400 to about 800 ml/min, about 600 to about 800 ml/min, about 100 ml/min, about 200 ml/min, about 300 ml/min, about 400 ml/min, about 500 ml/min, about 600 ml/min, about 700 ml/min, or about 800 ml/min).

In some embodiments, TFF can be performed until a particular endpoint is reached, forming a TFF-treated composition. An endpoint can be any appropriate endpoint. In some embodiments, an endpoint can be a percentage of residual plasma (e.g., less than or equal to about 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of residual plasma). In some embodiments, an endpoint can be a relative absorbance at 280 nm (A280). For example, an endpoint can be an A280 (e.g., using a path length of 0.5 cm) that is less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the A280 (e.g., using a path length of 0.5 cm) prior to TFF (e.g., of a starting material or of a diluted starting material). In some embodiments, an A280 can be relative to a system that measures 7.5% plasma=1.66 AU. In some embodiments, an instrument to measure A280 can be configured as follows: a 0.5 cm gap flow cell can be attached to the filtrate line of the TFF system. The flow cell can be connected to a photometer with fiber optics cables attached to each side of the flow cell (light source cable and light detector cable). The flow cell can be made with a silica glass lens on each side of the fiber optic cables. Apart from the relative protein concentration of proteins in the aqueous medium, the protein concentration in the aqueous medium can also be measured in absolute terms. In some embodiments, the protein concentration in the aqueous medium is less than or equal to 15%, or 14%, or 13%, or 12%, or 11%, or 10%, or 9%, or 8%, or 7%, or 6%, or 5%, or 4%, or 3%, or 2%, or 1%, or 0.1%, or 0.01%. In some exemplary embodiments, the protein concentration is less than 3% or 4%. In some embodiments, the protein concentration is in the range of 0.01-15%, or 0.1-15%, or 1-15%, or 1-10%, or 0.01-10%, or 3-12%, or 5-10% in the TFF-treated composition. In some embodiments, an endpoint can be an absolute A280 (e.g., using a path length of 0.5 cm). For example, an endpoint can be an A280 that is less than or equal to 2.50 AU, 2.40 AU, 2.30 AU, 2.20 AU, 2.10 AU, 2.0 AU, 1.90 AU, 1.80 AU, or 1.70 AU (e.g., less than or equal to 1.66, 1.6, 1.5, 1.4, 1.3, 1.2, 1.1, 1.0, 0.9, 0.8, 0.7, 0.6, 0.5, 0.4, 0.3, 0.2, or 0.1 AU) (e.g., using a path length of 0.5 cm). In some embodiments, a percentage of residual plasma, a relative A280, or an A280 can be determined based on the aqueous medium of a composition comprising platelets and an aqueous medium. In some embodiments, a percentage of residual plasma can be determined based on a known correlation to an A280. In some embodiments, an endpoint can be a platelet concentration, as TFF can include concentration or dilution of a sample (e.g., using a preparation agent). For example, an endpoint can be a platelet concentration of at least about 2000×10¹ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). As another example, an endpoint can be a platelet concentration of about 1000×10³ to about 2500 platelets/μL (e.g., about 1000×10³ to about 2000×10¹, about 1500×10³, to about 2300×10³, or about 1700×10³ to about 2300×10³ platelets/μL). In some embodiments, an endpoint can be a concentration of platelets in the TFF-treated composition are at least 100×10³ platelets/μL, 200×10³ platelets/μL, 400×10³ platelets/μL, 1000×10³ platelets/μL, 1250×10³ platelets/μL, 1500×10³ platelets/μL, 1750×10³ platelets/μL, 2000×10³ platelets/μL, 2250×10³ platelets/μL, 2500×10³ platelets/μL, 2750×10³ platelets/μL, 3000×10³ platelets/μL, 3250×10³ platelets/μL, 3500×10³ platelets/μL, 3750×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, 20,000×10³ platelets/μL. In some embodiments, the platelets or platelet derivatives in the TFF-treated composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL. In some embodiments, an endpoint can include more than one criterion (e.g., a percentage of residual plasma and a platelet concentration, a relative A280 and a platelet concentration, or an absolute A280 and a platelet concentration).

Typically, a TFF-treated composition is subsequently lyophilized, optionally with a thermal treatment step, to form a final blood product (e.g., platelets, cryopreserved platelets, freeze-dried platelets (e.g., thrombosomes)). However, in some cases, a TFF-treated composition can be considered to be a final blood product.

In some embodiments, a blood product, for example a blood product that includes platelets and/or platelet derivatives, can be prepared using centrifugation of a blood product (e.g., an unprocessed blood product (e.g., donor apheresis material (e.g., pooled donor apheresis material)), or a partially processed blood product (e.g., a blood product that has undergone TFF)), for example to isolate platelets or platelet derivatives away from some, most, virtually all, or all liquid blood components. Thus, methods provided herein that include providing, cryopreserving, and/or lyophilizing platelets, in some embodiments include centrifugation of suspensions such as blood or a platelet-containing fraction thereof, that include platelets, for example to isolate the platelets from some, most, virtually all, or all soluble components in the suspension. In some embodiments, a blood product, for example a blood product that includes platelets and/or platelet derivatives, can be prepared without centrifugation of a blood product (e.g., an unprocessed blood product (e.g., donor apheresis material), or a partially processed blood product (e.g., a blood product that has undergone TFF)), for example to isolate platelets or platelet derivatives away from some, most, virtually all, or all liquid blood components. Thus, methods provided herein that include providing, cryopreserving, and/or lyophilizing platelets, in some embodiments include using methods other than centrifugation, for example TFF, to process suspensions such as blood or a platelet-containing fraction thereof, that include platelets, for example to isolate the platelets from some, most, virtually all, or all soluble components in the suspension. Centrifugation can include any appropriate steps, typically such that platelets are pelleted and can be isolated away from/enriched from soluble components of a platelet-containing suspension such as blood or a fraction thereof, or a buffered solution comprising platelets or platelet derivatives. In some embodiments, centrifugation can include a slow acceleration, a slow deceleration, or a combination thereof. In some embodiments, centrifugation can include centrifugation at about 1400×g to about 1550×g (e.g., about 1400 to about 1450×g, about 1450 to about 1500×g, or 1500 to about 1550×g, about 1400×g, about 1410×g, about 1430×g, about 1450×g, about 1470×g, about 1490×g, about 1500×g, about 1510×g, about 1530×g, or about 1550×g). In some embodiments, the duration of centrifugation can be about 10 min to about 30 min, about 15 min to about 30 min, about 10 to about 20 min, about 20 to about 30 min, about 10 min, about 20 min, about 30 min, 10 min to 30 min, 15 min to 30 min, 10 to 20 min, 20 to 30 min, 10 min, 15 min, 20 min, or 30 min).

In some embodiments, a final blood product can be prepared using both TFF and centrifugation (e.g., TFF followed by centrifugation or centrifugation followed by TFF).

Also provided herein are compositions prepared by any of the methods described herein.

In some embodiments, a composition as described herein can be analyzed at multiple points during processing. In some embodiments, a starting material (e.g., donor apheresis material (e.g., pooled donor apheresis material)) can be analyzed for antibody content (e.g., HLA or HNA antibody content). In some embodiments, a starting material (e.g., donor apheresis material (e.g., pooled donor apheresis material)) can be analyzed for protein concentration (e.g., by absorbance at 280 nm (e.g., using a path length of 0.5 cm)). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

In some embodiments, a composition as described herein can be analyzed at multiple points during processing. In some embodiments, donor apheresis plasma can be analyzed for antibody content (e.g., HLA or HNA antibody content). In some embodiments, donor apheresis plasma can be analyzed for protein concentration (e.g., by absorbance at 280 nm). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

The protein concentration of a blood product can be measured by any appropriate method. In some embodiments, the protein concentration of a blood product can be measured using absorbance at 280 nm.

The antibody content (e.g., HLA or HNA antibody content) of a blood product can be measured by any appropriate method.

In some embodiments, a FLOWPRA™ Screening or a LABScreen Multi test kits from One Lambda, Thermo Fisher Scientific can be used as a method of HLA detection. Raw materials can be tested prior to the TFF or centrifugation processes to determine a baseline level of class I and II antibodies for Human Leukocyte Antigen (HLA) and Human Neutrophil Antigens (HNA). Testing can be repeated after processing by centrifugation or TFF to measure the removal of HLA and HNA. Additional testing points can be performed throughout the TFF procedure to maintain in-process control. Post-lyophilization and annealing, random samples can be selected from a batch and qualitative HLA/HNA antibody testing can be performed to ensure reduction and compliance with current FDA testing and acceptance requirements.

In some embodiments, the antibody content (e.g., HLA or HNA antibody content) of two blood products can be compared by determining the percentage of beads positive for a marker (e.g., HLA or HNA coated beads bound to HLA or HNA antibodies, respectively). Any appropriate comparative method can be used. In some embodiments, the antibody content of two blood products can be compared using a method as described herein. In some embodiments, such a method can be carried out as follows. An aliquot of plasma (e.g., about 1 mL) platelet-poor plasma can be obtained. In some embodiments, an aliquot of filtered (e.g., using a 0.2 μm filter) platelet-poor plasma (PPP) (e.g., about 1 mL) can be obtained. Beads coated with Class I HLA and/or beads coated with Class II HLA can be added to the plasma (e.g., about 5 μL of each type of bead to about 20 μL of PPP) to form a mixture of PPP and beads. The mixture of PPP and beads can be vortexed. The mixture of PPP and beads can be incubated to form an incubated mixture. Any appropriate incubation conditions can be used. For example, in some embodiments, incubation can occur for a time (e.g., for about 30 minutes) at a temperature (e.g., at room temperature) with other conditions (e.g., in the dark) to form an incubated mixture. In some embodiments, incubation can include agitation (e.g., gentle rocking). The beads in the incubated mixture can be washed using any appropriate conditions. In some embodiments, the beads in the incubated mixture can be washed with a wash buffer. Washed beads can be separated from the incubated mixture by any appropriate method. In some embodiments, the washed beads can be separated by centrifugation (e.g., at 9,000×g for 2 minutes) to obtain pelleted beads. In some embodiments, the washing step can be repeated. The beads can be resuspended to form a bead solution. An antibody (e.g., an antibody that will bind to the assayed antibody content (e.g., HLA or HNA antibody content)) conjugated to a detectable moiety can be added to the bead solution (e.g., an αIgG conjugated to a fluorescent reporter, such as FITC). The antibody can be incubated with the bead solution under any appropriate conditions. In some embodiments, the antibody can be incubated for a time (e.g., for about 30 minutes) at a temperature (e.g., at room temperature) with other conditions (e.g., in the dark) to form labeled beads. Labeled beads can be washed to remove unbound antibody conjugated to a detectable moiety. The labeled beads can be washed using any appropriate conditions. In some embodiments, the labeled beads can be washed with a wash buffer. Washed labeled beads can be separated by any appropriate method. In some embodiments, the washed labeled beads can be separated by centrifugation (e.g., at 9,000 g for 2 minutes) to obtain pelleted labeled beads. In some embodiments, the washing step can be repeated. Labeled beads can be detected by any appropriate method. In some embodiments, labeled beads can be detected by flow cytometry. In some embodiments, detection can include measurement of the percentage of beads that are positive for the detectable moiety as compared to a negative control. In some embodiments, a negative control can be prepared as above, using a PPP sample that is known to be negative for antibodies (e.g. HLA Class I, HLA Class II, or HNA antibodies).

In some embodiments, a blood product (e.g., platelets, cryopreserved platelets, freeze-dried platelets (e.g., thrombosomes)) can be analyzed at multiple points during processing. In some embodiments, a starting material (e.g., donor apheresis material) can be analyzed to determine the percent of positive beads (e.g., HLA or HNA coated beads). In some embodiments, a starting material (e.g., donor apheresis material) can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

In some embodiments, a blood product (e.g., platelets, cryopreserved platelets, freeze-dried platelets (e.g., thrombosomes)) can be analyzed at multiple points during processing. In some embodiments, donor apheresis plasma can be analyzed to determine the percent of positive beads (e.g., HLA or HNA coated beads). In some embodiments, donor apheresis plasma can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis plasma. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

A percentage of positive beads can be determined using any appropriate method. In some embodiments, positive beads can be determined compared to a negative control sample. A negative control sample can be any appropriate negative control sample. In some embodiments, a negative control sample can be used to determine positivity gating such that less than a certain percentage (e.g., between about 0.01% and about 1% (e.g., about 0.01% to about 0.05%, about 0.05% to about 0.1%, about 0.1% to about 0.5%, about 0.5% to about 1%, about 0.01%, about 0.05%, about 0.1%, about 0.5%, or about 1%)) of the negative control sample is present within the positivity gate. In some embodiments, a negative control sample can be a buffer (e.g., PBS). In some embodiments, a negative control sample can be a synthetic plasma composition. In some embodiments, a negative control sample can be a blood product known to be negative for the assayed antibodies (e.g., HLA or HNA antibodies).

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration.

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation.

In some embodiments of any of the methods described herein, the amount of an antibody (e.g., a HLA antibody (e.g., a HLA Class I antibody or a HLA Class II antibody) or a HNA antibody) in a composition (e.g., a blood product) can be reduced to below a reference level. A reference level can be any appropriate reference level. In some embodiments of any of the methods described herein, the percentage of beads positive an antibody (e.g., a HLA antibody (e.g., a HLA Class I antibody or a HLA Class II antibody) or a HNA antibody) in a composition (e.g., a blood product) can be reduced as compared to the blood product before undergoing the methods described herein. A percentage of beads positive for an antibody can be reduced by any appropriate amount. In some embodiments, a percentage of beads positive for an antibody can be reduced by at least 5% (e.g., reduced by at least 10%, 20%, 30%, 40%, 50%, 60%, 70%, 75%, 80%, 85%, 90%, 95%, 96%, 97%, 98%, 99%, or more) compared to the blood product before undergoing any of the methods described herein.

In some embodiments, a composition as described herein can undergo any appropriate additional processing steps. In some embodiments, a composition as described herein can be freeze-dried. In some embodiments, freeze-dried platelets can be thermally treated (e.g., at about 80° C. for about 24 hours).

For example, in some embodiments, a composition can be cryopreserved or freeze-dried. In some embodiments, a first composition (e.g., a composition comprising platelets and an aqueous medium as described herein) can be treated with a mixture. In some embodiments, a mixture can include a lyophilizing agent, including a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form a second composition comprising platelets. In some embodiments, a loading agent can be a saccharide. In some embodiments, a saccharide can be a monosaccharide. In some embodiments, a saccharide can be sucrose, maltose, trehalose, glucose (e.g., dextrose), mannose, or xylose. In some embodiments, the loading agent can be polysucrose.

In some embodiments, a first composition or a second composition can be dried. In some embodiments, a first composition or a second composition can be dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a third composition. In some embodiments, a cryoprotectant can be polysucrose.

In some embodiments, a first composition or a second composition can be freeze-dried. In some embodiments, a first composition or a second composition can be freeze-dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a fourth composition. In some embodiments freeze-drying can occur at a temperature of about −40° C. to about 5° C. In some embodiments, freeze-drying can occur over a gradient (e.g., about −40° C. to about 5° C.). In some embodiments, a secondary drying step can be carried out (e.g., at about 20° C. to about 40° C.).

Also provided herein are blood products (e.g., platelets, cryopreserved platelets, freeze-dried platelets (e.g., thrombosomes)) produced by any of the methods described herein.

In some embodiments, the percentage of beads positive for an antibody selected from the group consisting of HLA Class I antibodies, HLA Class II antibodies, and HNA antibodies, as determined for a composition as described herein by flow cytometry using beads coated with Class I HLAs, Class II HLAs, or HNAs, respectively, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class I antibodies, as determined for a composition as described herein by flow cytometry using beads coated with Class I HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class II antibodies, as determined for a composition as described herein by flow cytometry using beads coated with Class II HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HNA antibodies, as determined for a composition as described herein by flow cytometry using beads coated with HNAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

Within the process provided herein for making the compositions provided herein, optional addition of a lyophilizing agent can be the last step prior to drying. However, in some embodiments, the lyophilizing agent can be added at the same time or before other components of the composition, such as a salt, a buffer, optionally a cryoprotectant, or other components. In some embodiments, the lyophilizing agent is added to a preparation agent, thoroughly mixed to form a drying solution, dispensed into a drying vessel (e.g., a glass or plastic serum vial, a lyophilization bag), and subjected to conditions that allow for drying of a TFF-treated composition to form a dried composition.

In some embodiments, dried platelets or platelet derivatives (e.g., thrombosomes) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Tus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets or platelet derivatives (e.g., thrombosomes) for at least 30 minutes at a temperature higher than 30° C.; heating the dried platelets or platelet derivatives (e.g., thrombosomes) for at least 10 hours at a temperature higher than 50° C.; heating the dried platelets or platelet derivatives (e.g., thrombosomes) for at least 18 hours at a temperature higher than 75° C.; and heating the dried platelets or platelet derivatives (e.g., thrombosomes) for 24 hours at 80° C. In some embodiments, heating can be performed in sealed container, such as a capped vial. In some embodiments, a sealed container be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets or platelet derivatives (e.g., thrombosomes).

In some cases, compositions comprising platelets or platelet derivatives (e.g., thrombosomes) can be rehydrated with water (e.g., sterile water for injection) over about 10 minutes at about room temperature. In general, the rehydration volume is about equal to the volume used to fill each vial of thrombosomes prior to drying.

In some embodiments, the platelets or platelet derivatives (e.g., thrombosomes) prepared as disclosed herein have a storage stability that is at least about equal to that of the platelets prior to the preparation.

In some embodiments, the method further comprises cryopreserving the platelets or platelet derivatives prior to administering the platelets or platelet derivatives (e.g., with a preparation agent, e.g., a preparation agent described herein).

In some embodiments, the method further comprises drying a composition comprising platelets or platelet derivatives, (e.g., with a preparation agent e.g., a preparation agent described herein) prior to administering the platelets or platelet derivatives (e.g., thrombosomes). In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with a preparation agent e.g., a preparation agent described herein) prior to administering the platelets or platelet derivatives (e.g., thrombosomes) In some embodiments, the method may further comprise heating the composition following the freeze-drying step. In some embodiments, the method may further comprise rehydrating the composition following the freeze-drying step or the heating step.

In some embodiments, the method further comprises cold storing the platelets, platelet derivatives, or the thrombosomes prior to administering the platelets, platelet derivatives, or thrombosomes (e.g., with a preparation agent, e.g., a preparation agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets or platelet derivatives (e.g., thrombosomes) (e.g., with a preparation agent e.g., a preparation agent described herein) prior to administering the platelets or platelet derivatives (e.g., thrombosomes). For example, in some embodiments, the method further comprises drying (e.g., freeze-drying) a composition comprising platelets or platelet derivatives (e.g., with a preparation agent e.g., a preparation agent described herein) (e.g., to form thrombosomes) prior to administering the platelets or platelet derivatives (e.g., thrombosomes). In some embodiments, the method may further comprise rehydrating the composition obtained from the drying step.

In some embodiments, provided herein is a method for preparing a composition comprising platelets or platelet derivatives (e.g., thrombosomes). The method can include diluting a starting material comprising platelets with an approximately equal weight (10%) of a preparation agent (e.g., Buffer A, as provided in Example 1), concentrating the platelets to about 2250×103 cells/μL (±250×103) and then washed with 2-4 diavolumes (DV) (e.g., about 2 diavolumes) of the preparation agent to form a TFF-treated composition. The residual plasma percentage can be less than about 15% relative plasma (as determined by plasma protein content). Following washing, if the concentration of the cells in the TFF-treated composition is not about 2000×103 cells/μL (±300×103), the cells can be diluted with the preparation agent or can be concentrated to fall within this range. The method can further include lyophilizing the TFF-treated composition and subsequently treating the lyophilized composition comprising platelets or platelet derivatives (e.g., thrombosomes) at about 80° C. for about 24 hours. In some embodiments, the method can further include a pathogen reduction step, for example, before diluting the starting material.

### Aggregation Exhibited by Platelet Derivatives

Platelet derivative compositions, which in certain illustrative embodiments herein are FDPD compositions, comprise a population of platelet derivatives (e.g. FDPDs) having a reduced propensity to aggregate under aggregation conditions comprising an agonist but no fresh platelets, and in illustrative embodiments in the absence of divalent cations, compared to the propensity of fresh platelets and/or activated to aggregate under these conditions. Platelet derivatives (e.g., FDPDs) as described herein in illustrative embodiments, display a reduced propensity to aggregate under aggregation conditions comprising an agonist but no fresh platelets, compared to the propensity of fresh platelets and/or activated platelets to aggregate under these conditions. Surprisingly, such FDPDs have the ability to increase clotting and aggregation of platelets in in vitro and in vivo assays, in the presence of anti-thrombotic agents such as anti-coagulants and anti-platelet agents, under conditions where such anti-thrombotic agents reduce clotting and/or aggregation, including in the presence of two of such agents. It is noteworthy that aggregation of platelet derivatives is different from co-aggregation in that aggregation conditions typically do not include fresh platelets, whereas co-aggregation conditions include fresh platelets. Exemplary aggregation and co-aggregation conditions are provided in the Examples herein. Thus, in some embodiments, the platelet derivatives as described herein have a higher propensity to co-aggregate in the presence of fresh platelets and an agonist, while having a reduced propensity to aggregate in the absence of fresh platelets and an agonist, compared to the propensity of fresh platelets to aggregate under these conditions. In some embodiments, a platelet derivative composition comprises a population of platelet derivatives having a reduced propensity to aggregate, wherein no more than 2%, 3%, 4%, 5%, 7.5%, 10%, 12.5%, 15%, 17.5%, 20%, 22.5%, or 25% of the platelet derivatives in the population aggregate under aggregation conditions comprising an agonist but no platelets, in illustrative embodiments no fresh platelets. In some embodiments, the population of platelet derivatives aggregate in the range of 2-30%, 5-25%, 10-30%, 10-25%, or 12.5-25% of the platelet derivatives under aggregation conditions comprising an agonist but no platelets, in illustrative embodiments no fresh platelets.

As provided in Examples herein, exemplary aggregation conditions and related methods include treating FDPD sample preparations at room temperature with an agonist at a final agonist concentration of 20 μM ADP, 0.5 mg/mL arachidonic acid, 10 μg/mL collagen, 200 μM epinephrine, Img/mL ristocetin, and 10 μM TRAP-6 and measured by LTA for example 5 minutes after agonist addition to the FDPD sample, which can be compared to LTA measurements of the sample prior to agonist addition.

### Platelet Derivatives Exhibit Presence of Surface Markers

Platelets or platelet derivatives (e.g., FDPDs) as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×105 FDPDs/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein iIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, populations of platelet derivatives (e.g., FDPDs), such as those prepared by methods described herein and included in compositions herein, can have an average CD41 percent positivity of at least 55% (e.g., at least 60%, at least 65%, at least 67%, at least 70%, at least 75%, at least 80%, at least 85%, at least 90%, or at least 95%). In some embodiments, at least 90%, 91%, 92%, 93%, 94%, 95%, 96%, 97%, 98%, 99% platelet derivatives that are positive for CD 41 have a size in the range of 0.5-25 μm, 0.5-12.5 μm, or 0.5-2.5 μm in diameter. In some embodiments, at least 90%, 91%, 92%, 93%, 94%, 95%, 96%, 97%, 98%, or 99% platelet derivatives that are positive for CD 41 have a size in the range of 0.4-2.8 μm. In some embodiments, at least 90%, 91%, 92%, 93%, 94%, 95%, 96%, 97%, 98%, or 99% platelet derivatives that are positive for CD 41 have a size in the range of 0.3-3 μm.

As another example, platelets or platelet derivatives (e.g., FDPDs), such as those described herein, can have an average CD42 percent positivity of at least 65% (e.g., at least 67%, at least 70%, at least 75%, at least 80%, at least 85%, at least 90%, or at least 95%). In some embodiments, at least 90%, 91%, 92%, 93%, 94%, 95%, 96%, 97%, 98%, or 99% platelet derivatives that are positive for CD 42 have a size in the range of 0.5-2.5 μm in diameter by flow cytometry. In some embodiments, at least 90%, 91%, 92%, 93%, 94%, 95%, 96%, 97%, 98%, or 99% platelet derivatives that are positive for CD 42 have a size in the range of 0.4-2.8 μm. In some embodiments, at least 90%, 91%, 92%, 93%, 94%, 95%, 96%, 97%, 98%, or 99% platelet derivatives that are positive for CD 42 have a size in the range of 0.3-3 μm in diameter by flow cytometry.

As another example, platelets or platelet derivatives (e.g., FDPDs), such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.5-2.5 μm in diameter by flow cytometry. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.3-3 μm.

As yet another example, platelets or platelet derivatives (e.g., FDPDs), such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm in diameter by flow cytometry. In some embodiments, at least 90%, 91%, 92%, 93%, ⁹⁴%, 95%, ⁹⁶%, ⁹⁷%, ⁹⁸%, or 99% platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm.

In some embodiments, the platelet derivative composition comprises a population of platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm.

### Platelet Derivatives Exhibit Thrombin Generation Potency

Platelets or platelet derivatives (e.g., FDPDs) as described herein can be capable of generating thrombin, for example, when in the presence of a reagent containing tissue factor and phospholipids. For example, in some cases, platelets or platelet derivatives (e.g., FDPDs) (e.g., at a concentration of about 4.8×103 particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of a reagent containing tissue factor (e.g., at 0.25 μM, 0.5 μM, 1 μM, 2 μM, 5 μM or 10 μM) and optionally phospholipids. For example, in some cases, platelets or platelet derivatives (e.g., FDPDs) (e.g., at a concentration of about 4.8×103 particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of a reagent containing tissue factor and (e.g., at 0.25 μM, 0.5 μM, 1 μM, 2 μM, 5 μM or 10 μM) and optionally phospholipids. In some cases, platelets or platelet derivatives (e.g., FDPDs) (e.g., at a concentration of about 4.8×103 particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., FDPDs). In some cases, platelets or platelet derivatives (e.g., FDPDs) (e.g., at a concentration of about 4.8×103 particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., FDPDs).

Platelets or Platelet derivatives (e.g., FDPDs) as described herein can be capable of generating thrombin, for example, when in the presence of a reagent containing tissue factor and phospholipids. For example, in some cases, platelets or platelet derivatives (e.g., FDPDs) can have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 106 particles. For example, in some cases, platelets or platelet derivatives (e.g., FDPDs) can have a potency of between 1.2 and 2.5 TPGU per 106 particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TPGU per 106 particles). TPGU can be calculated as follows: TGPU/million particles=[TPH in nM]*[Potency Coefficient in IU/(nM)]/[0.576 million particles in the well]. Similarly, the Potency Coefficient for a sample of thrombin can be calculated as follows: Potency Coefficient=Calculated Calibrator Activity (IU)/Effective Calibrator Activity (nM). In some cases, the calibrator activity can be based on a WHO international thrombin standard.

Platelets or platelet derivatives (e.g., FDPDs) as described herein can be capable of clotting, as determined, for example, by using a total thrombus-formation analysis system (T-TAS®). In some cases, platelets or platelet derivatives as described herein, when at a concentration of at least 70×10³ particles/μL (e.g., at least 73×103, 100×103, 150×103, 173×103, 200×103, 250×103, or 255×103 particles/μL) can result in a T-TAS occlusion time (e.g., time to reach kPa of 80) of less than 14 minutes (e.g., less than 13.5, 13, 12.5, 12, 11.5, or 11 minutes), for example, in platelet-reduced citrated whole blood. In some cases, platelets or platelet derivatives as described herein, when at a concentration of at least 70×103 particles/μL (e.g., at least 73×103, 100×103, 150×103, 173×103, 200×103, 250×103, or 255×103 particles/μL) can result in an area under the curve (AUC) of at least 1300 (e.g., at least 1380, 1400, 1500, 1600, or 1700), for example, in platelet-reduced citrated whole blood.

Platelets or platelet derivatives (e.g., FDPDs) as described herein can be capable of thrombin-induced trapping in the presence of thrombin. In some cases, platelets or platelet derivatives (e.g., FDPDs) as described herein can have a percent thrombin-induced trapping of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of thrombin. In some cases, platelets or platelet derivatives (e.g., FDPDs) as described herein can have a percent thrombin-induced trapping of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of thrombin. Thrombin-induced trapping can be determined by any appropriate method, for example, light transmission aggregometry. Without being bound by any particular theory, it is believed that the thrombin-induced trapping is a result of the interaction of fibrinogen present on the surface of the platelet derivatives with thrombin.

Platelets or platelet derivatives (e.g., FDPDs) as described herein can be capable of co-aggregating, for example, in the presence of an aggregation agonist, and fresh platelets. Non-limiting examples of aggregation agonists include, collagen, epinephrine, ristocetin, arachidonic acid, adenosine di-phosphate, and thrombin receptor associated protein (TRAP). In some cases, platelets or platelet derivatives (e.g., FDPDs) as described herein can have a percent co-aggregation of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of an aggregation agonist, and fresh platelets. In some cases, platelets or platelet derivatives (e.g., FDPDs) as described herein can have a percent co-aggregation of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of an aggregation agonist. Percent co-aggregation can be determined by any appropriate method, for example, light transmission aggregometry.

### Platelet Derivatives Exhibit Presence of Thrombospondin (TSP) on the Surface

Thrombospondin is a glycoprotein secreted from the α-granules of platelets upon activation. In the presence of divalent cations, the secreted protein binds to the surface of the activated platelets and is responsible for the endogenous lectin-like activity associated with activated platelets. In some embodiments, the platelet derivatives have the presence of thrombospondin (TSP-1) on their surface at a level that is greater than that presence on the surface of resting platelets, activated platelets, or lyophilized fixed platelets. In some embodiments, the platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

### Platelet Derivatives Exhibit the Presence of Von Willebrand Factor on the Surface

Von Willebrand factor (vWF) is a multimeric glycoprotein that plays a major role in blood coagulation. vWF serves as a bridging molecule that promotes platelet binding to sub-endothelium and other platelets, thereby promoting platelet adherence and aggregation. vWF also binds to collagens to facilitate clot formation at sites of injury. In some embodiments, the platelet derivatives as described herein have the presence of von Willebrand factor (vWF) on their surface at a level that is greater than that on the surface of resting platelets, activated platelets, or lyophilized fixed platelets. In some embodiments, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

### Platelet Derivatives Exhibit an Inability to Increase Expression of a Platelet Activation Marker

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP). In some embodiments, the platelet activation marker is selected from the group consisting of Annexin V, and CD 62. In some embodiments, the platelet derivatives as described herein show an inability to increase expression of Annexin V in the presence of TRAP. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

### Platelet Derivatives Exhibit Compromised Membrane

Platelet derivatives, in illustrative embodiments FDPDs, in further illustrative aspects and embodiments herein are surrounded by a compromised plasma membrane. In these further illustrative aspects and embodiments, the platelet derivatives lack an integrated membrane around them. Instead, the membrane surrounding such platelet derivatives (e.g. FDPDs) comprises pores that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets. Furthermore, such platelet derivatives (e.g. FDPDs) are not believed to be capable of mitochondrial activation or glycolysis.

A compromised membrane can be identified through a platelet derivative's inability to retain more than 50% of lactate dehydrogenase (LDH) as compared to fresh platelets, or cold stored platelets, or cryopreserved platelets. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives exhibit an increased permeability to antibodies. In some embodiments, the antibodies can be IgG antibodies. The increased permeability can be identified by targeting IgG antibodies against a stable intracellular antigen. One non-limiting type of stable intracellular antigen is p tubulin. The compromised membrane of the platelet derivatives can also be determined by flow cytometry studies.

Platelet or platelet derivatives (e.g., FDPDs) as described herein can retain some metabolic activity, for example, as evidenced by lactate dehydrogenase (LDH) activity. In some cases, platelets or platelet derivatives (e.g., FDPDs) as described herein can retain at least about 10% (e.g., at least about 12%, 15%, 20%, 25%, 30%, 35%, 40%, or 45%) of the LDH activity of donor apheresis platelets. Without being bound by any particular theory, it is believed that the addition of increasing amounts of polysucrose increases the amount of LDH activity remained (e.g., products of a preparation agent with 8% polysucrose have more retained LDH activity than products of a preparation agent with 4% polysucrose). Similarly unbound by any particular theory, it is believed that thermal treatment of a lyophilized composition comprising platelets or platelet derivatives (e.g., FDPDs) increases the amount of LDH activity retained. As another example, metabolic activity can be evidenced by retained esterase activity, such as the ability of the cells to cleave the acetate groups on carboxyfluorescein diacetate succinimidyl ester (CFDASE) to unmask a fluorophore.

### Platelet Derivatives and Microparticles

Platelet derivatives herein have been observed to have numerous surprising properties, as disclosed in further detail herein. It will be understood, as illustrated in the Examples provided herein, that although platelet derivatives in some aspects and embodiments are in a solid, such as a powder form, the properties of such platelet derivatives can be identified, confirmed, and/or measured when a composition comprising such platelet derivatives is in liquid form.

In some embodiments, the platelets or platelet derivatives (e.g., FDPDs) have a particle size (e.g., diameter, max dimension) of at least about 0.5 μm (e.g., at least about at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets or platelet derivatives (e.g., FDPDs), have a particle size in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets or platelet derivatives (e.g., FDPDs), are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets or platelet derivatives (e.g., FDPDs) are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some illustrative embodiments, a microparticle can be a particle having a particle size (e.g., diameter, max dimension) of less than about 0.5 μm (less than about 0.45 μm or 0.4 μm) In some cases, a microparticle can be a particle having a particle size of about 0.01 μm to about 0.5 μm (e.g., about 0.02 μm to about 0.5 μm).

Compositions comprising platelets or platelet derivatives (e.g., FDPDs), such as those prepared according to methods described herein, can have a microparticle content that contributes to less than about 5.0% (e.g., less than about 4.5%, 4.0%, 3.5%, 3.0%, 2.5%, 2.0%, 1.5%, 1.0%, or 0.5%) of the total scattering intensity of all particles from about 1 nm to about 60,000 nm in radius in the composition. In some embodiments, the platelet derivative composition comprises a population of platelet derivatives comprising CD41-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD42-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. As used herein, a content of microparticles “by scattering intensity” refers to the microparticle content based on the scattering intensity of all particles from about 1 nm to about 60,000 nm in radius in the composition. The microparticle content can be measured by any appropriate method, for example, by dynamic light scattering (DLS). In some cases, the viscosity of a sample used for DLS can be at about 1.060 cP (or adjusted to be so), as this is the approximate viscosity of plasma. In some embodiments, the platelet derivative composition as per any aspects, or embodiments comprises a population of platelet derivatives, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, the platelet derivatives have a diameter in the range of 0.5-2.5 μm, and the microparticles have a diameter less than 0.5 μm.

Platelets or platelet derivatives (e.g., FDPDs) as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×105 FDPDs/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein iIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, platelets or platelet derivatives (e.g., FDPDs), such as those prepared by methods described herein, can have an average CD41 percent positivity of at least 55% (e.g., at least 60%, at least 65%, at least 67%, at least 70%, at least 75%, at least 80%, at least 85%, at least 90%, or at least 95%). In some embodiments, at least 90%, 91%, 92%, 93%, 94%, 95%, 96%, 97%, 98%, 99% platelet derivatives that are positive for CD 41 have a size in the range of 0.5-2.5 μm in diameter by flow cytometry. In some embodiments, at least 90%, 91%, 92%, 93%, 94%, 95%, 96%, 97%, 98%, or 99% platelet derivatives that are positive for CD 41 have a size in the range of 0.4-2.8 μm. In some embodiments, at least 90%, 91%, 92%, 93%, 94%, 95%, 96%, 97%, 98%, or 99% platelet derivatives that are positive for CD 41 have a size in the range of 0.3-3 μm.

As another example, platelets or platelet derivatives (e.g., FDPDs), such as those described herein, can have an average CD42 percent positivity of at least 65% (e.g., at least 67%, at least 70%, at least 75%, at least 80%, at least 85%, at least 90%, or at least 95%). In some embodiments, at least 90%, 91%, 92%, 93%, 94%, 95%, 96%, 97%, 98%, or 99% platelet derivatives that are positive for CD 42 have a size in the range of 0.5-2.5 μm in diameter by flow cytometry. In some embodiments, at least 90%, 91%, 92%, 93%, 94%, 95%, 96%, 97%, 98%, or 99% platelet derivatives that are positive for CD 42 have a size in the range of 0.4-2.8 μm. In some embodiments, at least 90%, 91%, 92%, 93%, 94%, 95%, 96%, 97%, 98%, or 99% platelet derivatives that are positive for CD 42 have a size in the range of 0.3-3 μm.

As another example, platelets or platelet derivatives (e.g., FDPDs), such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.5-2.5 μm in diameter by flow cytometry. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.3-3 μm.

As yet another example, platelets or platelet derivatives (e.g., FDPDs), such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm in diameter by flow cytometry. In some embodiments, at least 90%, 91%, 92%, 93%, ⁹⁴%, ⁹⁵%, ⁹⁶%, ⁹⁷%, ⁹⁸%, or 99/platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm.

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP). In some embodiments, the platelet activation marker is selected from the group consisting of Annexin V, and CD 62. In some embodiments, the platelet derivatives as described herein show an inability to increase expression of Annexin V in the presence of TRAP. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

As another example, platelets or platelet derivatives (e.g., FDPDs), such as those prepared by methods described herein, can have an average CD47 percent positivity of at least about 8% (e.g., at least about 10%, 15%, 20%, 25%, 30%, 35%, 40%, 45%, 50%, or 55%).

Platelet derivatives (e.g., thrombosomes) herein, in some embodiments, are dry platelet derivatives, or dry platelet derived particles. In some embodiments, such dry platelet derivatives are freeze-dried (i.e., lyophilized) platelets or platelet derivatives. One such non-limiting example of dry platelet derivatives are thrombosomes. Dry platelet derivatives are typically in the form of a platelet derivative powder. The dry platelet derivative powder when rehydrated typically form a rehydrated platelet derivative composition comprising particles. In some embodiments, compositions comprising a population of platelet derivatives, dry platelet derivatives, platelet derivative powder, or rehydrated platelet derivatives can be characterized by the presence of CD41 on or in at least 55%, 60%, 65% or higher platelet derivatives in the population. In some embodiments, compositions comprising a population of platelet derivatives, dry platelet derivatives, platelet derivative powder, or rehydrated platelet derivatives can be characterized by the presence of CD42 on or in at least 55%, 60%, 65% or higher platelet derivatives in the population. In some embodiments, dry platelet derivative particles herein can have at least one property selected from: (a) high expression of P-selectin (CD62P), for example, at least 2 fold higher than platelets, for example, apheresis platelets, or at least 10%, 20%, 30%, 40%, 50%, 60%, 70%, or higher platelet derivative particles are positive for CD62; (b) high expression of phosphatidyl serine (PS), for example, at least 2, 3, 4, 5, 6, 7, 8, 9, 10 fold or higher than the expression on platelets, for example, apheresis platelets, or at least 25%, 30%, 40%, 50%, 60%, 70%, or higher platelet derivative particles are positive for phosphatidyl serine; (c) high expression of von Willebrand Factor (vWF), for example, at least 2, 3, 4, 5, 6, 7, 8, 9, 10 fold or higher than the expression on platelets, for example, apheresis platelets; (d) high expression of fibrinogen, for example, at least 2, 3, 4, 5, 6, 7, 8, 9, 10 fold or higher than the expression on platelets, for example, apheresis platelets) high expression of thrombospondin (TSP), for example, at least 2, 3, 4, 5, 6, 7, 8, 9, 10 fold or higher than the expression on platelets, for example, apheresis platelets; (f) high expression of CD41, for example, at least 55%, 60%, 65%, 70%, 75%, 80%, 85%, 90%, or higher platelet derivative particles are positive for CD41; or (g) high expression of CD42, for example, at least 65%, 70%, 75%, 80%, 85%, 90%, 95%, or higher platelet derivative particles are positive for CD42. In some embodiments, the platelet derivative particles can have two or more properties as described herein, in illustrative embodiments, the platelet derivative particles can have all the properties as described herein.

### TFF Processing Methods

In some embodiments, the method can include an initial dilution step, for example, a starting material (e.g., an unprocessed blood product (e.g., donor apheresis material (e.g., pooled donor apheresis material)) can be diluted with a preparation agent (e.g., any of the preparation agents described herein) to form a diluted starting material. In some cases, the initial dilution step can include dilution with a preparation agent with a mass of preparation agent equal to at least about 10% of the mass of the starting material (e.g., at least about 15%, 25%, 50%, 75%, 100%, 150%, or 200% of the mass of the starting material. In some embodiments, an initial dilution step can be carried out using the TFF apparatus.

In some embodiments, the method can include concentrating (e.g., concentrating platelets) (e.g., concentrating a starting material or a diluted starting material) to form a concentrated platelet composition. For example, concentrated can include concentrating to a about 1000×103 to about 4000×103 platelets/μL (e.g., about 1000×103 to about 2000×103, about 2000×103 to about 3000×103, or about 4000×103 platelets/μL). In some embodiments, a concentration step can be carried out using the TFF apparatus.

The concentration of platelets or platelet derivatives (e.g., FDPDs) can be determined by any appropriate method. For example, a counter can be used to quantitate concentration of blood cells in suspension using impedance (e.g., a Beckman Coulter AcT 10 or an AcT diff 2).

In some embodiments, TFF can include diafiltering (sometimes called “washing”) of a starting material, a diluted starting material, a concentrated platelet composition, or a combination thereof. In some embodiments, diafiltering can include washing with at least 2 (e.g., at least 3, 4, 5, 6, 7, 8, 9, 10, or more) diavolumes. In some embodiments, TFF can include buffer exchange. In some embodiments, a buffer can be used in TFF. A buffer can be any appropriate buffer. In some embodiments, the buffer can be a preparation agent (e.g., any of the preparation agents described herein). In some embodiments, the buffer can be the same preparation agent as was used for dilution. In some embodiments, the buffer can be a different preparation than was used for dilution. In some embodiments, a buffer can include a lyophilizing agent, including a buffering agent, a base, a loading agent, optionally a salt, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof. A buffering agent can be any appropriate buffering agent. In some embodiments, a buffering agent can be HEPES ((4-(2-hydroxyethyl)-1-piperazineethanesulfonic acid). A base can be any appropriate base. In some embodiments, a base can be sodium bicarbonate. In some embodiments, a saccharide can be a monosaccharide. In some embodiments, a loading agent can be a saccharide. In some embodiments, a saccharide can include sucrose, maltose, trehalose, glucose (e.g., dextrose), mannose, or xylose. In some embodiments, a monosaccharide can be trehalose. In some embodiments, the loading agent can include polysucrose. A salt can be any appropriate salt. In some embodiments, a salt can be selected from the group consisting of sodium chloride (NaCl), potassium chloride (KCl), or a combination thereof.

In some embodiments, a membrane with a pore size of about 0.1 μm to about 1 μm (e.g., about 0.1 μm to about 1 μm, about 0.1 μm to about 0.5 μm, about 0.2 to about 0.45 μm, about 0.45 to about 1 μm, about 0.1 μm, about 0.2 μm, about 0.45 μm, about 0.65 μm, or about 1 μm) can be used in TFF. A membrane can be made from any appropriate material. In some cases, a membrane can be a hydrophilic membrane. In some embodiments, a membrane can be a hydrophobic membrane. In some embodiments, a membrane with a nominal molecular weight cutoff (NMWCO) of at least about 100 kDa (e.g., at least about 200, 300 kDa, 500 kDa, or 1000 kDa) can be used in TFF. The TFF can be performed with any appropriate pore size within the range of 0.1 μm to 1.0 μm with the aim of reducing the microparticles content in the composition and increasing the content of platelet derivatives in the composition. A skilled artisan can appreciate the required optimization of the pore size in order to retain104ehydratt“le“derivativ”s “nd allow the microparticle” t“pas” t”rough the membrane. The pore“si”e in illustrative embodiments, is such that the microparticles pass through the membrane allowing the TFF-treated composition to have less than 5% microparticles. The pore size in illustrative embodiments is such that a maximum of platelet derivatives gets retained in the process allowing the TFF-treated composition to have a concentration of the platelet derivatives in the range of 100×103 to 20,000×103. The pore size during the TFF process can be exploited to obtain a higher concentration of platelet derivatives in the platelet derivative composition such that a person administering the platelet derivatives to a subject in need has to rehydrate/reconstitute fewer vials, therefore, being efficient with respect to time and effort during the process of preparing such platelet derivatives for a downstream procedure, for example a method of treating provided herein. TFF can be performed at any appropriate temperature. In some embodiments, TFF can be performed at a temperature of about 20° C. to about 37° C. (e.g., about 20° C. to about 25° C., about 20° C. to about 30° C., about 25° C. to about 30° C., about 30° C. to about 35° C., about 30° C. to about 37° C., about 25° C. to about 35° C., or about 25° C. to about 37° C.). In some embodiments, TFF can be carried out at a flow rate (e.g., a circulating flow rate) of about 100 ml/min to about 800 ml/min (e.g., about 100 to about 200 ml/min, about 100 to about 400 ml/min, about 100 to about 600 ml/min, about 200 to about 400 ml/min, about 200 to about 600 ml/min, about 200 to about 800 ml/min, about 400 to about 600 ml/min, about 400 to about 800 ml/min, about 600 to about 800 ml/min, about 100 ml/min, about 200 ml/min, about 300 ml/min, about 400 ml/min, about 500 ml/min, about 600 ml/min, about 700 ml/min, or about 800 ml/min).

In some embodiments, TFF can be performed until a particular endpoint is reached, forming a TFF-treated composition. An endpoint can be any appropriate endpoint. In some embodiments, an endpoint can be a percentage of residual plasma (e.g., less than or equal to about 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of residual plasma). In some embodiments, an endpoint can be a relative absorbance at 280 nm (A280). For example, an endpoint can be an A280 (e.g., using a path length of 0.5 cm) that is less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the A280 (e.g., using a path length of 0.5 cm) prior to TFF (e.g., of a starting material or of a diluted starting material). In some embodiments, an A280 can be relative to a system that measures 7.5% plasma=1.66 AU. In some embodiments, an instrument to measure A280 can be configured as follows: a 0.5 cm gap flow cell can be attached to the filtrate line of the TFF system. The flow cell can be connected to a photometer with fiber optics cables attached to each side of the flow cell (light source cable and light detector cable). The flow cell can be made with a silica glass lens on each side of the fiber optic cables. Apart from the relative protein concentration of proteins in the aqueous medium, the protein concentration in the aqueous medium can also be measured in absolute terms. In some embodiments, the protein concentration in the aqueous medium is less than or equal to 15%, or 14%, or 13%, or 12%, or 11%, or 10%, or 9%, or 8%, or 7%, or 6%, or 5%, or 4%, or 3%, or 2%, or 1%, or 0.1%, or 0.01%. In some exemplary embodiments, the protein concentration is less than 3% or 4%. In some embodiments, the protein concentration is in the range of 0.01-15%, or 0.1-15%, or 1-15%, or 1-10%, or 0.01-10%, or 3-12%, or 5-10% in the TFF-treated composition. In some embodiments, an endpoint can be an absolute A280 (e.g., using a path length of 0.5 cm). For example, an endpoint can be an A280 that is less than or equal to 2.50 AU, 2.40 AU, 2.30 AU, 2.20 AU, 2.10 AU, 2.0 AU, 1.90 AU, 1.80 AU, or 1.70 AU (e.g., less than or equal to 1.66, 1.6, 1.5, 1.4, 1.3, 1.2, 1.1, 1.0, 0.9, 0.8, 0.7, 0.6, 0.5, 0.4, 0.3, 0.2, or 0.1 AU) (e.g., using a path length of 0.5 cm). In some embodiments, a percentage of residual plasma, a relative A280, or an A280 can be determined based on the aqueous medium of a composition comprising platelets and an aqueous medium. In some embodiments, a percentage of residual plasma can be determined based on a known correlation to an A280. In some embodiments, an endpoint can be a platelet concentration, as TFF can include concentration or dilution of a sample (e.g., using a preparation agent). For example, an endpoint can be a platelet concentration of at least about 2000×103 platelets/μL (e.g., at least about 2050×103, 2100×103, 2150×103, 2200×103, 2250×103, 2300×103, 2350×103, 2400×103, 2450×103, or 2500×103 platelets/μL). As another example, an endpoint can be a platelet concentration of about 1000×103 to about 2500 platelets/μL (e.g., about 1000×103 to about 2000×103, about 1500×103 to about 2300×103, or about 1700×103 to about 2300×103 platelets/μL). In some embodiments, an endpoint can be a concentration of platelets in the TFF-treated composition are at least 100×103 platelets/μL, 200×103 platelets/μL, 400×103 platelets/μL, 1000×103 platelets/μL, 1250×103 platelets/μL, 1500×103 platelets/μL, 1750×103 platelets/μL, 2000×103 platelets/μL, 2250×103 platelets/μL, 2500×103 platelets/μL, 2750×103 platelets/μL, 3000×103 platelets/μL, 3250×103 platelets/μL, 3500×103 platelets/μL, 3750×103 platelets/μL, 4000×103 platelets/μL, 4250×103 platelets/μL, 4500×103 platelets/μL, 4750×103 platelets/μL, 5000×103 platelets/μL, 5250×103 platelets/μL, 5500×103 platelets/μL, 5750×103 platelets/μL, 6000×103 platelets/μL, 7000×103 platelets/μL, 8000×103 platelets/μL, 9000×103 platelets/μL, 10,000×103 platelets/μL, 11,000×103 platelets/μL, 12,000×103 platelets/μL, 13,000×103 platelets/μL, 14,000×103 platelets/μL, 15,000×103 platelets/μL, 16,000×103 platelets/μL, 17,000×103 platelets/μL, 18,000×103 platelets/μL, 19,000×103 platelets/μL, 20,000×103 platelets/μL. In some embodiments, the platelets or platelet derivatives in the TFF-treated composition is in the range of 100×103-20,000×103 platelets/μL, or 1000×103-20,000×103 platelets/μL, or 1000×103-10,000×103 platelets/μL, or 500×103-5,000×103 platelets/μL, or 1000×103-5,000×103 platelets/μL, or 2000×103-8,000×103 platelets/μL, or 10,000×103-20,000×103 platelets/μL, or 15,000×103-20,000×103 platelets/μL.

In some embodiments, an endpoint can include more than one criterion (e.g., a percentage of residual plasma and a platelet concentration, a relative A280 and a platelet concentration, or an absolute A280 and a platelet concentration).

Typically, a TFF-treated composition is subsequently lyophilized, optionally with a thermal treatment step, to form a final blood product (e.g., platelets, cryopreserved platelets, FDPDs. However, in some cases, a TFF-treated composition can be considered to be a final blood product.

In some embodiments, a final blood product can be prepared using both TFF and centrifugation (e.g., TFF followed by centrifugation or centrifugation followed by TFF).

Also provided herein are compositions prepared by any of the methods described herein.

In some embodiments, a composition as described herein can be analyzed at multiple points during processing. In some embodiments, a starting material (e.g., donor apheresis material (e.g., pooled donor apheresis material)) can be analyzed for antibody content (e.g., HLA or HNA antibody content). In some embodiments, a starting material (e.g., donor apheresis material (e.g., pooled donor apheresis material)) can be analyzed for protein concentration (e.g., by absorbance at 280 nm (e.g., using a path length of 0.5 cm)). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product (e.g., (e.g., platelets, cryopreserved platelets, FDPDs can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, FDPDs can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product can have no detectable level of an antibody selected from the group consisting of HLA Class I antibodies, HLA Class II antibodies, and HNA antibodies.

In some embodiments, the aqueous medium of a composition as described herein can be analyzed as described herein.

### EXEMPLARY EMBODIMENTS

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In one aspect, provided herein is a composition comprising MRI agent-loaded cryopreserved platelets, wherein the MRI agent-loaded cryopreserved platelets comprise an MRI agent complex covalently bonded to the surface of the cryopreserved platelets. Typically, the MRI agent complex comprises an MRI agent, and a chelator. In illustrative embodiments, in the MRI agent-loaded cryopreserved platelets, the MRI agent is associated in a non-covalent bonding with the chelator, and the chelator is covalently bonded to the platelets, typically to protein molecules on the surface of the platelets. In some embodiments, the MRI agent-loaded cryopreserved platelets are capable of retaining at least 2%, 5%, 7.5%, 10%, 12%, 15%, 20%, 25%, 30%, 35%, 40% of the loaded MRI agent upon thawing.

In one aspect, provided herein is a composition comprising MRI agent-loaded platelet derivatives, wherein the MRI agent-loaded platelet derivatives comprise an MRI agent complex covalently bonded to the surface of the platelet derivatives. Typically, the MRI agent complex comprises an MRI agent, and a chelator. In illustrative embodiments, the MRI agent-loaded platelet derivatives are surrounded by a compromised plasma membrane. In further illustrative embodiments, at least 25%, 30%, 35%, 40%, 45%, or 50% of the MRI agent-loaded platelet derivatives are CD 41-positive platelet derivatives. In some embodiments, the MRI agent-loaded platelet derivatives are capable of retaining at least 2%, 5%, 7.5%, 10%, 12%, 15%, 20%, 25%, 30%, 35%, 40% of the loaded MRI agent upon rehydrating. In illustrative embodiments, in the MRI agent-loaded platelet derivatives, the MRI agent is associated in a non-covalent bonding with the chelator, and the chelator is covalently bonded to the platelets, typically to protein molecules on the surface of the platelets.

In one aspect, provided herein is a method for preparing a composition comprising MRI agent-loaded cryopreserved platelets, comprising:
(a) providing platelets;
(b) contacting the platelets with an MRI agent complex, to form MRI agent-loaded platelets; and
(c) cryopreserving the MRI agent-loaded platelets to form the composition comprising the MRI agent-loaded cryopreserved platelets. In some embodiments, MRI agent complex that is contacted to the platelets typically comprises an MRI agent, a chelator, and a linker. In illustrative embodiments, the MRI agent-loaded platelets, or the MRI agent-loaded cryopreserved platelets comprise MRI agent complex covalently bonded to the surface of the MRI agent-loaded platelets, or the MRI agent-loaded cryopreserved platelets. Typically, the MRI agent complex that is covalently bonded to the MRI agent-loaded platelets or the MRI agent-loaded cryopreserved platelets comprises the MRI agent, and the chelator.

In one aspect, provided herein is a method for preparing a composition comprising MRI agent-loaded platelet derivatives in a powder, comprising:
(a) providing platelets;
(b) contacting the platelets with an MRI agent complex, to form MRI agent-loaded platelets; and
(c) freeze-drying the MRI agent-loaded platelets to form the composition comprising the MRI agent-loaded platelet derivatives. In some embodiments, MRI agent complex that is contacted to the platelets typically comprises an MRI agent, a chelator, and a linker. In illustrative embodiments, the MRI agent-loaded platelets, or the MRI agent-loaded platelet derivatives comprise MRI agent complex covalently bonded to the surface of the MRI agent-loaded platelets, or the MRI agent-loaded platelet derivatives. Typically, the MRI agent complex that is covalently bonded to the MRI agent-loaded platelets or the MRI agent-loaded platelet derivatives comprises the MRI agent, and the chelator.

In one aspect, provided herein is a method for preparing a composition comprising MRI agent-loaded, freeze-dried platelet derivatives, the method comprising:
(a) performing tangential flow filtration (TFF) of a platelet composition with a preparation agent comprising saccharides, wherein said TFF is performed using a 0.3 to 1 micron filter, thereby preparing a TFF-treated composition comprising platelets in an aqueous medium having less than or equal to 7.5%, 10%, 15%, or 20% plasma protein, and having less than 30%, 25%, 20%, 15%, 12%, 10%, 8%, 7%, or 5.0% microparticles having a diameter less than 0.5 μm by scattering intensity;
(b) contacting the TFF-treated composition with an MRI agent complex comprising an MRI agent, a linker, and a chelator, at a temperature in the range of 15-45° C. for a time period in the range of 5 minutes to 48 hours, to form an MRI agent-loaded, TFF treated platelet composition; and
(c) freeze drying the MRI agent-loaded, TFF-treated platelet composition to form the composition comprising MRI agent-loaded, freeze-dried platelet derivatives. In some embodiments, the method further comprises heating the composition comprising MRI agent-loaded, freeze-dried platelet derivatives in a powder form at a temperature in the range of 60° C. to 90° C. for at least 1 hour to not more than 36 hours to thermally treat the MRI agent-loaded, freeze-dried platelet derivatives.

Aspects and embodiments herein include any compositions comprising platelet derivatives provided herein, wherein the platelet derivatives comprise, and are typically loaded with a magnetic resonance imaging (MRI) agent. Accordingly in one aspect, provided herein is a platelet derivative composition in the form of a powder, comprising a population of MRI-agent loaded platelet derivatives having a reduced propensity to aggregate, such that no more than 25%, and in non-limiting illustrative embodiments, no more than 10%, of the platelet derivatives in the population aggregate under aggregation conditions comprising an agonist but no platelets, and wherein the platelet derivatives are capable of generating thrombin, and in certain embodiments have a potency of at least 0.5, 1.0, and in non-limiting illustrative embodiments 1.5 thrombin generation potency units (TGPU) per 10⁶ platelet derivatives. In some embodiments, the platelet derivatives are freeze-dried platelet derivatives.

In one aspect, provided herein is a platelet derivative composition in the form of a powder, comprising a population of MRI-agent loaded platelet derivatives having a reduced propensity to aggregate, wherein no more than 25%, and in non-limiting illustrative embodiments, no more than 10%, of the platelet derivatives in the population aggregate under aggregation conditions comprising an agonist but no platelets; and having one or more, two or more, or all of the following characteristics of a super-activated platelet selected from: a. the presence of thrombospondin (TSP) on their surface at a level that is greater than on the surface of resting platelets; b. the presence of von Willebrand factor (vWF) on their surface at a level that is greater than on the surface of resting platelets; c. an inability to increase expression of a platelet activation marker in the presence of an agonist as compared to the expression of the platelet activation marker in the absence of an agonist.

In illustrative embodiments of any of the aspects herein, illustrative or target platelet derivatives are CD41 positive and/or CD42 positive. Platelets derivatives (e.g., thrombosomes) herein, in some embodiments, are dry platelet derivatives, or dry platelet derived particles. A skilled artisan will understand that most properties of such dry platelet derivatives are analyzed after the platelet derivatives are rehydrated. Dry platelet derivatives are typically present in a dried substance that includes other components (e.g., saccharides such as, for example, trehalose and/or polysucrose) present along with the platelet derivatives when they were dried. In illustrative platelet derivative compositions herein, less than 5% of the particles are microparticles having a diameter of less than 0.5 μm. In illustrative platelet derivative compositions herein, at least 90% of the particles therein are at least 0.5 μm in diameter. Furthermore, in illustrative embodiments, between 75% and 95% of the platelet derivatives or particles therein are CD41 positive, between 75% and 95% of the platelet derivatives or particles therein are CD42 positive, and less than 5% of the CD 41-positive platelet derivatives or particles therein are microparticles having a diameter of less than 0.5 μm. It will be understood that in such percent calculations, particles are only intended to cover those that can be detected for example by the instrument (e.g., flow cytometer) used to detect CD41 or CD42 or any surface marker.

In some embodiments or subembodiments, the platelet derivatives are FPHs. In some examples of such illustrative embodiments, the platelet derivatives have a potency of at least 1.5 thrombin generation potency units (TGPU) per 10⁶ platelet derivatives. In non-limiting illustrative embodiments, FDPD compositions, or illustrative FDPD compositions herein, such as those prepared according to Example 2 herein, are compositions that include illustrative or target platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and typically such compositions have the ability to generate thrombin in an in vitro thrombin generation assay and/or have the ability to occlude a collagen-coated microchannel in vitro. In illustrative embodiments, the platelet derivatives in such compositions have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the illustrative or target platelet derivatives are between 0.5 and 2.5 μm in diameter by flow cytometry or between 0.5 and 25.0 μm in diameter by dynamic light scattering.

In one aspect, provided herein is a platelet derivative composition in the form of a powder, comprising a population of MRI-agent loaded platelet derivatives comprising CD 41-positive platelet derivatives, wherein the population comprises platelet derivatives having a reduced propensity to aggregate such that no more than 25%, and in non-limiting illustrative embodiments, no more than 10%, of the platelet derivatives in the population aggregate under aggregation conditions comprising an agonist but no platelets, wherein the platelet derivatives have an inability to increase expression of a platelet activation marker in the presence of an agonist as compared to the expression of the platelet activation marker in the absence of the agonist, wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives; and wherein less than 15%, and in certain non-limiting illustrative embodiments less than 5% of the CD 41-positive platelet derivatives are microparticles having a diameter of less than 1 μm, and in certain non-limiting illustrative embodiments less than 0.5 μm.

In one aspect, provided herein is a platelet derivative composition in the form of a powder, comprising a population of MRI-agent loaded platelet derivatives having a reduced propensity to aggregate, such that no more than 25%, and in non-limiting illustrative embodiments, no more than 10%, of the platelet derivatives in the population aggregate under aggregation conditions comprising an agonist but no platelets, and further having one or both of: the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a platelet derivative composition in the form of a powder, comprising a population of MRI-agent loaded platelet derivatives comprising CD41-positive platelet derivatives, wherein less than 15%, and in certain non-limiting illustrative embodiments less than 5% of the CD41-positive platelet derivatives are microparticles having a diameter of less than 1 μm, and in certain non-limiting illustrative embodiments less than 0.5 μm, and comprising platelet derivatives having one or more of, two or more of, three or more of, and in illustrative embodiments all of the following: a reduced propensity to aggregate, in certain embodiments such that no more than 25%, and in illustrative embodiments no more than 10% of the platelet derivatives in the population aggregate under aggregation conditions comprising an agonist but no platelets; an inability to increase expression of a platelet activation marker in the presence of an agonist as compared to the expression of the platelet activation marker in the absence of the agonist; the presence of thrombospondin (TSP) on their surface at a level that is greater than on the surface of resting platelets; the presence of von Willebrand factor (vWF) on their surface at a level that is greater than on the surface of resting platelets; and are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a platelet derivative composition in the form of a powder, comprising trehalose in the range of 20-35% by weight, polysucrose in the range of 45-60% by weight, and MRI-agent loaded platelet derivatives in the range of 0.5-20% by weight, wherein the platelet derivative composition comprises a population of platelet derivatives having a reduced propensity to aggregate such that no more than 25%, and in non-limiting illustrative embodiments, no more than 10%, of the platelet derivatives in the population aggregate under aggregation conditions comprising an agonist but no platelets, and further having one or both of the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a plurality of containers each containing a platelet derivative composition in the form of a powder, wherein the platelet derivative composition in each container comprises a population of MRI-agent loaded platelet derivatives having a reduced propensity to aggregate such that no more than 25%, and in non-limiting illustrative embodiments, no more than 10% of the platelet derivatives in the population aggregate under aggregation conditions comprising an agonist but no platelets, wherein the platelet derivative compositions in each container are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, wherein the platelet derivatives have an inability to increase expression of a platelet activation marker in the presence of an agonist as compared to the expression of the platelet activation marker in the absence of the agonist, wherein the plurality of containers comprise the platelet derivative composition from at least 2 different lots in separate containers, and wherein one or more of: the amount of plasma protein in the powder of any two containers chosen from different lots, differs by less than 10%, 5%, 2%, or 1%, and the amount of microparticles that are less than 1 μm, and in certain non-limiting illustrative embodiments less than 0.5 μm in the powder of any two containers chosen from different lots, differs by less than 10%, 5%, 2%, or 1%.

Methods are provided herein for preparing platelet derivative compositions. Illustrative embodiments herein comprise loading platelet derivatives in such compositions with MRI agents either before or after preparing the platelet derivatives. In one aspect, provided herein is a process for preparing a platelet derivative composition, comprising performing tangential flow filtration (TFF) of a platelet composition with a preparation agent having a pH in the range of 5.5 to 8.0 and comprising 0.4 to 35% trehalose and 2% to 8% polysucrose, wherein said TFF is performed using a 0.3 to 1 micron filter, thereby preparing a TFF-treated composition comprising 100×10¹ to 20,000×10, platelets/μl in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivatives in the platelet derivative composition are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, and a reduced propensity to aggregate, wherein no more than 25%, and in non-limiting illustrative embodiments, no more than 10%, of the platelet derivatives in the population aggregate under aggregation conditions comprising an agonist but no platelets.

In one aspect, provided herein is a process for preparing a platelet derivative composition, comprising performing tangential flow filtration (TFF) of a platelet composition with a preparation agent having a pH in the range of 5.5 to 8.0 and comprising 0.4 to 35% trehalose and 2% to 8% polysucrose, wherein said TFF is performed using a 0.3 to 1 micron filter, thereby preparing a TFF-treated composition comprising 100×10³ to 20,000×10³ platelets/μl in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivative composition comprising a population of platelet derivatives having a reduced propensity to aggregate, such that no more than 25%, and in non-limiting illustrative embodiments, no more than 10%, of the platelet derivatives in the population aggregate under aggregation conditions comprising an agonist but no platelets and having one or both of: the presence of thrombospondin (TSP) on their surface at a level that is greater than on the surface of resting platelets; and the presence of von Willebrand factor (vWF) on their surface at a level that is greater than on the surface of resting platelets.

In one aspect, provided herein is a process for preparing a process for preparing a platelet derivative composition, comprising performing tangential flow filtration (TFF) of a platelet composition with a preparation agent having a pH in the range of 5.5 to 8.0 and comprising 0.4 to 35% trehalose and 2% to 8% polysucrose, wherein said TFF is performed using a 0.3 to 1 micron filter, thereby preparing a TFF-treated composition comprising 100×10³ to 20,000×10³ platelets/μl in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivatives in the platelet derivative composition display one or more of, two or more of, three or more of, four or more of, or all of the following properties: a reduced propensity to aggregate, wherein no more than 25%, and in non-limiting illustrative embodiments, no more than 10%, of the platelet derivatives in the population aggregate under aggregation conditions comprising an agonist but no platelets; an inability to increase expression of a platelet activation marker in the presence of an agonist as compared to the expression of the platelet activation marker in the absence of the agonist; the presence of thrombospondin (TSP) on their surface at a level that is greater than on the surface of resting platelets; the presence of von Willebrand factor (vWF) on their surface at a level that is greater than on the surface of resting platelets; and are capable of generating thrombin, such that in illustrative embodiments, the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a method for preparing a composition comprising MRI agent-loaded cryopreserved platelets or MRI agent-loaded platelet derivatives, comprising:
(a) providing cryopreserved platelets or rehydrated platelet derivatives; and
(b) contacting the cryopreserved platelets or the rehydrated platelet derivatives with an MRI agent complex comprising an MRI agent, a chelator, and a linker, to form the composition comprising MRI agent-loaded cryopreserved platelets or MRI agent-loaded platelet derivatives, wherein the MRI agent is associated with the chelator, and the chelator is covalently linked to the surface of the platelets.

In some embodiments of any of the aspects and embodiments herein that include a composition, or in some compositions used in or formed by a process, the composition(s) comprises a population of MRI-agent loaded platelet derivatives having a reduced propensity to aggregate. In certain embodiments, no more than 25%, 22.5%, 20%, 17.5%, 12.5%, 10%, 7.5%, 5%, 4%, 3%, or 2 of the platelet derivatives in the population aggregate under aggregation conditions. In an illustrative embodiment no more than 10% of the platelet derivatives in the population aggregate under aggregation conditions. Illustrative embodiments of exemplary aggregation conditions are provided herein. For example, in illustrative embodiments such aggregation conditions comprise an agonist but no platelets are present in the aggregation conditions. In some embodiments, the agonist is selected from the group consisting of collagen, epinephrine, ristocetin, arachidonic acid, adenosine di-phosphate, and thrombin receptor associated protein (TRAP). In some embodiments, the population of platelet derivatives aggregate in the range of 2-30%, 5-25%, 10-30%, 10-25%, or 12.5-25% of the platelet derivatives under aggregation conditions comprising an agonist but no platelets. It can be contemplated that aggregation conditions involve rehydrating the platelet derivative composition in an appropriate amount of water or an appropriate buffer.

In one aspect, provided herein is a platelet derivative composition in the form of a powder, comprising MRI-agent loaded platelet derivatives, wherein less than 15%, and in certain non-limiting illustrative embodiments less than 5% of the CD 41-positive platelet derivatives are microparticles, in non-limiting illustrative embodiments having a diameter of less than 1 μm, and in certain non-limiting illustrative embodiments less than 0.5 μm, and wherein the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives. In some embodiments, at least 25, 30, 40, 50, 60, 70, 75, 80, 85, 90, 95, 96, 97, 98, or 99% of the MRI-loaded platelet derivatives in the composition are at least 0.5 μm in diameter by scattering intensity. In some embodiments, at least 25, 30, 40, 50, 60, 70, 75, 80, 85, 90, 95, 96, 97, 98, or 99% of the MRI-loaded platelet derivatives in the composition are between 0.5 μm and 25 μm in diameter by scattering intensity or between 0.5 μm and 2.5 μm in diameter by flow cytometry.

In one aspect, provided herein is a platelet derivative composition in the form of a powder, comprising trehalose in the range of 20-35% by weight, polysucrose in the range of 45-60% by weight, and MRI-agent loaded platelet derivatives in the range of 0.5-20% by weight, wherein the platelet derivatives to microparticles have a numerical ratio of at least 95:1 in the platelet derivative composition, and wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives.

In one aspect, provided herein is a plurality of containers each filled with a platelet derivative composition in the form of a powder, wherein the platelet derivative composition comprises trehalose in the range of 20-35% by weight; polysucrose in the range of 45-60% by weight; and MRI-agent loaded platelet derivatives in the range of 0.5-20% by weight, wherein the platelet derivatives are capable of generating thrombin, such that, for example, in illustrative embodiments the platelet derivatives are capable of generating thrombin, such that, for example, the platelet derivatives have a potency of at least 0.5, 1.0, or in non-limiting illustrative embodiments 1.5 thrombin generation potency units (TGPU) per 10⁶ platelet derivatives, and a population of platelet derivatives comprising CD41-positive platelet derivatives, wherein less than 15%, and in certain non-limiting illustrative embodiments less than 5% of the CD41-positive platelet derivatives are microparticles having a diameter of less than 1 μm, and in certain non-limiting illustrative embodiments less than 0.5 μm.

In some embodiments of any aspects and embodiments herein that include MRI agent-loaded cryopreserved platelets, or platelet derivatives, the method further comprises thawing the MRI agent-loaded cryopreserved platelets to form thawed MRI agent-loaded platelets, and wherein the thawed MRI agent-loaded platelets retain at least 10% of the loaded MRI agent upon thawing. In some embodiments, the MRI agent-loaded platelets retain between 10% and 50% of the loaded MRI agent after the thawing. In some embodiments, the thawed MRI agent-loaded platelets retain at least 20%, at least 30%, at least 40%, or at least 50% of the loaded MRI agent upon thawing. In some embodiments, the thawed MRI agent-loaded platelets are capable of releasing the MRI agent upon stimulation by endogenous platelet activators. In some embodiments, the MRI agent-loaded cryopreserved platelets retain at least 10% of the MRI agent loaded on platelets before the step of cryopreserving. In some embodiments, the MRI agent-loaded cryopreserved platelets retain at least 20%, at least 30%, at least 40%, or at least 50% of the MRI agent loaded on platelets before the step of cryopreserving.

In some embodiments of any aspects and embodiments herein that include MRI agent-loaded platelet derivatives in a powder, the method further comprises rehydrating the MRI agent-loaded platelet derivatives in the powder to form rehydrated MRI agent-loaded platelet derivatives wherein the rehydrated MRI agent-loaded platelet derivatives, retain at least 10% of the loaded MRI agent upon thawing. In some embodiments, the MRI agent-loaded platelet derivatives in the powder retain at least 20%, at least 30%, at least 40%, or at least 50% of the loaded MRI agent upon rehydrating. In some embodiments, the rehydrated MRI agent-loaded platelet derivatives retain the loaded MRI agent upon rehydration. In some embodiments, the rehydrated MRI agent-loaded platelet derivatives retain at least 10% of the loaded MRI agent upon rehydration. In some embodiments, the rehydrated MRI agent-loaded platelet derivatives retain between 10% and 50% of the loaded MRI agent upon rehydration. In some embodiments, the rehydrated MRI agent-loaded platelet derivatives retain at least 20%, at least 30%, at least 40% or at least 50% of the loaded MRI agent upon rehydration. In some embodiments, the rehydrated MRI agent-loaded platelet derivatives are capable of releasing the MRI agent upon stimulation by endogenous platelet activators. In some embodiments, the rehydrated MRI agent-loaded platelet derivatives retain at least 10% of the MRI agent loaded on platelets before the step of lyophilizing. In some embodiments, the rehydrated MRI agent-loaded platelet derivatives retain at least 20%, at least 30%, at least 40% or at least 50% of the MRI agent-loaded platelets before the step of lyophilizing.

In some embodiments herein, that includes a composition, the MRI agent-loaded platelets, the MRI agent-loaded cryopreserved platelets, or the MRI agent-loaded platelet derivatives comprise MRI agent complex covalently bonded to the platelets, typically to the surface of the platelets. In some embodiments, the MRI agent complex comprises MRI agent. Typically, the MRI agent complex comprises MRI agent and another moiety that effectuates the association of the MRI agent with the platelets. In illustrative embodiments, the MRI agent is associated with the external surface of the cryopreserved platelets or the platelet derivatives or the platelets. In some embodiments, the MRI agent is associated with the surface of the cryopreserved platelets or the surface of the platelet derivatives via the chelator. In some embodiments, the chelator is covalently attached to the surface of the cryopreserved platelets or the surface of the platelet derivatives, or the surface of the platelets. In some embodiments, the linker is covalently bonded to the chelator in the MRI agent complex. In some embodiments, the MRI agent is associated with the chelator through an ionic interaction. In some embodiments, the MRI agent-loaded platelets, the MRI agent-loaded cryopreserved platelets, or the MRI agent-loaded platelet derivatives do not comprise a drug. In some embodiments, the MRI agent-loaded platelets, the MRI agent-loaded cryopreserved platelets, or the MRI agent-loaded platelet derivatives do not comprise a CPP. In some embodiments, the MRI agent-loaded platelets, the MRI agent-loaded cryopreserved platelets, or the MRI agent-loaded platelet derivatives further comprises a drug.

In some embodiments of any of the aspects and embodiments herein that include a composition or in some compositions used in or formed by a process, the MRI-agent loaded platelet derivatives, have a potency of at least 1.25, at least 1.5, at least 1.75, at least 2.0, at least 2.25, at least 2.5 thrombin generation potency units (TGPU) per 10⁶ particles. In some embodiments, the platelet derivatives have a potency in the range of 1.2 to 2.5, 1.2 to 2.0, 1.3 to 1.5, 1.5 to 2.25, 2 to 2.5, or 2.25 to 2.5 TGPU per 10⁶ particles.

In some embodiments of any of the aspects and embodiments herein that include a composition or in some compositions used in or formed by a process, the MRI-agent loaded platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives have the presence of thrombospondin (TSP-1) on their surface at a level that is at least 50%, 60%, 70%, 80%, 90%, or 100% higher than on the surface of activated platelets, or lyophilized fixed platelets. In some embodiments, the platelet derivatives have the presence of thrombospondin (TSP-1) on their surface at a level that is more than 100% higher than on the surface of activated platelets, or lyophilized fixed platelets. In some embodiments, the MRI-agent loaded platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the MRI-agent loaded platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the MRI-agent loaded platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the MRI-agent loaded platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the MRI-agent loaded platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

In some embodiments of any of the aspects and embodiments herein that include a composition or in some compositions used in or formed by a process, the MRI-agent loaded platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of von Willebrand factor (vWF) on their surface at a level that is more than 100% higher than on the surface of resting platelets, or lyophilized fixed platelets. In some embodiments, the MRI-agent loaded platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the MRI-agent loaded platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

In some embodiments of any of the aspects and embodiments herein that include a composition or in some compositions used in or formed by a process, the MRI-agent loaded platelet derivatives have an inability to increase expression of a platelet activation marker in the presence of an agonist as compared to the expression of the platelet activation marker in the absence of the agonist. In some embodiments, the platelet activation marker is selected from the group consisting of Annexin V, and CD 62. In some embodiments, the MRI-agent loaded platelet activation marker is Annexin V. In some embodiments, the platelet activation marker is CD 62. In some embodiments, the agonist is selected from the group consisting of collagen, epinephrine, ristocetin, arachidonic acid, adenosine di-phosphate, and thrombin receptor associated protein (TRAP).

In some embodiments of any of the aspects and embodiments herein that include a composition or in some compositions used in or formed by a process herein, or a method herein that comprises MRI-agent loaded platelet derivatives, the MRI-agent loaded platelet derivatives are positive for at least one platelet activation marker selected from the group consisting of Annexin V, and CD 62. In some embodiments, the MRI-agent loaded platelet derivatives are positive for at least one platelet marker selected from the group consisting of CD 41, CD 42, and CD 61. In some embodiments, the MRI-agent loaded platelet derivatives are positive for CD 47. In some embodiments, the MRI-agent loaded platelet derivatives are positive for Annexin V. In some embodiments, the MRI-agent loaded platelet derivatives are positive for Annexin V. In some embodiments, at least 25%, 50%, or 75% of the MRI-agent loaded platelet derivatives in the platelet derivative composition are Annexin V positive. In some embodiments, the platelet derivatives are positive for CD 41. In some embodiments, at least 30%, 35%, 40%, 45%, 50%, 55%, 60%, or 65% of the MRI-agent loaded platelet derivatives in the platelet derivative composition are CD41 positive. In some embodiments, at least 90%, 91%, 92%, 93%, 94%, 95% 96%, 97%, 98%, or 99% of the MRI-agent loaded platelet derivatives that are positive for CD 41 have a size in the range of 0.5-2.5 μm in diameter by flow cytometry. In some exemplary embodiments, at least 95% of the MRI-agent loaded platelet derivatives that are positive for CD 41 have a size in the range of 0.5-2.5 μm in diameter by flow cytometry. In some embodiments, the MRI-agent loaded platelet derivatives are positive for CD 42. In some embodiments, at least 65%, 80%, or 90/a of the MRI-agent loaded platelet derivatives in the platelet derivative composition are CD42 positive. In some embodiments, the MRI-agent loaded platelet derivatives are positive for CD 47. In some embodiments, at least 8%, 10%, 15%, or 20% of the platelet derivatives in the platelet derivative composition are CD47 positive. In some embodiments, the MRI-agent loaded platelet derivatives are positive for CD 62. In some embodiments, at least 10%, 50%, 80%, or 90% of the MRI-agent loaded platelet derivatives in the platelet derivative composition are CD62 positive. In some embodiments, the MRI-agent loaded platelet derivatives in the platelet derivative composition are positive for CD41, CD62, and Annexin V. In some embodiments, the MRI-agent loaded platelet derivatives in the platelet derivative composition are at least 50% platelet derivatives are positive for CD41, at least 70% platelet derivatives are positive for CD62, and at least 70% platelet derivatives are positive for Annexin V. In some embodiments herein, the platelet derivatives are FDHs. In some embodiments, the surface expression or the amount of CD42b in or on the therapeutically effective amount of platelet derivatives, rehydrated freeze-dried platelet derivatives or the rehydrated MRI agent-loaded platelet derivatives is about 95%, 90%, 85%, 80%, 75%, 70%, 65%, 60%, 55%, 50% or less than the surface expression or the amount of CD42b on platelets, apheresis platelets, or fresh platelets. In some embodiments, the amount of CD42b on or in the platelet derivatives, rehydrated freeze-dried platelet derivatives or the rehydrated MRI agent-loaded platelet derivates is between 10% and 65%, between 12% and 64%, or between 15% and 60% the amount of CD42b on or in fresh platelets. In some embodiments, the surface expression or the amount of CD42b on the platelet derivatives, the therapeutically effective amount of rehydrated freeze-dried platelet derivatives or the rehydrated MRI agent-loaded platelet derivates is about 40% or less than the surface expression or the amount of CD42b on platelets. In some embodiments, the surface expression or the amount of CD42b on the platelet derivatives, the therapeutically effective amount of rehydrated freeze-dried platelet derivatives or the rehydrated MRI agent-loaded platelet derivates is about 25% or less than the surface expression or the amount of CD42b on platelets.

In some embodiments of any of the aspects and embodiments herein that include a composition or in some compositions used in or formed by a process, the MRI-agent loaded platelet derivatives lack an integrated membrane as compared to platelets. In some embodiments, the MRI-agent loaded platelet derivatives are surrounded by a compromised plasma membrane. In some embodiments, the MRI-agent loaded platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the MRI-agent loaded platelet derivatives can retain 35%-75%, 40-70%, 45-65%, or 35-50% lactate dehydrogenase as compared to fresh platelets, or cold stored platelets, or cryopreserved platelets. In some embodiments, the MRI-agent loaded platelet derivatives exhibit an increased permeability to antibodies. In some embodiments, the antibodies can be IgG antibodies.

In one aspect, provided herein is a process for preparing a platelet derivative composition, comprising performing tangential flow filtration (TFF) of a platelet composition with a preparation agent having a pH in the range of 5.5 to 8.0 and comprising 0.4 to 35% trehalose and 2% to 8% polysucrose, wherein the TFF is performed using a 0.3 to 1 micron filter, thereby preparing a TFF-treated composition comprising 100×10³ to 20,000×10³ platelets/μl in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm, freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried platelet composition to form the platelet derivative composition, wherein the platelet derivative composition is: a) negative for HLA Class I antibodies based on a regulatory agency approved test for HLA Class I antibodies; b) negative for HLA Class II antibodies based on a regulatory agency approved test for HLA Class II antibodies; and c) negative for HNA antibodies based on a regulatory agency approved test for HNA antibodies.

In some embodiments of any of the aspects and embodiments herein that include a composition or in some compositions used in or formed by a process that includes a population of MRI-agent loaded platelet derivatives in a hydrated or rehydrated form, the composition comprises less than 10%, 7.5%, 7%, 6%, 5%, 4.5%, 4%, 3.5%, 3%, 2.5%, 2%, 1.5%, 1.0%, 0.75%, 0.5%, 0.25%, or 0.1% (by scattering intensity) microparticles. In some embodiments, the composition comprises microparticles (by scattering intensity) in the range of 0.01%-10%, 0.01%-7.5%, 0.01/6-5%, 0.1%-10%, 0.1%-5%, 1%-10%, 1%-5%, 0.01%-4%, 0.1%-4%, 1%-4%, 0.1%-3%, or 1%-3%.

In some embodiments, the microparticles have a diameter less than 1 μm. In illustrative embodiments, the microparticles have a diameter less than 0.5 μm. In some embodiments, the microparticles have a diameter in the range of 0.01-0.5 μm, 0.1-0.5 μm, or 0.1-0.49 μm, 0.1-0.47 μm, or 0.1-0.45 μm, or 0.1-0.4 μm, or 0.2-0.49 μm, or 0.25-0.49 μm, or 0.3-0.47 μm. In some embodiments, the diameter of the microparticles is measured using flow cytometry.

In some embodiments of any of the aspects and embodiments herein that include a platelet derivative composition in a powdered form, the platelet derivative composition comprises a population of MRI-agent loaded platelet derivatives comprising CD41-positive platelet derivatives, wherein less than 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm 0.4 μm, 0.3 μm, 0.2 μm, or 0.1 μm. In some embodiments, the platelet derivative composition comprises a population of MRI-agent loaded platelet derivatives comprising CD42-positive platelet derivatives, wherein less than 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, the platelet derivative composition comprises a population of MRI-agent loaded platelet derivatives comprising CD61-positive platelet derivatives, wherein less than 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some illustrative embodiments, the microparticles are having a diameter of less than 0.5 μm. In some embodiments of any of the aspects and embodiments herein that include a platelet derivative composition in a powdered form diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. In some embodiments, the diameter of the microparticles is determined by flow cytometry.

In some embodiments of any of the aspects and embodiments herein that include a composition, or in some compositions used in or formed by a process that includes a plurality of containers each filled with a platelet derivative composition in the form of a powder, the amount of microparticles that are less than 0.5 μm in the powder of any two containers chosen from different lots, differs by less than 50%, 40%, 30%, 20%, 10%, 5%, 2%, 1%, or 0.5%.

In one aspect, provided herein is a composition comprising MRI agent-loaded cryopreserved platelets or MRI agent-loaded platelet derivatives, wherein the MRI agent-loaded cryopreserved platelets or the MRI agent-loaded platelet derivatives comprise a magnetic resonance imaging (MRI) agent coupled to a cell penetrating peptide (CPP). In some embodiments, the MRI agent-loaded cryopreserved platelets, the MRI agent-loaded platelet derivatives, or the rehydrated MRI agent-loaded platelet derivatives do not comprise a cell penetrating peptide (CPP).

In one aspect, provided herein is a composition comprising MRI agent-loaded cryopreserved platelets or MRI agent-loaded platelet derivatives, wherein the MRI agent-loaded cryopreserved platelets or the MRI agent-loaded platelet derivatives comprise an MRI agent coupled to a CPP, and the MRI agent-loaded cryopreserved platelets or the MRI agent-loaded platelet derivatives do not comprise a drug.

In one aspect, provided herein is a composition comprising MRI agent-loaded cryopreserved platelets or MRI agent-loaded platelet derivatives in a dried powder, wherein the MRI agent-loaded cryopreserved platelets or the MRI agent-loaded platelet derivatives comprise an MRI agent complex covalently bonded to the surface of the cryopreserved platelets or the platelet derivatives.

In one aspect, provided herein is a composition comprising MRI agent-loaded platelet derivatives, wherein the MRI agent-loaded platelet derivatives comprise a magnetic resonance imaging (MRI) agent coupled to a cell penetrating peptide (CPP) or the MRI agent-loaded platelet derivatives comprise an MRI agent complex covalently bonded to the surface of the platelet derivatives, and wherein the platelet derivatives are freeze-dried platelet derivatives having one, two, three, or more properties as described herein.

In one aspect, provided herein is a composition comprising MRI agent-loaded platelet derivatives, wherein the MRI agent-loaded platelet derivatives comprise a magnetic resonance imaging (MRI) agent coupled to a cell penetrating peptide (CPP) or the MRI agent-loaded platelet derivatives comprise an MRI agent complex covalently bonded to the surface of the platelet derivatives, and wherein the MRI agent-loaded platelet derivatives do not comprise a drug, and wherein the platelet derivatives are freeze-dried platelet derivatives having one, two, three, or more properties as described herein.

In one aspect, provided herein is a method for preparing a composition comprising MRI agent-loaded cryopreserved platelets or MRI agent-loaded platelet derivatives in a powder, comprising: (a) providing platelets; (b) contacting the platelets with an MRI agent coupled to a cell penetrating peptide, and a buffer comprising a salt, a base, a loading agent, and optionally at least one organic solvent, to form MRI agent-loaded platelets; and (c) cryopreserving the MRI agent-loaded platelets to form the composition comprising the MRI agent-loaded cryopreserved platelets or lyophilizing the MRI agent-loaded platelets to form the composition comprising the MRI agent-loaded platelet derivatives.

In one aspect, provided herein is a method for preparing a composition comprising MRI agent-loaded cryopreserved platelets or MRI agent-loaded platelet derivatives, comprising: (a) providing cryopreserved platelets or rehydrated platelet derivatives; and (b) contacting the cryopreserved platelets or the rehydrated platelet derivatives with an MRI agent coupled to a cell penetrating peptide, to form the composition comprising MRI agent-loaded cryopreserved platelets or MRI agent-loaded platelet derivatives.

In one aspect, provided herein is a method for preparing a composition comprising MRI agent-loaded cryopreserved platelets or MRI agent-loaded platelet derivatives in a powder, comprising: (a) providing platelets; (b) contacting the platelets with an MRI agent coupled to a cell penetrating peptide, and a buffer comprising a salt, a base, a loading agent, and optionally at least one organic solvent, to form MRI agent-loaded platelets; and (c) cryopreserving the MRI agent-loaded platelets to form the composition comprising the MRI agent-loaded cryopreserved platelets or lyophilizing the MRI agent-loaded platelets to form the composition comprising MRI agent-loaded platelet derivatives, wherein the MRI agent-loaded cryopreserved platelets or the MRI agent-loaded platelet derivatives do not comprise a drug.

In one aspect, provided herein is a method for preparing a composition comprising MRI agent-loaded cryopreserved platelets or MRI agent-loaded platelet derivatives, comprising: (a) providing cryopreserved platelets or rehydrated platelet derivatives; and (b) contacting the cryopreserved platelets or the rehydrated platelet derivatives with an MRI agent coupled to a cell penetrating peptide, to form the composition comprising MRI agent-loaded cryopreserved platelets and MRI agent-loaded platelet derivatives, wherein the MRI agent-loaded cryopreserved platelets or the MRI agent-loaded platelet derivatives do not comprise a drug.

In one aspect, provided herein is a method for preparing a composition comprising MRI agent-loaded cryopreserved platelets or MRI agent-loaded platelet derivatives in a powder, comprising: (a) providing platelets; (b) contacting the platelets with an MRI agent complex comprising an MRI agent, a chelator, and a linker, to form MRI agent-loaded platelets; and (c) cryopreserving or lyophilizing the MRI agent-loaded platelets to form the composition comprising the MRI agent-loaded cryopreserved platelets or the MRI agent-loaded platelet derivatives. In illustrative embodiments, the MRI agent-loaded cryopreserved platelets or the MRI agent-loaded platelet derivatives comprise MRI agent complex covalently bonded to the cryopreserved platelets or to the platelet derivatives, and the covalently bonded MRI agent complex comprises the MRI agent, and the chelator.

In one aspect, provided herein is a method for preparing a composition comprising MRI agent-loaded cryopreserved platelets or MRI agent-loaded platelet derivatives, comprising: (a) providing cryopreserved platelets or rehydrated platelet derivatives; and (b) contacting the cryopreserved platelets or the rehydrated platelet derivatives with an MRI agent complex such that the MRI agent complex comprising an MRI agent, a chelator, and a linker, to form the composition comprising MRI agent-loaded cryopreserved platelets or MRI agent-loaded platelet derivatives.

In one aspect, provided herein is a process for preparing a composition comprising MRI agent-loaded platelet derivatives as described in any of the aspects herein, the process comprising: (a) performing tangential flow filtration (TFF) of a platelet composition with a preparation agent having a pH in the range of 5.5 to 8.0 and comprising 0.4 to 35% trehalose and/or 2% to 8% polysucrose, wherein said TFF is performed using a 0.3 to 1 micron filter, thereby preparing a TFF-treated composition comprising 100×10³ to 20,000×10¹ platelets/μl in an aqueous medium having less than or equal to 15% plasma protein, and having less than 5.0% microparticles having a diameter less than 0.5 μm, by scattering intensity; (b) contacting the TFF-treated composition with an MRI agent coupled to a cell penetrating peptide, and a buffer comprising a salt, a base, a loading agent, and optionally at least one organic solvent to form MRI agent-loaded TFF-treated composition or (b′) contacting the TFF-treated composition with an MRI agent complex at a temperature in the range of 15-45° C. for a time period in the range of 5 minutes to 48 hours, to form MRI agent-loaded TFF treated composition such that the MRI agent complex is covalently linked to the platelets in the MRI agent-loaded TFF treated composition; (c) freeze drying the MRI agent-loaded TFF-treated composition of step (b) or (b′) comprising platelets in the aqueous medium to form a freeze-dried composition comprising platelet derivatives; and (d) heating the freeze-dried composition at a temperature in the range of 60° C. to 90° C. for at least 1 hour to not more than 36 hours to thermally treat the platelet derivatives in the freeze-dried composition to form the MRI agent-loaded platelet derivatives in a powder.

In one aspect, provided herein is a process for preparing a composition comprising MRI agent-loaded platelet derivatives as described in any of the aspects herein, the process comprising: (a) performing tangential flow filtration (TFF) of a platelet composition with a preparation agent having a pH in the range of 5.5 to 8.0 and comprising 0.4 to 35% trehalose and/or 2% to 8% polysucrose, wherein said TFF is performed using a 0.3 to 1 micron filter, thereby preparing a TFF-treated composition comprising 100×10³ to 20,000×10³ platelets/μl in an aqueous medium having less than or equal to 15% plasma protein, and having less than 5.0% microparticles having a diameter less than 0.5 μm, by scattering intensity; (b) freeze drying the TFF-treated composition of step (a) comprising platelets in the aqueous medium to form a freeze-dried composition comprising platelet derivatives; (c) heating the freeze-dried composition at a temperature in the range of 60° C. to 90° C. for at least 1 hour to not more than 36 hours to thermally treat the platelet derivatives in the freeze-dried composition to form the platelet derivatives in a powder; and (d) rehydrating the platelet derivatives of step (c) to form rehydrated platelet derivatives and contacting the rehydrated platelet derivatives with an MRI agent coupled to a cell penetrating peptide, and a buffer comprising a salt, a base, a loading agent, and optionally at least one organic solvent to form MRI agent-loaded platelet derivatives or (d′) rehydrating the platelet derivatives of step (c) to form rehydrated platelet derivatives and contacting the rehydrated platelet derivatives with an MRI agent complex at a temperature in the range of 15-45° C. for a time period in the range of 5 minutes to 48 hours, to form MRI agent-loaded TFF treated composition such that the MRI agent complex is covalently linked to the platelets in the MRI agent-loaded TFF treated composition.

In one aspect, provided herein is a method of delivering an MRI agent in a subject, comprising administering an effective amount of the composition comprising MRI agent-loaded platelets, MRI agent-loaded cryopreserved platelets or MRI agent-loaded platelet derivatives of any of the aspects or embodiments described herein, or the composition prepared by the process of any of the aspects or embodiments described herein.

In one aspect, provided herein is a method for detecting site of bleeding in a subject, comprising: (a) administering an effective amount of the composition comprising MRI agent loaded platelets, MRI agent-loaded cryopreserved platelets or MRI agent-loaded platelet derivatives as described in any of the aspects or embodiments herein, or the composition prepared by the process as described in any of the aspects or embodiments herein, to the subject; and (b) detecting the site of the MRI agent-loaded cryopreserved platelets or MRI agent-loaded platelet derivatives for detecting the site of bleeding in the subject. In some embodiments, a method for detecting an MRI agent well known in the art can be used herein for detecting the site of bleeding in a subject after the administration of MRI agent loaded platelets, MRI agent-loaded cryopreserved platelets or MRI agent-loaded platelet derivatives.

In one aspect, provided herein is a composition comprising MRI agent-loaded platelets, MRI agent-loaded cryopreserved platelets, or MRI agent-loaded platelet derivatives as described herein, for use in the treatment of a subject having an indication selected from the group consisting of Acute lymphoblastic leukemia (ALL), Acute myeloid leukemia (AML), Breast cancer, Gastric cancer, Hodgkin lymphoma, Neuroblastoma, —Non-Hodgkin lymphoma, Ovarian cancer, Cervical cancer, Small cell lung cancer, Non-small cell lung cancer (NSCLC), Soft tissue and bone sarcomas, Thyroid cancer, Transitional cell bladder cancer, Wilms tumor Neuroendocrine tumors, Pancreatic cancer, Multiple myeloma, Renal cancer, Glioblastoma Prostate cancer, Sarcoma, Colon cancer, Melanoma, Colitis, Chronic inflammatory demyelinating polyneuropathy, Guil-ain-Barre syndrome, Immune Thrombocytopenia, Kawasaki disease, Lupus, Multiple Sclerosis, Myasthenia gravis, Myositis, Cirrhosis with refractory ascites, Hepatorenal syndrome, Nephrotic syndrome, Organ transplantation, Paracentesis, Hypovolemia, Aneurysms, Artherosclerosis, Cancer, Cardiovascular diseases, Genetic disorders, Infectious diseases, Metabolic diseases, Neoangiogenesis, Opthalmic conditions, Hypercholesterolemia, and Pulmonary hypertension.

In one aspect, provided herein is a composition comprising MRI agent-loaded platelets, MRI agent-loaded cryopreserved platelets, or MRI agent-loaded platelet derivatives as described herein, for use in the treatment of a subject having an indication selected from the group consisting of Von Willebrand disease, Immune thrombocytopenia, Hermansky Pudlak Syndrome (HPS), Chemotherapy induced thrombocytopenia (CM, Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alex'nder's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof.

In some embodiments of any of the aspects provided herein that relates to a composition or a process or a method comprising MRI agent-loaded platelets, MRI agent-loaded cryopreserved platelets, or MRI agent-loaded platelet derivatives, such MRI agent loaded platelets, MRI agent-loaded cryopreserved platelets, or MRI agent-loaded platelet derivatives comprise a CPP that is coupled to an MRI agent. In some embodiments, a composition comprising MRI agent-loaded platelets/cryopreserved platelets/platelet derivatives do not comprise a drug. In some embodiments, a drug can be any drug known in the art that can be used to treat a subject. For example, a drug can be any drug that is or has been listed as an active ingredient in the FDA Orange Book or Purple Book. In some embodiments, a composition comprises MRI agent-loaded platelet derivatives that can be freeze-dried platelet derivatives (FDPDs) as described in any of the aspects or embodiments herein having one, two, three, or more properties of FDPDs as described herein.

In some embodiments of any of the aspects provided herein that relates to a composition or a process or a method comprising MRI agent-loaded platelets, MRI agent-loaded cryopreserved platelets, or MRI agent-loaded platelet derivatives, wherein the MRI agent-loaded platelets, MRI agent-loaded cryopreserved platelets or the MRI agent-loaded platelet derivatives comprise an MRI agent complex covalently bonded to the surface of the platelets, cryopreserved platelets or the platelet derivatives. In some embodiments, an MRI agent complex comprises an MRI agent, a linker, and a chelator. In illustrative embodiments, an MRI agent complex comprises an MRI agent, and a chelator. In some embodiments, a composition comprising MRI agent-loaded platelets/cryopreserved platelets/platelet derivatives having an MRI agent complex covalently bonded to the surface do not comprise a drug. In some embodiments, a drug can be any drug known in the art that can be used to treat a subject. In some embodiments, a composition comprising MRI agent-loaded platelets/cryopreserved platelets/platelet derivatives having an MRI agent complex covalently bonded to the surface do not comprise a CPP. In some embodiments, a composition comprising MRI agent-loaded platelet derivatives having an MRI agent complex covalently bonded to the surface can be freeze-dried platelet derivatives as described in any of the aspects or embodiments herein having one, two, three, or more properties of FDPDs as described herein.

In some embodiments of any of the aspects or embodiments that describe a composition or a process or a method comprising MRI agent-loaded platelets, MRI agent-loaded cryopreserved platelets, or MRI agent-loaded platelet derivatives, wherein the MRI agent-loaded platelets, MRI agent-loaded cryopreserved platelets or the MRI agent-loaded platelet derivatives comprise an MRI agent complex covalently bonded to the surface of the platelets, cryopreserved platelets or the platelet derivatives, a chelator can be a well-known chelator in the art. In some embodiments, a chelator is selected from the group consisting of dodecane tetra acetic acid (DOTA), diethylenetriaminepentaacetic acid (DTPA), 4-Carboxy-5,8,11-tris(carboxymethyl)-1-phenyl-2-oxa-5,8,11-triazatridecan-13-oic acid (BOPTA), Ethylenediaminetetraacetic acid (EDTA), and 1,4,7,10-tetraazacyclododecane-1,4,7-tetracetic acid (DO3A). In other embodiments, a chelator can be a chelator that is known to suppress any toxic effects of an MRI agent.

In some embodiments of any of the aspects or embodiments that describe a composition or a process or a method comprising MRI agent-loaded platelets, MRI agent-loaded cryopreserved platelets, or MRI agent-loaded platelet derivatives, wherein the MRI agent-loaded platelets, MRI agent-loaded cryopreserved platelets or the MRI agent-loaded platelet derivatives comprise an MRI agent complex covalently bonded to the surface of the platelets, cryopreserved platelets or the platelet derivatives, a linker can be any linker molecule/moiety that can effectuate a covalent bonding to protein(s). In some embodiments, a linker can be selected from the group consisting of a compound having sulfhydryl reactive groups, such as maleimides and haloacetyl derivatives, amine reactive groups, such as isothiocyanates, succinimidyl esters, and sulfonyl halides, and carbodiimide reactive groups, such as carboxyl and amino groups. In illustrative embodiments, a linker can be NHS ester. Typically, the linker moiety is released after effectuating the bonding between the chelator and the primary amine that typically can come from the proteins present on the surface of platelets/cryopreserved platelets/platelet derivatives. In some embodiments, the NHS forms an ester with DOTA.

In some embodiments of any of the aspects or embodiments that describe a composition or a process or a method comprising MRI agent-loaded platelets, MRI agent-loaded cryopreserved platelets, or MRI agent-loaded platelet derivatives, wherein an MRI agent can be any contrast agents known in the art that can facilitate imaging when administered to a subject. In some embodiments, an MRI agent is selected from the group consisting of a superparamagnetic contrast agent, a diamagnetic agent, or combinations thereof. In some embodiments, the superparamagnetic metal ion is selected from the group consisting of Gd(III), Fe(III), Mn(II and III), Cr(III), Cu(II), Dy(III), Tb(III and IV), Ho(III), Er(III), Pr(III) and Eu(II and III). In some embodiments, an MRI agent is selected from the group consisting of metal ions with atomic numbers 21-29, 39-47, or 57-83. In illustrative embodiments, an MRI agent is gadolinium.

In some embodiments of any of the aspects provided herein that relates to a composition or a process or a method comprising MRI agent-loaded platelets, MRI agent-loaded cryopreserved platelets, or MRI agent-loaded platelet derivatives, such MRI agent loaded platelets, MRI agent-loaded cryopreserved platelets, or MRI agent-loaded platelet derivatives comprise a CPP that is coupled to an MRI agent, wherein a CPP can be any CPP that is known to get across plasma membrane. In some embodiments, a CPP can be a protein-derived CPP, a synthetic CPP, or a mixed CPP. In some embodiments, a protein-derived CPPs is selected from the group consisting of Pep-1, penetratin, TAT peptide (49-57 amino acids), TAT peptide (48-60 amino acids), calcitonin-derived CPP, nuclear localization sequences, new polybasic CPPs, N-terminal repetitive domain of maize gamma-zein, peptides from gp41 fusion sequence, preS2-TLM, signal-sequence hydrophobic region (SSHR), pVEC, Vpr, VP22, Human integrin b3 signal sequence, gp41 fusion sequence, Caiman crocodylus Ig(v) light chain, hCT derived peptide, Kaposi FGF signal sequences, CPP from pestivirus envelope glycoprotein, CPP derived from the prion protein, Yeast PRP6 (129-144), Phi21 N (12-29), Delta N (1-22), FHV coat (35-49), BMV Gag (7-25), HTLV-II Rex (4-16), HIV-1 Rev (9-20), RSG-1.2, Lambda-N(48-62), SV40 NLS, Bipartite, Nucleoplasmin (155-170), NLS, Herpesvirus, 8 k8 protein (124-135), Buforin-II (20-36), Magainin, PDX-1-PTD, crotamine, pIs1, SynB1, Fushi-tarazu (254-313), and Engrailed (454-513). In some embodiments, a CPP can be selected from the group consisting of penetratin, calcitonin-derived CPP, nuclear localization sequences, new polybasic CPPs, N-terminal repetitive domain of maize gamma-zein, peptides from gp41 fusion sequence, preS2-TLM, signal-sequence hydrophobic region (SSHR), pVEC, Vpr, VP22, Human integrin b3 signal sequence, gp41 fusion sequence, Caiman crocodylus Ig(v) light chain, hCT derived peptide, Kaposi FGF signal sequences, CPP from pestivirus envelope glycoprotein, CPP derived from the prion protein, Yeast PRP6 (129-144), Phi21 N (12-29), Delta N (1-22), FHV coat (35-49), BMV Gag (7-25), HTLV-II Rex (4-16), HIV-1 Rev (9-20), RSG-1.2, Lambda-N(48-62), SV40 NLS, Bipartite, Nucleoplasmin (155-170), NLS, Herpesvirus, 8 k8 protein (124-135), Buforin-II (20-36), Magainin, PDX-1-PTD, crotamine, pIs1, SynB1, Fushi-tarazu (254-313), and Engrailed (454-513). In some embodiments, synthetic and mixed CPP is selected from the group consisting of transportan, polyarginine CPPs, poly-d-arginine, KLAL peptide/model amphipathic peptide (MAP), KALA model amphipathic peptide, modeled Tat peptide, Loligomer, b-sheet-forming peptide, retro-inverso forms of established CPPs, W/R penetratin, MPG, Pep-1, Signal-sequence-based peptides (I), Signal-sequence-based peptides (II), Carbamate 9, PTD-4, PTD-5, RSV-A9, CTP-512, and U2AF.

In some embodiments of any of the aspects or embodiments that describe a composition comprising or a process/method forming a population of MRI agent-loaded platelet derivatives/freeze-dried platelet derivatives/cryopreserved platelets, wherein the composition comprises a population of platelet derivatives/freeze-dried platelet derivatives/cryopreserved platelets having a reduced propensity to aggregate such that no more than 10% of the platelet derivatives/freeze-dried platelet derivatives/cryopreserved platelets in the population aggregate under aggregation conditions comprising an agonist but no platelets.

In some embodiments of any of the aspects or embodiments that describe a composition comprising or a process/method forming a population of MRI agent-loaded platelet derivatives/freeze-dried platelet derivatives/cryopreserved platelets, wherein the platelet derivatives freeze-dried platelet derivatives/cryopreserved platelets have a potency of at least 1.5 thrombin generation potency units (TGPU) per 10⁶ platelet derivatives/freeze-dried platelet derivatives/cryopreserved platelets.

In some embodiments of any of the aspects or embodiments that describe a composition comprising or a process/method forming a population of MRI agent-loaded platelet derivatives/freeze-dried platelet derivatives/cryopreserved platelets, wherein the platelet derivatives, freeze-dried platelet derivatives/cryopreserved platelets are capable of generating thrombin in an in vitro thrombin generation assay (also referred to herein as a thrombin formation assay).

In some embodiments of any of the aspects or embodiments that describe a composition comprising or a process/method forming a population of MRI agent-loaded platelet derivatives/freeze-dried platelet derivatives/cryopreserved platelets, wherein the platelet derivatives, freeze-dried platelet derivatives/cryopreserved platelets are capable of occluding a collagen coated channel in a T-TAS assay. In some embodiments, the MRI agent-loaded cryopreserved platelets are capable of forming aggregates such that when at a concentration of 80×10³ particles/μl, a T-TAS occlusion time of less than 30, 28, 25, or in illustrative embodiments less than 23 minutes is achieved. In some embodiments, the MRI agent-loaded platelet derivatives are capable of forming aggregates such that when at a concentration of at 80×10³ particles/μl, a T-TAS occlusion time of less than 25, 22, 20, 18, or in illustrative embodiments less than 15 minutes is achieved.

In some embodiments of any of the aspects or embodiments that describe a composition comprising or a process/method forming a population of MRI agent-loaded platelet derivatives/freeze-dried platelet derivatives/cryopreserved platelets, wherein at least 50% of the platelet derivatives/freeze-dried platelet derivatives/cryopreserved platelets are CD 41-positive platelet derivatives/freeze-dried platelet derivatives/cryopreserved platelets, wherein less than 5% of the CD 41-positive platelet derivatives/freeze-dried platelet derivatives/cryopreserved platelets are microparticles having a diameter of less than 0.5 μm.

In some embodiments of any of the aspects or embodiments that describe a composition comprising or a process/method forming a population of MRI agent-loaded platelet derivatives/freeze-dried platelet derivatives/cryopreserved platelets, wherein the platelet derivatives/freeze-dried platelet derivatives/cryopreserved platelets have one or more characteristics of a super-activated platelet selected from A) the presence of thrombospondin (TSP) on their surface at a level that is greater than on the surface of resting platelets; B) the presence of von Willebrand factor (vWF) on their surface at a level that is greater than on the surface of resting platelets; and C) an inability to increase expression of a platelet activation marker in the presence of an agonist as compared to the expression of the platelet activation marker in the absence of an agonist.

In some embodiments of any of the aspects or embodiments that describe a composition comprising or a process/method forming a population of MRI agent-loaded platelet derivatives/freeze-dried platelet derivatives, wherein MRI agent-loaded platelet derivatives/freeze-dried platelet derivatives have a reduced propensity to aggregate such that no more than 10% of the MRI agent-loaded platelet derivatives in the population aggregate under aggregation conditions comprising an agonist but no platelets, and wherein the platelet derivatives have a potency of at least 1.5 thrombin generation potency units (TGPU) per 10⁶ platelet derivatives.

In some embodiments of any of the aspects or embodiments that describe a composition comprising or a process/method forming a population of MRI agent-loaded platelet derivatives/freeze-dried platelet derivatives, wherein less than 5% of the CD 41-positive platelet derivatives are microparticles having a diameter of less than 0.5 μm, and wherein the platelet derivatives have a potency of at least 1.5 thrombin generation potency units (TGPU) per 10⁶ platelet derivatives or are capable of generating thrombin in an in vitro thrombin generation assay or are capable of occluding a collagen coated channel in a T-TAS assay.

In some embodiments of any of the aspects or embodiments that describe a composition comprising or a process/method forming a population of MRI agent-loaded platelet derivatives/freeze-dried platelet derivatives, wherein the composition comprises a population of platelet derivatives having a reduced propensity to aggregate such that no more than 10% of the platelet derivatives in the population aggregate under aggregation conditions comprising an agonist but no platelets; and having one or more characteristics of a super-activated platelet selected from A) the presence of thrombospondin (TSP) on their surface at a level that is greater than on the surface of resting platelets; B) the presence of von Willebrand factor (vWF) on their surface at a level that is greater than on the surface of resting platelets; and C) an inability to increase expression of a platelet activation marker in the presence of an agonist as compared to the expression of the platelet activation marker in the absence of an agonist.

In some embodiments of any of the aspects or embodiments that describe a composition comprising or a process/method forming a population of MRI agent-loaded platelet derivatives/freeze-dried platelet derivatives, wherein the composition comprises a population of platelet derivatives having a reduced propensity to aggregate such that no more than 10% of the platelet derivatives in the population aggregate under aggregation conditions comprising an agonist but no platelets, wherein the platelet derivatives have an inability to increase expression of a platelet activation marker in the presence of an agonist as compared to the expression of the platelet activation marker in the absence of the agonist, wherein the platelet derivatives have a potency of at least 1.5 thrombin generation potency units (TGPU) per 10⁶ platelet derivatives or are capable of generating thrombin in an in vitro thrombin generation assay or are capable of occluding a collagen coated channel in a T-TAS assay; and wherein less than 5% of the CD 41-positive platelet derivatives are microparticles having a diameter of less than 0.5 μm.

In some embodiments of any of the aspects or embodiments that describe a composition comprising or a process/method forming a population of MRI agent-loaded platelet derivatives/freeze-dried platelet derivatives, wherein the composition comprises a population of platelet derivatives having a reduced propensity to aggregate such that no more than 10% of the platelet derivatives in the population aggregate under aggregation conditions comprising an agonist but no platelets, and further having one or both of: the presence of thrombospondin (TSP) on their surface at a level that is greater than on the surface of resting platelets; and the presence of von Willebrand factor (vWF) on their surface at a level that is greater than on the surface of resting platelets.

In some embodiments of any of the aspects or embodiments that describe a composition comprising or a process/method forming a population of MRI agent-loaded platelet derivatives/freeze-dried platelet derivatives, wherein the composition comprises a population of platelet derivatives comprising CD 41-positive platelet derivatives, wherein less than 5% of the CD 41-positive platelet derivatives are microparticles having a diameter of less than 0.5 μm, and comprising platelet derivatives having: a reduced propensity to aggregate such that no more than 10% of the platelet derivatives in the population aggregate under aggregation conditions comprising an agonist but no platelets; an inability to increase expression of a platelet activation marker in the presence of an agonist as compared to the expression of the platelet activation marker in the absence of the agonist; the presence of thrombospondin (TSP) on their surface at a level that is greater than on the surface of resting platelets; the presence of von Willebrand factor (vWF) on their surface at a level that is greater than on the surface of resting platelets; and a potency of at least 1.5 thrombin generation potency units (TGPU) per 10⁶ platelet derivatives or are capable of generating thrombin in an in vitro thrombin generation assay or are capable of occluding a collagen coated channel in a T-TAS assay.

In some embodiments of any of the aspects or embodiments herein that relate to a process/method for forming MRI agent-loaded platelets/platelet derivatives/cryopreserved platelets/FDPDs, wherein an MRI agent in the MRI agent complex or in MRI agent coupled to a CPP, has a concentration in the range of 2 mM to 100 mM. In some embodiments, an MRI agent during any step of preparing as per any of the aspects, or embodiments herein has a concentration of at least 0.1 mM, 1 mM, 5 mM, 10 mM, 50 mM, 100 mM, 150 mM, 200 mM, 250 mM, 300 mM, 350 mM, 400 mM, 450 mM, 500 mM, or higher. In some embodiments, an MRI agent has a concentration in the range of 0.1 to 500 mM, 1 to 400 mM, 20 to 400 mM, 50 to 400 mM, 75 to 350 mM, 1 to 350 mM, 2 to 300 mM, 4 to 250 mM, or 5 to 150 mM.

In some embodiments of any of the aspects or embodiments herein that relate to a process/method for forming MRI agent-loaded platelets/platelet derivatives/cryopreserved platelets/FDPDs, wherein an MRI agent complex has a concentration in the range of 5 to 500 μM. In some embodiments, an MRI agent complex has a concentration of at least 1 μM, 5 μM, 10 μM, 50 μM, 100 μM, 200 μM, 300 μM, 400 μM, 500 μM, 700 μM, 800 μM, or higher. In some embodiments, an MRI agent complex has a concentration in the range of 1 to 500 μM, 5 to 500 μM, 10 to 500 μM, 20 to 400 μM, 25 to 300 μM, 30 to 500 μM, or 35 to 250 μM.

In some embodiments of any of the aspects or embodiments herein that relate to a process/method for forming MRI agent-loaded platelets/platelet derivatives/cryopreserved platelets/FDPDs that involves CPP, wherein CPP has a concentration in the range of 5 μM to 500 μM. In some embodiments, CPP has a concentration of at least 1 μM, 5 μM, 10 μM, 50 μM, 100 μM, 200 μM, 300 μM, 400 μM, 500 μM, 700 μM, 800 μM, or higher. In some embodiments, CPP has a concentration in the range of 1 to 500 μM, 5 to 500 μM, 10 to 500 μM, 20 to 400 μM, 25 to 300 μM, 30 to 500 μM, or 35 to 250 μM. In some embodiments, CPP has a concentration in the range of 1 to 500 mM, 5 to 500 mM, 10 to 500 mM, 20 to 400 mM, 25 to 300 mM, 30 to 500 mM, or 35 to 250 mM.

In some embodiments of any of the aspects or embodiments herein that relate to a process/method for forming MRI agent-loaded platelets/platelet derivatives/cryopreserved platelets/FDPDs, wherein platelets has a concentration in the range of 1000 platelets/μl to 300×10⁶ platelets/μl. In some embodiments, platelets has a concentration of at least 1000, 2000, 3000, 5000, 7500, 10⁴, 5×10⁴, or higher. In some embodiments, platelets has a concentration in the range of 1000 to 500×10⁶, 1500 to 500×10⁶, 2000 to 500×10⁵, 2000 to 1×10⁶, 2000 to 500×10⁴, 3000 to 1×10⁶, 4000 to 50×10⁵, or 5000 to 500×10⁶.

In some embodiments of any of the aspects or embodiments that describe a composition comprising or a process/method forming a population of MRI agent-loaded platelet derivatives/freeze-dried platelet derivatives/cryopreserved platelets, wherein the MRI agent-loaded platelet derivatives/freeze-dried platelet derivatives/cryopreserved platelets have less than 10% crosslinking of platelet membranes via proteins and/or lipids present on the membranes. In some embodiments, have less than 9%, 8%, 7%, 6%, 5%, 4%, 3%, 2%, 1%, 0.5%, or 0.1%. In some embodiments, have crosslinking in the range of 0.1-10%, 0.1-2%, 1-10%, 2-10%, 1-8%, 1-7%, 1-5%, or 1-3%. In some embodiments, the platelet derivatives, or the rehydrated freeze-dried platelet derivatives have 0.001-5%, 0.001-4%, 0.001-3%, 0.01-5%, 0.01-4%, or 0.01-3% crosslinking of platelet membranes via proteins and/or lipids present on the membranes. In some embodiments, the platelet derivatives, or the rehydrated freeze-dried platelet derivatives have less than 5%, 4%, 3%, 2%, 1%, 0.1% or 0.01% crosslinking of platelet membranes via proteins and/or lipids present on the membranes. In some embodiments the platelet derivatives, or the rehydrated freeze-dried platelet derivatives have no exogenous crosslinking of proteins. In some embodiments, the platelet derivatives, or the freeze-dried platelet derivatives have no exogenous crosslinking of membranes proteins.

In some embodiments of any of the aspects or embodiments herein that relate to a process/method for forming MRI agent-loaded platelets/platelet derivatives/cryopreserved platelets/FDPDs, wherein contacting the platelets, the cryopreserved platelets, the platelet derivatives, or the TFF-treated composition with the MRI agent coupled to a cell penetrating peptide or with the MRI agent complex is done at a temperature in the range of 15 to 45° C. In some embodiments, contacting is done at a temperature in the range of 10 to 45° C., 15 to 45° C., 20 to 45° C., 25 to 45° C., 30 to 45° C., or 32 to 42° C.

In some embodiments of any of the aspects or embodiments herein that relate to a process/method for forming MRI agent-loaded platelets/platelet derivatives/cryopreserved platelets/FDPDs, wherein contacting the platelets, the cryopreserved platelets, the platelet derivatives, or the TFF-treated composition with an MRI agent coupled to a cell penetrating peptide or with the MRI agent complex is done for a time period in the range of 5 minutes to 48 hours. In some embodiments, time period is in the range of 1 minutes to 72 hours, 5 minutes to 60 hours, 5 minutes to 52 hours, 5 minutes to 45 hours, 10 minutes to 40 hours, 10 minutes to 30 hours, 15 minutes to 25 hours, 15 minutes to 15 hours, 15 minutes to 10 hours, 15 minutes to 5 hours, 20 minutes to 3 hours, 20 minutes to 1 hour, or 20 minutes to 45 minutes.

In some embodiments of any of the aspects or embodiments herein that relate to a process/method for forming MRI agent-loaded platelets/platelet derivatives/cryopreserved platelets/FDPDs include (a) contacting platelets with an MRI agent complex in the presence of a buffer comprising a salt, a base, a loading agent, and optionally at least one organic solvent, at a temperature in the range of 15-45° C. for a time period in the range of 5 minutes to 48 hours, to form MRI agent-loaded platelets such that the MRI agent complex is covalently linked to the platelets in the MRI agent-loaded platelets; and (b) cryopreserving the MRI agent-loaded platelets, to form the composition comprising MRI agent-loaded cryopreserved platelets or lyophilizing the MRI agent-loaded platelets, to form the composition comprising MRI agent-loaded platelet derivatives.

In some embodiments of any of the aspects or embodiments herein that relate to a process/method for forming MRI agent-loaded platelets/platelet derivatives/cryopreserved platelets/FDPDs include (a) providing platelets suspended in a buffer comprising a salt, a base, a loading agent, and optionally at least one organic solvent; (b) contacting an MRI agent with a conjugate having a chelator conjugated, for example covalently bonded, to a linker, to form an MRI agent complex; (c) contacting the platelets with the MRI agent complex at a temperature in the range of 15-45° C. for a time period in the range of 5 minutes to 48 hours, to form MRI agent-loaded platelets such that the MRI agent complex is covalently linked to the platelets in the MRI agent-loaded platelets; and (d) cryopreserving the MRI agent-loaded platelets, to form the composition comprising MRI agent-loaded cryopreserved platelets or lyophilizing the MRI agent-loaded platelets, to form the composition comprising MRI agent-loaded platelet derivatives.

In some embodiments of any of the aspects or embodiments herein that relate to a process/method for forming MRI agent-loaded platelets/platelet derivatives/cryopreserved platelets/FDPDs include (a) providing platelets; (b) contacting the platelets with an MRI agent coupled to a cell penetrating peptide, and a buffer comprising a salt, a base, a loading agent, and optionally at least one organic solvent, to form MRI agent-loaded platelets; and (c) cryopreserving the MRI agent-loaded platelets to form the composition comprising the MRI agent-loaded cryopreserved platelets or lyophilizing the MRI agent-loaded platelets to form the composition comprising the MRI agent-loaded platelet derivatives.

In some embodiments of any of the aspects or embodiments herein that relate to a process/method for forming MRI agent-loaded platelets/platelet derivatives/cryopreserved platelets/FDPDs include (a) providing cryopreserved platelets or rehydrated platelet derivatives; and (b) contacting the cryopreserved platelets or the rehydrated platelet derivatives with an MRI agent coupled to a cell penetrating peptide, to form the composition comprising MRI agent-loaded cryopreserved platelets or MRI agent-loaded platelet derivatives.

In some embodiments of any of the aspects or embodiments herein that relate to a process/method for forming MRI agent-loaded platelets/platelet derivatives/cryopreserved platelets/FDPDs include (a) providing platelets; (b) contacting the platelets with an MRI agent coupled to a cell penetrating peptide, and a buffer comprising a salt, a base, a loading agent, and optionally at least one organic solvent, to form MRI agent-loaded platelets; and (c) cryopreserving the MRI agent-loaded platelets to form the composition comprising the MRI agent-loaded cryopreserved platelets or lyophilizing the MRI agent-loaded platelets to form the composition comprising MRI agent-loaded platelet derivatives, wherein the MRI agent-loaded cryopreserved platelets or the MRI agent-loaded platelet derivatives do not comprise a drug.

In some embodiments of any of the aspects or embodiments herein that relate to a process/method for forming MRI agent-loaded platelets/platelet derivatives/cryopreserved platelets/FDPDs include (a) providing cryopreserved platelets or rehydrated platelet derivatives; and (b) contacting the cryopreserved platelets or the rehydrated platelet derivatives with an MRI agent coupled to a cell penetrating peptide, to form the composition comprising MRI agent-loaded cryopreserved platelets and MRI agent-loaded platelet derivatives, wherein the MRI agent-loaded cryopreserved platelets or the MRI agent-loaded platelet derivatives do not comprise a drug.

In some embodiments of any aspects or embodiments herein that include administering MRI agent-loaded platelets or platelet derivatives, or use of a composition of MRI agent-loaded platelets or platelet derivatives herein, the effective dose of the MRI agent-loaded platelets or platelet derivatives is at least 10¹¹/kg, for example, at least 10¹²/kg, 10¹³/kg, 10¹⁴/kg, 10¹⁵/kg, or at least 10¹⁶/kg or more. In some embodiments, the effective dose of the platelet derivatives is in the range of 1.0×10⁷ to 1.0×10¹⁶/kg of the subject, for example, the effective dose is in the range of 1.0×10⁷ to 1.0×10¹⁵/kg of the subject, 1.0×10⁷ to 1.0×10¹⁴/kg, 1.0×10⁷ to 1.0×10¹³/kg, 1.0×10⁷ to 1.0×10¹²/kg, 1.6×10⁷ to 5.1×10¹¹/kg, 1.6×10⁷ to 3.0×10¹¹/kg, 1.6×10⁷ to 1.0×10¹²/kg, 1.5×10⁹ to 1.1×10¹⁴/kg, 3.0×10⁹ to 1.0×10¹³/kg, 3.0×10⁹ to 1.0×10¹²/kg, 1×10¹³ to 1.0×10¹⁴/kg, or 5.0×10¹² to 1.0×10¹⁴/kg of the subject.

In some embodiments, provided herein is a method of enhancing diagnosis and treatment of a disease as disclosed herein, comprising administering MRI agent-loaded platelets, MRI agent-loaded platelet derivatives, or MRI agent-loaded thrombosomes as disclosed herein. In some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein. In some embodiments, the disease is cancer. In some embodiments, the disease is Traumatic Brain injury. In some embodiments, the disease is cancer. In some embodiments, the disease is Traumatic Brain injury. In some embodiments, the disease is stroke. In some embodiments, the disease is an embolism. In some embodiments, the disease is a hemorrhage.

In one aspect, provided herein are methods of preparing MRI agent-loaded platelets, MRI agent-loaded platelet derivatives, MRI agent-loaded cryopreserved platelets, or MRI agent-loaded thrombosomes (e.g., freeze-dried platelet derivatives), comprising: contacting platelets, platelet derivatives, or thrombosomes with an MRI agent, and at least one loading agent and optionally one or more plasticizers such as organic solvents, such as organic solvents selected from the group consisting of ethanol, acetic acid, acetone, acetonitrile, dimethylformamide, dimethyl sulfoxide, dioxane, methanol, n-propanol, isopropanol, tetrahydrofuran (TIF), N-methyl pyrrolidone, dimethylacetamide (DMAC), or combinations thereof, to form the MRI agent-loaded platelets, MRI agent-loaded platelet derivatives, MRI agent-loaded cryopreserved platelets or MRI agent-loaded thrombosomes.

In some embodiments, the methods of preparing MRI agent-loaded platelets can include contacting the platelets, the platelet derivatives, and/or the thrombosomes with the MRI agent and with one loading agent. In some embodiments, the methods of preparing MRI agent-loaded platelets, MRI agent-loaded platelet derivatives, or MRI agent-loaded thrombosomes can include contacting the platelets, the platelet derivatives, or the thrombosomes with the MRI agent and with multiple loading agents.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents include, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. The presence of organic solvents, such as ethanol, can be beneficial in the processing of platelets, platelet derivatives, and/or thrombosomes. In some embodiments, the organic solvent may open up and/or increase the flexibility of the plasma membrane of the platelets, platelet derivatives, and/or thrombosomes.

In some embodiments, provided herein is a method of preparing MRI agent-loaded platelets, MRI agent-loaded platelet derivatives, or MRI agent-loaded thrombosomes, comprising: contacting platelets, platelet derivatives, or thrombosomes with a MRI agent, and a loading buffer comprising a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form the MRI agent-loaded platelets, the MRI agent-loaded platelet derivatives, or the MRI agent-loaded thrombosomes.

In some embodiments, provided herein is a method of preparing MRI agent-loaded platelets, MRI agent-loaded platelet derivatives, or MRI agent-loaded thrombosomes, comprising: contacting platelets, platelet derivatives, or thrombosomes with a MRI agent and a loading buffer comprising a salt, a base, a loading agent, and optionally at least one organic solvent to form the MRI agent-loaded platelets, MRI agent-loaded platelet derivatives, or the MRI agent-loaded thrombosomes.

In some embodiments, provided herein is a method of preparing MRI-loaded platelets, MRI-loaded platelet derivatives, or MRI-loaded thrombosomes, comprising: contacting platelets, platelet derivatives, or thrombosomes with an MRI and with a loading agent and optionally at least one organic solvent to form the MRI-loaded platelets, the MRI-loaded platelet derivatives, or the MRI-loaded thrombosomes.

Thus, in some embodiments provided herein is a method of preparing MRI-loaded platelets, the MRI-loaded platelet derivatives, or the MRI-loaded thrombosomes, comprising: contacting the platelets, the platelet derivatives, or the thrombosomes with a MRI in the presence of a buffer comprising a salt, a base, a loading agent, and optionally ethanol, to form the MRI-loaded platelets, the MRI-loaded platelet derivatives, or the MRI-loaded thrombosomes.

In some embodiments, the methods further include drying the MRI-loaded platelets or the MRI-loaded platelet derivatives. In some embodiments, the methods further include freeze-drying the MRI-loaded platelets or the MRI-loaded platelet derivatives. In such embodiments, the methods further include rehydrating the MRI-loaded platelets or the MRI-loaded platelet derivatives obtained from the drying step.

In some embodiments, the methods that further include drying the MRI-loaded platelets or the MRI-loaded platelet derivatives and rehydrating the MRI-loaded platelets or the MRI-loaded platelet derivatives obtained from the drying step provides rehydrated platelets or the thrombosomes comprising at least 10% of the amount of the MRI prior to loading.

In some embodiments of the methods of preparing cargo-loaded platelets, such as MRI agent-loaded platelets, as provided herein, the methods do not comprise contacting platelets, platelet derivatives, or thrombosomes with ethanol.

In some embodiments of the methods of preparing cargo-loaded platelets, such as MRI agent-loaded platelets, as provided herein, the methods do not comprise contacting platelets, platelet derivatives, or thrombosomes with a solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments of the methods of preparing cargo-loaded platelets, such as MRI agent-loaded platelets, as provided herein, the methods do not comprise contacting platelets, platelet derivatives, or thrombosomes with an organic solvent.

In some embodiments of the methods of preparing cargo-loaded platelets, such as MRI agent-loaded platelets, as provided herein, the methods do not comprise contacting platelets, platelet derivatives, or thrombosomes with a solvent.

In some embodiments of the methods of preparing cargo-loaded platelets, such as MRI agent-loaded platelets, as provided herein, the methods comprise contacting platelets, platelet derivatives, or thrombosomes with a solvent, such as an organic solvent, such as organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, such as ethanol.

In some embodiments, platelets, platelet derivatives, or thrombosomes are pooled from a plurality of donors. Such platelets, platelet derivatives, and thrombosomes pooled from a plurality of donors may be also referred herein to as pooled platelets, platelet derivatives, or thrombosomes. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

In some embodiments, the methods of preparing MRI agent-loaded platelets, MRI agent-loaded platelet derivatives, or MRI agent-loaded thrombosomes that include pooling platelets, platelet derivatives, or thrombosomes from a plurality of donors include a viral inactivation step.

In some embodiments, the methods of preparing MRI agent-loaded platelets, MRI agent-loaded platelet derivatives, or MRI agent-loaded thrombosomes that include pooling platelets, platelet derivatives, or thrombosomes from a plurality of donors do not include a viral inactivation step.

In some embodiments, the platelets, the platelet derivatives, or the thrombosomes are loaded with the MRI agent in a period of time of about less than 1 minute to 48 hours, such as 5 minutes to 24 hours, such as 20 minutes to 12 hours, such as 30 minutes to 6 hours, such as 1 hour to 3 hours, such as about 2 hours. In some embodiments, platelets, platelet derivatives, or thrombosomes are loaded with the MRI agent for a time of about 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer, or any time period range therein. In some embodiments, platelets, platelet derivatives, or thrombosomes are loaded with the MRI agent for a time of less than one minute. In some embodiments, a concentration of MRI agent from about 0.1 nM to about 10 μM, such as about 1 nM to about 1 μM, such as about 10 nM to 10 μM, such as about 100 nM is loaded in a period of time of about less than 1 minute to 48 hours, such as 5 minutes to 24 hours, such as 20 minutes to 12 hours, such as 30 minutes to 6 hours, such as 1 hour minutes to 3 hours, such as about 2 hours.

In some embodiments, provided herein are MRI agent-loaded platelets, MRI agent-loaded platelet derivatives, or MRI agent-loaded thrombosomes prepared according to any of the variety of methods disclosed herein. In some embodiments provided herein are rehydrated platelets, platelet derivatives, or thrombosomes prepared as according to any of the variety of methods disclosed herein.

In some embodiments, MRI agent-loaded platelets, MRI agent-loaded platelet derivatives, or MRI agent-loaded thrombosomes protect the MRI from metabolic degradation or inactivation. MRI delivery with MRI-loaded platelets, MRI-loaded platelet derivatives, or MRI-loaded thrombosomes may therefore be advantageous in diagnosing diseases such as cancer, since MRI-loaded platelets, MRI-loaded platelet derivatives, or MRI-loaded thrombosomes facilitate targeting of cancer cells while mitigating systemic side effects. MRI-loaded platelets, MRI-loaded platelet derivatives, or MRI-loaded thrombosomes may be used in any therapeutic setting in which expedited healing process is required or advantageous.

Accordingly, in some embodiments, provided herein is a method of enhancing diagnosis of a disease (e.g., any of the variety of diseases disclosed herein), comprising administering any of the variety of MRI agent-loaded platelets, MRI agent-loaded platelet derivatives, or MRI agent-loaded thrombosomes disclosed herein. Accordingly, in some embodiments, provided herein is a method of diagnosing a disease (e.g., any of the variety of diseases disclosed herein), comprising administering cold stored, room temperature stored, cryopreserved thawed, rehydrated, and/or lyophilized platelets, platelet derivatives, or thrombosomes as disclosed herein. In some embodiments, the disease is cancer. In some embodiments, the disease is Traumatic Brain injury. In some embodiments, the disease is stroke. In some embodiments, the disease is an embolism. In some embodiments, the disease is a hemorrhage.

Embodiment 1 is a method of preparing MRI agent-loaded platelets, comprising: treating platelets with an MRI agent coupled to a cell penetrating peptide; and a loading buffer comprising a salt, a base, a loading agent, and optionally at least one organic solvent, to form the MRI agent-loaded platelets.

Embodiment 2 is a method of preparing MRI agent-loaded platelets, comprising: providing platelets; and treating the platelets with an MRI agent coupled to a cell penetrating peptide; and a loading buffer comprising a salt, a base, a loading agent, and optionally at least one organic solvent to form the MRI agent-loaded platelets.

Embodiment 3 is the method of any one of the preceding embodiments, wherein the platelets are treated with the MRI agent coupled to a cell penetrating peptide and with the loading buffer sequentially, in either order.

Embodiment 4 is method of preparing MRI agent-loaded platelets, comprising: treating the platelets with a loading buffer comprising a salt, a base, a loading agent, and optionally at least one organic solvent to form a first composition; and treating the first composition with an MRI agent coupled to a cell penetrating peptide, to form the MRI agent-loaded platelets.

Embodiment 5 is the method of embodiment 1 or 2, wherein the platelets are treated with the MRI agent coupled to the cell penetrating peptide and with the loading buffer concurrently.

Embodiment 6 is a method of preparing MRI agent-loaded platelets, comprising: treating the platelets with an MRI agent in the presence of a cell penetrating peptide and a loading buffer comprising a salt, a base, a loading agent, and optionally at least one organic solvent to form the MRI agent-loaded platelets.

Embodiment 7 is the method of any one of the preceding embodiments, wherein the platelets are pooled from a plurality of donors.

Embodiment 8 is a method of preparing MRI agent-loaded platelets comprising (A) pooling platelets from a plurality of donors; and treating the platelets from step (A) with an MRI agent coupled to a cell penetrating peptide; and (B) with a loading buffer comprising a salt, a base, a loading agent, and optionally at least one organic solvent, to form the MRI agent-loaded platelets.

Embodiment 9 is a method of preparing MRI agent-loaded platelets comprising (A) pooling platelets from a plurality of donors; and (B) treating the platelets from step (A) with an MRI agent coupled to a cell penetrating peptide to form a first composition; and (C) treating the first composition with a loading buffer comprising a salt, a base, a loading agent, and optionally at least one organic solvent, to form the MRI agent-loaded platelets.

Embodiment 10 A method of preparing MRI agent-loaded platelets comprising (A) pooling platelets from a plurality of donors; and (B) treating the platelets from step (A) with a loading buffer comprising a salt, a base, a loading agent, and optionally at least one organic solvent, to form a first composition; and (C) treating the first composition with an MRI agent coupled to a cell penetrating peptide to form the MRI agent-loaded platelets.

Embodiment 11 is a method of preparing MRI agent-loaded platelets comprising pooling platelets from a plurality of donors; and treating the platelets with an MRI agent coupled to a cell penetrating peptide and a loading buffer comprising a salt, a base, a loading agent, and optionally at least one organic solvent, to form the MRI agent-loaded platelets.

Embodiment 12 is the method of any one of the preceding embodiments, wherein the loading buffer comprises optionally at least one organic solvent.

Embodiment 13 is the method of any one of the preceding embodiments, wherein the loading agent is a monosaccharide or a disaccharide.

Embodiment 14 is the method of any one of the preceding embodiments, wherein the loading agent is sucrose, maltose, dextrose, trehalose, glucose, mannose, or xylose.

Embodiment 15 is the method of any one of the preceding embodiments, wherein the platelets are isolated prior to a treating step.

Embodiment 16 is the method of any one of the preceding embodiments, wherein the platelets are selected from the group consisting of fresh platelets, stored platelet, and any combination thereof.

Embodiment 17 is the method of any one of the preceding embodiments, wherein the MRI agent comprises Gadolinium.

Embodiment 18 is the method of any one of the preceding embodiments, wherein the MRI agent comprises a nanoparticle.

Embodiment 19 is the method of any one of the preceding embodiments, wherein the cell penetrating peptide is Tat, or a portion thereof.

Embodiment 20 is the method of any one of the preceding embodiments, wherein the platelets are loaded with the MRI agent in a period of time of 1 minute to 48 hours.

Embodiment 21 is the method of any one of the preceding embodiments, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

Embodiment 22 is the method of any one of the preceding embodiments, further comprising cold storing, cryopreserving, freeze-drying, thawing, rehydrating, and combinations thereof the MRI agent-loaded platelets.

Embodiment 23 The method of embodiment 22, wherein the drying step comprises freeze-drying the MRI agent-loaded platelets.

Embodiment 24 is the method of embodiment 22 or 23, further comprising rehydrating the MRI agent-loaded platelets obtained from the drying step.

Embodiment 25 are MRI agent-loaded platelets prepared by the method of any one of the preceding embodiments.

Embodiment 26 are MRI agent-loaded platelets prepared by a method comprising rehydrating the MRI agent-loaded platelets of embodiment 25.

Embodiment 27 is the method of any one of the preceding embodiments, wherein the method does not comprise treating the platelets with an organic solvent.

Embodiment 28 is the method of any one of embodiments 4, 9, or 10, wherein the method does not comprise treating the first composition with an organic solvent.

Embodiment 29 is the method of any one of the preceding embodiments, wherein the method comprises treating the platelets with Prostaglandin E1.

Embodiment 30 is the method of any one of the preceding embodiments, wherein the method does not comprise treating the platelets with Prostaglandin E1.

Embodiment 31 is the method of any one of the preceding embodiments, wherein the method comprises treating the plates with GR144053.

Embodiment 32 is the method of any one of the preceding embodiments, wherein the method does not comprise treating the platelets with GR144053.

Embodiment 33 is the method of any one of the preceding embodiments, wherein the method comprises treating the platelets with eptifibatide.

Embodiment 34 is the method of any one of the preceding embodiments, wherein the method does not comprise treating the platelets with eptifibatide.

In some embodiments, subjects who are administered any of the imaging agent-loaded, for example MRI agent-loaded platelets or platelet derivatives herein, are afflicted with one or more of having an indication selected from the group consisting of Acute lymphoblastic leukemia (ALL), Acute myeloid leukemia (AML), Breast cancer, Gastric cancer, Hodgkin lymphoma, Neuroblastoma, Non-Hodgkin lymphoma, Ovarian cancer, Cervical cancer, Small cell lung cancer, Non-small cell lung cancer (NSCLC), Soft tissue and bone sarcomas, Thyroid cancer, Transitional cell bladder cancer, Wilms tumor Neuroendocrine tumors, Pancreatic cancer, Multiple myeloma, Renal cancer, Glioblastoma Prostate cancer, Sarcoma, Colon cancer, Melanoma, Colitis, Chronic inflammatory demyelinating polyneuropathy, Guillain-Barre syndrome, Immune Thrombocytopenia, Kawasaki disease, Lupus, Multiple Sclerosis, Myasthenia gravis, Myositis, Cirrhosis with refractory ascites, Hepatorenal syndrome, Nephrotic syndrome, Organ transplantation, Paracentesis, Hypovolemia, Aneurysms, Artherosclerosis, Cancer, Cardiovascular diseases, Genetic disorders, Infectious diseases, Metabolic diseases, Neoangiogenesis, Opthalmic conditions, Hypercholesterolemia, and Pulmonary hypertension.

In some embodiments, subjects who are administered any of the imaging agent-loaded, for example MRI agent-loaded platelets or platelet derivatives herein, are afflicted with one or more of the following: Aneurysm(s), artherosclerosis, cancer, cardiovascular diseases (post-myocardial infarction remodeling, cardiac regeneration, cardiac fibrosis, viral myocarditis, cardiac hypertrophy, pathological cardiac remodeling), genetic disorder(s), metabolic disease(s), neoangiogenesis, ophthalmic conditions (retinal angiogenesis), and pulmonary hypertension.

In some embodiments of any aspect herein that includes a CPP, the CPP can comprise, consist essentially of, or consist of any one of SEQ ID NOs: 1 to 87.

In one aspect, provided herein is a method for administering platelet derivatives, rehydrated platelet derivatives, or in illustrative embodiments, rehydrated freeze-dried platelet derivatives, to a mammalian subject, said method comprising:
administering an effective dose of a rehydrated composition comprising the rehydrated freeze-dried platelet derivatives to the mammalian subject,
wherein the mammalian subject has one or more of the following properties:
is being, was, or will be administered a therapeutic agent capable of binding amyloid beta and/or oligomers thereof, and/or plaques thereof;
comprises the therapeutic agent capable of binding amyloid beta and/or oligomers thereof, and/or plaques thereof;
is afflicted with Alzheimer's Disease;
has been diagnosed with Alzheimer's Disease;
has been subjected to MRI and amyloid-related imaging abnormalities (ARIA) were detected; and
has amyloid beta deposits on their brain.

In some embodiments, the mammalian subject is further administered a platelet composition comprising MRI agent-loaded cryopreserved platelets. In some embodiments, the mammalian subject has one or more sites of bleeding in their brain. In some embodiments, the mammalian subject has been subjected to MRI and amyloid-related imaging abnormalities (ARIA) were detected. In some embodiments, the ARIA detected comprises ARIA-H. In some embodiments, the mammalian subject is afflicted with Alzheimer's Disease.

In one aspect, provided herein is a method for treating brain bleeding in a mammalian subject, said method comprising:
administering an effective dose of a composition comprising platelet derivatives, rehydrated platelet derivatives, or in illustrative embodiments rehydrated freeze-dried platelet derivatives, to the mammalian subject. In some embodiments, the mammalian subject has one or more of the following properties:
is being, was, or will be administered a therapeutic agent capable of binding beta amyloid and/or oligomers thereof, and/or plaques thereof;
comprises the therapeutic agent capable of binding amyloid beta and/or oligomers thereof, and/or plaques thereof;
is afflicted with Alzheimer's Disease;
has been diagnosed with Alzheimer's Disease;
has ARIA in their brain
has been subjected to MRI and amyloid-related imaging abnormalities (ARIA) were detected on the brain of the mammalian subject; and
has amyloid beta deposits on their brain.

In one aspect, provided herein is a method for delivering an MRI agent to the brain of a mammalian subject, comprising:
administering to the mammalian subject, an effective dose of a rehydrated freeze-dried platelet derivative composition comprising MRI agent-loaded platelet derivatives. In some embodiments, the MRI agent-loaded platelet derivatives have a compromised plasma membrane, and at least 50% of the MRI agent-loaded platelet derivatives are CD 41-positive platelet derivative. In some embodiments, the mammalian subject has one or more of the following properties:
is being, was, or will be administered a therapeutic agent capable of binding amyloid beta and/or oligomers thereof, and/or plaques thereof;
comprises the therapeutic agent capable of binding amyloid beta and/or oligomers thereof, and/or plaques thereof;
is afflicted with Alzheimer's Disease;
has been diagnosed with Alzheimer's Disease;
has ARIA in their brain
has been subjected to MRI and amyloid-related imaging abnormalities (ARIA) were detected in the brain of the mammalian subject; and
has amyloid beta deposits on their brain.

In one aspect, provided herein is a method for delivering an MRI agent to the brain of a mammalian subject, comprising:
administering to the mammalian subject, an effective dose of
a) a platelet composition comprising MRI agent-loaded cryopreserved platelets, and/or
b) a platelet derivative composition comprising MRI agent-loaded platelet derivatives. In some embodiments, the mammalian subject administered the effective dose of a) and/or b), has one or more of the following properties:
is being, was, or will be administered a therapeutic agent capable of binding amyloid beta and/or oligomers thereof, and/or plaques thereof;
comprises the therapeutic agent capable of binding amyloid beta and/or oligomers thereof, and/or plaques thereof;
is afflicted with Alzheimer's Disease;
has been diagnosed with Alzheimer's Disease;
has ARIA in their brain
has been subjected to MRI and amyloid-related imaging abnormalities (ARIA) were detected on the brain of the mammalian subject; and
has amyloid beta deposits on their brain.

In any of the aspects and embodiments, the mammalian subject has two, three, four, five or more, or all of the following properties: is being, was, or will be administered a therapeutic agent capable of binding amyloid beta and/or oligomers thereof, and/or plaques thereof; comprises the therapeutic agent capable of binding amyloid beta and/or oligomers thereof, and/or plaques thereof; is afflicted with Alzheimer's Disease; has been diagnosed with Alzheimer's Disease; has ARIA; has been subjected to MRI and amyloid-related imaging abnormalities (ARIA) were detected in the brain of the mammalian subject; and has amyloid beta deposits on their brain. In some embodiments, the mammalian subject is being, was, or will be administered a therapeutic agent capable of binding amyloid beta and/or oligomers thereof, and/or plaques thereof. In some embodiments, the mammalian subject comprises the therapeutic agent capable of binding amyloid beta and/or oligomers thereof, and/or plaques thereof. In some embodiments, the mammalian subject is afflicted with Alzheimer's Disease, or has been diagnosed with Alzheimer's Disease. In some embodiments, the mammalian subject has been subjected to MRI and amyloid-related imaging abnormalities (ARIA) were detected in the brain of the mammalian subject, or has amyloid beta deposits on their brain.

In some embodiments, the mammalian subject has pathologic amyloid beta deposits on their brain.

In any of the aspects and embodiments herein, the mammalian subject is being, was, or will be administered a therapeutic agent capable of binding amyloid beta and/or oligomers thereof, and/or plaques thereof; or the brain of the mammalian subject comprises the therapeutic agent capable of binding amyloid beta and/or oligomers thereof, and/or plaques thereof.

In some embodiments, the therapeutic agent is a biologic agent. In some embodiments, the biologic agent is or comprises a monoclonal antibody. In some embodiments, the therapeutic agent is a small molecule.

In any of the aspects and embodiments, the mammalian subject is afflicted with Alzheimer's Disease. In some embodiments, the mammalian subject has been diagnosed with Alzheimer's Disease and has been administered a therapeutic agent capable of binding beta amyloid and/or oligomers thereof, and/or plaques thereof. In some embodiments, the administering of the rehydrated platelet derivative composition is by infusion. In some embodiments, the mammalian subject is a human.

In any of the aspects and embodiments, the administering reduces the number of sites of brain bleeds in the subject. In some embodiments, the administering ceases bleeding in one or more sites in the brain of the subject. In some embodiments, the administering ceases bleeding in all of the sites in the brain of the subject. In some embodiments, the administering reduces the amount of brain bleeding in the subject. In some embodiments, the method further comprises detecting the MRI-agent loaded FDPDs or the FDPDs in the brain of the subject. In some embodiments the methods herein further comprise using MRI to detect the MRI-agent loaded FDPDs in the brain of the subject. In some embodiments, the antibody capable of binding amyloid beta is selected from donanemab, aducanumab, bapineuzumab, gantenerumab, solanezumab, and lecanemab.

In any of the aspects and embodiments herein that includes administering platelet derivatives, cryopreserved platelets, MRI agent-loaded platelet derivatives, or MRI agent-loaded cryopreserved platelets to a recipient or a subject herein, a dose, an effective dose, dose for continuous infusion procedure (/min), therapeutically effective dose or amount of the platelet derivatives in a platelet derivative composition or for administering is in the range of 1.0×10¹ to 1.0×10¹²/kg of the subject. In some embodiments, the administering can be as a continuous infusion procedure and the dose can be in the range of 1.0×10⁷ to 1.0×10¹²/kg/min of the subject. In some embodiments, the administering can be the effective amount or the dose is in the range of 1.0×10⁹ to 10×10¹², 1.2×10⁹ to 1.0×10¹², 1.4×10⁹ to 1.0×10¹², 1.6×10⁹ to 1.0×10¹², 1.8×10⁹ to 1.0×10¹², 2.0×10⁹ to 1.0×10¹², 3.0×10⁹ to 1.0×10¹², 3.5×10⁹ to 1.0×10¹², 4.0×10⁹ to 1.0×10¹², 5.0×10⁹ to 1.0×10¹², 5.5×10⁹ to 1.0×10¹², 6.0×10⁹ to 1.0×10¹², 6.2×10⁹ to 1.0×10¹², 6.4×10⁹ to 1.0×10¹², or 6.5×10⁹ to 1.0×10¹²/kg of the subject.

In any of the aspects and embodiments herein that includes administering platelet derivatives, cryopreserved platelets, MRI agent-loaded platelet derivatives, or MRI agent-loaded cryopreserved platelets a dose, an effective dose, dose for continuous infusion procedure (/min), or a therapeutically effective dose can be in the range of 1.0×10¹ to 1.0×10¹²/kg of the subject. In some embodiments, administering can be done as a continuous infusion procedure, and the dose can be in the range of 1.0×10⁵ to 1.0×10¹²/kg/min. In some embodiments, a continuous infusion procedure using any dose herein, for example, a dose from 1.0×10⁵ to 1.0×10¹²/kg/min can be done for at least 15 minutes, 30 minutes, 1 hour, 2 hours, 3 hours, 4 hours, 5 hours, 6 hours, 8 hours, 10 hours, 12 hours, 18 hours, 24 hours. In some embodiments, continuous infusion using any dose herein, for example, a dose from 1.0×10⁵ to 1.0×10¹²/kg/min can be done for a time period in the range of 30 minutes to 72 hours, 30 minutes to 48 hours, 30 minutes to 24 hours, 30 minutes to 18 hours, 30 minutes to 12 hours, or 30 minutes to 6 hours. In some embodiments continuous infusion procedure can be done with platelet derivatives, cryopreserved platelets, MRI agent-loaded platelet derivatives, or MRI agent-loaded cryopreserved platelets at a dose in the range of 1.0×10¹ to 1.0×10⁷/kg/min, 1.0×10⁵ to 1.0×10⁸/kg/min, 1.0×10⁵ to 1.0×10⁹/kg/min, 1.0×10⁵ to 1.0×10¹⁰/kg/min, 1.0×10⁵ to 1.0×10¹¹/kg/min, 1.0×10⁶ to 1.0×10¹²/kg/min, 1.0×10⁸ to 1.0×10¹²/kg/min, or 1.0×10¹⁰ to 1.0×10¹²/kg/min. In some embodiments, administering can be done multiple times by administering a single, double, triple, or more doses, in illustrative embodiments between 1.0×10^{≡}, to 1.0×10¹²/kg at a frequency, for example, at every 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, or 24 hours. In certain illustrative embodiments, dosing is performed at a time interval between every 5 minutes and every 60 minutes. In some embodiments, a single, double, triple, or more doses can be administered at a frequency of a time within the range of 2 minutes to 24 hours, 2 minutes to 12 hours, 2 minutes to 6 hours, 2 minutes to 3 hours, 2 minutes to 2 hours, 2 minutes to 1 hour, 2 minutes to 45 minutes, 2 minutes to 30 minutes, 2 minutes to 20 minutes, 2 minutes to 15 minutes, 2 minutes to 10 minutes, or 2 minutes to 5 minutes. In some embodiments, the dosage in a single, double, triple or more doses can vary as per the time from the administration of the first dose. In some embodiments, the number of doses administered over a frequency of time herein can vary from 1 dose to 10 doses, 1 dose to 8 doses, 1 dose to 6 doses, 1 dose to 4 doses, or 1 dose to 3 doses. In some embodiments, administering can be done in multiple times, and the dose can vary in the range of 1.0×10⁵ to 1.0×10¹⁰/kg, 1.0×10⁵ to 1.0×10⁸/kg, 1.0×10⁵ to 1.0×10⁷/kg, 1.0×10⁸ to 1.0×10¹²/kg, 1.0×10⁹ to 1.0×10¹²/kg, 1.0×10¹⁰ to 1.0×10¹²/kg, or 1.0×10¹¹ to 1.0×10¹²/kg of the subject. In some embodiments, the continuous infusion procedure or providing doses at a time frequency herein can be done depending on whether bleeding is reduced to a satisfactory level, for example such that it is no longer considered life-threatening, or no longer considered severe or serious, or continued until the bleeding is mild, or stops. For example, administering can be performed at a frequency of at least one dose every 2 to 5 minutes or more frequently starting from the first dose until the bleeding potential of the subject is reduced as compared to the bleeding potential before the administering. In some embodiments, administering of platelet derivatives, FDPDs, or FPH herein can be performed as a mixed procedure in which the continuous infusion can be interrupted with a specific dose of platelet derivatives followed by a specific interval as per the requirement, for example, status of bleeding of the subject or the recipient. In some embodiments, administering platelet derivatives, FDPDs, FPH, cryopreserved platelets, MRI agent-loaded platelet derivatives, or MRI agent-loaded cryopreserved platelets as a continuous infusion procedure, or providing single or multiple doses as per a time frequency as disclosed herein can be done as a part of a surgical procedure. In other words, a continuous infusion procedure or single or multiple dose administration can be done at a time frequency disclosed herein for a subject undergoing surgery. For example, administering platelet derivatives, FDPDs, FPH, cryopreserved platelets, MRI agent-loaded platelet derivatives, or MRI agent-loaded cryopreserved platelets can be done as a continuous infusion procedure during a surgery, such that any dose as disclosed herein can be infused 10, 20, 30, 40, 50, or 60 minutes, within 1 hour before, during, or within one hour after a surgery, or before a surgery is scheduled to end. In other embodiments, any dose can be provided as a continuous infusion procedure starting 60, 50, 40, 30, 20, or 10 minutes within 1 hour before, during or within 1 hour after a surgery, or before a surgery is scheduled to end. In some embodiments, the continuous infusion procedure can be done during the entire duration of the surgery. In some embodiments, the continuous infusion procedure can be done whenever there might be a risk of increased bleeding during the surgery. In some embodiments, platelet derivatives, FDPDs, FPH, cryopreserved platelets, MRI agent-loaded platelet derivatives, or MRI agent-loaded cryopreserved platelets can be administered every 2, 3, 4, 5, 6, 7, 8, or 9 minutes within 1 hour before, during, or within 1 hour after the surgery. In some embodiments, platelet derivatives, FDPDs, FPH, cryopreserved platelets, MRI agent-loaded platelet derivatives, or MRI agent-loaded cryopreserved platelets can be administered every 10, 20, 30, 40, 50, or 60 minutes within 1 hour before, during, or within 1 hour after the surgery. In some embodiments, the subject is bleeding at the start of administering, and the administering leads to a decrease in bleeding within 24 hours after the start of the administering. In some embodiments, the administering is performed until the bleeding stops.

In some embodiments, the methods herein comprise one, two, three or more of the following:
less than 5% of the CD 41-positive platelet derivatives are microparticles having a diameter of less than 0.5 μm,
at least 65% of the platelet derivates are CD 42 positive,
the rehydrated MRI agent-loaded platelet derivatives are capable of generating thrombin in an in vitro thrombin generation assay,
the composition comprises a population of rehydrated MRI agent-loaded platelet derivatives having a reduced propensity to aggregate such that no more than 10% of the platelet derivatives in the population aggregate under aggregation conditions comprising an agonist but no platelets, and no divalent cation,
the composition is capable of occluding a collagen coated channel in a Total-Thrombus formation system (T-TAS);
at least 50% of the MRI-loaded platelet derivatives in the composition are at least 0.5 μm in diameter by scattering intensity; and
at least 50% of the MRI-loaded platelet derivatives in the composition are between 0.5 μm and 25 μm in diameter by scattering intensity.

In some embodiments, the methods herein further comprise performing tangential flow filtration (TFF) of a platelet composition with a preparation agent to prepare a TFF-treated composition comprising the platelets, before contacting the platelets with the MRI agent complex, and wherein contacting the TFF-treated composition comprising platelets with the MRI agent complex forms MRI agent-loaded TFF-treated composition.

While the embodiments of the invention are amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

### EXAMPLES

### Example 1. MRI-Agent Loaded Platelets

Protocol 1: Loading Platelets with an MRI Agent

Platelet pool was acidified to pH 6.6-6.8 using 4 μL 1M Acid Citrate Dextrose solution per 1 mL pooled platelet rich plasma.

Platelet count in solution was obtained using Coulter AcT Diff hematology analyzer.

Platelets were isolated via centrifugation at 845×g for 10 minutes at room temperature, with gentle acceleration and braking.

Incubation solutions of FITC-CPP (FITC-TAT) or FITC-labeled magnetic nanoparticles were prepared.

Platelets were resuspended in loading buffer (Table 1) or desired incubation solution at a concentration of 500,000 platelets/μL and incubated at 37° C. with low frequency agitation on a rocker for up to 3 hours.

Platelets were washed with loading buffer (Table 1) 3× to remove unloaded agents (e.g., FITC-TAT, FITC-labeled magnetic nanoparticles) and use the remaining sample for plate reader, flow cytometer, and/or microscope analysis.

Platelets were isolated and pooled by centrifugation to adjust concentration that the isolation technology can achieve. The typical concentration is 5×10⁶ platelets/μl. The platelet medium can be altered to change the proportion of excipients, or to exchange excipients for similar products. The platelet medium is then replaced with a buffer composed of:

TABLE 1
Loading Buffer:

| | Concentration | |
|---|---|---|
| | (mM, except where | |
| Component | otherwise indicated) | |
| NaCl | 750 | |
| KCl | 48 | |
| HEPES | 95 | |
| NaHCO₃ | 120 | |
| Dextrose | 3 | |
| Trehalose | 0.1M | |
| Ethanol | 1.00% | (v/v) |
| Dansyl-EACA/EACA | 0, 50, or 100 | |
| (1:1000) | | |

After resuspension the platelets were incubated with either 5-100 μM CPP (L-TAT 49-57, See Mishra., R., (2009)) conjugated to FITC and Gd-DOTA or FeO₃ nanoparticles at about between 1×10⁻¹⁹ and about 1×10⁻¹⁵ nanoparticles/mL buffer (average particle diameter of about 20-30 nn, labeled with FITC) in the loading buffer for up to 4 hours at 37° C. The loaded platelets are then used for applications which include, but are not limited to, cryopreservation, lyopreservation, and immediate use in therapeutic functional or diagnostic assays.

The results for the above formulation are provided herein. A Tecan Infinite M200 Pro plate reader was used for quantification of MRI agent loading. A Novocyte flow cytometer was used to determine both MRI agent loading and percent of platelets effectively loaded. An Olympus microscope was used to visualize the loading into platelets.

Although the steps involving processing of blood products, such as, pooled platelet rich plasma was done by centrifugation, the processing can also be done by TFF methods as disclosed in the instant invention that avoids the centrifugation steps.

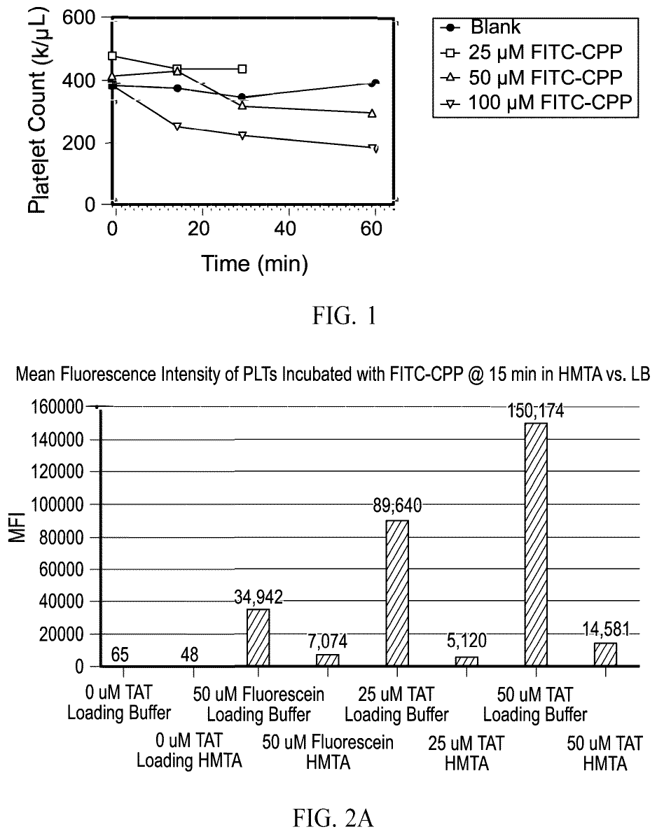

FIG. 1 shows pooled apheresis platelets incubated with FITC-labeled TAT peptide in loading buffer. The platelets were incubated for 15 to 60 minutes at 37° C. with low frequency agitation on a rocker. After incubation, the platelet counts were analyzed on an AcT-Diff hemacytometer. The data show that platelet counts are stable over time up to a concentration of 50 μM FITC-TAT.

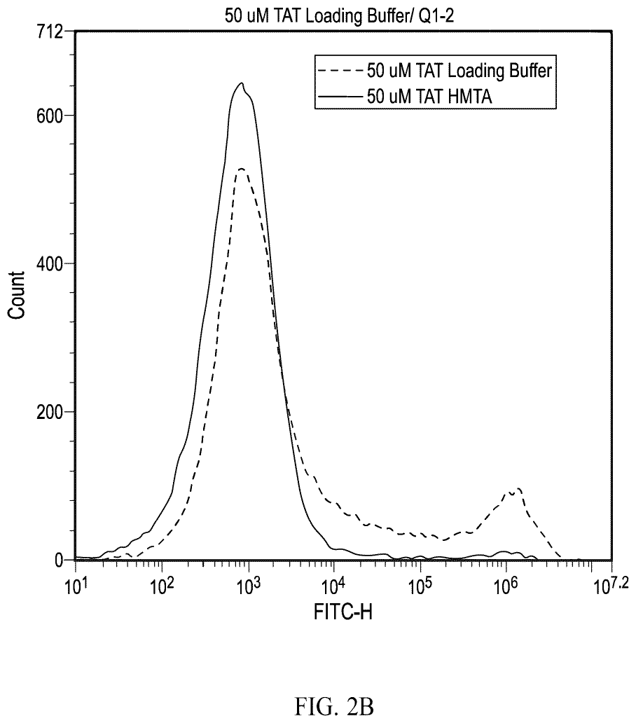

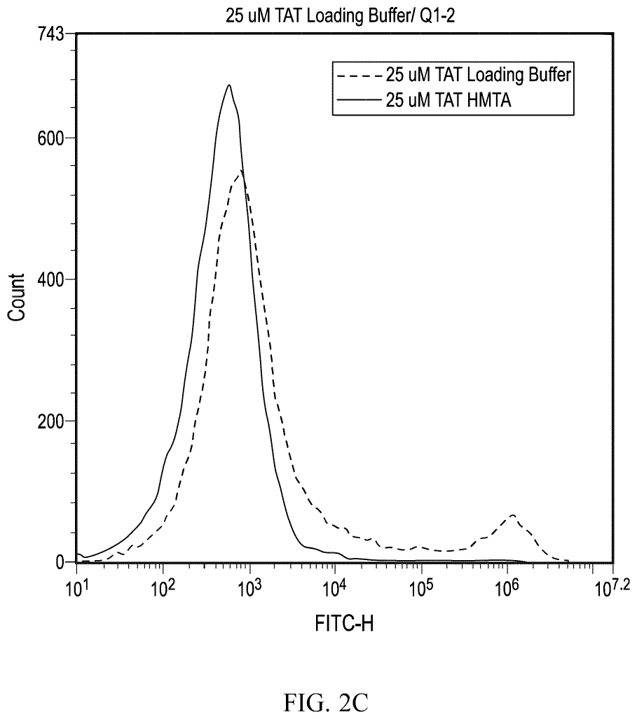

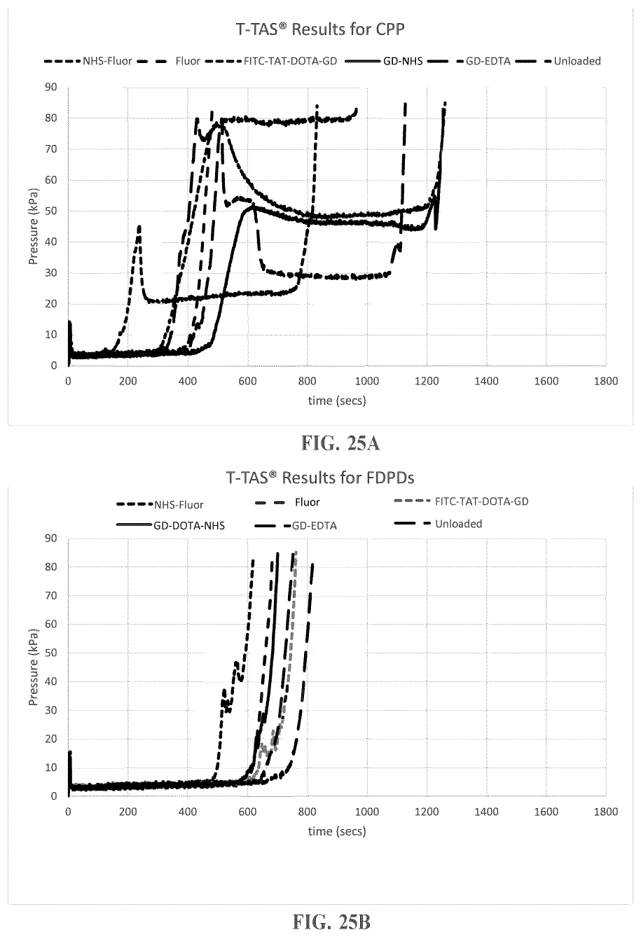

Pooled apheresis platelets were incubated with FITC-labeled TAT peptide in either HMTA or loading buffer. Platelets are incubated for 15 minutes at 37° C. with low frequency agitation on a rocker. After washing, the platelets were analyzed by flow cytometry for FITC-TAT loading. FIG. 2A shows the mean fluorescence values for each sample. FIGS. 2B and 2C are histograms are for 50 μM (FIG. 2B) or 25 μM (FIG. 2C) FITC-TAT concentrations during loading, respectively. Negative controls included 0 μM FITC-TAT in both loading buffer (Table 1) and HMTA and 50 μM fluorescein in both loading buffer and HMTA.

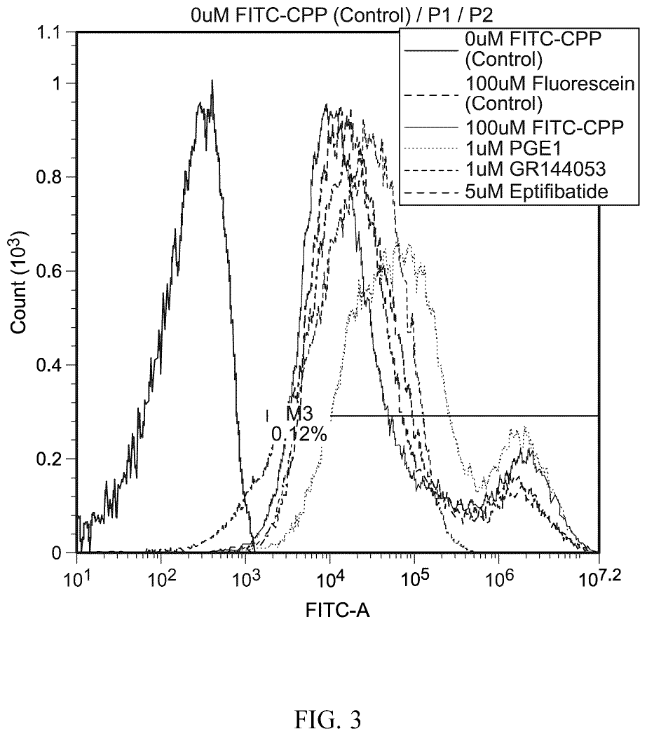

FIG. 3 shows a flow cytometry histograms of samples incubated with 100 μM FITC-CPP for 60 minutes in the presence of platelet anti-aggregation compounds, including PGE1, GR144053, and eptifibatide. Negative controls included 0 μM FITC-TAT in loading buffer and 50 μM fluorescein in loading buffer. All samples were incubated in loading buffer at 37° C. with low frequency agitation on a rocker.

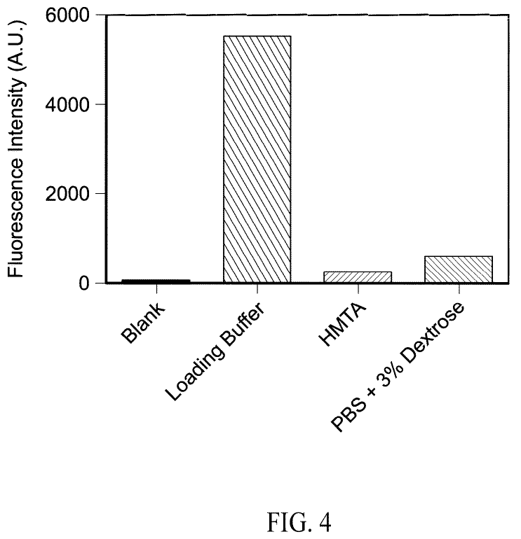

FIG. 4 shows the effect of different buffers on FITC-TAT loading into platelets as measured by fluorescence intensity. Minimal fluorescence was detected with HMTA buffer and PBS with 3% dextrose.

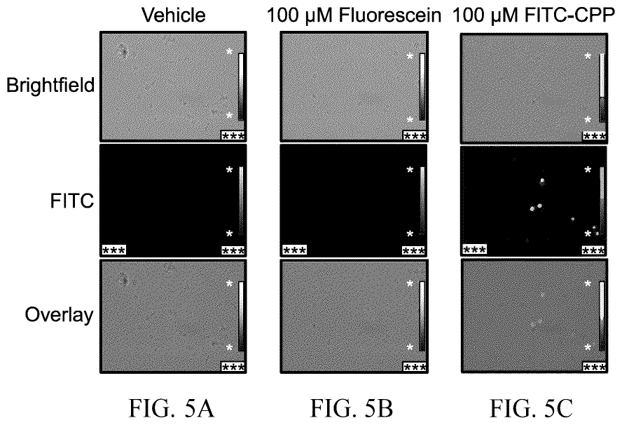

FIGS. 5A-5C shows brightfield, FITC, and overlaid images of 0 μM FITC-TAT (“Vehicle”) (FIG. 5A), 100 μM fluorescein (FIG. 5B), and 100 μM FITC-TAT (“FITC-CPP”) (FIG. 5C) incubated for 30 minutes at 37° C. with low frequency agitation on a rocker in loading buffer containing the additive indicated herein. Each image is representative. The scale bars are 10 μm. The overlay image in the bottom right corner shows FITC-TAT co-localizing with platelets.

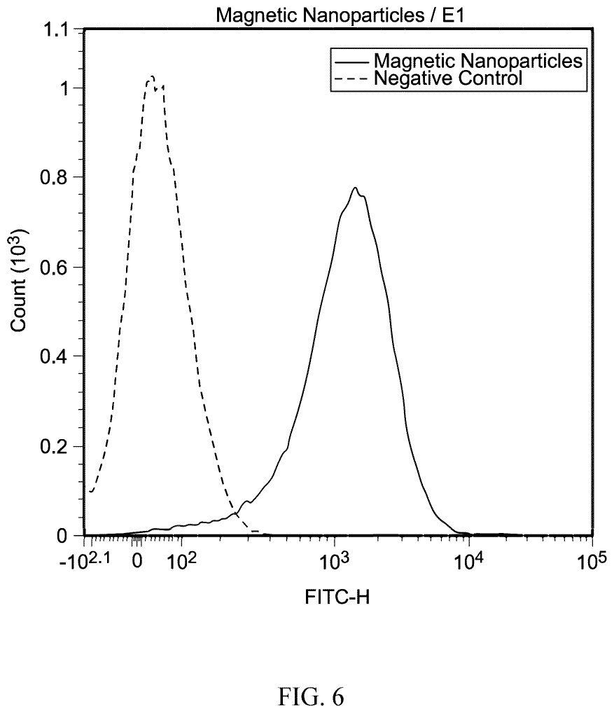

Flow cytometry histograms of samples incubated with either loading buffer or a solution of magnetic nanoparticles (size range between 20-30 nm) labeled with FITC. The data show that platelets are capable of endocytosing magnetic nanoparticles (FIG. 6). All samples were incubated in loading buffer at 37° C. with low frequency agitation on a rocker.

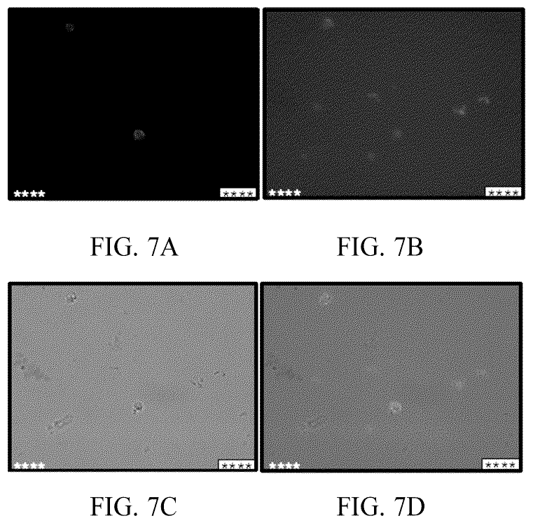

FIGS. 7A-7D shows Texas Red (FIG. 7A), brightfield (FIG. 7C), FITC conjugated nanoparticles (FIG. 7B), and overlaid (FIG. 7D) images of samples incubated with nanoparticles (size range between 20-30 nm) labeled with FITC-TAT for 3 hours at 37° C. with low frequency agitation on a rocker in loading buffer. Each image is representative. The scale bars are 10 μm.

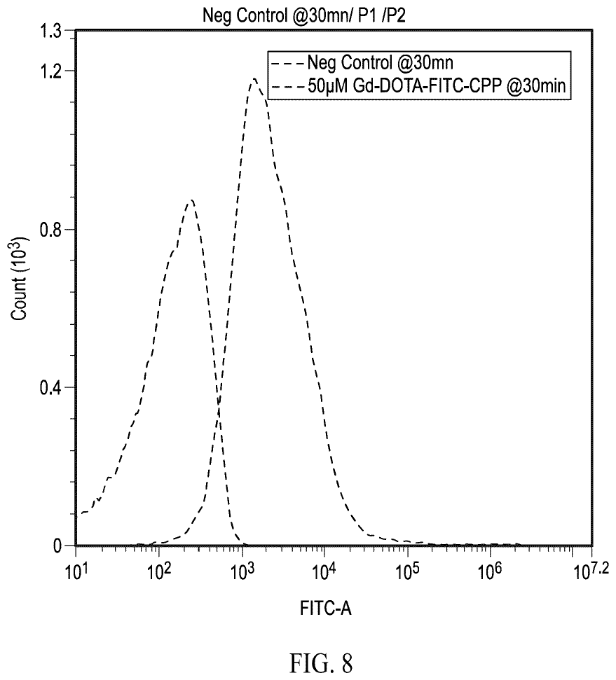

FIG. 8 shows flow cytometry histograms of platelet samples incubated with either loading buffer (left peak) or a 50 μM FITC-CPP-Gd-DOTA solution (right peak) for 30 minutes. The data show that 76% of flow events showed fluorescent signal above background. All platelet samples were incubated in loading buffer at 37° C. with low frequency agitation on a rocker.

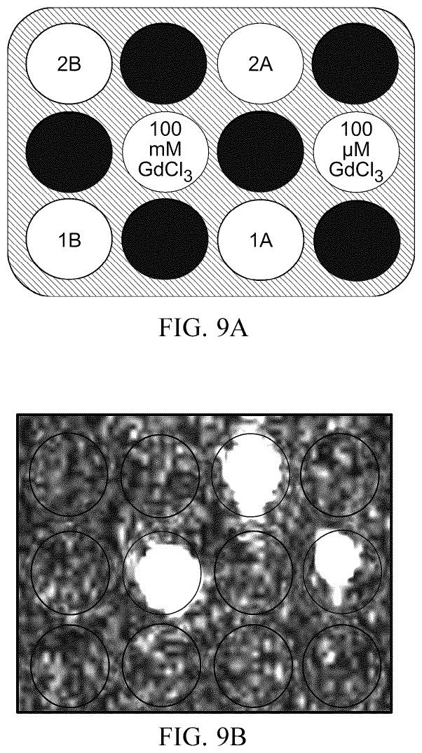

FIGS. 9A-9B show a schematic (FIG. 9A) and magnetic resonance imaging (FIG. 9B). As shown in FIG. 9A, samples 1A, 1B, and 2B are negative controls, sample 2A includes Gd-DOTA-FITC-CPP with platelets (400K/μL), samples indicated with either 100 mM or 100 μM GdCl₃ are positive controls. Sample 1A included loading buffer and platelets (400K/μL), sample 1B included loading buffer alone, sample 2A included loading buffer, Gd-DOTA-CPP-FITC, and platelets (400K/μL), and sample 2B included loading buffer and Gd-DOTA-FITC-CPP only (washed) as another negative control. The magnetic resonance imaging data shown in FIG. 9B shows detection in the two positive control samples and also sample 2A which included loading buffer, Gd-DOTA-CPP-FITC, and platelets (400K/μL), thus showing loading of Gd-DOTA-CPP-FITC into platelets.

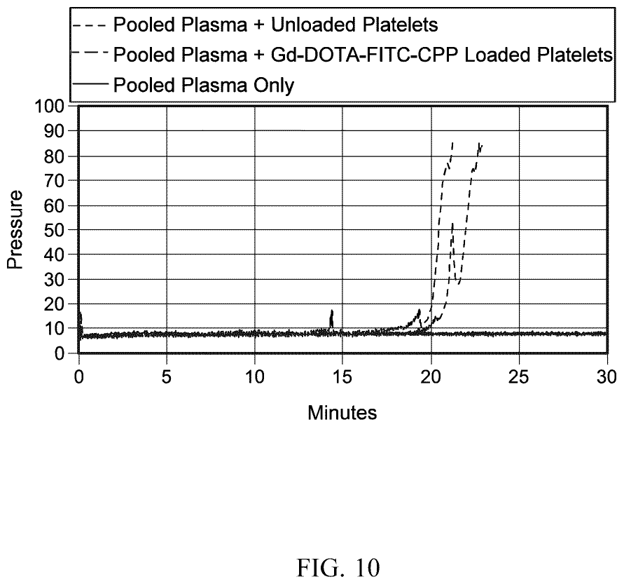

FIG. 10 is a graph showing post-cryopreservation occlusion time of platelets loaded with Gd-DOTA-FITC-CPP with plasma only (negative control) (shown as bottom line), pooled, unloaded platelets (positive control), and Gd-DOTA-FITC-CPP loaded platelets. The occlusion times were as follows: negative control=0 minutes, positive control (21 minutes), and Gd-DOTA-FITC-CPP loaded platelets (22 minutes). Thus, Gd-DOTA-FITC-CPP loaded platelets had a similar occlusion time to unloaded platelets indicating that such loaded platelets retain hemostatic function.

### Example 2. Tangential Flow Filtration (TFF) Method of Platelet Derivative Preparation

Apheresis platelets underwent tangential flow filtration in accordance with a standard operating procedure, including the following process steps: platelet dilution, platelet concentration and platelet washing.

The platelet donor units were initially pooled into a common vessel. The platelets may or may not be initially diluted with an acidified washing buffer (e.g., a control buffer) to reduce platelet activation during processing. The platelets can undergo two processing pathways; either washed with control buffer until a desired residual component is reached (e.g., donor plasma) before being concentrated to a final product concentration or the platelets are concentrated to a final product concentration before being washed with control buffer until a desired residual component is reached (e.g., donor plasma). In both the processing pathways, the platelet donor units were initially diluted or further diluted 1:1 in Buffer A before being loaded onto a TFF machine for further processing. TFF processed platelets are then filled into vials, lyophilized and thermally treated.

One particular protocol follows.

For all steps of the TFF process in this Example, Buffer A was used. The process was carried out at a temperature of 18-24° C.

Buffer A

| Component | Value (±1%) | |
|---|---|---|
| HEPES | 7.6 | mM |
| NaCl | 60 | mM |
| KCl | 3.84 | mM |
| Dextrose | 2.4 | mM |
| NaHCO₃ | 9.6 | mM |
| Trehalose | 80 | mM |
| Ethanol | 0.8% | |
| Polysucrose | 6% | (w/v) |

| | |
|---|---|
| pH | 6.6-6.8 |

Platelets were initially diluted in Buffer A (1:1) and loaded onto the TFF (PendoTECH controller system), which was prepared with a Repligen TFF Cassette (XPM45LO1E). The TFF process was performed using a membrane with a pore size of 0.45 μm. The platelets were diluted with an equal weight (±10%) of Buffer A. The platelets were concentrated to about 2250×10³ cells/μL (±250×10³) and then washed with approximately 2 diavolumes (DV) of Buffer A. The target plasma percentage was typically less than 15% relative plasma (as determined by plasma protein content). Removal of plasma proteins was monitored through 280 nm UV absorbance against known correlations. Following washing, if the concentration of the cells was not 2000×10³ cells/μL (±300×10³), the cells were either diluted with Buffer A or were concentrated to fall within this range. Under all circumstances whenever the cells are contacted with the buffer A, it was done at a temperature in the range of 18-24° C. For a better clarity, the cells were loaded with the reagents of the buffer A at a temperature in the range of 18-24° C. The cells were typically then freeze-dried (lyophilized) and subsequently heated (thermally treated) at 80° C. for 24 hours, thereby forming thrombosomes, but sometimes the cells were used before lyophilization (sometimes called thrombosomes ‘pre-lyo’).

In order to perform studies related to the quantification of microparticles, quantification of expression or presence of various surface markers (like Annexin V, CD 41, CD 42, CD 47, CD 62, thrombospondin, von-Willebrand factor), thrombin generation studies (TGPU), aggregation studies, the thrombosomes were typically rehydrated with water over 10 minutes at room temperature. In general, the rehydration volume is equal to the volume used to fill each vial of thrombosomes prior to drying. The thrombosomes which were heated (thermally treated) after lyophilization are also referred to as baked thrombosomes. Whereas the thrombosomes which were not heated (thermally treated) after lyophilization are referred to as unbaked thrombosomes.

Platelet derivatives are also referred to herein as thrombosomes. It would be clear to a skilled artisan that the thrombosomes which are obtained after lyophilization in the form of a powder can be used for commercial application, like providing the platelet derivative composition (thrombosomes) in dried form in vials to, for example, a medical practitioner who can rehydrate the vials with an appropriate amount of a liquid.

In some cases, samples were drawn at UV readings correlating to about 51% relative plasma volume, about 8.1% relative plasma volume, about 6.0% relative plasma volume, and about 1.3% relative plasma volume. Low volume aliquots were sampled throughout each processing step with the about 6.0% and under samples.

### Example 3. Surface Markers and Thrombin Generation

FDPDs batch were produced by the TFF method described in Example 2 and assayed for cell surface marker expression or presence or absence using flow cytometry.

Flow cytometry was used to assess FDPDs for expression or presence or presence of CD41, CD62, and phosphatidylserine (PS). Samples included approximately 270,000/μL FDPDs during staining and were diluted approximately 1:34 before the sample was analyzed in the cytometer. FDPD samples were rehydrated and diluted 1:2 in deionized water. A stock of anti-CD41 was diluted by adding 47.6 μL of antibody to 52.4 μL of HMTA. Samples stained with anti-CD41 were made by adding 10 μL of diluted FDPDs to 10 μL HMTA and 10 μL of diluted CD41 antibody. An anti-CD62 master mix was prepared by combining 12 μL anti-CD62 with 23.8 μL anti-CD41 and 64.2 μL of HMTA. An isotype control mix was made in the same manner. Samples stained with anti-CD62 were made by adding 10 μL of diluted FDPDs to 20 μL of the anti-CD62 master. The isotype master mix was used to make isotype control samples in the same manner. An annexin V (AV) master mix was prepared by combining 11.7 μL of AV with 83.3 μL of anti-CD41 and 80 μL of HMTA. Sample stained with AV were made by adding 20 μL of diluted FDPDs containing 50 mM GPRP to 20 μL of HMTA containing 15 mM CaCl₂ and 20 μL of the AV master mix. Negative gating control samples were made in the same manner using HMTA without calcium to prevent AV binding to PS. All samples were incubated at room temperature for 20 minutes. After incubation 1 mL HBS was added to all samples. HBS used to dilute AV test samples contained 5 mM CaCl₂ Anti-CD41 binding was used to identify the population of interest. CD62 and PS expression or presence was assessed by anti-CD62 and AV binding within the CD41 positive population.

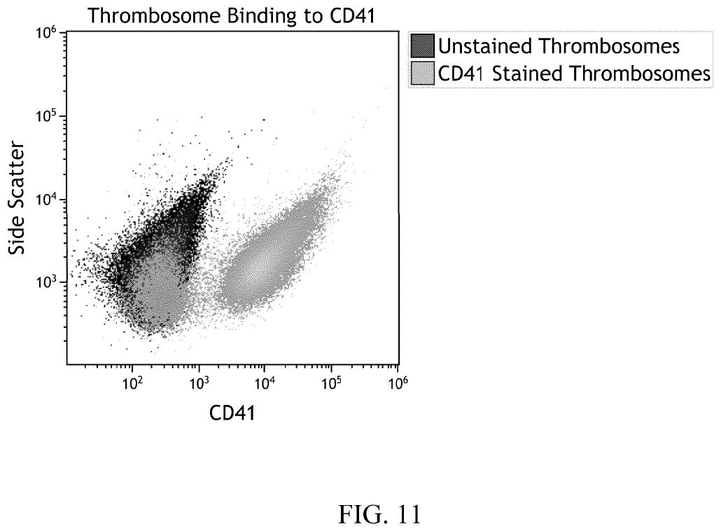

Glycoprotein IIb (GPIIb, also known as antigen CD41) expression or presence was assayed using an anti-CD41 antibody (4.8 μL, Beckman Coulter part #IM1416U). The assayed FDPDs demonstrated CD41 positivity (Table 2; FIG. 11)

TABLE 2

| Batch | CD41 Positivity (%) |
|---|---|
| 1 | 81.5 |
| 2 | 79.4 |
| 3 | 85.7 |
| 4 | 78.2 |
| 5 | 81.5 |
| 6 | 84.0 |
| 7 | 78.5 |
| Mean | 81.3 |

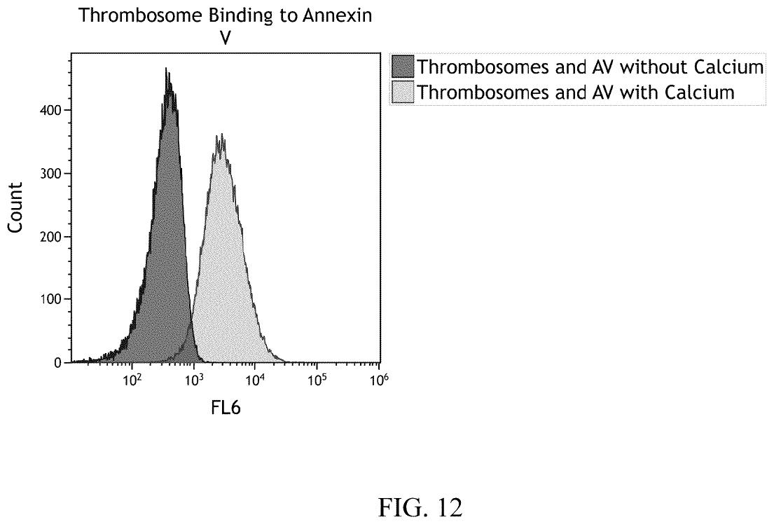

Phosphatidylserine (PS) expression or presence was assayed using annexin V (AV) (1.3 μL, BD Biosciences Cat. No. 550475). AV is a calcium-dependent phospholipid binding protein. The assayed FDPDs demonstrated AV positivity (Table 3; FIG. 12).

TABLE 3

| Batch | AV Positivity (%) |
|---|---|
| 1 | 96.7 |
| 2 | 89.9 |
| 3 | 95.3 |
| 4 | 95.4 |
| 5 | 95.9 |
| 6 | 96.2 |
| 7 | 93.5 |
| Mean | 94.7 |

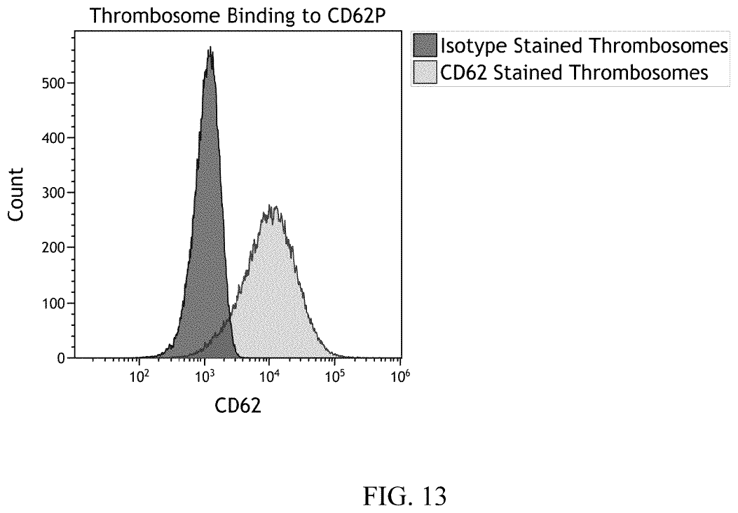

P-selectin (also called CD62P) expression or presence was assayed using an anti-CD62P antibody (2.4 μL, BD Biosciences Cat. No. 550888). The assayed FDPDs demonstrated CD62 positivity (Table 4, FIG. 13)

TABLE 4

| Batch | CD62 Positivity (%) |
|---|---|
| 1 | 94.2 |
| 2 | 93.1 |
| 3 | 89.8 |
| 4 | 92.4 |
| 5 | 92.5 |
| 6 | 87.3 |
| 7 | 90.7 |
| Mean | 91.4 |

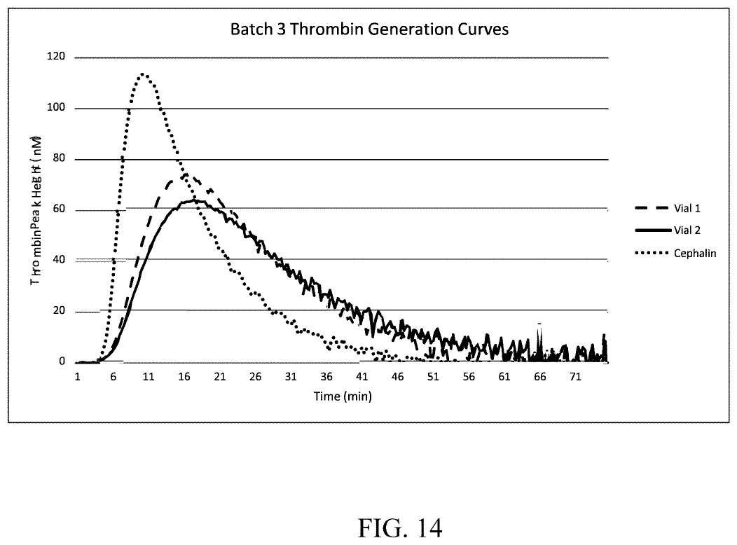

Thrombin generation was measured at 4.8×10³ FDPDs/μl in the presence of PRP Reagent containing tissue factor and phospholipids using the below protocol. On average, the Thrombin Peak Height (TPH) for a FDPDs sample was 60.3 nM. Cephalin was used as a positive control. (Table 6; FIG. 14)

For each vial tested, a rehydrated sample of FDPDs was diluted to 7,200 particles per μL based on the flow cytometry particle count using 30% solution of Octaplas in control buffer. In a 96 well plate, sample wells were generated by adding 20 μL of PRP reagent (Diagnostica Stago Catalog No. 86196) and 80 μL of diluted FDPDs. Calibrator wells were generated by adding 20 μL of Thrombin Calibrator reagent (Diagnostica Stago Catalog No. 86197) to 80 μL of diluted FDPDs. The plate was loaded into the plate reader and incubated in the dark at 40° C. for 10 minutes. During sample incubation, FluCa solution was prepared by adding 40 μL of FluCa substrate (Diagnostica Stago Catalog No. 86197) to 1.6 mL of Fluo-Buffer (Diagnostica Stago Catalog No. 86197) warmed to 37° C. and vortexed to mix. The FluCa solution was aspirated in to the dispensing syringe and 20 μL was mechanically dispensed in to each reaction well, bringing the final FDPDs concentration in each well to 4,800 particles per μL and starting the thrombin generation reaction. Thrombin generation was measured via fluorescence in each well over the course of 75 minutes.

An exemplary step-by-step protocol follows:
1. Opened CAT software; set up instrument; and prepared PRP reagent (including Tissue Factor and some phospholipids), calibrator, and fluo-buffer and fluo-substrate according to manufacturer guidelines.
2. Thawed Octaplas and TGA dilution buffer in 37° C. water bath for 10 minutes.
3. Added thawed Octaplas to TGA dilution buffer to create a buffer containing 30% Octaplas.
4. Used the 30% Octaplas mix to dilute reconstituted cephalin 1:50 to be used as a positive control.
5. Rehydrated FDPDs with cell culture grade water for 10 minutes then diluted with 30% Octaplas to 7,200 FDPDs/μL.
6. Using a multichannel pipette, added 20 μL of PRP reagent to each test well. Added 20 μL of Calibrator to each calibration well.
7. Added 80 μL of sample to each test and calibration well. Added 80 μL of 30% Octaplas to negative control wells and 1:50 cephalin to positive control wells.
8. Inserted plate into tray and incubated for 10 minutes at 40° C. After incubation, dispensed fluo-buffer and fluo-substrate mixture (including a fluorescent-labeled peptide, that when cleaved by thrombin, generates a fluorescent signal) into active wells.
9. Read plate for 75 minutes at 20 s intervals to capture full thrombin generation profile.

TABLE 6

| Batch | TPH (nM) |
|---|---|
| 1 | 61.5 |
| 2 | 71.4 |
| 3 | 67.8 |
| 4 | 52.0 |
| 5 | 60.2 |
| 6 | 54.7 |
| 7 | 54.4 |
| Mean | 60.3 |

Data from these assays is summarized in Table 7.

TABLE 7
TFF Batches

| | Average | Average | Average AV | Average CD62 |
|---|---|---|---|---|
| | TPH | CD41 | Positivity | Positivity |
| Batch | (nM) | Positivity | (0.5 μm-2.5 μm)¹ | (0.5 μm-2.5 μm)¹ |
| Batch B | 61.5 | 81.5 | 96.7 | 94.2 |
| Batch C | 71.4 | 79.4 | 89.9 | 93.1 |
| Batch D | 67.8 | 85.7 | 95.3 | 89.8 |
| Batch E | 52.0 | 78.2 | 95.4 | 92.4 |
| Batch F | 60.2 | 81.5 | 95.9 | 92.5 |
| Batch G | 54.7 | 84.0 | 96.2 | 87.3 |
| Batch H | 54.4 | 78.5 | 93.5 | 90.7 |
| Mean | 60.3 | 81.3 | 94.7 | 91.4 |

¹Particle diameter as assessed using sizing beats on the flow cytometry forward scatter.

### Example 4. Inability of FDPDs to Aggregate in the Presence of Agonists and Absence of Fresh Platelets

Light transmission aggregometry (LTA) was used to observe FDPD aggregation in the presence of known platelet aggregation agonists. Such methods provide exemplary aggregation conditions for aggregation analysis of FDPDs. The FDPD aggregation data was compared to aggregation data of fresh platelets.

FDPDs, also referred as “TFF FDPDs”, were produced by the TFF method described in Example 2. Fresh platelets in Platelet Rich Plasma (PRP) were prepared from whole blood collected in acid-citrate-dextrose (ACD) collection tubes (BD Vacutainer ACD Solution A Blood Collection Tubes ref #364606). Platelet rich plasma (PRP) was prepared by centrifugation of ACD-whole-blood at 180 g for 15 minutes at 22° C. using a Beckman Coulter Avanti J-15R centrifuge. Platelet poor plasma (PPP) was prepared by centrifugation of ACD-whole-blood at 2000 g for 20 minutes at 22° C.

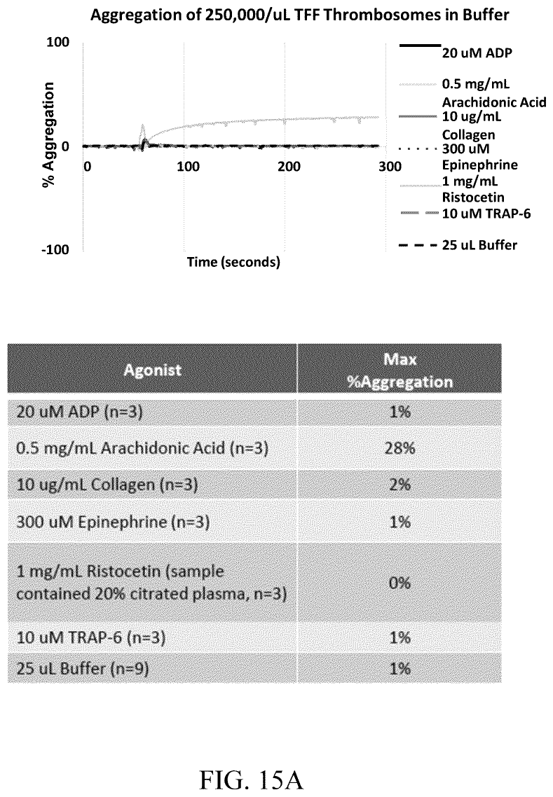

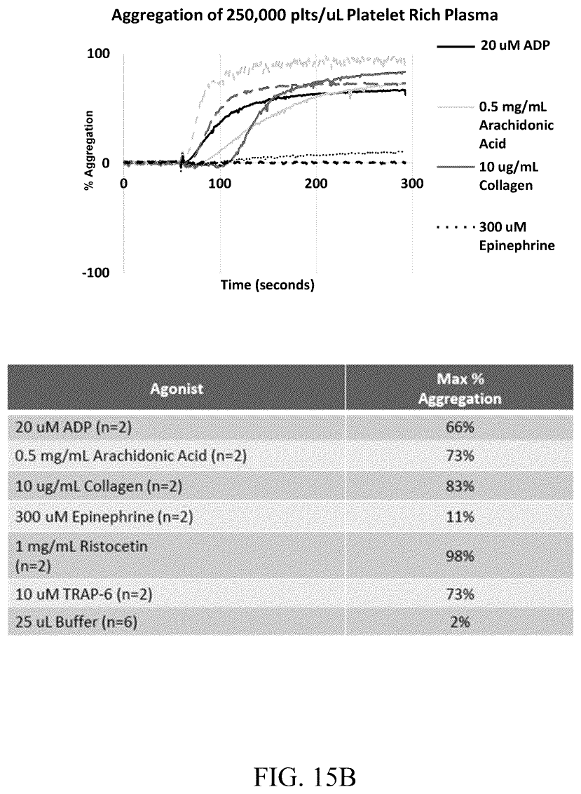

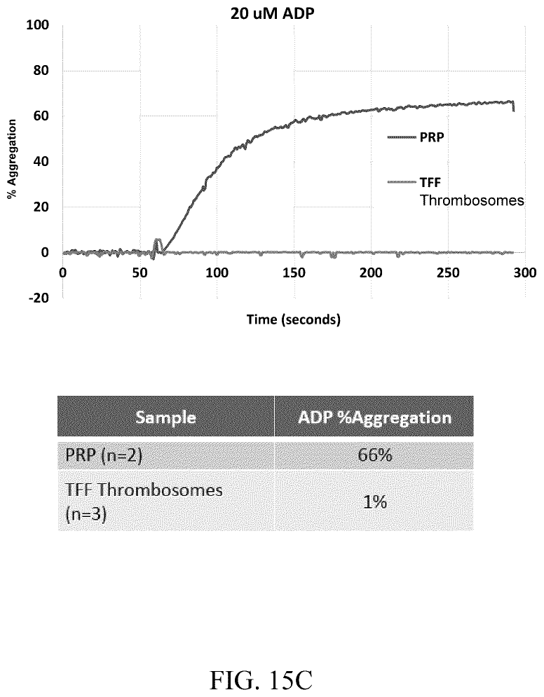

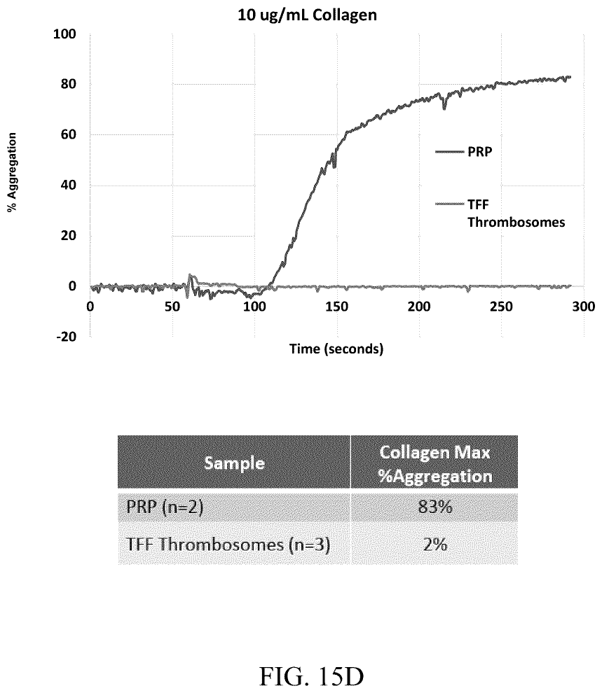

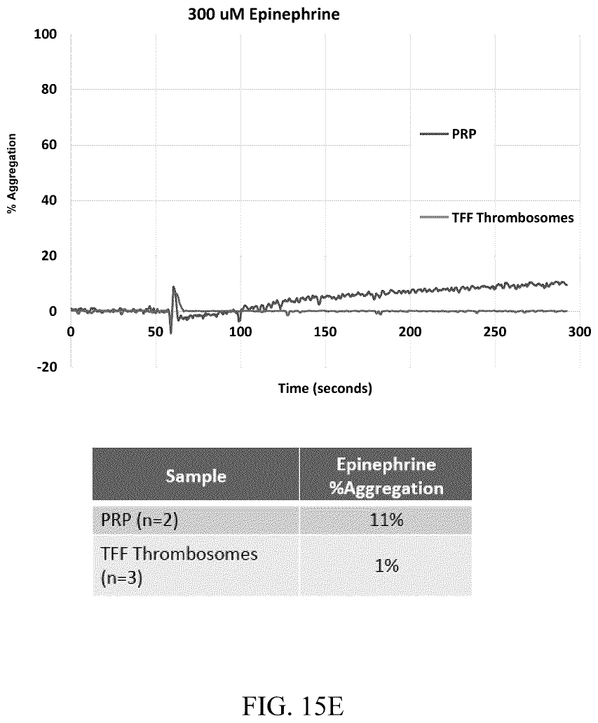

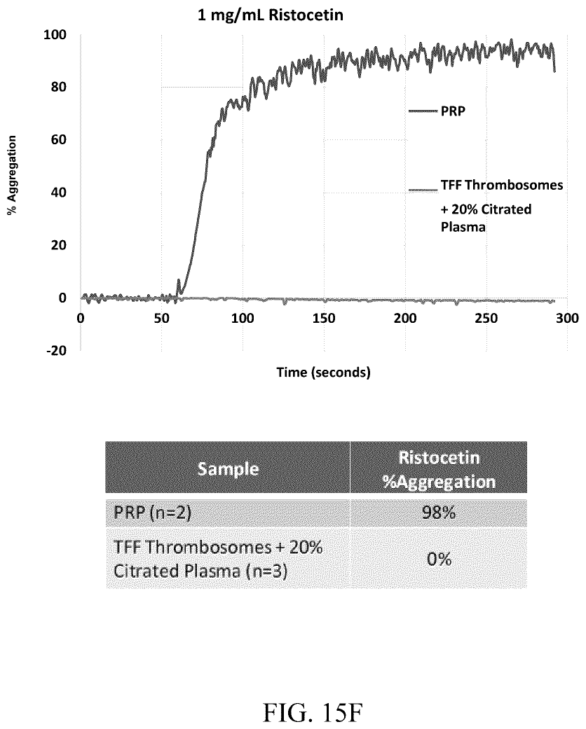

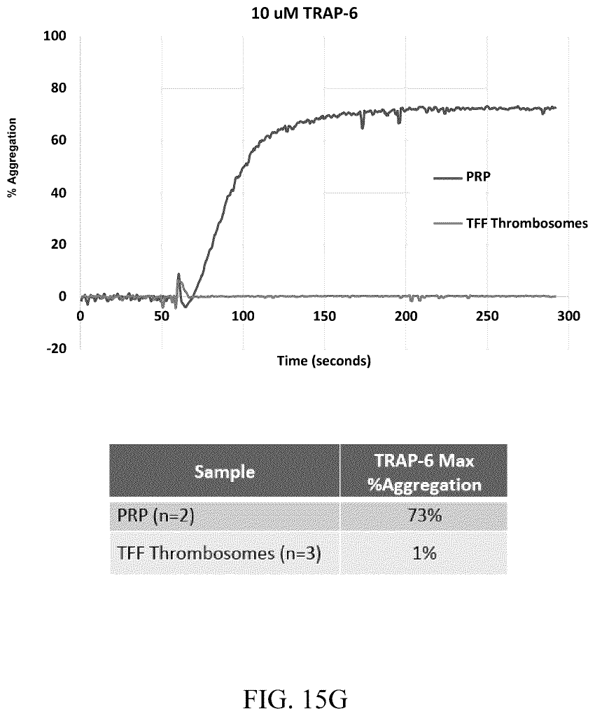

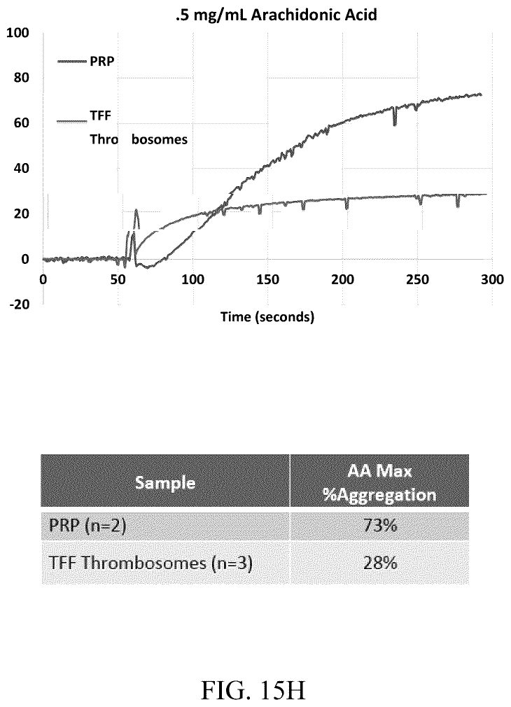

For sample preparation for aggregometry studies, PRP was diluted with PPP to a platelet concentration (plt count) of 250,000 plts/μL. Platelet count was determined using a Coulter Ac⋅T diff2 Hematology Analyzer. TFF FDPDs, lyophilized and thermally treated, were prepared using tangential flow filtration as described in Example 2. A 30 mL vial of FDPDs was rehydrated using 30 mL of cell culture grade water (Corning Cat #25-055-CI). The vial was incubated at room temperature for a total of 10 minutes. During the 10-minute rehydration period, the vial was gently swirled at 0, 5, and 10 minutes to promote dissolution of the lyophilizate. The aggregometry studies as per the present Example was carried out in the absence of fresh platelets. Therefore, the aggregometry studies supported only aggregation ability of the FDPDs, but not the co-aggregation ability. For sample preparation for aggregometry studies, rehydrated FDPDs were diluted in a buffer to a platelet count of 250,000/μL. FDPDs sample preparations used for ristocetin aggregation studies were composed of 20% citrated plasma (George King Bio-Medical, Inc. Pooled Normal Plasma product #0010-1) and buffer. Light transmission aggregometry (LTA) (Bio/Data PAP-8E Platelet Aggregometer catalog #106075) at 37° C. was used to observe the aggregation response of FDPDs (FIG. 15A) and PRP samples (FIG. 15B) from a final concentration of 20 μM ADP, 10 μg/mL collagen, 200 μM epinephrine (ADP, collagen, and epinephrine reagents from Helena Laboratories Platelet Aggregation Kit cat. #5369), 0.5 mg/mL arachidonic acid (Helena Arachidonic Acid Reagent cat.), lmg/mL ristocetin (Helena Ristocetin for Aggregation Assays cat.), and 10 μM thrombin receptor activator peptide 6 (TRAP-6) (Sigma Aldrich Cat #T1573-5MG). PPP, buffer, or buffer with 20% citrated plasma were used as blanks for the PRP, FDPDs, and FDPDs with 20% citrated plasma samples, respectively. Prior to agonist treatment, 225 μL of FDPDs or PRP sample was reverse pipetted in a test tube containing a stir bar. The test tube was then placed into the aggregometer's non-stirred incubation well for 1 minute. The sample was then placed into a stirred incubation well for 1 minute. The sample was then placed into the stirred test well and the aggregation test was initiated. After 1-minute of baseline observation the sample was treated with agonist and the aggregation response was recorded. Using the same procedure as the test runs, a negative control of 25 μL buffer was included simultaneously with all runs to determine spontaneous baseline-aggregation responses of all sample groups.

FDPD sample preparations in 1.7 mL microcentrifuge tubes, at room temperature, were treated with an agonist at a final agonist concentration of 20 μM ADP, 0.5 mg/mL arachidonic acid, 10 μg/mL collagen, 200 μM epinephrine, 1 mg/mL ristocetin, and 10 μM TRAP-6 or 25 μL buffer.

FDPD counts were determined prior to and 5-minutes after agonist treatment. ADP (FIG. 15C), collagen (FIG. 15D), epinephrine (FIG. 15E), ristocetin (FIG. 15F), and TRAP-6 (FIG. 15G) did not cause an aggregation response in TFF FDPDs when measured by LTA. TFF FDPDs' response from the aforementioned agonists was equivalent to baseline aggregation values that would be obtained from no agonist or a negative control of buffer. When TFF FDPDs were treated with arachidonic acid (AA) and observed by LTA (FIG. 15H) there was an apparent aggregation response, however after visual inspection of the aggregometry cuvette it was observed that the solution had become visibly clear and aggregates were not observed, indicating that the apparent aggregation response was from lysis of FDPDs and not AA induced aggregation. Determining aggregation by cell count for TFF FDPDs produced similar results to the LTA results for all agonists. Agonists' functionality was confirmed by performing LTA on fresh PRP (FIG. 15B). ADP, arachidonic acid, collagen, epinephrine, ristocetin and TRAP-6 caused normal aggregation profiles and magnitudes that are representative of a strong aggregation response in PRP. The aggregation response from epinephrine in PRP was reduced, however epinephrine was still able to elicit an aggregation response that was above baseline aggregation. The negative control of buffer in PRP indicated that the PRP was not activated prior to agonists additions. Visual inspection of the PRP samples after the aggregation tests indicated that no cell lysis had occurred and platelet aggregates were visually observed in the aggregation cuvettes for all agonists, indicating that all aggregations responses were from platelet aggregation. The aggregation percentage of FDPDs and fresh PRP observed in the presence of the afore-mentioned agonists have been captured in Table 8.

TABLE 8

| | TFF FDPDs | PRP % Aggregation |
|---|---|---|
| Agonist | % Aggregation (n = 3) | (n = 2) |
| 20 μM ADP | 1% | 66% |
| 0.5 mg/mL | 28%* | 73% |
| Arachidonic Acid | | |
| 10 μg/mL Collagen | 2% | 83% |
| 300 μM Epinephrine | 1% | 11% |
| 1 mg/mL Ristocetin | 0% | 98% |
| 10 μM TRAP-6 | 1% | 73% |
| 25 μL Buffer | 1% | 2% |

*Due to lysis of FDPDs and not aggregation

### Example 5. FDPDs are Maximally Activated—Binding of Annexin V to FDPDs in the Presence of TRAP

FDPDs, prepared using the TFF process and treated with TRAP-6, were tested for the presence of phosphatidylserine (PS), indicative of an activated platelet, on the surface of the FDPDs. The presence of PS was assessed by analysis of Annexin V (AV) binding to the FDPDs.

One 30 mL vial of FDPDs prepared using the TFF process as described in the Example 2 was rehydrated using 30 mL of cell culture grade water (Corning Cat #25-055-CI). After water was added to the vial, the vial was incubated for 10 minutes at room temperature. Gentle swirling of the vial was performed every 2 minutes during the 10-minute period to promote full dissolution of the cake. Once the FDPDs were fully rehydrated, two 475 μL aliquots were transferred to two separate 1.7 mL microcentrifuge tubes. Twenty-five microliters of HEPES Modified Tryode's Albumin buffer (HMTA) (Cellphire RGT-004) was added to the sample in the first tube to generate FDPDs without TRAP-6. Twenty-five microliters of 400 μM Thrombin Receptor Activating Peptide 6 (TRAP-6) (Sigma Aldrich Cat #T1573-5MG) was added to the second tube to generate FDPDs with TRAP-6. The final concentration of TRAP-6 during incubation was 20 μM. Both tubes were inverted 5 times to mix and incubated at room temperature for 10 minutes.

After incubation with HMTA buffer or TRAP-6, the samples were further diluted 1:20 by adding 10 μL of the FDPD sample to 190 μL HMTA. These diluted samples of FDPDs incubated with HMTA and FDPDs incubated with TRAP-6 were both stained in 1.7 mL microcentrifuge tubes as follows: unstained control samples were generated by combining 10 μL of FDPDs and 20 μL HMTA; calcium free control samples were generated by combining 10 μL of FDPDs, 5 μL of Annexin V—ACP (BD Pharmingen Cat #550475), and 15 μL HMTA; Annexin V (AV) stained test samples were generated by combining 10 μL of FDPDs, 5 μL of AV-ACP, and 15 μL HMTA supplemented with 9 mM CaCl₂) (Cellphire RGT-012 Lot #LAB-0047-21). The final concentration of CaCl₂) in the AV-stained test samples was 3 mM. All stained samples for both FDPDs incubated with HMTA and FDPDs incubated with TRAP-6 were generated in triplicate. The samples were incubated at room temperature, protected from light, for 20 minutes.

After incubation, 500 μL of HEPES buffered saline (HBS) (Cellphire RGT-017) was added to all unstained control and calcium free control samples. Five hundred microliters of HBS supplemented with 3 mM CaCl₂) was added to the AV-stained test samples. One hundred microliters from each sample was transferred to an individual well in a 96 well plate, and the samples were analyzed using an Agilent Quanteon flow cytometer.

TRAP-6 activity was confirmed by measuring CD62P expression in human apheresis platelets with and without exposure to TRAP-6. Two 475 μL aliquots of apheresis platelets were transferred to two separate 1.7 mL microcentrifuge tubes. Twenty-five microliters of HMTA buffer was added to the sample in the first tube to generate apheresis platelets without TRAP-6. Twenty-five microliters of 400 μM TRAP-6 was added to the second tube to generate FDPDs with TRAP-6. The final concentration of TRAP-6 during incubation was 20 μM. Both tubes were inverted 5 times to mix and incubated at room temperature for 10 minutes.

After incubation with HMTA buffer or TRAP-6, the samples were further diluted 1:20 by adding 10 μL of apheresis platelets to 190 μL HMTA. These diluted samples of apheresis platelets incubated with HMTA and apheresis platelets incubated with TRAP-6 were both stained in 1.7 mL microcentrifuge tubes as follows: unstained control samples were generated by combining 10 μL of apheresis platelets and 20 μL HMTA; Anti-CD62P stained test samples were generated by combining 10 μL of apheresis platelets, 5 μL of anti-CD62P-PE antibody (BD Pharmingen Cat #550561 Lot #6322976), and 15 μL HMTA. All stained samples for both apheresis platelets incubated with HMTA and apheresis platelets incubated with TRAP-6 were generated in triplicate. The samples were incubated at room temperature, protected from light, for 20 minutes.

After incubation, 500 μL of phosphate buffered saline (PBS) (Corning Cat #21-040-CV1) was added to all samples. One hundred microliters from each sample was transferred to an individual well in a 96 well plate, and the samples were analyzed using an Agilent Quanteon flow cytometer.

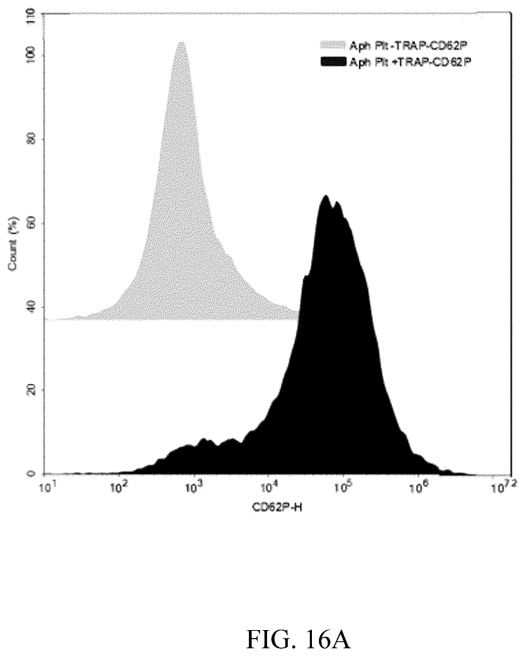

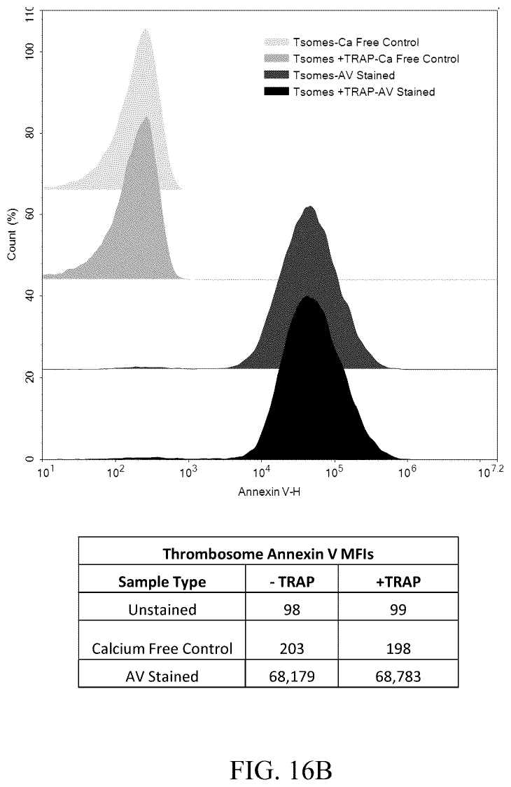

FDPDs manufactured using the TFF process were incubated with either TRAP-6 or buffer and stained with Annexin V (AV) to determine the relative presence of phosphatidylserine (PS). Apheresis platelets were used to confirm TRAP-6 activity (FIG. 16A, and Table 9), and increased expression of CD62P after the exposure to TRAP-6 confirms that TRAP-6 is capable of promoting platelet expression. PS expression on the exterior membrane leaflet is a hallmark of platelet activation and increases in membrane expression of PS result in greater amounts of AV binding. Unstained samples and samples stained with AV but without the addition of calcium were analyzed on the flow cytometer as negative controls. Unstained samples generated little to no fluorescent signal, indicating that FDPDs were not auto fluorescent at the wavelength selected to measure AV (FIG. 16B). The calcium free control samples also generated little to no fluorescent signal. Since AV binding to PS is dependent on the presence of calcium ions, a lack of signal from the calcium free control samples demonstrates that the AV-ACP conjugate was not associating with the FDPD membrane in a nonspecific manner. All samples stained with AV in the presence of calcium provided a strong fluorescent signal that was, on average, approximately 695 times brighter than the unstained controls. This result indicates that all FDPDs samples were expressing, or comprised, PS. Incubating the FDPDs with TRAP-6 did not cause a notable increase in AV binding as measured by mean fluorescent intensity (MFI) (FIG. 16B). The average MFI values for FDPDs incubated with buffer and FDPDs incubated with TRAP-6 were 68,179 and 68,783, respectively (Table 10).

TABLE 9
Apheresis Platelet CD62P MFI

| Sample Type | −TRAP | +TRAP |
|---|---|---|
| Unstained | 100 | 107 |
| CD62P Stained | 2,351 | 126,598 |

TABLE 10
FDPD Annexin V MFIs

| Sample Type | −TRAP | +TRAP |
|---|---|---|
| Unstained | 98 | 99 |
| Calcium Free Control | 203 | 198 |
| AV Stained | 68,179 | 68,783 |

FDPDs, manufactured using the TFF process, were shown to contain phosphatidylserine (PS) on the membrane as evident by the binding of Annexin V (AV) to the FDPDs. The binding of AV to activated platelets is a calcium dependent binding and therefore the calcium ion dependency of AV binding to the rehydrated FDPDs provides further support that the AV conjugate was not associating with the membrane of the FDPD in a nonspecific manner.

While TRAP-6 was shown to activate apheresis platelets, as evident by increased CD62P expression, and increased the binding of AV to the activated platelet, it was not the case for the FDPDs. The FDPDs with or without a TRAP-6 incubation exhibited same high level of AV binding, and indicate that TRAP-6 does not promote further surface expression of PS for FDPDs, likely because the FDPDs are maximally activated during the lyophilization and/or rehydration process, and further stimulation/activation is not possible.

### Example 6. Presence of Thrombospondin (TSP1) on the Surface of the FDPDs

Thrombospondin (TSP1), a glycoprotein typically found to coat external membranes of activated platelets, was found to coat FDPDs without activation. The presence of TSP1 was detected by fluorescence of anti-Thrombospondin-1 (TSP-1) antibody.

Fresh platelet rich plasma (PRP) was isolated by centrifuging whole blood collected in acid citrate dextrose (ACD) at 180 g for 10 minutes. Isolated PRP was centrifuged again at 823 g for an additional 10 minutes. The plasma was then removed and discarded, and the platelet pellet was resuspended in HEPES Modified Tyrode's Albumin (HMTA) buffer. An aliquot of the resulting washed platelet sample was activated by incubated the platelets at room temperature for 10 minutes in the presence of 2 mM GPRP peptide (BaChem Cat #H-1998.0025), 2 mM CaCl₂, 0.5 U/mL thrombin (EDM Millipore Cat #605190-1000U), and 0.5 μg/mL collagen (ChronoPar Cat #385). A separate aliquot of washed platelets was set aside to be used as a resting negative control.

All samples of FDPDs were manufactured using the TFF process as described in Example 2. The FDPDs studied in this example were baked FDPDs which were heated after lyophilization at 80° C. for 24 hours. All vials were rehydrated using the appropriate amount of cell culture grade water. After water was added, the vials were incubated for 10 minutes at room temperature. Gentle swirling of the vials was performed every 2 minutes during the 10-minute period to promote full dissolution of the cake. Once rehydrated, samples of FDPDs from each vial, along with samples from both the resting and activated fresh washed platelet aliquots, were diluted 1:500 in triplicate using phosphate buffered saline (PBS) (Corning Cat #21-040-CV). The diluted samples were analyzed on the Quanteon flow cytometer and the concentrations of the platelets and FDPDs were determined. Based on these concentrations, an aliquot of each FDPDs or fresh platelet sample was diluted to a concentration of 100,000 FDPDs per microliter.

Stained samples from each vial of FDPDs and the resting and activated fresh platelets were generated by adding 10 μL of diluted FDPDs or platelets to 20 μL of HMTA containing 4 g/mL of anti-Thrombospondin-1 (TSP-1) antibody (Santa Cruz Biotech Cat #sc-59887 AF594). Unstained control samples were generated by adding 10 μL of diluted FDPDs or platelets to 20 μL of HMTA. All The samples were incubated at room temperature, protected from light, for 20 minutes. After incubation, 500 μL of PBS was added to all samples. One hundred microliters from each sample were transferred to an individual well in a 96 well plate, and the samples were analyzed using an Agilent Quanteon flow cytometer.

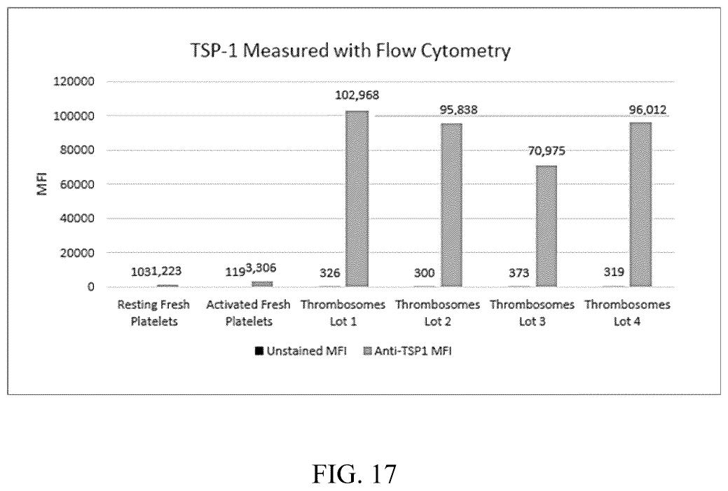

Unstained samples of fresh platelets and FDPDs generated little to no fluorescent signal, indicating that the samples were not auto fluorescent at the wavelength selected to measure TSP-1 expression or presence. Binding of the anti-TSP-1 antibody to fresh platelets increased slightly after activation with collagen and thrombin as shown by an increase in mean fluorescent intensity (MFI) when analyzed using flow cytometry (1,223 vs 3,306). Expression or presence of TSP-1 on FDPD samples varied from lot to lot with an average MFI value of 91,448 (FIG. 17). For all FDPD samples tested, the fluorescent signal was significantly higher than the signal generated by either resting or fresh platelets, indicating high amounts of TSP-1 may be bound to the surface of rehydrated FDPDs. This data suggests that the FDPDs without the requirement of an activation step exhibit properties which in certain embodiments and applications are superior to activated platelet properties.

### Example 7. Presence of Von Willebrand Factor (vWF) on the Surface of the FDPDs

Fresh platelet rich plasma (PRP) was isolated by centrifuging whole blood collected in acid citrate dextrose (ACD) at 180 g for 10 minutes. Isolated PRP was centrifuged again at 823 g for an additional 10 minutes. The plasma was then removed and discarded, and the platelet pellet was resuspended in HEPES Modified Tyrode's Albumin (HMTA) buffer. An aliquot of the resulting washed platelet sample was activated by incubating the platelets at room temperature for 10 minutes in the presence of 2 mM GPRP peptide (BaChem Cat #H-1998.0025), 2 mM CaCI₂), 0.5 U/mL thrombin (EDM Millipore Cat #605190-1000U), and 0.5 μg/mL collagen (ChronoPar Cat #385). A separate aliquot of washed platelets was set aside to be used as a resting negative control. All samples of FDPDs were prepared using the TFF process as described in the Example 1. The FDPDs studied in this example were baked FDPDs which were heated after lyophilization at 80° C. for 24 hours. All vials were rehydrated using the appropriate amount of cell culture grade water (Corning Cat #25-055-CI). After water was added, the vials were incubated for 10 minutes at room temperature. Gentle swirling of the vials was performed every 2 minutes during the 10-minute period to promote full dissolution of the cake. Once rehydrated, samples of FDPDs from each vial, along with samples from both the resting and activated fresh washed platelet aliquots, were diluted 1:500 in triplicate using phosphate buffered saline (PBS). The diluted samples were analyzed on the Quanteon flow cytometer and the concentrations were determined. Based on these concentrations, an aliquot of each FDPDs or fresh platelet sample was diluted to a concentration of 100,000 FDPDs per microliter.

Prior to staining, the anti-Von Willebrand Factor antibody (Novus Biologicals Cat #NBP2-54379PE) was diluted by a factor of 10. Stained samples from each vial of FDPDs and the resting and activated fresh platelets were generated by adding 10 μL of diluted FDPDs or platelets to 10 μL of diluted antibody and 10 μL of HMTA. Unstained control samples were generated by adding 10 μL of diluted FDPDs or platelets to 20 μL of HMTA. All The samples were incubated at room temperature, protected from light, for 20 minutes. After incubation, 500 μL of PBS was added to all samples. One hundred microliters from each sample was transferred to an individual well in a 96 well plate, and the samples were analyzed using an Agilent Quanteon flow cytometer.

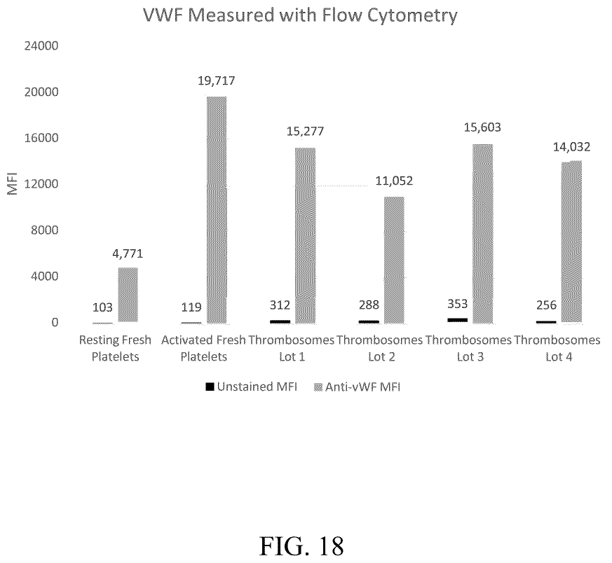

Unstained samples of fresh platelets and FDPDs generated little to no fluorescent signal, indicating that the samples were not auto fluorescent at the wavelength selected to measure vWF expression or presence. Binding of the anti-vWF antibody to fresh platelets increased after activation with collagen and thrombin as shown by an increase in mean fluorescent intensity (MFI) when analyzed using flow cytometry (4,771 vs 19,717). Expression or presence of vWF on FDPD samples varied from lot to lot with an average MFI value of 13,991 (FIG. 18). For all FDPD samples tested, the fluorescent signal fell between the signals generated by resting and activated platelets. This suggests that vWF is present on the surface of rehydrated FDPDs, and that the amount of vWF present is greater than that seen on resting platelets. The data suggests that even in the absence of any activation, the FDPDs exhibit properties that is superior to resting platelets and similar to the activated platelets.

### Example 8. Lyophilized Fixed Platelet and FDPDs Flow Cytometry

The presence of von Willebrand factor, Thrombospondin-1, and fibrinogen, which can be desired properties for some uses of platelet derivatives, were analyzed by respective antibody binding and the MFI (mean fluorescence intensity) assay for lyophilized fixed platelets and thrombosomes.

### Method

All samples of thrombosomes were prepared using the TFF process as described in Example 2. All vials of thrombosomes were rehydrated using the appropriate amount of cell culture grade water. After water was added, the vials were incubated for 10 minutes at room temperature. Gentle swirling of the vials was performed every 2 minutes during the 10-minute period to promote full dissolution of the cake. Fixed lyophilized platelets (Chrono-Log Corp Cat #299-9-) were rehydrated using Tris buffered saline (TBS) (Chrono-Log Corp Cat #299-5) according to the manufacturer's instruction. Once rehydrated, samples of thrombosomes from each vial, along with a sample of the lyophilized fixed platelets, were diluted 1:500 in triplicate using phosphate buffered saline (PBS) (Corning Cat #21-040-CV). The diluted samples were analyzed on the Quanteon flow cytometer and the concentrations were determined. Based on these concentrations, an aliquot of each thrombosomes or fresh platelet sample was diluted to a concentration of 100,000 thrombosomes per microliter. Prior to staining, the anti-VWF antibody was diluted 1:10 (part to whole) in HMTA and the anti-TSP-1 antibody was diluted 1:5 (part to whole) in HMTA. Single stained samples from each vial of thrombosomes or fixed lyophilized platelets were generated by combining 10⁶ total cells diluted in HEPES Modified Tyrode's Albumin (HMTA) buffer (Boston Bioproducts, Inc. Cat #C-9234C) with one of the following: 2 μL anti-CD42b antibody (Milli Mark Cat #FCMAB196P), 10 μL anti-von Willebrand Factor antibody (Novus Biologicals CAT #NBP2-54379PE), 3 μL anti-Thrombospondin-1 (TSP-1) antibody (Santa Cruz Biotech Cat #sc-59887 AF594), or 5 μL anti-fibrinogen antibody (BioCytex Cat #5009-F100T). All The samples were incubated at room temperature, protected from light, for 20 minutes. After incubation, 500 μL of PBS was added to all samples. One hundred microliters from each sample was transferred to an individual well in a 96 well plate, and the samples were analyzed using an Agilent Quanteon flow cytometer.

### Results

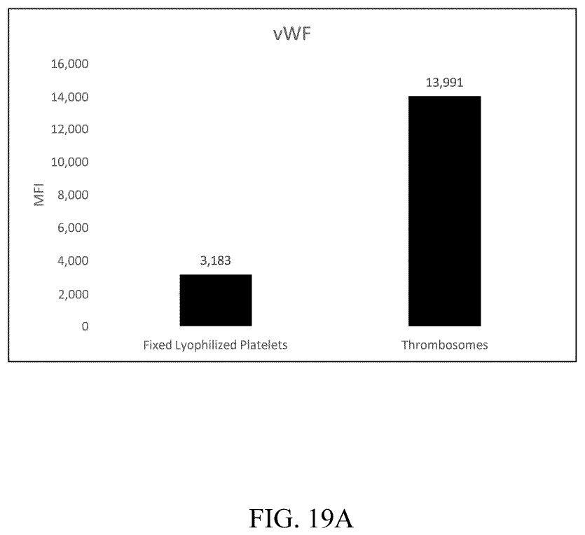

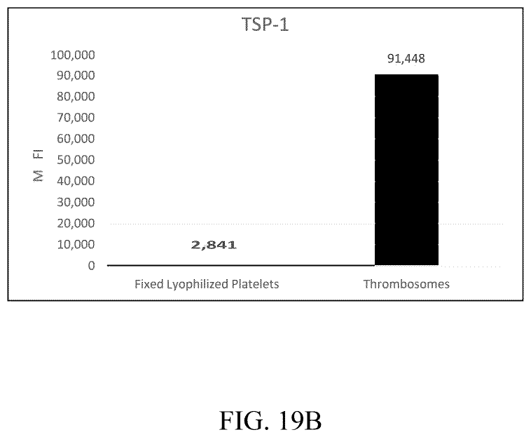

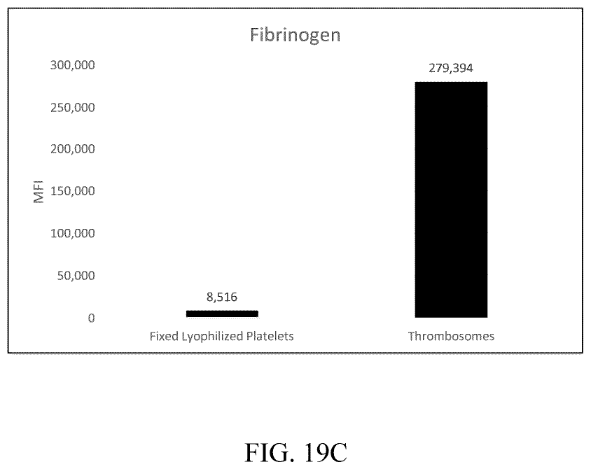

The resulting MFI values from these experiments are shown in FIGS. 19A-19C The data suggests that the addition of the fixation step appears to reduce the amount of pro-adhesive molecules that bind to the cell membrane during the lyophilization and/or rehydration process. Specifically, this experiment demonstrated the thrombosome surface contains greater quantities of von Willebrand factor, Thrombospondin-1, and fibrinogen as compared to that of the fixed human platelets.

### Example 9. FDPDs—Compromised Membrane

Membrane integrity of FDPDs, either heated at 80° C. for 24 hours (baked FDPDs) or not heated (unbaked FDPDs) after lyophilization, was tested. The baked and unbaked FDPDs of the standard formulation were analyzed by forward scatter against pre-lyophilization material and by the use of an antibody against a stable intracellular antigen, β-tubulin, to determine if FDPDs were permeable to IgGs (150 kDa). Forward scatter is a flow cytometry measurement of laser scatter along the path of the laser. Forward scatter (FSC) is commonly used as an indication of cell size as larger cells will produce more scattered light. However, forward scatter also can indicate the membrane integrity of the sample via optical density (i.e., light transmission); a cell with less cytosolic material and a porous membrane would transmit more light (have a lower FSC) than the same cell if intact, despite being the same size.

The FDPDs of Example 2 were studied to determine if FDPDs were permeable to IgGs (150 kDa) by the use of an antibody against a stable intracellular antigen, β-tubulin. Fresh platelets, unbaked FDPDs, and baked FDPDs were fixed and stained with anti-β tubulin IgG with and without cell permeabilization. Fresh platelets showed a dramatic increase in IgG binding with permeabilization, whereas both baked and unbaked FDPDs showed no change in response to permeabilization (Table 11). Results from fresh platelets and FDPDs that were fixed and then either permeabilized with 0.2% Triton-X 100 or not permeabilized and then stained with anti-β tubulin IgG conjugated to the fluorophore AF594. Unstained samples are included for background fluorescence.

TABLE 11

| Sample | Mean FSC-H | AF594 MFI |
|---|---|---|
| Platelets Unstained | 120,301 | 115 |
| Platelets | 118,782 | 636 |
| Permeabilized Platelets | 49,062 | 9,009 |
| Unbaked Thrombosomes | 23,140 | 75 |
| Unstained | | |
| Unbaked Thrombosomes | 23,280 | 546 |
| Permeabilized Unbaked | 7,069 | 562 |
| Thrombosomes | | |
| Baked Thrombosomes | 49,740 | 362 |
| Unstained | | |
| Baked Thrombosomes | 49,587 | 2,720 |
| Permeabilized Baked | 27,527 | 2,523 |
| Thrombosomes | | |

The IgG binding studies suggest that the membrane integrity of FDPDs is severely impaired such that large molecules can pass through the cell membrane. Of additional note, permeabilization induced decreases in forward scatter value, corroborating the proposed relationship between membrane integrity and optical density for particles of the same size.

Additionally, the mean intensity of forward light scattering of FDPDs prepared by TFF method as described in Example 2 was compared to in-date human platelet apheresis units. The method is as described below.

All samples of FDPDs were manufactured using the TFF process. All vials were rehydrated using the appropriate amount of cell culture grade water (Corning Cat #25-055-CI). After water was added, the vials were incubated for 10 minutes at room temperature. Gentle swirling of the vials was performed every 2 minutes during the 10-minute period to promote full dissolution of the cake. Once rehydrated, samples of FDPDs from each vial, along with samples from both in-date human platelet apheresis units, were diluted 1:500 in triplicate using phosphate buffered saline (PBS) (Corning Cat #21-040-CV). The diluted samples were acquired on the Quanteon flow cytometer and the concentrations were determined. Based on these concentrations, an aliquot of each FDPDs or apheresis platelet sample was diluted to a concentration of 100,000 FDPDs per microliter in HEPES Modified Tyrode's Albumin (HMTA) buffer (Cellphire RGT-004).

Unstained samples of FDPDs and human apheresis platelets containing 10⁶ total cells in HMTA were diluted with 500 μL of PB. One hundred microliters from each sample were transferred to an individual well in a 96 well plate, and the samples were analyzed using an Agilent Quanteon flow cytometer.

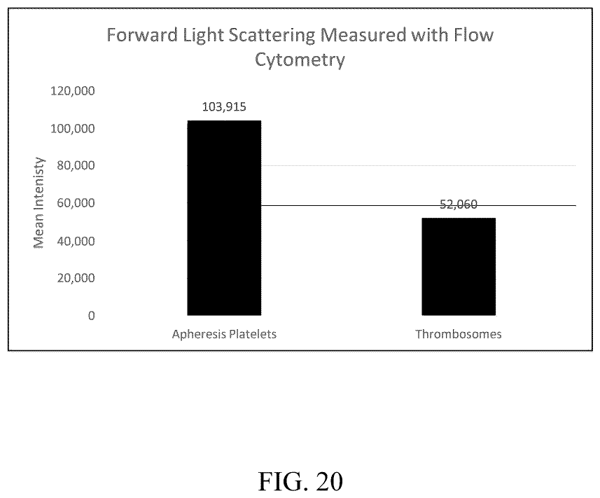

The mean intensity is depicted in FIG. 20. It can be observed that the mean intensity of forward light scattering measured with flow cytometry is distinctly lower (about 50%) for FDPDs as compared to the apheresis plasma. Therefore, corroborating with the previous result of Table 11 that a cell with less cytosolic material and a porous membrane would transmit more light (have a lower FSC) than the same cell if intact, despite being the same size.

The overall results suggest that membrane integrity is substantially degraded in FDPDs; the platelet intracellular contents have been released (e.g. LDH) and large molecules can enter the cellular cytosol (e.g. anti β-tubulin IgG). The plasma membrane of FDPDs is likely damaged by the drying (sublimation) or rehydration processes as freezing in cryopreserved platelets appears to be insufficient to induce severe membrane dysfunction. These results also imply that signal transduction from the outside of the cell is not possible in FDPDs, which is corroborated by lack of aggregation response (as observed in Example 4). Baking, although it produced an increase in optical density, did not appear to improve membrane integrity significantly (e.g., IgG β-tubulin binding). The results discussed in the present example thus show that the platelet derivatives as disclosed herein have a compromised plasma membrane.

### Example 10. Gadolinium Loaded Freeze Dried Platelet Derivatives and Gadolinium Loaded Cryopreserved Platelets

Gadolinium (MRI agent) was loaded onto platelets to create gadolinium loaded freeze dried platelet derivatives (FDPDs) and gadolinium loaded cryopreserved platelets. Two loading mechanism were performed: (1) Using a cell-penetrating peptide—TAT; and (2) covalently binding an MRI agent complex via surface protein conjugation via NSH ester (linker). The TAT peptide that was used herein had a sequence as represented by [K]LRKKRRQRRR (SEQ ID NO: 2).

Initial Reagents:
1. 3 Human Donor Apheresis Platelets
2. 10 mg DOTA (chelator) chelated gadolinium (MRI agent) TAT-peptide (CPP) with FITC label (FITC-TAT-DOTA-GD) obtained from Thermo Fisher (MRI agent coupled to a CPP)
3. 100 mg DOTA-NHS-ester obtained from Macrocyclics
4. Gadolinium (III) chloride hexahydrate obtained from Sigma Aldrich
5. Dimethyl Sulfoxide (DMSO)
6. Ethylenediaminetetraacetic acid (EDTA) obtained from Sigma Aldrich
7. Loading Buffer or buffer as shown in table below.

| | Concentration | |
|---|---|---|
| | (mM, except where | |
| Component | otherwise indicated) | |
| NaCl | 750 | |
| KCl | 48 | |
| HEPES | 95 | |
| NaHCO₃ | 120 | |
| Dextrose | 3 | |
| Trehalose | 0.1M | |
| Ethanol | 1.00% | (v/v) |
| Dansyl-EACA/EACA | 0, 50, or 100 | |
| (1:1000) | | |

8. 30% Polysucrose
9. NHS-Fluorescein obtained from Thermo Fisher
10. Fluorescein sodium salt obtained Sigma Aldrich

Reagent preparation was performed:

### 1. FITC-TAT-DOTA-GD

5 mg of FITC-TAT-DOTA-GD peptide was rehydrated with 2 ml water to create a 1 mM stock of FITC-TAT-DOTA-GD peptide in water. The excess was stored at −20 Celsius.

### 2. GD-DOTA-NHS-Ester

20 mg of DOTA-NHS-ester was rehydrated with 2.63 ml of DMSO to create a 10 mM stock of DOTA-NHS-ester in DMSO. 8.8 mg of gadolinium chloride was added to the DOTA-NHS-ester in DMSO to achieve a 9 mM concentration. The excess was stored at −20 Celsius.

### 3. NHS-Fluorescein

20 mg of NHS-Fluorescein was rehydrated with 4.22 ml of DMSO to create a 10 mM stock of NHS-Fluorescein in DMSO. The excess was stored at −20 Celsius.

### 4. GD-EDTA Complex

0.1 g of EDTA was rehydrated with 2.69 ml of water to create 0.1M stock of EDTA in water. The 0.1M stock of EDTA in water was then diluted in 1:10 in water. Next 10 mg of gadolinium chloride was added to achieve a 9 mM concentration. The excess was stored at −20 Celsius.

### 5. Fluorescein Solution

20 mg of fluorescein sodium salt was rehydrated with 10.64 ml of DMSO to create a 5 mM stock of fluorescein in DMSO. The excess was stored at −20 Celsius.

### Gadolinium Loaded Freeze Dried Platelet Derivatives and Gadolinium Loaded Cryopreserved Platelets Manufacturing.

Human Apheresis platelet units were pooled to equal volume yielding a total target volume of 300 ml.

The pooled platelets were acidified to a pH of 6.6-6.8 using 4 μL 1M Acid Citrate Dextrose solution per 1 mL pooled platelet rich plasma.

The acidified pooled platelet rich plasma was transferred to conical tubes to be centrifuged at 1500 g for 20 minutes.

The platelet pool plasma (PPP) was aspirated and discarded. The remaining solution was resuspended in a control buffer. The control buffer (CB) is 4-part loading buffer (LB) plus 1-part polysucrose (CB=0.8LB+6% polysucrose). The target count was 500×10\hat{ }3/πl.

The solution was divided into 6 groups (target 40-50 mL per group). Five groups were loaded with different combinations of MRI agent and/or fluorescent marker and one group was kept unloaded as a control.

MRI agent along with a chelator linked to cell penetrating peptide—FITC-TAT-DOTA-GD;

MRI agent complex for covalent bonding—GD-DOTA-NHS-ester;

Fluorescent reporter with a linker—NHS-Fluorescein;

MRI agent with a chelator—GD-EDTA Complex;

Fluorescent marker—Fluorescein solution.

1 mM of stock solution FITC-TAT-DOTA-GD was added to achieve a final concentration of 50 μM. Incubation was done at 37° C. for 30 minutes.

10 mM of stock solution GD-DOTA-NHS-ester was added to achieve a final concentration of 50 μM. Incubation was done at 37° C. for 30 minutes.

10 mM of stock solution NHS-Fluorescein was added to achieve a final concentration of 50 μM. Incubation was done at 37° C. for 30 minutes.

10 mM of stock solution GD-EDTA Complex was added to achieve a final concentration of 50 μM. Incubation was done at 37° C. for 30 minutes.

10 mM of stock solution Fluorescein solution was added to achieve a final concentration of 50 μM. Incubation was done at 37° C. for 30 minutes.

Unloaded control group was incubated at 37° C. for 30 minutes.

5 μl 1M citric acid per 1 ml platelet was added to each group and each group was then centrifuged at 1000 g for 10 minutes. The supernatant was aspirated and discarded. The six groups were resuspended in control buffer for a target count of 500×10\hat{ }3/πl. A final liquid formulated material (pre-lyo) platelet count was measured using Beckman CoulterAc⋅T diff2 Hematology Analyzer. Each of the six groups was split to be further processed into freeze-dried platelets derivatives and cryopreserved platelets.

For the cryopreserved group of 6 samples, 1% DMSO was added, 1 ml was aliquoted into 1.7 ml microcentrifuge tubes and frozen at −80° C. For the freeze-dried platelet derivative group of six samples 1 ml was aliquoted into 5 ml serum vials. Lyophilization was done using the below protocol. After lyophilization cycle was complete, all vials were stoppered and capped. The samples were then transferred to an 80° C. oven for baking (heat-treatment) for 24 hours+/−15 minutes.

### Protocol for Lyophilization (Milrock Lyophilizer)

Pre-Freezing: The condenser was turned on and the temperature was set to −40° C. before loading the lyophilizer. The lyophilizer was equilibrated in approximately 1-2 hours.

### Freezing:

Step 1—Shelf was ramped to −40° C. for 0 minutes.
Step 2—Shelf was held at −40° C. for 180 minutes.

### Final Freezing:

Shelf at −40° C. at 0 minutes; pressure was triggered at 100 m Torr.

### Primary Drying:

Step 1—Shelf was ramped to −10° C. for 360 minutes (pressure at 0 m Torr)
Step 2—Shelf was held at −10° C. for 360 minutes (pressure at 0 m Torr)
Step 3—Shelf was ramped to +5° C. for 180 minutes (pressure at 0 m Torr)
Step 4—Shelf was held at +5° C. for 360 minutes (pressure at 0 m Torr)
Step 5—Shelf was ramped to +30° C. for 300 minutes (pressure at 0 m Torr)

### Secondary Drying:

Step 1—Shelf was held at +30° C. for 720 minutes (pressure at 0 m Torr)
Step 2—Shelf was held at +30° C. for 720 minutes (pressure at 200 m Torr)
Step 3—Shelf was held at +30° C. for 9999 minutes (pressure at 0 m Torr)

The step 3 of secondary drying was held for a minimum of 1 hour.

### Sample Group Analysis

The six FDPDs sample groups and six cryopreserved sample groups were analyzed using Beckman Coulter Ac⋅T diff2 Hematology Analyzer, NovoCyte Quanteon Flow Cytometer, CLARIOstar Plus Microplate Reader, and T-TAS®01.

Preparation of FDPDs and Cryopreserved platelets for analysis.

The FDPD samples were rehydrated with sterile water equivalent to fill volume prior lyophilization. for analysis. For instance, vials were filled with 1 ml liquid product were rehydrated with 1 ml sterile water. The cryopreserved samples thawed in a 37° C. water bath for 8-10 minutes.

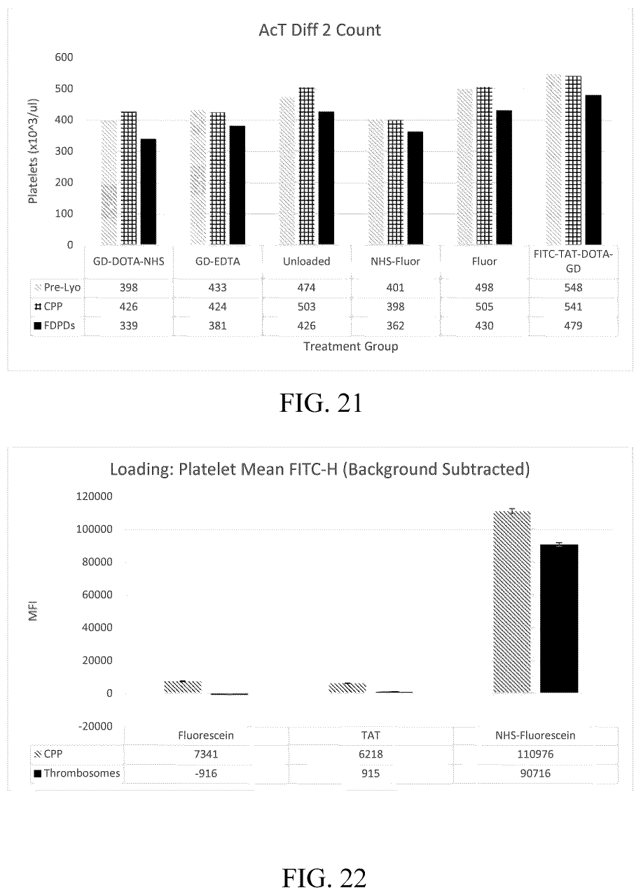

FIG. 21 is a comparison of the pre-lyo, CPP, and FDPD platelet count for the six sample group using Beckman Coulter Ac⋅T Diff2 Hematology Analyzer. FIG. 21 shows both cryopreserved and FDPDs gave a good recovery from the formulated liquid material (pre-lyo) to the reconstituted final product. There were only minor differences in the counts between the different treatment groups, suggesting that none of the treatment groups introduced significant cytotoxicity.

FIG. 22 are the flow cytometry measurements of mean fluorescence intensity (MFI) in the FITC channel for all six samples of FDPDs and CPP. Average values are presented with the unloaded product value subtracted out as background. Error bars are the standard deviation of measurements. NovoCyte Quanteon flow cytometer was used to take measurements. Loading of fluorescent markers (NHS-fluorescein, fluorescein, and TAT-FITC-DOTA-GD) was confirmed by flow cytometry (FIG. 22). As expected, the fluorescein without any loading agent gave very low fluorescence. The NHS-fluorescein that covalently bonds amine groups on proteins gave strong fluorescence; interestingly, the fluorescence was lower in cryopreserved platelets than in FDPDs. The fluorescence intensity from the TAT-FITC-DOTA-GD was low and decreased from cryopreservation to FDPDs.

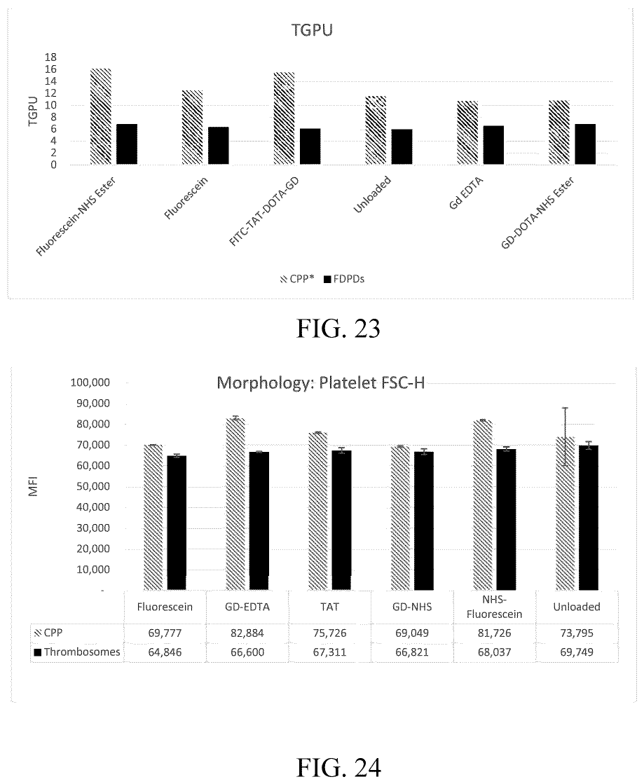

FIG. 23 are the flow cytometry measurements of forward scatter for all six samples of FDPDs and CPP. Average values are presented. Error bars are the standard deviation of measurements. NovoCyte Quanteon flow cytometer was used to take measurements. Forward scatter results did not indicate any significant defects in the cryopreserved platelet or FDPDs (FIG. 23)

FIG. 24 is a graph of the Thrombin generation results for all six samples of FDPDs and CPP using the CLARIOstar Plus microplate reader. Thrombin generation potency (TGPU) are equivalent to NIH Units of Thrombin per 1 million particles. Thrombin generation results confirmed that all products maintained hemostatic activity.

FIGS. 25A and 25B are the Total Thrombus System (T-TAS®) results for the six samples of cryopreserved platelet (FIG. 25A) and six samples of FDPDs (FIG. 25B). The measurements were taken at a platelet concentration of 80 k/μl in Octoplas® using a collagen and tissue factor coated AR chip. T-TAS® results confirmed that all products maintained hemostatic activity.

Although the steps involving processing of blood products, such as, pooled platelet rich plasma was done by centrifugation, the processing can also be done by TFF methods as disclosed in the instant invention that avoids the centrifugation steps.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A composition comprising MRI agent-loaded cryopreserved platelets, wherein the MRI agent-loaded cryopreserved platelets comprise an MRI agent complex covalently bonded to the surface of the cryopreserved platelets, wherein the MRI agent complex comprises an MRI agent, and a chelator.

2. The composition of claim 1, wherein the MRI agent-loaded cryopreserved platelets are capable of retaining at least 10% of the loaded MRI agent upon thawing.

3. A composition comprising MRI agent-loaded platelet derivatives, wherein the MRI agent-loaded platelet derivatives comprise an MRI agent complex covalently bonded to the surface of the platelet derivatives, wherein the MRI agent complex comprises an MRI agent, and a chelator, wherein the MRI agent-loaded platelet derivatives are surrounded by a compromised plasma membrane, and wherein at least 50% of the MRI agent-loaded platelet derivatives are CD 41-positive platelet derivatives.

4. The composition of claim 3, wherein the MRI agent-loaded platelet derivatives are capable of retaining at least 10% of the loaded MRI agent upon rehydrating.

5. A method for preparing a composition comprising MRI agent-loaded cryopreserved platelets or MRI agent-loaded platelet derivatives in a powder, comprising:
(a) providing platelets;
(b) contacting the platelets with an MRI agent complex comprising an MRI agent, a linker, and a chelator, to form MRI agent-loaded platelets; and
(c) cryopreserving or freeze-drying the MRI agent-loaded platelets to form the composition comprising the MRI agent-loaded cryopreserved platelets or the MRI agent-loaded platelet derivatives.

6. The method of claim 5, wherein the MRI agent is associated with the chelator, and the chelator is covalently linked to the surface of the platelets.

7. The method of claim 5, wherein the contacting the platelets is done in the presence of a buffer comprising a salt, a base, and a loading agent, at a temperature in the range of 15-45° C. for a time period in the range of 5 minutes to 48 hours, to form the MRI agent-loaded platelets.

8. The method of claim 5, wherein before contacting the platelets with the MRI agent complex, an MRI agent is contacted with a conjugate having a chelator conjugated to a linker, to form the MRI agent complex.

9. The method of claim 5, wherein the method further comprises performing tangential flow filtration (TFF) of a platelet composition with a preparation agent to prepare a TFF-treated composition comprising the platelets, before contacting the platelets with the MRI agent complex, and wherein contacting the TFF-treated composition comprising platelets with the MRI agent complex forms MRI agent-loaded TFF-treated composition.

10. The method of claim 9, further comprising freeze-drying the MRI agent-loaded TFF-treated composition comprising the platelets to form a composition comprising MRI agent-loaded, freeze-dried platelet derivatives.

11. A method for preparing a composition comprising MRI agent-loaded, freeze-dried platelet derivatives, the method comprising:
(a) performing tangential flow filtration (TFF) of a platelet composition with a preparation agent having a pH in the range of 5.5 to 8.0 and comprising 0.4 to 35% saccharides, wherein said TFF is performed using a 0.3 to 1 micron filter, thereby preparing a TFF-treated composition comprising platelets in an aqueous medium having less than or equal to 15% plasma protein, and having less than 5.0% microparticles having a diameter less than 0.5 μm by scattering intensity;
(b) contacting the TFF-treated composition with an MRI agent complex comprising an MRI agent, a linker, and a chelator, at a temperature in the range of 15-45° C. for a time period in the range of 5 minutes to 48 hours, to form an MRI agent-loaded, TFF treated platelet composition; and
(c) freeze drying the MRI agent-loaded, TFF-treated platelet composition to form the composition comprising MRI agent-loaded, freeze-dried platelet derivatives.

12. The method of claim 11, wherein the method further comprises after the freeze drying,
(d) heating the composition comprising MRI agent-loaded, freeze-dried platelet derivatives in a powder form at a temperature in the range of 60° C. to 90° C. for at least 1 hour to not more than 36 hours to thermally treat the MRI agent-loaded, freeze-dried platelet derivatives.

13. The method of claim 11, wherein the preparation agent comprises 0.4 to 35% trehalose and/or 2% to 8% polysucrose.

14. The method of claim 11, wherein the preparation agent comprises 0.4 to 35% trehalose and 2% to 8% polysucrose.

15. The method of claim 11, wherein the TFF-treated composition comprises 100×10¹ to 20,000×10³ platelets/μl when the TFF-treated composition is contacted with the MRI agent complex.

16. The method of any one of claims 5 to 12, wherein the MRI agent complex in step (b) has a concentration in the range of 5 to 500 μM.

17. The method of any one of claims 5 to 10, wherein the platelets have a concentration in the range of 1000 platelets/μl to 300×10⁶ platelets/μl when they are contacted with the MRI agent complex.

18. The method of any one of claims 5 to 8, wherein the cryopreserving is performed on the MRI agent-loaded platelets to form the composition comprising the MRI agent-loaded cryopreserved platelets.

19. The method of any one of claims 5 to 8, wherein the freeze-drying is performed on the MRI agent-loaded platelets to form the composition comprising the MRI agent-loaded platelet derivatives.

20. The method of any one of claims 5 to 15, further comprising thawing the MRI agent-loaded cryopreserved platelets to form thawed MRI agent-loaded platelets or rehydrating the MRI agent-loaded platelet derivatives in the powder to form rehydrated MRI agent-loaded platelet derivatives, wherein the thawed MRI agent-loaded platelets or the rehydrated MRI agent-loaded platelet derivatives, retain at least 10% of the loaded MRI agent upon thawing or upon rehydrating.

21. The method of claim 20, wherein the MRI agent-loaded cryopreserved platelets or the MRI agent-loaded platelet derivatives retain between 10% and 50% of the loaded MRI agent after the thawing or the rehydrating.

22. The composition of any one of claims 1 to 4 or the method of any one of claims 5 to 15, wherein the MRI agent-loaded platelet derivatives or the MRI agent-loaded cryopreserved platelets are capable of releasing the MRI agent upon stimulation by endogenous platelet activators.

23. The composition of any one of claims 3 or 4 or the method of any one of claims 10-15, or 19, wherein the MRI agent-loaded platelet derivatives are freeze-dried platelet derivatives.

24. The composition of any one of claims 1 to 4, or the method of any one of claims 5 to 15, wherein the MRI agent is associated with a surface of the cryopreserved platelets or the platelet derivatives.

25. The composition or the method of claim 24, wherein the MRI agent is associated with the external surface of the cryopreserved platelets or the platelet derivatives.

26. The composition or the method of claim 24, wherein the MRI agent is associated with the surface of the cryopreserved platelets or the surface of the platelet derivatives via the chelator.

27. The composition or the method of claim 24, wherein the chelator is covalently attached to the surface of the cryopreserved platelets or the surface of the platelet derivatives.

28. The method of any one of claims 5 to 15, wherein the linker is covalently bonded to the chelator in the MRI agent complex.

29. The composition of any one of claims 1 to 4, or the method of any one of claims 5 to 15, wherein the MRI agent is associated with the chelator through an ionic interaction.

30. The composition of any one of claims 1 to 4, or the method of any one of claims 5 to 15, wherein the MRI agent-loaded cryopreserved platelets or the MRI agent-loaded platelet derivatives do not comprise a cell penetrating peptide (CPP).

31. The composition of any one of claims 1 to 4, or the method of any one of claims 5 to 15, wherein the MRI agent-loaded cryopreserved platelets or the MRI agent-loaded platelet derivatives do not comprise a drug.

32. The composition of any one of claims 1 to 4, or the method of any one of claims 5 to 15, wherein the MRI agent-loaded cryopreserved platelets or MRI agent-loaded platelet derivatives further comprises a drug.

33. The composition of any one of claims 3 or 4, wherein the composition comprising MRI agent-loaded platelet derivatives is a dry powder.

34. The composition of any one of claims 1 to 4, or the method of any one of claims 5 to 15, wherein the MRI agent-loaded cryopreserved platelets or the MRI agent-loaded platelet derivatives are capable of releasing the MRI agent upon stimulation by endogenous platelet activators.

35. The composition of any one of claims 1 to 4, or the method of any one of claims 5 to 15, wherein the composition comprises a population of MRI agent-loaded platelet derivatives or MRI agent-loaded cryopreserved platelets are capable of occluding a collagen coated channel in a Total-Thrombus formation system (T-TAS).

36. The composition of any one of claims 1 to 4, or the method of any one of claims 5 to 15, wherein the MRI agent-loaded cryopreserved platelets, or the MRI agent-loaded platelet derivatives are capable of generating thrombin in an in vitro thrombin generation assay.

37. The composition of any one of claims 1 to 4, or the method of any one of claims 5 to 15, wherein the MRI agent-loaded cryopreserved platelets or the MRI agent-loaded platelet derivatives are capable of retaining between 10% and 50% of the loaded MRI agent upon thawing or rehydrating.

38. The composition of any one of claims 1 to 4, or the method of any one of claims 5 to 15, wherein the MRI agent-loaded platelets or the MRI agent-loaded platelet derivatives are capable of retaining at least 20% of the loaded MRI agent upon thawing or rehydrating.

39. The method of any one of claims 10 to 15, wherein the MRI agent-loaded platelet derivatives are surrounded by a compromised plasma membrane, and wherein at least 50% of the MRI agent-loaded platelet derivatives are CD 41-positive platelet derivatives.

40. The composition of any one of claims 3 to 4, wherein the composition when rehydrated comprises less than or equal to 15% plasma protein, and wherein the platelet derivatives have less than 5.0% microparticles having a diameter less than 0.5 μm by scattering intensity.

41. The composition of any one of claim 3 to 4, or the method of claim 39, wherein one or more of the following:
less than 5% of the CD 41-positive platelet derivatives are microparticles having a diameter of less than 0.5 μm,
at least 65% of the platelet derivates are CD 42 positive,
the MRI agent-loaded platelet derivatives are capable of generating thrombin in an in vitro thrombin generation assay,
the composition comprises a population of MRI agent-loaded platelet derivatives having a reduced propensity to aggregate such that no more than 10% of the platelet derivatives in the population aggregate under aggregation conditions comprising an agonist but no platelets, and no divalent cation,
the composition is capable of occluding a collagen coated channel in a Total-Thrombus formation system (T-TAS);
at least 50% of the MRI-loaded platelet derivatives in the composition are at least 0.5 μm in diameter by scattering intensity; and
at least 50% of the MRI-loaded platelet derivatives in the composition are between 0.5 μm and 25 μm in diameter by scattering intensity.

42. The composition of any one of claim 3 to 4, or the method of claim 39, wherein two or more of the following:
less than 5% of the CD 41-positive platelet derivatives are microparticles having a diameter of less than 0.5 μm,
at least 65% of the platelet derivates are CD 42 positive,
the MRI agent-loaded platelet derivatives are capable of generating thrombin in an in vitro thrombin generation assay, and
the composition comprises a population of MRI agent-loaded platelet derivatives having a reduced propensity to aggregate such that no more than 10% of the platelet derivatives in the population aggregate under aggregation conditions comprising an agonist but no platelets, and no divalent cation,
the composition is capable of occluding a collagen coated channel in a Total-Thrombus formation system (T-TAS).

43. The composition of any one of claim 3 to 4, or the method of claim 39, wherein:
less than 5% of the CD 41-positive platelet derivatives are microparticles having a diameter of less than 0.5 μm,
at least 65% of the platelet derivates are CD 42 positive,
the MRI agent-loaded platelet derivatives are capable of generating thrombin in an in vitro thrombin generation assay,
the composition comprises a population of MRI agent-loaded platelet derivatives having a reduced propensity to aggregate such that no more than 10% of the platelet derivatives in the population aggregate under aggregation conditions comprising an agonist but no platelets, and no divalent cation, and
the composition is capable of occluding a collagen coated channel in a Total-Thrombus formation system (T-TAS).

44. The composition of any one of claims 3 or 4, or the method of claim 39, wherein the composition comprises a population of MRI agent-loaded platelet derivatives having one or more characteristics of a super-activated platelet selected from
A) the presence of thrombospondin (TSP) on their surface at a level that is greater than on the surface of resting platelets;
B) the presence of von Willebrand factor (vWF) on their surface at a level that is greater than on the surface of resting platelets; and
C) an inability to increase expression of a platelet activation marker in the presence of an agonist as compared to the expression of the platelet activation marker in the absence of an agonist.

45. The composition of any one of claims 3 or 4, or the method of claim 39, wherein the composition comprises a population of MRI agent-loaded platelet derivatives having one or both of:
the presence of thrombospondin (TSP) on their surface at a level that is greater than on the surface of resting platelets; and
the presence of von Willebrand factor (vWF) on their surface at a level that is greater than on the surface of resting platelets.

46. The composition of any one of claims 1 to 4, or the method of any one of claims 5 to 15, wherein the MRI agent complex comprises an MRI agent selected from the group consisting of a paramagnetic metal ion, superparamagnetic metal ion, a diamagnetic metal ion, and combinations thereof.

47. The composition or the method of claim 46, wherein the superparamagnetic metal ion is selected from the group consisting of Gd(III), Fe(III), Mn(II and III), Cr(III), Cu(II), Dy(III), Tb(III and IV), Ho(III), Er(III), Pr(III) and Eu(II and III).

48. The composition of any one of claims 1 to 4, or the method of any one of claims 5 to 15, wherein the MRI agent complex comprises an MRI agent selected from the group consisting of metal ions with atomic numbers 21-29, 39-47, and 57-83.

49. The composition of any one of claims 1 to 4, or the method of any one of claims 5 to 15, wherein the MRI agent comprises Gadolinium (Gd).

50. The composition of any one of claims 1 to 4, or the method of any one of claims 5 to 15, wherein the chelator is selected from the group consisting of dodecane tetra acetic acid (DOTA), diethylenetriaminepentaacetic acid (DTPA), 4-Carboxy-5,8,11-tris(carboxymethyl)-1-phenyl-2-oxa-5,8,11-triazatridecan-13-oic acid (BOPTA), Ethylenediaminetetraacetic acid (EDTA), and 1,4,7,10-tetraazacyclododecane-1,4,7-tetracetic acid (DO3A).

51. The method of any one of claims 5 to 15, wherein the linker is selected from the group consisting of a compound having sulfhydryl reactive groups, such as maleimides and haloacetyl derivatives, amine reactive groups, such as isothiocyanates, succinimidyl esters, and sulfonyl halides, and carbodiimide reactive groups, such as carboxyl and amino groups.

52. The method of any one of claims 5 to 15, wherein the MRI agent complex comprises an MRI agent that comprises Gadolinium (Gd), a chelator comprising dodecane tetra acetic acid (DOTA), and a linker comprising an amine reactive group.

53. The method of claim 52, wherein the amine reactive group is a succinimidyl ester, and wherein the succinimidyl ester is N-Hydroxysuccinimide (NHS) ester or ester formed by NHS.

54. The method of claim 53, wherein the NHS forms an ester with DOTA.

55. A method of delivering an MRI agent to a subject, comprising administering an effective dose of the composition comprising MRI agent-loaded cryopreserved platelets or MRI agent-loaded platelet derivatives of any one of claims 1 to 4, or the composition prepared by the method of any one of claims 5 to 15 to the subject.

56. A method for detecting a site of inflamed, diseased or compromised blood vessels in a subject, comprising:
(a) administering an effective dose of the composition comprising MRI agent-loaded cryopreserved platelets or MRI agent-loaded platelet derivatives of any one of claims 1 to 4, or the composition prepared by the method of any one of claims 5 to 15 to the subject; and
(b) detecting the site of the MRI agent-loaded cryopreserved platelets or the MRI agent-loaded platelet derivatives, thereby detecting the site of inflamed, diseased or compromised blood vessels in the subject.

57. The composition of any one of claims 1 to 4, for use in the treatment of a subject having an indication selected from the group consisting of Acute lymphoblastic leukemia (ALL), Acute myeloid leukemia (AML), Breast cancer, Gastric cancer, Hodgkin lymphoma, Neuroblastoma, Non-Hodgkin lymphoma, Ovarian cancer, Cervical cancer, Small cell lung cancer, Non-small cell lung cancer (NSCLC), Soft tissue and bone sarcomas, Thyroid cancer, Transitional cell bladder cancer, Wilms tumor Neuroendocrine tumors, Pancreatic cancer, Multiple myeloma, Renal cancer, Glioblastoma Prostate cancer, Sarcoma, Colon cancer, Melanoma, Colitis, Chronic inflammatory demyelinating polyneuropathy, Guillain-Barre syndrome, Immune Thrombocytopenia, Kawasaki disease, Lupus, Multiple Sclerosis, Myasthenia gravis, Myositis, Cirrhosis with refractory ascites, Hepatorenal syndrome, Nephrotic syndrome, Organ transplantation, Paracentesis, Hypovolemia, Aneurysms, Artherosclerosis, Cancer, Cardiovascular diseases, Genetic disorders, Infectious diseases, Metabolic diseases, Neoangiogenesis, Opthalmic conditions, Hypercholesterolemia, and Pulmonary hypertension.

58. Use of the composition of any one of claims 1 to 4, or the composition obtained by the method of any one of claims 5 to 15, in the preparation of a medicament for treating a subject having an indication selected from the group consisting of Acute lymphoblastic leukemia (ALL), Acute myeloid leukemia (AML), Breast cancer, Gastric cancer, Hodgkin lymphoma, Neuroblastoma, Non-Hodgkin lymphoma, Ovarian cancer, Cervical cancer, Small cell lung cancer, Non-small cell lung cancer (NSCLC), Soft tissue and bone sarcomas, Thyroid cancer, Transitional cell bladder cancer, Wilms tumor Neuroendocrine tumors, Pancreatic cancer, Multiple myeloma, Renal cancer, Glioblastoma Prostate cancer, Sarcoma, Colon cancer, Melanoma, Colitis, Chronic inflammatory demyelinating polyneuropathy, Guillain-Barre syndrome, Immune Thrombocytopenia, Kawasaki disease, Lupus, Multiple Sclerosis, Myasthenia gravis, Myositis, Cirrhosis with refractory ascites, Hepatorenal syndrome, Nephrotic syndrome, Organ transplantation, Paracentesis, Hypovolemia, Aneurysms, Artherosclerosis, Cancer, Cardiovascular diseases, Genetic disorders, Infectious diseases, Metabolic diseases, Neoangiogenesis, Opthalmic conditions, Hypercholesterolemia, and Pulmonary hypertension.

59. The composition of any one of claims 1 to 4, for use in the treatment of a subject having an indication selected from the group consisting of Von Willebrand disease, Immune thrombocytopenia, Hermansky Pudlak Syndrome (HPS), Chemotherapy induced thrombocytopenia (CM, Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorriagia, Chronic myeloid leukemia, and any combinations thereof.

60. The method of claim 56, wherein the site of inflamed, diseased or compromised blood vessels is a site of bleeding.

61. The method of any one of claims 55 or 56, wherein the subject is afflicted with Acute lymphoblastic leukemia (ALL), Acute myeloid leukemia (AML), Breast cancer, Gastric cancer, Hodgkin lymphoma, Neuroblastoma, Non-Hodgkin lymphoma, Ovarian cancer, Cervical cancer, Small cell lung cancer, Non-small cell lung cancer (NSCLC), Soft tissue and bone sarcomas, Thyroid cancer, Transitional cell bladder cancer, Wilms tumor Neuroendocrine tumors, Pancreatic cancer, Multiple myeloma, Renal cancer, Glioblastoma Prostate cancer, Sarcoma, Colon cancer, Melanoma, Colitis, Chronic inflammatory demyelinating polyneuropathy, Guillain-Barre syndrome, Immune Thrombocytopenia, Kawasaki disease, Lupus, Multiple Sclerosis, Myasthenia gravis, Myositis, Cirrhosis with refractory ascites, Hepatorenal syndrome, Nephrotic syndrome, Organ transplantation, Paracentesis, Hypovolemia, Aneurysms, Artherosclerosis, Cancer, Cardiovascular diseases, Genetic disorders, Infectious diseases, Metabolic diseases, Neoangiogenesis, Opthalmic conditions, Hypercholesterolemia, and Pulmonary hypertension.

62. The method of any one of claims 55 or 56, wherein the subject is afflicted with Von Willebrand disease, Immune thrombocytopenia, Hermansky Pudlak Syndrome (HPS), Chemotherapy induced thrombocytopenia (CM, Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof.

63. The method of any one of claims 55 or 56, wherein the effective dose of the MRI agent-loaded platelet derivatives or the MRI agent-loaded cryopreserved platelets is in the range of 1.0×10⁷ to 1.0×10¹⁴/kg of the subject.

64. The method of any one of claims 55 or 56, wherein the effective dose of the MRI agent-loaded platelet derivatives or the MRI agent-loaded cryopreserved platelets is in the range of 1.0×10⁷ to 1.0×10¹³/kg of the subject.

65. The method of any one of claims 55 or 56, wherein the effective dose of the MRI agent-loaded platelet derivatives or the MRI agent-loaded cryopreserved platelets is in the range of 1.6×10⁷ to 1.0×10¹²/kg of the subject.

66. The method of any one of claims 55 or 56, wherein the effective dose of the MRI agent-loaded platelet derivatives or the MRI agent-loaded cryopreserved platelets is in the range of 5.0×10¹² to 1.0×10¹⁴/kg of the subject.

67. The method, the composition, or the use of any one of claims 55 or 56, wherein the subject is a mammal.

68. The method, the composition, or the use of claim 67, wherein the mammal is a human.

69. The method of claim 55 or 56, wherein the subject is afflicted with Alzheimer's disease.


SEQUENCE LISTING (87 sequences)

SEQ ID NO: 1 - protein, 86 aa, Human immunodeficiency virus type 1
  source 1..86: note = Human immunodeficiency virus type 1 TAT protein Full length
MEPVDPRLEPWKHPGSQPKTACTTCYCKKCCFHCQVCFTTKALGISYGRKKRRQRRRPPQGSQTHQVSLSKQPTSQPRGDPTGPKE

SEQ ID NO: 2 - protein, 11 aa, synthetic construct
  REGION 1..11: note = TAT Functional Fragment
KLRKKRRQRRR

SEQ ID NO: 3 - protein, 12 aa, synthetic construct
  REGION 1..12: note = TAT Functional Fragment
GRKKRRQRRRPQ

SEQ ID NO: 4 - protein, 14 aa, synthetic construct
  REGION 1..14: note = Tat47-60 Functional Fragment
YGRKKRRQRRRPPQ

SEQ ID NO: 5 - protein, 11 aa, synthetic construct
  REGION 1..11: note = Tat47-57 Functional Fragment
YGRKKRRQRRR

SEQ ID NO: 6 - protein, 9 aa, synthetic construct
  REGION 1..9: note = Tat49-57 Functional Fragment
RKKRRQRRR

SEQ ID NO: 7 - protein, 13 aa, synthetic construct
  REGION 1..13: note = Tat48-60 Functional Fragment
GRKKRRQRRRPPQ

SEQ ID NO: 8 - protein, 21 aa, synthetic construct
  REGION 1..21: note = Pep-1
KETWWETWWTEWSQPKKKRKV

SEQ ID NO: 9 - protein, 15 aa, synthetic construct
  REGION 1..15: note = Pep-7
SDLWEMMMVSLACQY

SEQ ID NO: 10 - protein, 16 aa, synthetic construct
  REGION 1..16: note = Penetratin
RQIKIWFQNRRMKWKK

SEQ ID NO: 11 - protein, 7 aa, synthetic construct
  REGION 1..7: note = Short Penetratin
RRMKWKK

SEQ ID NO: 12 - protein, 24 aa, synthetic construct
  REGION 1..24: note = Calcitonin derived CPP
LGTYTQDFNKFHTFPQTAIGVGAP

SEQ ID NO: 13 - protein, 10 aa, synthetic construct
  REGION 1..10: note = nuclear localization sequence
VQRKRQKLMP

SEQ ID NO: 14 - protein, 8 aa, synthetic construct
  REGION 1..8: note = nuclear localization sequence
SKKKKTKV

SEQ ID NO: 15 - protein, 8 aa, synthetic construct
  REGION 1..8: note = nuclear localization sequence
GRKRKKRT

SEQ ID NO: 16 - protein, 9 aa, synthetic construct
  REGION 1..9: note = new polybasic CPP
RRRERRAEK

SEQ ID NO: 17 - protein, 9 aa, synthetic construct
  REGION 1..9: note = new polybasic CPP
KCPSRRPKR

SEQ ID NO: 18 - protein, 6 aa, synthetic construct
  REGION 1..6: note = N-terminal repetitive domain of maize gamma-zein
VRLPPP

SEQ ID NO: 19 - protein, 6 aa, synthetic construct
  REGION 1..6: note = N-terminal repetitive domain of maize gamma-zein
VHLPPP

SEQ ID NO: 20 - protein, 6 aa, synthetic construct
  REGION 1..6: note = N-terminal repetitive domain of maize gamma-zein
VKLPPP

SEQ ID NO: 21 - protein, 17 aa, synthetic construct
  REGION 1..17: note = peptides from gp41 fusion sequence
AVGAIGALFLGFLGAAG

SEQ ID NO: 22 - protein, 12 aa, synthetic construct
  REGION 1..12: note = preS2-TLM
PLSSIFSRIGDP

SEQ ID NO: 23 - protein, 16 aa, synthetic construct
  REGION 1..16: note = signal-sequence hydrophobic region (SSHR)
AAVALLPAVLLALLAP

SEQ ID NO: 24 - protein, 15 aa, synthetic construct
  REGION 1..15: note = signal-sequence hydrophobic region (SSHR)
VTVLALGALAGVGVG

SEQ ID NO: 25 - protein, 18 aa, synthetic construct
  REGION 1..18: note = pVEC
IAARIKLRSRQHIKLRHL

SEQ ID NO: 26 - protein, 27 aa, synthetic construct
  REGION 1..27: note = Vpr
DTWPGVEALIRILQQLLFIHFRIGCQH

SEQ ID NO: 27 - protein, 34 aa, synthetic construct
  REGION 1..34: note = VP22
DAATATRGRSAASRPTERPRAPARSASRPRRPVD

SEQ ID NO: 28 - protein, 17 aa, synthetic construct
  REGION 1..17: note = gp41 fusion sequence
GALFLGWLGAAGSTMGA

SEQ ID NO: 29 - protein, 17 aa, synthetic construct
  REGION 1..17: note = Caiman crocodylus Ig(v) light chain
MGLGLHLLVLAAALQGA

SEQ ID NO: 30 - protein, 12 aa, synthetic construct
  REGION 1..12: note = Kaposi FGF signal sequences
AAVLLPVLLAAP

SEQ ID NO: 31 - protein, 27 aa, synthetic construct
  REGION 1..27: note = CPP from pestivirus envelope glycoprotein
RQGAARVTSWLGRQLRIAGKRLEGRSK

SEQ ID NO: 32 - protein, 28 aa, synthetic construct
  REGION 1..28: note = CPP derived from the prion protein
MANLGYWLLALFVTMWTDVGLCKKRPKP

SEQ ID NO: 33 - protein, 16 aa, synthetic construct
  REGION 1..16: note = Yeast PRP6 (129-144)
TRRNKRNRIQEQLNRK

SEQ ID NO: 34 - protein, 18 aa, synthetic construct
  REGION 1..18: note = Phi21 N (12-29)
TAKTRYKARRAELIAERR

SEQ ID NO: 35 - protein, 22 aa, synthetic construct
  REGION 1..22: note = Delta N (1-22)
MDAQTRRRERRAEKQAQWKAAN

SEQ ID NO: 36 - protein, 15 aa, synthetic construct
  REGION 1..15: note = FHV coat (35-49)
RRRRNRTRRNRRRVR

SEQ ID NO: 37 - protein, 19 aa, synthetic construct
  REGION 1..19: note = BMV Gag (7-25)
KMTRAQRRAAARRNRWTAR

SEQ ID NO: 38 - protein, 13 aa, synthetic construct
  REGION 1..13: note = HTLV-II Rex (4-16)
TRRQRTRRARRNR

SEQ ID NO: 39 - protein, 12 aa, synthetic construct
  REGION 1..12: note = HIV-1 Rev (9 to 20)
RQARRNRRRRWR

SEQ ID NO: 40 - protein, 22 aa, synthetic construct
  REGION 1..22: note = RSG-1.2
DRRRRGSRPSGAERRRRRAAAA

SEQ ID NO: 41 - protein, 15 aa, synthetic construct
  REGION 1..15: note = Lambda-N (48 to 62)
QTRRRERRAEKQAQW

SEQ ID NO: 42 - protein, 7 aa, synthetic construct
  REGION 1..7: note = SV40 NLS
PKKKRKV

SEQ ID NO: 43 - protein, 16 aa, synthetic construct
  REGION 1..16: note = Bipartite, Nucleoplasmin (155 to 170), NLS
KRPAAIKKAGQAKKKK

SEQ ID NO: 44 - protein, 12 aa, synthetic construct
  REGION 1..12: note = Herpesvirus, 8 k8 protein (124 to 135)
TRRSKRRSHRKF

SEQ ID NO: 45 - protein, 17 aa, synthetic construct
  REGION 1..17: note = Buforin-II (20 to 36)
RAGLQFPVGRVHRLLRK

SEQ ID NO: 46 - protein, 21 aa, synthetic construct
  REGION 1..21: note = Magainin
TRSSRAGLQFPVGRVHRLLRK

SEQ ID NO: 47 - protein, 16 aa, synthetic construct
  REGION 1..16: note = PDX-1-PTD
RHIKIWFQNRRMKWKK

SEQ ID NO: 48 - protein, 42 aa, synthetic construct
  REGION 1..42: note = crotamine
YKQCHKKGGHCFPKEKICLPPSSDFGKMDCRWRWKCCKKGSG

SEQ ID NO: 49 - protein, 16 aa, synthetic construct
  REGION 1..16: note = pIsl
RVIRVWFQNKRCKDKK

SEQ ID NO: 50 - protein, 18 aa, synthetic construct
  REGION 1..18: note = SynB1
RGGRLSYSRRRFSTSTGR

SEQ ID NO: 51 - protein, 60 aa, synthetic construct
  REGION 1..60: note = Fushi-tarazu (254-313)
SKRTRQTYTRYQTLELEKEFHFNRYITRRRRIDIANALSLSERQIKIWFQNRRMKSKKDR

SEQ ID NO: 52 - protein, 60 aa, synthetic construct
  REGION 1..60: note = Engrailed (454-513)
EKRPRTAFSSEQLARLKREFNENRYLTERRRQQLSSELGLNEAQIKIWFQNKRAKIKKST

SEQ ID NO: 53 - protein, 27 aa, synthetic construct
  REGION 1..27: note = transportan
GWTLNSAGYLLGKINLKALAALAKKIL

SEQ ID NO: 54 - protein, 8 aa, synthetic construct
  REGION 1..8: note = polyarginine CPP
RRRRRRRR

SEQ ID NO: 55 - protein, 9 aa, synthetic construct
  REGION 1..9: note = poly-d-arginine
RRRRRRRRR

SEQ ID NO: 56 - protein, 18 aa, synthetic construct
  REGION 1..18: note = KLAL peptide/model amphipathic peptide (MAP)
KLALKLALKALKAALKLA

SEQ ID NO: 57 - protein, 30 aa, synthetic construct
  REGION 1..30: note = KALA model amphipathic peptide
WEAKLAKALAKALAKHLAKALAKALKACEA

SEQ ID NO: 58 - protein, 11 aa, synthetic construct
  REGION 1..11: note = modeled Tat peptide
YARAAARQARA

SEQ ID NO: 59 - protein, 136 aa, synthetic construct
  REGION 1..136: note = Loligomer
TPPKKKRKVEDPKKKKKTPPKKKRKVEDPKKKKKTPPKKKRKVEDPKKKKKTPPKKKRKVEDPKKKKKTPPKKKRKVEDPKKKKKTPPKKKRKVEDPKKKKKTPPKKKRKVEDPKKKKKTPPKKKRKVEDPKKKKK

SEQ ID NO: 60 - protein, 27 aa, synthetic construct
  REGION 1..27: note = b-sheet-forming peptide
DPKGDPPKGVTVTVTVTVTGKGDPKPD

SEQ ID NO: 61 - protein, 16 aa, synthetic construct
  REGION 1..16: note = retro inverso forms of established CPPs
KKWKMRRNQFWVRVQR

SEQ ID NO: 62 - protein, 17 aa, synthetic construct
  REGION 1..17: note = W/R penetratin
RRWRRWWWRRWWRRWRR

SEQ ID NO: 63 - protein, 28 aa, synthetic construct
  REGION 1..28: note = MPG
GALFLGFLGAAGSTMGAWSQPKSKRKVC

SEQ ID NO: 64 - protein, 27 aa, synthetic construct
  REGION 1..27: note = Signal-sequence-based peptides (I)
GALFLGWLGAAGSTMGAWSQPKKKRKV

SEQ ID NO: 65 - protein, 12 aa, synthetic construct
  REGION 1..12: note = PTD-5
RRQRRTSKLMKR

SEQ ID NO: 66 - protein, 10 aa, synthetic construct
  REGION 1..10: note = RSV-A9
RRIPNRRPRR

SEQ ID NO: 67 - protein, 11 aa, synthetic construct
  REGION 1..11: note = CTP-512
YGRRARRRRRR

SEQ ID NO: 68 - protein, 12 aa, synthetic construct
  REGION 1..12: note = U2AF
SQMTRQARRLYV

SEQ ID NO: 69 - protein, 23 aa, synthetic construct
  REGION 1..23: note = Type of CPP
WLRRIKAWLRRIKALNRQLGVAA

SEQ ID NO: 70 - protein, 20 aa, synthetic construct
  REGION 1..20: note = Type of CPP
VKRKKKPALWKTLLKKVLKA

SEQ ID NO: 71 - protein, 16 aa, synthetic construct
  REGION 1..16: note = Type of CPP
KTVLLRKLLKLLVRKI

SEQ ID NO: 72 - protein, 19 aa, synthetic construct
  REGION 1..19: note = Type of CPP
KKKKKKKKKKKKKKKKKKK

SEQ ID NO: 73 - protein, 16 aa, synthetic construct
  REGION 1..16: note = Type of CPP
KLALKLALKALKAALK

SEQ ID NO: 74 - protein, 22 aa, synthetic construct
  REGION 1..22: note = Type of CPP
RQARRNRRRALWKTLLKKVLKA

SEQ ID NO: 75 - protein, 16 aa, synthetic construct
  REGION 1..16: note = Type of CPP
LLKKRKVVRLIKFLLK

SEQ ID NO: 76 - protein, 23 aa, synthetic construct
  REGION 1..23: note = Type of CPP
LIRLWSHLIHIWFQNRRLKWKKK

SEQ ID NO: 77 - protein, 24 aa, synthetic construct
  REGION 1..24: note = Type of CPP
LNSAGYLLGKINLKALAALAKKIL

SEQ ID NO: 78 - protein, 20 aa, synthetic construct
  REGION 1..20: note = Type of CPP
CWKKKKKKKKKKKKKKKKKK

SEQ ID NO: 79 - protein, 16 aa, synthetic construct
  REGION 1..16: note = Type of CPP
YTAIAWVKAFIRKLRK

SEQ ID NO: 80 - protein, 20 aa, synthetic construct
  REGION 1..20: note = Type of CPP
PKKKRKVALWKTLLKKVLKA

SEQ ID NO: 81 - protein, 20 aa, synthetic construct
  REGION 1..20: note = Type of CPP
GLWRALWRALRSLWKLKRKV

SEQ ID NO: 82 - protein, 20 aa, synthetic construct
  REGION 1..20: note = Type of CPP
GLWRALWRGLRSLWKKKRKV

SEQ ID NO: 83 - protein, 20 aa, synthetic construct
  REGION 1..20: note = Type of CPP
GLWRALWRGLRSLWKLKRKV

SEQ ID NO: 84 - protein, 18 aa, synthetic construct
  REGION 1..18: note = Type of CPP
KALAKALAKLWKALAKAA

SEQ ID NO: 85 - protein, 18 aa, synthetic construct
  REGION 1..18: note = Type of CPP
KLAAALLKKWKKLAAALL

SEQ ID NO: 86 - protein, 20 aa, synthetic construct
  REGION 1..20: note = Type of CPP
GLFKALLKLLKSLWKLLLKA

SEQ ID NO: 87 - protein, 18 aa, synthetic construct
  REGION 1..18: note = Type of CPP
KLALKLALKAWKAALKLA